SEQUENCE LISTING (492 sequences)

SEQ ID NO: 1 - DNA, 33 bp, Homo sapiens
taactgcacttaattcyatgcaggtaggaaact

SEQ ID NO: 2 - DNA, 33 bp, Homo sapiens
acaccagtcaattatcwcttttgtcattgccat

SEQ ID NO: 3 - DNA, 33 bp, Homo sapiens
cctaactggtgtgggamtagcacgtctgaattc

SEQ ID NO: 4 - DNA, 33 bp, Homo sapiens
ggcctgtctccaagtayggtcaccttctgagtt

SEQ ID NO: 5 - DNA, 33 bp, Homo sapiens
cctgaaattgaatggasagcctgagtgctacaa

SEQ ID NO: 6 - DNA, 33 bp, Homo sapiens
tccaccaaaatcataarctcacttccaatgaag

SEQ ID NO: 7 - DNA, 33 bp, Homo sapiens
ctactcttcacaaacartcctgagaagctggat

SEQ ID NO: 8 - DNA, 33 bp, Homo sapiens
aaagcaaaatacgacaraatagaacacagaaaa

SEQ ID NO: 9 - DNA, 33 bp, Homo sapiens
ctaagggatttatgtayagattgctaatgccgt

SEQ ID NO: 10 - DNA, 33 bp, Homo sapiens
atttataaattaaatckcatactgccaataaaa

SEQ ID NO: 11 - DNA, 33 bp, Homo sapiens
aaaaactggatctctaygtcatcctttgtccct

SEQ ID NO: 12 - DNA, 33 bp, Homo sapiens
gggcctgtgttatgaawcctgtgcagtggattt

SEQ ID NO: 13 - DNA, 33 bp, Homo sapiens
gtggggtgttggaaacrgttcccaattaggagc

SEQ ID NO: 14 - DNA, 33 bp, Homo sapiens
agtgaatcacagtgcaraacaaaaacactaaat

SEQ ID NO: 15 - DNA, 33 bp, Homo sapiens
ttctaagtacaacagamcaggctgtttgcaata

SEQ ID NO: 16 - DNA, 33 bp, Homo sapiens
atatattagtgaagtcsatgaaataaaggattg

SEQ ID NO: 17 - DNA, 33 bp, Homo sapiens
gagcctcttcattgccragatctgattgtgcaa

SEQ ID NO: 18 - DNA, 33 bp, Homo sapiens
atgctggaagtatggaracagtcaatgaaaact

SEQ ID NO: 19 - DNA, 33 bp, Homo sapiens
atttctggcatggaaasatcattaaagaaccaa

SEQ ID NO: 20 - DNA, 33 bp, Homo sapiens
ctgatgtcagaattccraaattccaagttaggg

SEQ ID NO: 21 - DNA, 33 bp, Homo sapiens
ctcatcttcctccccartatggtctataaggtc

SEQ ID NO: 22 - DNA, 33 bp, Homo sapiens
gagatgtctggtgcaayacaataggcactaaat

SEQ ID NO: 23 - DNA, 33 bp, Homo sapiens
agagggcaatatttaawgacagaattttgggtg

SEQ ID NO: 24 - DNA, 33 bp, Homo sapiens
taataccgcaataaaaygggtcacacgaatttt

SEQ ID NO: 25 - DNA, 33 bp, Homo sapiens
aaattcctttttgtcasaatggatcagagtcat

SEQ ID NO: 26 - DNA, 33 bp, Homo sapiens
accttttcatccccccratggataaacttatta

SEQ ID NO: 27 - DNA, 33 bp, Homo sapiens
gccaaaaagccaaatgrcaaatgatagatattt

SEQ ID NO: 28 - DNA, 33 bp, Homo sapiens
cctttccttctatcccyattgcaccatgctgtc

SEQ ID NO: 29 - DNA, 33 bp, Homo sapiens
tgtttgtcaagtcacartatgagcaaatctatc

SEQ ID NO: 30 - DNA, 33 bp, Homo sapiens
ccactctccaaatacarcattctctcttactct

SEQ ID NO: 31 - DNA, 33 bp, Homo sapiens
atttctttgattttaayggctaaacagtattcc

SEQ ID NO: 32 - DNA, 33 bp, Homo sapiens
tttgaaaaagttcccartaacaatgacattttt

SEQ ID NO: 33 - DNA, 33 bp, Homo sapiens
gagtgtatattatttaratggtcaccaaaatct

SEQ ID NO: 34 - DNA, 33 bp, Homo sapiens
gtgatctggaaagctasagtttctcgtttctgt

SEQ ID NO: 35 - DNA, 33 bp, Homo sapiens
gttagcagacagcggarctatgaattagacttg

SEQ ID NO: 36 - DNA, 33 bp, Homo sapiens
tcaggtccagtgaatcraaatctgcatcttcac

SEQ ID NO: 37 - DNA, 33 bp, Homo sapiens
tggggaggtgtgtctcraagccaagggatgaca

SEQ ID NO: 38 - DNA, 33 bp, Homo sapiens
aaacttttgtgatctaygtagtgcatcacaaat

SEQ ID NO: 39 - DNA, 33 bp, Homo sapiens
cctgatgaagtttcacrctgtcccactccagcc

SEQ ID NO: 40 - DNA, 33 bp, Homo sapiens
aactgaaactgcagaartggaaaccgtggataa

SEQ ID NO: 41 - DNA, 33 bp, Homo sapiens
aaccatgaataatgaasacatctaaatctccta

SEQ ID NO: 42 - DNA, 33 bp, Homo sapiens
gccagggcttaagagcyaagttaatagccctgg

SEQ ID NO: 43 - DNA, 33 bp, Homo sapiens
agcctcagatttcagargctaatcagaggtgac

SEQ ID NO: 44 - DNA, 33 bp, Homo sapiens
acttttatggaaacaaytaaaccatggcacttt

SEQ ID NO: 45 - DNA, 33 bp, Homo sapiens
ctgtgtagtataattcracagtgcaaaccacca

SEQ ID NO: 46 - DNA, 33 bp, Homo sapiens
acttttcattacctacygcagcaaatccaaaat

SEQ ID NO: 47 - DNA, 33 bp, Homo sapiens
ttcagacgtctaggtcsatcacagacctactga

SEQ ID NO: 48 - DNA, 33 bp, Homo sapiens
aatattctttgttttcyccacttaggatgggat

SEQ ID NO: 49 - DNA, 33 bp, Homo sapiens
aaatggcagagaatgaractttcaagtaattgg

SEQ ID NO: 50 - DNA, 33 bp, Homo sapiens
ttcttactagctctgckaacttggacagtaact

SEQ ID NO: 51 - DNA, 33 bp, Homo sapiens
ttccctgctgcgctgawaattgactttccctga

SEQ ID NO: 52 - DNA, 33 bp, Homo sapiens
caagagcatcagcatarcagaacattaaaatgg

SEQ ID NO: 53 - DNA, 33 bp, Homo sapiens
acactgggatttcttayaggttgctccacaaag

SEQ ID NO: 54 - DNA, 33 bp, Homo sapiens
ttatcagctttactgaratttctcatttgctcc

SEQ ID NO: 55 - DNA, 33 bp, Homo sapiens
gaaataaaccttggacmgagaaaagcaggcaaa

SEQ ID NO: 56 - DNA, 33 bp, Homo sapiens
gccccagcactaacatragtaccccacagagac

SEQ ID NO: 57 - DNA, 33 bp, Homo sapiens
tcctattggacactcayatagcacttatgctat

SEQ ID NO: 58 - DNA, 33 bp, Homo sapiens
ttcccaaaggcttttgraagagttgaaattcat

SEQ ID NO: 59 - DNA, 33 bp, Homo sapiens
ttggggaaggaaagagragacagcaatcacagg

SEQ ID NO: 60 - DNA, 33 bp, Homo sapiens
gactataaacggctcasaatggattgcacactg

SEQ ID NO: 61 - DNA, 33 bp, Homo sapiens
acttcagattatgagcraaggagaagccaatgg

SEQ ID NO: 62 - DNA, 33 bp, Homo sapiens
acttgccaaccagggaytaaagctattatcttg

SEQ ID NO: 63 - DNA, 33 bp, Homo sapiens
acttaattattcagtayagtcctggaaatgttt

SEQ ID NO: 64 - DNA, 33 bp, Homo sapiens
aactatgtttcattcaygttaatgctcaaattg

SEQ ID NO: 65 - DNA, 33 bp, Homo sapiens
aggagagctgtcaccartaacctgaccatgatg

SEQ ID NO: 66 - DNA, 33 bp, Homo sapiens
gatttctcaatggtagratccatcttctattta

SEQ ID NO: 67 - DNA, 33 bp, Homo sapiens
taaacaaagggtaatamattggaagagcagtag

SEQ ID NO: 68 - DNA, 33 bp, Homo sapiens
acaaaacaacatttacsctgaggaaggaacact

SEQ ID NO: 69 - DNA, 33 bp, Homo sapiens
tgctttgctttcagtarcattagctgaagtaat

SEQ ID NO: 70 - DNA, 33 bp, Homo sapiens
ttcaaaagatgacaacraaatgacactttctaa

SEQ ID NO: 71 - DNA, 33 bp, Homo sapiens
tttttctgatatttaaygtaccaacatatgata

SEQ ID NO: 72 - DNA, 33 bp, Homo sapiens
aaaatcacctataggcrataatctggcatacat

SEQ ID NO: 73 - DNA, 33 bp, Homo sapiens
aaaagttgtctttaaartcttccaacaaaaagt

SEQ ID NO: 74 - DNA, 33 bp, Homo sapiens
gtcctttgatgcagagyagtgtggtacccagca

SEQ ID NO: 75 - DNA, 33 bp, Homo sapiens
tccaagtggccagttaraagtctaaaccagggg

SEQ ID NO: 76 - DNA, 33 bp, Homo sapiens
tatttcagattgagaarcattatgcaagatagc

SEQ ID NO: 77 - DNA, 33 bp, Homo sapiens
gggaaaatggtgcatascgattaagtgacctgt

SEQ ID NO: 78 - DNA, 33 bp, Homo sapiens
agcaatatttgaagaartgaagactaagaatta

SEQ ID NO: 79 - DNA, 33 bp, Homo sapiens
aaaaggagtgttgacaraatcaaaatatttgtt

SEQ ID NO: 80 - DNA, 33 bp, Homo sapiens
tgtggatggaaactaastaacttctagcaaagg

SEQ ID NO: 81 - DNA, 33 bp, Homo sapiens
ttcttgtcgagcagaaraccagttgagaccact

SEQ ID NO: 82 - DNA, 33 bp, Homo sapiens
ctgtgaatgtgtggacmcttacaacattttatg

SEQ ID NO: 83 - DNA, 33 bp, Homo sapiens
gacacttagactgaaaytattgcccaagacttg

SEQ ID NO: 84 - DNA, 33 bp, Homo sapiens
aaattagctaagatggscaatgtattgatctag

SEQ ID NO: 85 - DNA, 33 bp, Homo sapiens
acctataggtcatttcrctctaaacttaaaaaa

SEQ ID NO: 86 - DNA, 33 bp, Homo sapiens
ctgtgtagagcaatgawtcacaattatttttgc

SEQ ID NO: 87 - DNA, 33 bp, Homo sapiens
agaagacagtcgagctmaaagatgggaagataa

SEQ ID NO: 88 - DNA, 33 bp, Homo sapiens
cctggccaaatcttgaygttaatatatctgttt

SEQ ID NO: 89 - DNA, 33 bp, Homo sapiens
ctcatactcacatttaragtcatttcttcaggg

SEQ ID NO: 90 - DNA, 33 bp, Homo sapiens
gttcatacacctagagwctccttctatctgaag

SEQ ID NO: 91 - DNA, 33 bp, Homo sapiens
ttctggtgttctgttayacagtaggattgctat

SEQ ID NO: 92 - DNA, 33 bp, Homo sapiens
tatgtaagatgtaaggmaggactctatctgcag

SEQ ID NO: 93 - DNA, 33 bp, Homo sapiens
ttctgcaagcaacatcraatttttcaagatctg

SEQ ID NO: 94 - DNA, 33 bp, Homo sapiens
atggctttcttgaccartaagtcttgttccttg

SEQ ID NO: 95 - DNA, 33 bp, Homo sapiens
gtaagtcccagagataraaggcaccatttctac

SEQ ID NO: 96 - DNA, 33 bp, Homo sapiens
tcatcattttattccaytcatttgtttgagtgt

SEQ ID NO: 97 - DNA, 33 bp, Homo sapiens
agattatagcctttaakaacacagacacttata

SEQ ID NO: 98 - DNA, 33 bp, Homo sapiens
acaaaccagtgagtgakaaccatcctggatgcc

SEQ ID NO: 99 - DNA, 33 bp, Homo sapiens
agtgctttgtggacaarataactctttaatacc

SEQ ID NO: 100 - DNA, 33 bp, Homo sapiens
gtcacttgtgaagtgaygggccttaaataccca

SEQ ID NO: 101 - DNA, 33 bp, Homo sapiens
gaaggccgaatatatayggaatgaacgtgtcaa

SEQ ID NO: 102 - DNA, 33 bp, Homo sapiens
gctagtattgcaaacakggtgaatgaagagcag

SEQ ID NO: 103 - DNA, 33 bp, Homo sapiens
tttattttaagcatctsagaactggtatcagcc

SEQ ID NO: 104 - DNA, 33 bp, Homo sapiens
aatgaggcgacaacatraaacgaaggtaatgtg

SEQ ID NO: 105 - DNA, 33 bp, Homo sapiens
tgcacaaacatctgctwacttcatggccaccaa

SEQ ID NO: 106 - DNA, 33 bp, Homo sapiens
ttttcgatttttggatkaggcatactcaatctg

SEQ ID NO: 107 - DNA, 33 bp, Homo sapiens
accgttctgagcactgkaggagaaaaatgaaaa

SEQ ID NO: 108 - DNA, 33 bp, Homo sapiens
agaaatattcaggatgkaaaactcggaggatgc

SEQ ID NO: 109 - DNA, 33 bp, Homo sapiens
gaggaccgaagtggaaygcttgtagaggactag

SEQ ID NO: 110 - DNA, 33 bp, Homo sapiens
ttctgtgtttctgggcmataaactagtgctatg

SEQ ID NO: 111 - DNA, 33 bp, Homo sapiens
gtttagaacattgtacygaaaagtcttgagcca

SEQ ID NO: 112 - DNA, 33 bp, Homo sapiens
ctcatgtggctattgargaagggcatagcggct

SEQ ID NO: 113 - DNA, 33 bp, Homo sapiens
gttcactgatgcttgaygcttattgcaggtctg

SEQ ID NO: 114 - DNA, 33 bp, Homo sapiens
tttctcatgatttccaygtgttaagaataacgc

SEQ ID NO: 115 - DNA, 33 bp, Homo sapiens
tccattacctccttcawctctggcaacacaata

SEQ ID NO: 116 - DNA, 33 bp, Homo sapiens
atttgtgtggtgaatayaattctctccttctta

SEQ ID NO: 117 - DNA, 33 bp, Homo sapiens
ggcaaatgatgctgtcycatagttgctttaatg

SEQ ID NO: 118 - DNA, 33 bp, Homo sapiens
agaaaagaggcatttgyatttctctaagaatac

SEQ ID NO: 119 - DNA, 33 bp, Homo sapiens
tgcaaatatttctgtargatagagccctaaaaa

SEQ ID NO: 120 - DNA, 33 bp, Homo sapiens
tcaaaagtacagtcacragaaatgttaggtgtt

SEQ ID NO: 121 - DNA, 33 bp, Homo sapiens
catgtttcaggcactayggtacttgctgggaga

SEQ ID NO: 122 - DNA, 33 bp, Homo sapiens
agggctgtaaatgctaygcaagtggtatgtgcc

SEQ ID NO: 123 - DNA, 33 bp, Homo sapiens
tggcattcttttcccaycctcatcgtaggtata

SEQ ID NO: 124 - DNA, 33 bp, Homo sapiens
atcatgtcctgccagartatttactccctacaa

SEQ ID NO: 125 - DNA, 33 bp, Homo sapiens
gattggtgtgcatgtgycaagcaaactagcaga

SEQ ID NO: 126 - DNA, 33 bp, Homo sapiens
ctccctatctcctccgyacagaaaccacaataa

SEQ ID NO: 127 - DNA, 33 bp, Homo sapiens
acttcttatggtaacaytatgtttaggcaaact

SEQ ID NO: 128 - DNA, 33 bp, Homo sapiens
atttccagtgaaagatkagcatgcttgtgtttt

SEQ ID NO: 129 - DNA, 33 bp, Homo sapiens
tccgtatttatccctamggcgtgccatctataa

SEQ ID NO: 130 - DNA, 33 bp, Homo sapiens
atattatggttggattyaaggatgattagtacg

SEQ ID NO: 131 - DNA, 33 bp, Homo sapiens
aatgcagagaaagtggwaaggtagtaaactagt

SEQ ID NO: 132 - DNA, 33 bp, Homo sapiens
gcagggttggttcataytagagattgtgaggga

SEQ ID NO: 133 - DNA, 33 bp, Homo sapiens
ttatttggtactccaarcttctgaagatttaaa

SEQ ID NO: 134 - DNA, 33 bp, Homo sapiens
ggaacgtagaactgtaygaagaggtcatttgag

SEQ ID NO: 135 - DNA, 33 bp, Homo sapiens
tcttcaacacagtaccraaattgctagccagag

SEQ ID NO: 136 - DNA, 33 bp, Homo sapiens
atgagttggcttaaaayagtaattcaagtcaca

SEQ ID NO: 137 - DNA, 33 bp, Homo sapiens
gaaagaaaagttaaacmaattagcagagacacc

SEQ ID NO: 138 - DNA, 33 bp, Homo sapiens
aggccatacctttgaawtctcactgttcatatg

SEQ ID NO: 139 - DNA, 33 bp, Homo sapiens
tatttgaaattgaatgyaacgaatgattttata

SEQ ID NO: 140 - DNA, 33 bp, Homo sapiens
ttacctgcttttctaaycttatgtctcaccaca

SEQ ID NO: 141 - DNA, 33 bp, Homo sapiens
cagagccgaaacccaasgaaacaagggacaagt

SEQ ID NO: 142 - DNA, 33 bp, Homo sapiens
tgttaagaaagaatgasattgatctgtatatgg

SEQ ID NO: 143 - DNA, 33 bp, Homo sapiens
atttcacagaaggtgaygtcggcctttttgata

SEQ ID NO: 144 - DNA, 33 bp, Homo sapiens
gagtagcaggtgaagasagatgaaccaaggatg

SEQ ID NO: 145 - DNA, 33 bp, Homo sapiens
caaagtttcagcaatgsaggatcaatagaagct

SEQ ID NO: 146 - DNA, 33 bp, Homo sapiens
agggatacacagatgasaacacttaattcagaa

SEQ ID NO: 147 - DNA, 33 bp, Homo sapiens
tggaaacttaggagacrgtagaaaagacacatg

SEQ ID NO: 148 - DNA, 33 bp, Homo sapiens
atttctactagtctgakaaacccattcaagcca

SEQ ID NO: 149 - DNA, 33 bp, Homo sapiens
agttaataaatccatcraaagcccgtccccaaa

SEQ ID NO: 150 - DNA, 33 bp, Homo sapiens
caacctgttgtctcacraagtgatctcctgcca

SEQ ID NO: 151 - DNA, 33 bp, Homo sapiens
tgggcaggtggaagacratgggtcaaggataag

SEQ ID NO: 152 - DNA, 33 bp, Homo sapiens
atcaattcttccttcaktaatgcctcagaaaaa

SEQ ID NO: 153 - DNA, 33 bp, Homo sapiens
aaagacaaatgaaatartaagtctgtggtattg

SEQ ID NO: 154 - DNA, 33 bp, Homo sapiens
cgctcatacgaaatgaracctgcctatgccttg

SEQ ID NO: 155 - DNA, 33 bp, Homo sapiens
taaaatacaattgctarctgaaatctgcagtag

SEQ ID NO: 156 - DNA, 33 bp, Homo sapiens
gaaggattggacaataraagcaatttaaatgtg

SEQ ID NO: 157 - DNA, 33 bp, Homo sapiens
ggtgtggggtggagaargtgctagtagcccacc

SEQ ID NO: 158 - DNA, 33 bp, Homo sapiens
tgtgttctccccgaaasttctctctgtcctgtg

SEQ ID NO: 159 - DNA, 33 bp, Homo sapiens
ccaaggctgaaaatacygatactcaacaaacag

SEQ ID NO: 160 - DNA, 33 bp, Homo sapiens
ccattgtcatgctccartctgttcaagcctttg

SEQ ID NO: 161 - DNA, 33 bp, Homo sapiens
gcctggcctgcagaagraccacggagcgttgtg

SEQ ID NO: 162 - DNA, 33 bp, Homo sapiens
aatgtggagcaactacrgacatgcaaaatgata

SEQ ID NO: 163 - DNA, 33 bp, Homo sapiens
agataagaaagtccaayaaatgtggagcaacta

SEQ ID NO: 164 - DNA, 33 bp, Homo sapiens
ggaagccacatcttgayaaatgcagaggcaatt

SEQ ID NO: 165 - DNA, 33 bp, Homo sapiens
catggcttcaccgcagyagtgcttttattatta

SEQ ID NO: 166 - DNA, 33 bp, Homo sapiens
ctcctggcagactcccyctagagctgctttgag

SEQ ID NO: 167 - DNA, 33 bp, Homo sapiens
gcaggcggaacacaaastcaggagatcgagacc

SEQ ID NO: 168 - DNA, 33 bp, Homo sapiens
acaacaagtgactacaycgtaatgagaaataga

SEQ ID NO: 169 - DNA, 33 bp, Homo sapiens
ttcacgtcctgagttayaaaccctgcaggtccc

SEQ ID NO: 170 - DNA, 33 bp, Homo sapiens
cagttcatgatctggayggattgagtgagatgc

SEQ ID NO: 171 - DNA, 33 bp, Homo sapiens
aataaaaagaaaccaaytactactacgtacaac

SEQ ID NO: 172 - DNA, 33 bp, Homo sapiens
tcactgctcaaggtccycaagctttcccctctt

SEQ ID NO: 173 - DNA, 33 bp, Homo sapiens
ttttcaaggctgaacartggacttgcttttgga

SEQ ID NO: 174 - DNA, 33 bp, Homo sapiens
cagcaatgagttaccartatgtgaatttccagc

SEQ ID NO: 175 - DNA, 33 bp, Homo sapiens
ataaatgagacgtgacygtactttaaggagtgc

SEQ ID NO: 176 - DNA, 33 bp, Homo sapiens
taatttagcagataggratccaaagaatatgtg

SEQ ID NO: 177 - DNA, 33 bp, Homo sapiens
gttttaaaggtcagagyatggcgtccattcaca

SEQ ID NO: 178 - DNA, 33 bp, Homo sapiens
attaatgctttgctctraccaactgggcaaatc

SEQ ID NO: 179 - DNA, 33 bp, Homo sapiens
gtcagtcaaggtggaaratcactgcagggcaca

SEQ ID NO: 180 - DNA, 33 bp, Homo sapiens
atgtacccatttaaccygcaacatcaccaggtg

SEQ ID NO: 181 - DNA, 33 bp, Homo sapiens
aaggccttttctctggrcacaggcagggagagc

SEQ ID NO: 182 - DNA, 33 bp, Homo sapiens
aatgtcgtctgcccagsaatgcagagtgtagat

SEQ ID NO: 183 - DNA, 33 bp, Homo sapiens
gttttcttgaaatacaktactcactcttgggat

SEQ ID NO: 184 - DNA, 33 bp, Homo sapiens
ctctgggcacctgacaygtataaactcaccaga

SEQ ID NO: 185 - DNA, 33 bp, Homo sapiens
aggagactaagcctcgragaagtgagttgtttg

SEQ ID NO: 186 - DNA, 33 bp, Homo sapiens
gattctaacccaaaccygatggcaaaggctatc

SEQ ID NO: 187 - DNA, 33 bp, Homo sapiens
taattttgctttaccgmagatttctaaacttta

SEQ ID NO: 188 - DNA, 33 bp, Homo sapiens
gcatttgagcttgctakaataagactgactttt

SEQ ID NO: 189 - DNA, 33 bp, Homo sapiens
atcgacgtgggactaascaaggaacactttcgt

SEQ ID NO: 190 - DNA, 33 bp, Homo sapiens
aggaaactggaggtgawgtatatttccatcacc

SEQ ID NO: 191 - DNA, 33 bp, Homo sapiens
caggaatgaggggccaragaaatgtattataaa

SEQ ID NO: 192 - DNA, 33 bp, Homo sapiens
taatagattcttttgargagcagattctttttt

SEQ ID NO: 193 - DNA, 33 bp, Homo sapiens
cactggaagatgaagasgtaatgatcacatttc

SEQ ID NO: 194 - DNA, 33 bp, Homo sapiens
accaagatttcaaagasgtgaataaaagctcac

SEQ ID NO: 195 - DNA, 33 bp, Homo sapiens
ttcatatttttggaaastgacttaatttctatg

SEQ ID NO: 196 - DNA, 33 bp, Homo sapiens
cttcaagtgatacctcrgagagcctcaggttct

SEQ ID NO: 197 - DNA, 33 bp, Homo sapiens
aatagagattatatgcyccaacctaatatgagt

SEQ ID NO: 198 - DNA, 33 bp, Homo sapiens
gaattagaagtaaatgraaactcgtaactacat

SEQ ID NO: 199 - DNA, 33 bp, Homo sapiens
gatactatgaaaatcartccaaaacatcgtgat

SEQ ID NO: 200 - DNA, 33 bp, Homo sapiens
tgtaatagaaagggtawctgagggtacaaacat

SEQ ID NO: 201 - DNA, 33 bp, Homo sapiens
gtaataaccgtttgtcmaatcaatgaatgacaa

SEQ ID NO: 202 - DNA, 33 bp, Homo sapiens
tagttaacccttcaaasagtaggcatcctgccc

SEQ ID NO: 203 - DNA, 33 bp, Homo sapiens
tgggaatgtctgtgggraagaagaggctctgct

SEQ ID NO: 204 - DNA, 33 bp, Homo sapiens
aagactgatacaggacraagggtggctgtggcc

SEQ ID NO: 205 - DNA, 33 bp, Homo sapiens
ggtgcccacacctatayaatgggggtaataaag

SEQ ID NO: 206 - DNA, 33 bp, Homo sapiens
cctgtgctggtcatccyctcactgcccctgcac

SEQ ID NO: 207 - DNA, 33 bp, Homo sapiens
gaactcctgacttccarcctggtgcttttccta

SEQ ID NO: 208 - DNA, 33 bp, Homo sapiens
gattttgattgatgggycatatattagcctggt

SEQ ID NO: 209 - DNA, 33 bp, Homo sapiens
caccctctagttgtgarattcaaaaatgtctcc

SEQ ID NO: 210 - DNA, 33 bp, Homo sapiens
catacaccaatcagaargggactgttcagaaaa

SEQ ID NO: 211 - DNA, 33 bp, Homo sapiens
acctgcttgcctgcaayatgagttttacaccat

SEQ ID NO: 212 - DNA, 33 bp, Homo sapiens
cataaagcagagatgartaaaagccagagggag

SEQ ID NO: 213 - DNA, 33 bp, Homo sapiens
tctctggaacaatcacwgtctttctctcgctac

SEQ ID NO: 214 - DNA, 33 bp, Homo sapiens
gatccatcttttagcargaggtacatttcctta

SEQ ID NO: 215 - DNA, 33 bp, Homo sapiens
gacataaaaggttacayattgtatgatgctact

SEQ ID NO: 216 - DNA, 33 bp, Homo sapiens
aacttaatggaatagamggtagcaaccacacat

SEQ ID NO: 217 - DNA, 33 bp, Homo sapiens
gtgtatacttgtgaggscatgagcatgattaag

SEQ ID NO: 218 - DNA, 33 bp, Homo sapiens
agtaaagcttaagacarctctgcttcaaattct

SEQ ID NO: 219 - DNA, 33 bp, Homo sapiens
agataaaaattaaaagrcttagatcctgcattc

SEQ ID NO: 220 - DNA, 33 bp, Homo sapiens
agagattgatgtggtayatttagctgaattttg

SEQ ID NO: 221 - DNA, 33 bp, Homo sapiens
acaatttctccttcackgccagtttgagataag

SEQ ID NO: 222 - DNA, 33 bp, Homo sapiens
aactcactggggttacratggatccaaaaccaa

SEQ ID NO: 223 - DNA, 33 bp, Homo sapiens
gagctttgcgattgtayagaaggggagtcaagt

SEQ ID NO: 224 - DNA, 33 bp, Homo sapiens
gaaaatcacctaattayagcaaatgccctgatg

SEQ ID NO: 225 - DNA, 33 bp, Homo sapiens
ccaccgcgcctggcgayaaattcttttaaatat

SEQ ID NO: 226 - DNA, 33 bp, Homo sapiens
aaatataatactctaaycaccaatgacaaatac

SEQ ID NO: 227 - DNA, 33 bp, Homo sapiens
ctccctgtgctctttaraggtccaggagtaggc

SEQ ID NO: 228 - DNA, 33 bp, Homo sapiens
gaccagtgaggcacaartgaaggttttctagcg

SEQ ID NO: 229 - DNA, 33 bp, Homo sapiens
aaagtgcgggctgctgraagcccagggatggaa

SEQ ID NO: 230 - DNA, 33 bp, Homo sapiens
aggaatggcagtttacycacaatttctgccctg

SEQ ID NO: 231 - DNA, 33 bp, Homo sapiens
acaaagctctatgtcayctaaactgataattat

SEQ ID NO: 232 - DNA, 33 bp, Homo sapiens
tccttcttatccaaacraggaagtcagtattca

SEQ ID NO: 233 - DNA, 33 bp, Homo sapiens
catctctttgttcatcmctgaattctctctagt

SEQ ID NO: 234 - DNA, 33 bp, Homo sapiens
tccatatccaccacagmatcaaatgaaactgta

SEQ ID NO: 235 - DNA, 33 bp, Homo sapiens
caagtgttttgttcacrcacctcaagagaggag

SEQ ID NO: 236 - DNA, 33 bp, Homo sapiens
tctgccatttccatcarctattactggacatga

SEQ ID NO: 237 - DNA, 33 bp, Homo sapiens
ttgaagatgcttgctaraccaggaaatatgatt

SEQ ID NO: 238 - DNA, 33 bp, Homo sapiens
agactgcaagaagagaraacctttaaactgatc

SEQ ID NO: 239 - DNA, 33 bp, Homo sapiens
gagttgtgcaatcatcrctgtaacctaatttta

SEQ ID NO: 240 - DNA, 33 bp, Homo sapiens
ctgtcactgaaagcccrcagttctggagatgac

SEQ ID NO: 241 - DNA, 33 bp, Homo sapiens
caggagaggttgttacragaggttgtcaagaag

SEQ ID NO: 242 - DNA, 33 bp, Homo sapiens
aatggctgatttgacasagtagacgcccattgg

SEQ ID NO: 243 - DNA, 33 bp, Homo sapiens
ttaaacccagtaaggaycttgaacagtagacat

SEQ ID NO: 244 - DNA, 33 bp, Homo sapiens
ttgtagctttcaagacragaacaaagaaaatgc

SEQ ID NO: 245 - DNA, 33 bp, Homo sapiens
actgctgtattccgaawctctagaacgtctggc

SEQ ID NO: 246 - DNA, 33 bp, Homo sapiens
acatcttcactctacayggtagcaaagtgccag

SEQ ID NO: 247 - DNA, 33 bp, Homo sapiens
agccctctcactgttgraaacatggctgtcttt

SEQ ID NO: 248 - DNA, 33 bp, Homo sapiens
cctcaaacagcttacarcatgggcgaaaaggac

SEQ ID NO: 249 - DNA, 33 bp, Homo sapiens
atccacaatatttaaayggctgttttagtcact

SEQ ID NO: 250 - DNA, 33 bp, Homo sapiens
tgtggataaccaaactracatgtggaatctgac

SEQ ID NO: 251 - DNA, 33 bp, Homo sapiens
ttgtgcacaggaaagtsaatcagctgtgatctc

SEQ ID NO: 252 - DNA, 33 bp, Homo sapiens
aacatgtgagatgcaamttagcgtattcttgaa

SEQ ID NO: 253 - DNA, 33 bp, Homo sapiens
atcatagctgaacccaygagaatgaaagaaagg

SEQ ID NO: 254 - DNA, 33 bp, Homo sapiens
gcaggcttctcagtaargaacagacttggggtg

SEQ ID NO: 255 - DNA, 33 bp, Homo sapiens
tattccttccttctaaytgaacatttttgaggc

SEQ ID NO: 256 - DNA, 33 bp, Homo sapiens
caccctccatcggtaasaagatcaccaggcatt

SEQ ID NO: 257 - DNA, 33 bp, Homo sapiens
cctgtgcatatgctacrccaggggtcctcaacc

SEQ ID NO: 258 - DNA, 33 bp, Homo sapiens
ctatattaaccatagawtcatccataatagttc

SEQ ID NO: 259 - DNA, 33 bp, Homo sapiens
agccctgctgagattargtgaactccagttgat

SEQ ID NO: 260 - DNA, 33 bp, Homo sapiens
cagatgtaacactcaaytgaagcattctcaggt

SEQ ID NO: 261 - DNA, 33 bp, Homo sapiens
ttatataacttttggayatacagcctgatttga

SEQ ID NO: 262 - DNA, 33 bp, Homo sapiens
gttgttgaaaggattamaatgacagagtaaata

SEQ ID NO: 263 - DNA, 33 bp, Homo sapiens
acagattgaattgctamaacatacaagactggc

SEQ ID NO: 264 - DNA, 33 bp, Homo sapiens
tgagaccagctggaaayagatactgaaatcttg

SEQ ID NO: 265 - DNA, 33 bp, Homo sapiens
caaaatgcttcacttaycaaagcaagatttcag

SEQ ID NO: 266 - DNA, 33 bp, Homo sapiens
agagaaaggtgataacraaatgaaagcataatc

SEQ ID NO: 267 - DNA, 33 bp, Homo sapiens
ctatttaagacagcaarcgtacattaagactaa

SEQ ID NO: 268 - DNA, 33 bp, Homo sapiens
agaggagcttattttcycttagtcagttttgaa

SEQ ID NO: 269 - DNA, 33 bp, Homo sapiens
taagaagaaaattttgrattccaatagtgactg

SEQ ID NO: 270 - DNA, 33 bp, Homo sapiens
aaataagccagaaaacyagcggtcacatacaaa

SEQ ID NO: 271 - DNA, 33 bp, Homo sapiens
aaggaagagttttaaayggtagagtactatgtt

SEQ ID NO: 272 - DNA, 33 bp, Homo sapiens
ttgttaccattcccacrggtttgagttagctct

SEQ ID NO: 273 - DNA, 33 bp, Homo sapiens
cctttcttcacccaggmcaacgagtttgtggaa

SEQ ID NO: 274 - DNA, 33 bp, Homo sapiens
gttgcactaagtgtcayataccatgcaggttgg

SEQ ID NO: 275 - DNA, 33 bp, Homo sapiens
gtaactaataagtaaargagcaagttatcctgc

SEQ ID NO: 276 - DNA, 33 bp, Homo sapiens
cctcagctgtaaaataygccttaagtttgggtg

SEQ ID NO: 277 - DNA, 33 bp, Homo sapiens
ggcactcttgctatcartcatcctgtctccaca

SEQ ID NO: 278 - DNA, 33 bp, Homo sapiens
agttgaagatgtggacmcctgcagttgctactg

SEQ ID NO: 279 - DNA, 33 bp, Homo sapiens
gatactagaacagaacraggatcgcagcaagaa

SEQ ID NO: 280 - DNA, 33 bp, Homo sapiens
aacccttttggtgtgcmaattttggaatgcagt

SEQ ID NO: 281 - DNA, 33 bp, Homo sapiens
aaagctgatagaaggayagcttaccgagaaaaa

SEQ ID NO: 282 - DNA, 33 bp, Homo sapiens
ttcatacaaggctatgyaggggataggaagata

SEQ ID NO: 283 - DNA, 33 bp, Homo sapiens
gccagagaacttgattsaaaagcaccccaaaga

SEQ ID NO: 284 - DNA, 33 bp, Homo sapiens
tggagggcacacataaragtaatatggaagaac

SEQ ID NO: 285 - DNA, 33 bp, Homo sapiens
aggcatatttttcttayggttggcacagatata

SEQ ID NO: 286 - DNA, 33 bp, Homo sapiens
aatctgtgtgcccaaamaatccaaaatatataa

SEQ ID NO: 287 - DNA, 33 bp, Homo sapiens
aatgacatttgctgcasgttgataattagtttt

SEQ ID NO: 288 - DNA, 33 bp, Homo sapiens
tcatgctaaagtctgaraaccattagcctatat

SEQ ID NO: 289 - DNA, 33 bp, Homo sapiens
acgcacacacacgaaaragaatcttctgactta

SEQ ID NO: 290 - DNA, 33 bp, Homo sapiens
agtgcacgacttccaaygtcctggtggaaatgt

SEQ ID NO: 291 - DNA, 33 bp, Homo sapiens
gattaccaaatgttcaraactcagctcctgccc

SEQ ID NO: 292 - DNA, 33 bp, Homo sapiens
ttaacactgttttgagsatttgagatagatcag

SEQ ID NO: 293 - DNA, 33 bp, Homo sapiens
ggattctaagggctacratgaaagtctctgcta

SEQ ID NO: 294 - DNA, 33 bp, Homo sapiens
tgaaaggttcgcaaaastagctctgaagaagtg

SEQ ID NO: 295 - DNA, 33 bp, Homo sapiens
ctacagttggctggaamaaaggaggatttggga

SEQ ID NO: 296 - DNA, 33 bp, Homo sapiens
cagcatgtgagaaggcygaattaaaaggaaggt

SEQ ID NO: 297 - DNA, 33 bp, Homo sapiens
aggatgacgccttcacratgggactggtagctc

SEQ ID NO: 298 - DNA, 33 bp, Homo sapiens
tagccatgaaattaccrcatcatgaaaccttca

SEQ ID NO: 299 - DNA, 33 bp, Homo sapiens
caggttcacactgtacractccagaaggtgcca

SEQ ID NO: 300 - DNA, 33 bp, Homo sapiens
tctaccagatgttgagycccttgaacctaatgg

SEQ ID NO: 301 - DNA, 33 bp, Homo sapiens
aagacttctatcagacratccctgaagttcctt

SEQ ID NO: 302 - DNA, 33 bp, Homo sapiens
catttgtttcctgaaakttctgattgtttaaac

SEQ ID NO: 303 - DNA, 33 bp, Homo sapiens
gaagttttaacatagcrcaggaaagaagggcat

SEQ ID NO: 304 - DNA, 33 bp, Homo sapiens
ctctaaaagttacataytatagccttaactcac

SEQ ID NO: 305 - DNA, 33 bp, Homo sapiens
gtcattacgcccaaacragtcaatttcaaggat

SEQ ID NO: 306 - DNA, 33 bp, Homo sapiens
ggaaaatttggctttcrgaaaatggctgaccta

SEQ ID NO: 307 - DNA, 33 bp, Homo sapiens
tgcctaggtaggaaaasgaaacttagtgaagga

SEQ ID NO: 308 - DNA, 33 bp, Homo sapiens
ggtgtccctgtctggaygggtttagtcagcatc

SEQ ID NO: 309 - DNA, 33 bp, Homo sapiens
gttcaagggctgaaccwaaggacaaaagaaggg

SEQ ID NO: 310 - DNA, 33 bp, Homo sapiens
tatttagcatttattargaacctcctgtgggct

SEQ ID NO: 311 - DNA, 33 bp, Homo sapiens
tgttacctttcctggayacatgagggtttcttt

SEQ ID NO: 312 - DNA, 33 bp, Homo sapiens
ttttaattttagcacargctgttacttctacaa

SEQ ID NO: 313 - DNA, 33 bp, Homo sapiens
ttctagaaagatggccractaaagacacgctca

SEQ ID NO: 314 - DNA, 33 bp, Homo sapiens
gacttagagaatttcasaagagtagtagtacaa

SEQ ID NO: 315 - DNA, 33 bp, Homo sapiens
aagtgggaaaaactaaycaacgtgcttgaacat

SEQ ID NO: 316 - DNA, 33 bp, Homo sapiens
taacggatgggaaaaawtggtttgtagatggag

SEQ ID NO: 317 - DNA, 33 bp, Homo sapiens
gcaaaatctgtgttcakgttgagttaggttatg

SEQ ID NO: 318 - DNA, 33 bp, Homo sapiens
tgtcaatgttatttaayccgaataagaacatta

SEQ ID NO: 319 - DNA, 33 bp, Homo sapiens
cattactctcaaagtaygccagccctcccagat

SEQ ID NO: 320 - DNA, 33 bp, Homo sapiens
ttggagggaatacattsaaacagtagcaaattt

SEQ ID NO: 321 - DNA, 33 bp, Homo sapiens
actgtcagaaaataaaygactgctcttcataaa

SEQ ID NO: 322 - DNA, 33 bp, Homo sapiens
tgaaaaatcattcaaasaaatttgtcttagtct

SEQ ID NO: 323 - DNA, 33 bp, Homo sapiens
tattagttatccatccraagaaactaagcagtg

SEQ ID NO: 324 - DNA, 33 bp, Homo sapiens
atgggttcataaatgcyagtcctagggtatggc

SEQ ID NO: 325 - DNA, 33 bp, Homo sapiens
ggtagctggtatgtgaractagagcccagggaa

SEQ ID NO: 326 - DNA, 33 bp, Homo sapiens
gccccagataatacaamaaaatggccaacgttt

SEQ ID NO: 327 - DNA, 33 bp, Homo sapiens
ttctgcaggctgtaaakcatttggaagagaagt

SEQ ID NO: 328 - DNA, 33 bp, Homo sapiens
ccagctagaggggcaayttaaaggcatcaggta

SEQ ID NO: 329 - DNA, 33 bp, Homo sapiens
tctctcctgcacatacyggaggactactgtgtt

SEQ ID NO: 330 - DNA, 33 bp, Homo sapiens
ttcatcagagcagtggkaaggggtcttttgtga

SEQ ID NO: 331 - DNA, 33 bp, Homo sapiens
agccaagacaggatacrggttaggcacctcctt

SEQ ID NO: 332 - DNA, 33 bp, Homo sapiens
tataggattcaaatgakagctttatttcaattt

SEQ ID NO: 333 - DNA, 33 bp, Homo sapiens
agaaagttgtaggagcygcagtgggagctggaa

SEQ ID NO: 334 - DNA, 33 bp, Homo sapiens
tattttttccactgaamtcttcagtccctcaaa

SEQ ID NO: 335 - DNA, 33 bp, Homo sapiens
aaatgtaaccaggaaawgtaatgttgattacaa

SEQ ID NO: 336 - DNA, 33 bp, Homo sapiens
atcccagccattcttamcagcgggagttatcaa

SEQ ID NO: 337 - DNA, 33 bp, Homo sapiens
ctcagccgtgatgggakaccatggttgtggaga

SEQ ID NO: 338 - DNA, 33 bp, Homo sapiens
gacataaaaggcagtaragaccgggcacagtgg

SEQ ID NO: 339 - DNA, 33 bp, Homo sapiens
gagaaccactggtccasggaagaacagtcttat

SEQ ID NO: 340 - DNA, 33 bp, Homo sapiens
tcaagagtggaagcagyaagagagttaagaggc

SEQ ID NO: 341 - DNA, 33 bp, Homo sapiens
atggcagccagagctawctgatcaaatgtagag

SEQ ID NO: 342 - DNA, 33 bp, Homo sapiens
gggaaatgggcagtaaygatcacgggaaatttg

SEQ ID NO: 343 - DNA, 33 bp, Homo sapiens
caagcccagctgagacrctatccttctggcggt

SEQ ID NO: 344 - DNA, 33 bp, Homo sapiens
tcattaattcaatttcmaatcccacactgcagc

SEQ ID NO: 345 - DNA, 33 bp, Homo sapiens
gtgtaacatggaaatamcaggtttagccaacct

SEQ ID NO: 346 - DNA, 33 bp, Homo sapiens
aggcactgagagagaawattaggaagacaacac

SEQ ID NO: 347 - DNA, 33 bp, Homo sapiens
gtcagatttgcataagkacacaccacaaggctg

SEQ ID NO: 348 - DNA, 33 bp, Homo sapiens
tgcatctttggacgaarctagaaaagaagctga

SEQ ID NO: 349 - DNA, 33 bp, Homo sapiens
atgggcccttttcagartaaaacatatagactt

SEQ ID NO: 350 - DNA, 33 bp, Homo sapiens
aagcggggaaggggcayggatatctttaccgca

SEQ ID NO: 351 - DNA, 33 bp, Homo sapiens
gaggaatatagcaagamcctgcatctataataa

SEQ ID NO: 352 - DNA, 33 bp, Homo sapiens
catggtcaattagtccrcacatactgttaactt

SEQ ID NO: 353 - DNA, 33 bp, Homo sapiens
tacagtgagtaggaagwattgtttgagtggagc

SEQ ID NO: 354 - DNA, 33 bp, Homo sapiens
attttatggctaattaraaatgatgattgttca

SEQ ID NO: 355 - DNA, 33 bp, Homo sapiens
atgtggccttatttagyaatggtaccattttgt

SEQ ID NO: 356 - DNA, 33 bp, Homo sapiens
aactcaaacttaaacasatgatgacttgcaatt

SEQ ID NO: 357 - DNA, 33 bp, Homo sapiens
tgggatagatgcctaarccatgatgcctgccca

SEQ ID NO: 358 - DNA, 33 bp, Homo sapiens
aataaaggcttgaacaytatttgtctgtttctg

SEQ ID NO: 359 - DNA, 33 bp, Homo sapiens
ccttcacaggagttaakagagtcctccaaaacc

SEQ ID NO: 360 - DNA, 33 bp, Homo sapiens
aactgcttttattgacraccacataaaatcacc

SEQ ID NO: 361 - DNA, 33 bp, Homo sapiens
ttcaaaaatagacagawataaatgatggtgttg

SEQ ID NO: 362 - DNA, 33 bp, Homo sapiens
gttgactggctagtgayccttcatgccccagga

SEQ ID NO: 363 - DNA, 33 bp, Homo sapiens
ataaaaacaagatgaaygtaaccagccatggct

SEQ ID NO: 364 - DNA, 33 bp, Homo sapiens
atagcaacccaaagcgwcatatgttcaagttaa

SEQ ID NO: 365 - DNA, 33 bp, Homo sapiens
tgtgtggcttagaaaascattagcctctaggga

SEQ ID NO: 366 - DNA, 33 bp, Homo sapiens
caggctgatgatttgcractagtaaaaataata

SEQ ID NO: 367 - DNA, 33 bp, Homo sapiens
gaccatcaaatacttayggctagatgctccagt

SEQ ID NO: 368 - DNA, 33 bp, Homo sapiens
gtagacgagttaccctktcttcttccatgtacc

SEQ ID NO: 369 - DNA, 33 bp, Homo sapiens
ttccccttgctctgcawatcaggtcaaacactt

SEQ ID NO: 370 - DNA, 33 bp, Homo sapiens
attaagtgcttagtgayctctatgacagaaaag

SEQ ID NO: 371 - DNA, 33 bp, Homo sapiens
caattagttggagtcaytctagattttattttg

SEQ ID NO: 372 - DNA, 33 bp, Homo sapiens
aaattaatgaaggagaygatttcaggataacat

SEQ ID NO: 373 - DNA, 33 bp, Homo sapiens
gtgttctgggtcattayatattatgttactatt

SEQ ID NO: 374 - DNA, 33 bp, Homo sapiens
atcatcataatttagamcttttttgtgtgtgtt

SEQ ID NO: 375 - DNA, 33 bp, Homo sapiens
atcccatgcagcaaaaycactgttccttcttgt

SEQ ID NO: 376 - DNA, 33 bp, Homo sapiens
gttggatatgagagcaytgaattgtggagctta

SEQ ID NO: 377 - DNA, 33 bp, Homo sapiens
tttactgacaattacamggatgcaaacaaacaa

SEQ ID NO: 378 - DNA, 33 bp, Homo sapiens
cattttagaactgcacwccacttacaaaggtaa

SEQ ID NO: 379 - DNA, 33 bp, Homo sapiens
gtatccattacaaaaamgtcaattgcaaatggt

SEQ ID NO: 380 - DNA, 33 bp, Homo sapiens
ttagagcttgaaactaygaattttgagtggatg

SEQ ID NO: 381 - DNA, 33 bp, Homo sapiens
agagattaccgtggccratcttctatgagtgca

SEQ ID NO: 382 - DNA, 33 bp, Homo sapiens
ggcctagtgcaggtagmagctgggctaaagttt

SEQ ID NO: 383 - DNA, 33 bp, Homo sapiens
tgcttatttttgttgcwatcagataatgcgtta

SEQ ID NO: 384 - DNA, 33 bp, Homo sapiens
ggctgtttctcaaatayatcactctgcagaaaa

SEQ ID NO: 385 - DNA, 33 bp, Homo sapiens
actaactagatgaatcrcccagtttttcccttt

SEQ ID NO: 386 - DNA, 33 bp, Homo sapiens
ctacccaaaggaaataygtctgtatgcaggagg

SEQ ID NO: 387 - DNA, 33 bp, Homo sapiens
tcagctcaggctgttaygacaaaaataccacag

SEQ ID NO: 388 - DNA, 33 bp, Homo sapiens
ccattcgaacatatgcyaatgaaatgtgtggaa

SEQ ID NO: 389 - DNA, 33 bp, Homo sapiens
atgtccacgtcagaaakctgctgaatatcttcg

SEQ ID NO: 390 - DNA, 33 bp, Homo sapiens
gtaagtatttccgctcycaggaagtctgcatta

SEQ ID NO: 391 - DNA, 33 bp, Homo sapiens
atcacaatatcaatacrgaggcttattgtgttg

SEQ ID NO: 392 - DNA, 33 bp, Homo sapiens
gtagacagtacatcccragaaagagaggattca

SEQ ID NO: 393 - DNA, 33 bp, Homo sapiens
tttgcttcccatcctckaggtgcacagtgtcca

SEQ ID NO: 394 - DNA, 33 bp, Homo sapiens
gtcatgtgacctactcygaaaaaacacataatt

SEQ ID NO: 395 - DNA, 33 bp, Homo sapiens
tgacaattttcacttgrcaaacatttattcctt

SEQ ID NO: 396 - DNA, 33 bp, Homo sapiens
gctatggcagtcctagmacagtttcaattcttc

SEQ ID NO: 397 - DNA, 33 bp, Homo sapiens
aggaggaagagagagakatacctcttgttttct

SEQ ID NO: 398 - DNA, 33 bp, Homo sapiens
ctttaatgcactaaacygactcagtgaaaattt

SEQ ID NO: 399 - DNA, 33 bp, Homo sapiens
atgaacatatgagtacmggtatgtttttgatgt

SEQ ID NO: 400 - DNA, 33 bp, Homo sapiens
ctgagaagtcgtgtggwccaacctttgctcatt

SEQ ID NO: 401 - DNA, 33 bp, Homo sapiens
ttctgagaaaatattgragaccatctttatgcc

SEQ ID NO: 402 - DNA, 33 bp, Homo sapiens
acaaaaacaagcctacmataaaaaatgccagtg

SEQ ID NO: 403 - DNA, 33 bp, Homo sapiens
aggttggctcttgttcyattaacactgatgctc

SEQ ID NO: 404 - DNA, 33 bp, Homo sapiens
aaatttactagaaaccraacaatgattgccact

SEQ ID NO: 405 - DNA, 33 bp, Homo sapiens
cagcagtgacttctaartgactatgtggaggag

SEQ ID NO: 406 - DNA, 33 bp, Homo sapiens
atgaactaaattttggmatgacaaagacagaat

SEQ ID NO: 407 - DNA, 33 bp, Homo sapiens
actggtaatgtatcacrcaggaaaaatggtaat

SEQ ID NO: 408 - DNA, 33 bp, Homo sapiens
gttttgttggatttcgkattaactatactcact

SEQ ID NO: 409 - DNA, 33 bp, Homo sapiens
acactttaaattttcayaattgccttggaccat

SEQ ID NO: 410 - DNA, 33 bp, Homo sapiens
gtttcactccactaackacacttggcttaatcc

SEQ ID NO: 411 - DNA, 33 bp, Homo sapiens
gactttcagttttgtawccctagagctgccttg

SEQ ID NO: 412 - DNA, 33 bp, Homo sapiens
cccctatgccacatcarccatcgagcccggtgc

SEQ ID NO: 413 - DNA, 33 bp, Homo sapiens
aagtaatcataacctcrcaaagcttgagagtgt

SEQ ID NO: 414 - DNA, 33 bp, Homo sapiens
gaaaactgtggtttaasagtgagaaaggatcat

SEQ ID NO: 415 - DNA, 33 bp, Homo sapiens
ctaatccagcacccacrattaacataaaggaaa

SEQ ID NO: 416 - DNA, 33 bp, Homo sapiens
ctaataaagcaacaaawctattttatgtcatgg

SEQ ID NO: 417 - DNA, 33 bp, Homo sapiens
ttacaggatcctaagtsaaaacggtcatccgcc

SEQ ID NO: 418 - DNA, 33 bp, Homo sapiens
ttgtccttatccattasacttcattttacagaa

SEQ ID NO: 419 - DNA, 33 bp, Homo sapiens
atcaacaaatggataamcattataccacatgaa

SEQ ID NO: 420 - DNA, 33 bp, Homo sapiens
gtggtacaagcaggcaytagtttcatgggggtc

SEQ ID NO: 421 - DNA, 33 bp, Homo sapiens
acctttatctcacatayagtctctttcaatagc

SEQ ID NO: 422 - DNA, 33 bp, Homo sapiens
ggttgaggtactatgarctcacatttaggattt

SEQ ID NO: 423 - DNA, 33 bp, Homo sapiens
tttttccaatccattaraaatcattctgtatta

SEQ ID NO: 424 - DNA, 33 bp, Homo sapiens
agcaaactcagaataaytgaaattgcaatagca

SEQ ID NO: 425 - DNA, 33 bp, Homo sapiens
caccttcatgtggaaastgtacaggcccctttc

SEQ ID NO: 426 - DNA, 33 bp, Homo sapiens
cagcaactatccaagartacaacttcatttgct

SEQ ID NO: 427 - DNA, 33 bp, Homo sapiens
tgccaagattaaaaaayggtgcattcttgttta

SEQ ID NO: 428 - DNA, 33 bp, Homo sapiens
ttggcaacatcttgggrcatggttagggccatg

SEQ ID NO: 429 - DNA, 33 bp, Homo sapiens
cccagggtgtcagctayatccaaggctagaaaa

SEQ ID NO: 430 - DNA, 33 bp, Homo sapiens
ggttatgatgcgcatgkatctggttaagttaca

SEQ ID NO: 431 - DNA, 33 bp, Homo sapiens
tacccagctatatccayagttgcaaaacaaagt

SEQ ID NO: 432 - DNA, 33 bp, Homo sapiens
ctatgatgtaaatgtarcaaatatgacttgatt

SEQ ID NO: 433 - DNA, 33 bp, Homo sapiens
tatccataatcaatcayacaaaatacaagtaaa

SEQ ID NO: 434 - DNA, 33 bp, Homo sapiens
gggcctgggtggatccmaaataagatattactg

SEQ ID NO: 435 - DNA, 33 bp, Homo sapiens
cttaagaacttctcaaygaaatgaattatttag

SEQ ID NO: 436 - DNA, 33 bp, Homo sapiens
ataacaaaaaaccaatmagtctgtagaaaaatg

SEQ ID NO: 437 - DNA, 33 bp, Homo sapiens
accatcaaatgttattwaagagagctataaaaa

SEQ ID NO: 438 - DNA, 33 bp, Homo sapiens
aacaaatgcctagaaamtattggagaattagaa

SEQ ID NO: 439 - DNA, 33 bp, Homo sapiens
tttaaataacttgagasaatttcttcttgaaaa

SEQ ID NO: 440 - DNA, 33 bp, Homo sapiens
cctaatattttctagarccctctccaaaaggtc

SEQ ID NO: 441 - DNA, 33 bp, Homo sapiens
ggagggagagagagcasacctaaatcttctacc

SEQ ID NO: 442 - DNA, 33 bp, Homo sapiens
tcaacacggaagccccraaagaatcttcagaca

SEQ ID NO: 443 - DNA, 33 bp, Homo sapiens
ctgcttcttcctggtcrcctattctcaatcctt

SEQ ID NO: 444 - DNA, 33 bp, Homo sapiens
ataaaagtctataatawgaggcacctatgcaaa

SEQ ID NO: 445 - DNA, 33 bp, Homo sapiens
ttcgggcattatatacyggtgttttggacaaag

SEQ ID NO: 446 - DNA, 33 bp, Homo sapiens
tgctcataggagagaayataagaactctgagtg

SEQ ID NO: 447 - DNA, 33 bp, Homo sapiens
agcagaataatgtgacscaaaatcaagagggaa

SEQ ID NO: 448 - DNA, 33 bp, Homo sapiens
aaataatagagatccawcctagggagccatttg

SEQ ID NO: 449 - DNA, 33 bp, Homo sapiens
gagtacactaaccagayggatataaacatgagg

SEQ ID NO: 450 - DNA, 33 bp, Homo sapiens
agccataaggaaatgcraggtcactgcttccag

SEQ ID NO: 451 - DNA, 33 bp, Homo sapiens
gtatttagtttggtgcmgaagtaacgcacaatt

SEQ ID NO: 452 - DNA, 33 bp, Homo sapiens
tactgcaaagaacacakgaattacacctttctt

SEQ ID NO: 453 - DNA, 33 bp, Homo sapiens
tacacactggaattaayaaaagcttttgcatta

SEQ ID NO: 454 - DNA, 33 bp, Homo sapiens
atcacaagagggtaaamtcagtgtcacagcaga

SEQ ID NO: 455 - DNA, 33 bp, Homo sapiens
tttggtaaactttgaarcactactttatctact

SEQ ID NO: 456 - DNA, 32 bp, Homo sapiens
ttacgagttcagtaaytgtgggttgatgatgg

SEQ ID NO: 457 - DNA, 33 bp, Homo sapiens
taatgattgaccttatkacaactctatatgact

SEQ ID NO: 458 - DNA, 33 bp, Homo sapiens
agatggtcatgtttcayggaatttacataataa

SEQ ID NO: 459 - DNA, 33 bp, Homo sapiens
ggatttatcctacaaasgcatataaatgggaaa

SEQ ID NO: 460 - DNA, 33 bp, Homo sapiens
aaaagtatacaagaacyctatggcaatgtcaag

SEQ ID NO: 461 - DNA, 33 bp, Homo sapiens
ttctgttctcttcccarctgacaaggaaacttc

SEQ ID NO: 462 - DNA, 33 bp, Homo sapiens
acacatatttgcgttcrccacatacagtacaca

SEQ ID NO: 463 - DNA, 33 bp, Homo sapiens
tttctcttcactcccarctaaaacgtaagctca

SEQ ID NO: 464 - DNA, 33 bp, Homo sapiens
gaattctctcaaaaacycaaaataaagttgttc

SEQ ID NO: 465 - DNA, 33 bp, Homo sapiens
atgctatcttcaaaagscaataataccaaagga

SEQ ID NO: 466 - DNA, 33 bp, Homo sapiens
aagagcacctggatacyggggtcatgttagcgc

SEQ ID NO: 467 - DNA, 33 bp, Homo sapiens
atgtccagagaatagcraggaagagaattgtag

SEQ ID NO: 468 - DNA, 33 bp, Homo sapiens
taaatgcaaaaataaasgtagaaacaatgtgct

SEQ ID NO: 469 - DNA, 33 bp, Homo sapiens
taagtgcaattaggaaygagtggaaacaatgag

SEQ ID NO: 470 - DNA, 33 bp, Homo sapiens
cccaaaactcgaagtcyaaagtcatatccaaga

SEQ ID NO: 471 - DNA, 33 bp, Homo sapiens
tcaacactacagcaacmaagttattagctagta

SEQ ID NO: 472 - DNA, 33 bp, Homo sapiens
agaggcttaggatataygccctgttcaatattt

SEQ ID NO: 473 - DNA, 33 bp, Homo sapiens
tttgctaacgaggctawgtggtagatttttttt

SEQ ID NO: 474 - DNA, 33 bp, Homo sapiens
caaagggcagaacgtgragcagcatttgaggct

SEQ ID NO: 475 - DNA, 33 bp, Homo sapiens
ctaattcttttggatastaaaccatgaaaaatg

SEQ ID NO: 476 - DNA, 33 bp, Homo sapiens
gagcttcctcttctgayggcaggagtttgaagc

SEQ ID NO: 477 - DNA, 33 bp, Homo sapiens
atgtatccattacccasatttaagaaataatat

SEQ ID NO: 478 - DNA, 33 bp, Homo sapiens
gtttgcatacatcacargttatttggggaagta

SEQ ID NO: 479 - DNA, 33 bp, Homo sapiens
tgtgttgcacgttcagrcaatggctcctaaaat

SEQ ID NO: 480 - DNA, 33 bp, Homo sapiens
ctgccctcattgaacaraggaaaactgagatgc

SEQ ID NO: 481 - DNA, 33 bp, Homo sapiens
caccactggagaggtaygacacactggtgtgtg

SEQ ID NO: 482 - DNA, 33 bp, Homo sapiens
tcacttggatgtataamcgtgaacaaactactt

SEQ ID NO: 483 - DNA, 33 bp, Homo sapiens
tgaacaaaatggatgargttctatcttcctgag

SEQ ID NO: 484 - DNA, 33 bp, Homo sapiens
ccaaatataacaaaaamcagatcataaacctca

SEQ ID NO: 485 - DNA, 33 bp, Homo sapiens
cctgtgtggagactgcraaatacagacttttta

SEQ ID NO: 486 - DNA, 33 bp, Homo sapiens
aggatgctgagcttccragaaattgatggacct

SEQ ID NO: 487 - DNA, 33 bp, Homo sapiens
ctggtgtatcttggtaraagctttagaaagttt

SEQ ID NO: 488 - DNA, 33 bp, Homo sapiens
ttagcaaatgaatgaaraattatttagcagctg

SEQ ID NO: 489 - DNA, 33 bp, Homo sapiens
actacctcattatacgwaagacctaattcaatc

SEQ ID NO: 490 - DNA, 33 bp, Homo sapiens
tggggatagagggaaamaggattctgagctagg

SEQ ID NO: 491 - DNA, 33 bp, Homo sapiens
tttggctttagggaatkaaattatttctggctg

SEQ ID NO: 492 - DNA, 33 bp, Homo sapiens
aaaaaaaggaaaagccrgaatgctatggactga

(19) United States
(12) Patent Application Publication
(10) Pub. No.: US 2008/0305967 A1
(43) Pub. Date: Dec. 11, 2008

(54) Genetic Markers Associated with Endometriosis and Use Thereof

(75) Inventors: Kenneth Ward, Salt Lake City, UT (US); Hans Albertsen, Salt Lake City, UT (US)

Correspondence Address: Michael R. Schramm;Juneau Biosciences, LLC, 2749 East Parleys Way, Suite 200, Salt Lake City, UT, 84109, US

(73) Assignee: JUNEAU BIOSCIENCES, LLC, Salt Lake City, UT (US)

(21) Appl. No.: 12/056,754

(22) Filed: Mar. 27, 2008

Related U.S. Application Data
(60) Provisional application No. 60/943,193, filed on Jun. 11, 2007.

(51) Int. Cl. C40B 40/06; C12Q 1/68; C07H 21/04

(52) U.S. Cl. 506/16; 435/6; 536/23.1; 536/24.5

(57) ABSTRACT

The present invention relates to novel genetic markers associated with endometriosis and risk of developing endometriosis, and methods and materials for determining whether a human subject has endometriosis or is at risk of developing endometriosis.

### CROSS REFERENCE TO RELATED APPLICATION

This application claims the benefit under 35 U.S.C. §119(e) of U.S. Provisional Patent Application No. 60/943,193, filed Jun. 11, 2007, which is incorporated herein in its entirety by this reference.

### FIELD OF THE INVENTION

The present invention relates to endometriosis diagnosis and therapy. In particular, the present invention relates to specific single nucleotide polymorphisms (SNPs) in the human genome, and their association with endometriosis and related pathologies.

### BACKGROUND OF THE INVENTION

Endometriosis in one instance refers to autoimmune endometriosis, mild endometriosis, moderate endometriosis or severe endometriosis. For the purpose of this invention the term endometriosis is used to describe any of these conditions.

Endometriosis is most generally defined as the presence of endometrium (glands and stroma) at sites outside of the uterus (ectopic endometrial tissues rather than eutopic or within the uterus). The most common sites are the ovaries, pelvic peritoneum, uterosacral ligaments, pouch of Douglas, and rectovaginal septum although implants have been identified on the peritoneal surfaces of the abdomen (these may grow into the intestines, ureters or bladder), in the thorax, at the umbilicus, and at incision sites of prior surgeries (Child T J, Tan S L (2001) Endometriosis: aetiology, pathogenesis and treatment. Drugs 61:1735-1750; Giudice et al. (1998) Status of current research on endometriosis. The Journal of reproductive medicine 43:252-262).

Endometriosis is a common gynecologic disorder. The prevalence is difficult to know. It has been estimated that it affects approximately 14% of all women (range 1-43%), 40-60% of women with pelvic pain and 30%-50% of infertile women (Di Blasio et al. (2005) Genetics of endometriosis. Minerva ginecologica 57:225-236; Schindler A E (2004) Pathophysiology, diagnosis and treatment of endometriosis. Minerva ginecologica 56:419-435).

Studies of the inheritance of endometriosis have been hampered by methodological problems related to disease definition and control selection. General population incidence during the 1970s in this country has been suggested to be 1.6 per 1000 white females aged 15-49, while a more current study based upon hospital discharges finds endometriosis as a first listed diagnosis in 1.3 per 1000 discharges in women aged 15-44. There is a clinical impression that blacks have lower rates of endometriosis and Orientals have higher rates than whites. Separate work has suggested a polygenic/multifactorial inheritance (Vigano P, Somigliana E, Vignali M, Busacca M, Blasio A M (2007) Genetics of endometriosis: current status and prospects. Front Biosci 12:3247-3255). Affected sib-pair studies have also performed (Kennedy et al. (2001) Affected sib-pair analysis in endometriosis. Human reproduction update 7:411-418; Treloar et al. (2005) Genomewide linkage study in 1,176 affected sister pair families identifies a significant susceptibility locus for endometriosis on chromosome 10q26. Am J Hum Genet 77:365-376).

Specific genes with polymorphisms have been investigated for an association with endometriosis. Some association studies implicated GALT (a gene involved in galactose metabolism), and GSTM1 and NAT2 (genes encoding for the detoxification enzymes) as possible disease susceptibility genes. Recent findings have added to the evidence for the involvement of GSTM1 and NAT2, but have cast doubt on the role of GALT. The p21 gene codon 31 arginine/serine polymorphism is not associated with endometriosis.

Polymorphisms of the arylhydrocarbon receptor (AHR) gene and related genes were examined, and in at least one study, no association was found. However, the design of many genetic and epidemiological studies has been inadequate with respect to sample size, consistency in phenotype definition, and the choice of control populations. To identify genomic changes involved in the development of endometriosis (Gogusevet al. (1999). “Detection of DNA copy number changes in human endometriosis by comparative genomic hybridization.” Hum Genet 105(5): 444-51) examined endometriotic tissues by comparative genomic hybridization and detected losses of 1p and 22q in 50% of the cases. Additional common losses included 7p (22%). Dual-color FISH using probes for the deleted regions on chromosomes 1, 7, and 22 supported the CGH data. Treloar et al. (Treloar et al. (2005). “Genomewide linkage study in 1,176 affected sister pair families identifies a significant susceptibility locus for endometriosis on chromosome 10q26.” Am J Hum Genet 77(3): 365-76) conducted a linkage study of 1,176 families (931 Australian and 245 from the U.K.), each with at least 2 affected family members, usually affected sister pairs, with surgically diagnosed disease. They identified a region of significant linkage on 10q26 (maximum lod score=3.09; genomewide P=0.047) and another region of suggestive linkage on 20p13; minor peaks were found on 8 other chromosomes.

Endometriosis is a genetically inherited disease. Genetic variation in DNA sequences is often associated with heritable phenotypes, such as an individual's propensity towards complex disorders. Single nucleotide polymorphisms are the most common form of genetic sequence variations. Detection and analysis of specific genetic mutations, such as single nucleotide polymorphisms (SNPs), which are associated with endometriosis risk, may therefore be used to determine risk of endometriosis, the presence of endometriosis or the progression of endometriosis. Genetic markers that are prognostic for endometriosis can be genotyped early in life and could predict individual response to various risk factors and treatment. Genetic predisposition revealed by genetic analysis of susceptibility genes can provide an integrated assessment of the interaction between genotypes and environmental factors, resulting in synergistically increased prognostic value of diagnostic tests. Thus, pre-symptomatic and early symptomatic genetic testing is expected to be the cornerstone of the paradigmatic shift from late surgical interventions to earlier preventative therapies.

Thus, there is an urgent need for novel genetic markers that are predictive of endometriosis and endometriosis progression, particularly in treatment decisions for individuals who are recognized as having endometriosis. Such genetic markers may enable prognosis of endometriosis in much larger populations compared with the populations which can currently be evaluated by using existing risk factors and biomarkers. The availability of a genetic test may allow, for example, early diagnosis and prognosis of endometriosis, as well as early clinical intervention to mitigate progression of the disease. The use of these genetic markers will also allow selection of subjects for clinical trials involving novel treatment methods. The discovery of genetic markers associated with endometriosis will further provide novel targets for therapeutic intervention or preventive treatments of endometriosis and enable the development of new therapeutic agents for treating endometriosis.

### SUMMARY OF THE INVENTION

The present invention relates to the identification of novel SNPs, unique combinations of such SNPs, and haplotypes of SNPs that are associated with endometriosis and related pathologies. The polymorphisms disclosed herein are directly useful as targets for the design of diagnostic reagents and the development of therapeutic agents for use in the diagnosis and treatment of endometriosis and related pathologies.

Based on the identification of SNPs associated with endometriosis, the present invention also provides methods of detecting these variants as well as the design and preparation of detection reagents needed to accomplish this task. The invention specifically provides novel SNPs in genetic sequences involved in endometriosis, methods of detecting these SNPs in a test sample, methods of identifying individuals who have an altered risk of developing endometriosis and for suggesting treatment options for endometriosis based on the presence of a SNP(s) disclosed herein or its encoded product and methods of identifying individuals who are more or less likely to respond to a treatment.

In one embodiment of the invention, the present invention provides SNPs, as set forth in Tables 1-2 having significant allelic association with endometriosis or by being co-located within the same LD blocks as the SNPs listed in Tables 1 and 2, and set forth in Tables 3-196.

Tables 1-196 provide information identifying SNPs of the present invention, including SNP “rs” identification numbers (a reference SNP or RefSNP accession ID number) or “SNP-A” identification numbers (as used by Affymetrix, Santa Clara, Calif.), chromosome number, and base position number of the SNP.

In a specific embodiment of the present invention, naturally-occurring SNPs in the human genome are provided that are associated with endometriosis. Such SNPs can have a variety of uses in the diagnosis and/or treatment of endometriosis. One aspect of the present invention relates to an isolated nucleic acid molecule comprising a nucleotide sequence in which at least one nucleotide is a SNP disclosed in Table 1 or Table 2. In an alternative embodiment, a nucleic acid of the invention is an amplified polynucleotide, which is produced by amplification of a SNP-containing nucleic acid template.

In yet another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest.

Also provided in the invention are kits comprising SNP detection reagents, and methods for detecting the SNPs disclosed herein by employing detection reagents. In a specific embodiment, the present invention provides for a method of identifying an individual having an increased or decreased risk of developing endometriosis by detecting the presence or absence of a SNP allele disclosed herein. In another embodiment, a method for diagnosis of endometriosis by detecting the presence or absence of a SNP allele disclosed herein is provided. In yet another embodiment a method for predicting endometriosis sub-classification by detecting the presence or absence of a SNP allele disclosed herein is provided.

In yet another embodiment, the invention also provides a kit comprising SNP detection reagents, and methods for detecting the SNPs disclosed herein by employing detection reagents and a questionnaire of non-genetic clinical factors. In one embodiment, the questionnaire would be completed by a medical professional based on medical history physical exam or other clinical findings. In yet another embodiment, the questionnaire would include any other non-genetic clinical factors known to be associated with the risk of developing endometriosis.

Many other uses and advantages of the present invention will be apparent to those skilled in the art upon review of the detailed description of the preferred embodiments herein. Solely for clarity of discussion, the invention is described in the sections below by way of non-limiting examples.

### DETAILED DESCRIPTION OF THE INVENTION

Definitions

“Haplotype” means a combination of genotypes on the same chromosome occurring in a linkage disequilibrium block. Haplotypes serve as markers for linkage disequilibrium blocks, and at the same time provide information about the arrangement of genotypes within the blocks. Typing of only certain SNPs which serve as tags can, therefore, reveal all genotypes for SNPs located within a block. Thus, the use of haplotypes greatly facilitates identification of candidate genes associated with diseases and drug sensitivity.

The term “linkage disequilibrium” or “LD” means that a particular combination of alleles (alternative nucleotides) or genetic markers at two or more different SNP sites within a given chromosomal region are non-randomly co-inherited, meaning that the combination of alleles at the different SNP sites occurs more or less frequently in a population than the separate frequencies of occurrence of each allele or the frequency of a random formation of haplotypes from alleles in a given population. LD” differs from “linkage,” which describes the association of two or more loci on a chromosome with limited recombination between them. LD is also used to refer to any non-random genetic association between allele(s) at two or more different SNP sites. Therefore, when a SNP is in LD with other SNPs, the particular allele of the first SNP often predicts which alleles will be present in those SNPs in LD. LD is generally due to the physical proximity of the two loci along a chromosome. Hence, genotyping one of the SNP sites will give almost the same information as genotyping the other SNP site that is in LD. LD is caused by fitness interactions between genes or by such non-adaptive processes as population structure, inbreeding, and stochastic effects.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome. The average LD block size in Caucasians has been estimated to 16.3 kb occasionally extending across several hundred kb. LD blocks may also vary in size between ethnic groups (The International HapMap Consortium “A haplotype map of the human genome.” Nature (2005) 437: 1299-1320). Conservatively, LD can be defined as SNPs that have a D prime value of 1 and a LOD score greater than 2.0 or an r-squared value greater than 0.8.

“Linkage disequilibrium block” or “LD block” means a region of the genome that contains multiple SNPs located in proximity to each other and that are transmitted as a block.

“D prime” or “D′” (also referred to as the “linkage disequilibrium measure” or “linkage disequilibrium parameter”) means the deviation of the observed allele frequencies from the expected, and is a statistical measure of how well a biometric system can discriminate between different individuals. The larger the D′ value, the better a biometric system is at discriminating between individuals.

“LOD score” is the “logarithm of the odd” score, which is a statistical estimate of whether two genetic loci are physically near enough to each other (or “linked”) on a particular chromosome that they are likely to be inherited together. A LOD score of three or more is generally considered statistically significant evidence of linkage.

“R-squared” or “r²” (also referred to as the “correlation coefficient”) is a statistical measure of the degree to which two markers are related. The nearer to 1.0 the r² value is, the more closely the markers are related to each other. R² cannot exceed 1.0. D prime and LOD scores generally follow the above definition for SNPs in LD. R², however, displays a more complex pattern and can vary between about 0.0003 and 1.0 in SNPs that are in LD.

The present invention provides SNPs associated with endometriosis, nucleic acid molecules containing SNPs, methods and reagents for the detection of the SNPs disclosed herein, uses of these SNPs for the development of detection reagents, and assays or kits that utilize such reagents. The SNPs disclosed herein are useful for diagnosing, screening for, and evaluating predisposition to endometriosis and progression of endometriosis. Additionally, such SNPs are useful in the determining individual subject treatment plans and design of clinical trials of devices for possible use in the treatment of endometriosis. Furthermore, such SNPs and their encoded products are useful targets for the development of therapeutic agents. Furthermore, such SNPs combined with other non-genetic clinical factors are useful for diagnosing, screening, evaluating predisposition to endometriosis, assessing risk of progression of endometriosis, determining individual subject treatment plans and design of clinical trials of devices for possible use in the treatment of endometriosis.

SNPs

As used herein, the term “SNP” refers to single nucleotide polymorphisms in DNA. SNPs are usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel”.

A synonymous codon change, or silent mutation SNP (terms such as “SNP,” “polymorphism,” “mutation,” “mutant,” “variation,” and “variant” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. An indel that occur in a coding DNA segment gives rise to a frameshift mutation. While SNPs can be bi-, tri-, or tetra-allelic, the vast majority of the SNPs are bi-allelic, and are thus often referred to as “bi-allelic markers,” or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases.

Causative SNPs are those SNPs that produce alterations in gene expression or in the structure and/or function of a gene product, and therefore are predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic endometriosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions and miRNA recognition sites. Some SNPs that are not causative SNPs nevertheless are in close association with, and therefore segregate with, a disease-causing sequence. In this situation, the presence of a SNP correlates with the presence of, or predisposition to, or an increased risk in developing the endometriosis. These SNPs, although not causative, are nonetheless also useful for diagnostics, endometriosis predisposition screening, endometriosis progression risk and other uses.

An association study of a SNP and a specific disorder involves determining the presence or frequency of the SNP allele in biological samples from individuals with the disorder of interest, such as endometriosis, and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP may be screened in tissue samples or any biological sample obtained from an affected individual, and compared to control samples, and selected for its increased (or decreased) occurrence in a specific pathological condition, such as pathologies related to endometriosis. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype. Association studies may be conducted within the general population and are not limited to studies performed on related individuals in affected families (linkage studies). For diagnostic and prognostic purposes, if a particular SNP site is found to be useful for diagnosing a disease, such as endometriosis, other SNP sites which are in LD with this SNP site would also be expected to be useful for diagnosing the condition. Linkage disequilibrium is described in the human genome as blocks of SNPs along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r² used to determine linkage disequilibrium.

Tables 1 and 2 disclose SNPs that have been shown in case-control studies to be associated with endometriosis. Table 1 specifically shows groups of 2 or more SNPs from the 500K GeneChip that all showed significant association with endometriosis and are positioned 50 kb or less from each other (referred to as “anchors”). Table 2 shows SNPs having significant association with endometriosis but for which no other SNP present on the 500K GeneChip and located within 50 kb showed significant association with endometriosis (referred to as “singletons”). Tables 1 and 2 provide identifying information regarding each SNP in columns labeled “dbSNPrsID” (the NCBI reference SNP identifier, “Chr” (the Chromosome where the SNP is located; note that the chromosome numbered “23” is used interchangeably for chromosome “X”), “Position” (the basepair position on the chromosome indicated), “P-Value” (the p-value calculated by PLINK), “OR” (the Odds Ratio for the SNP in question), “F_A” (the minor allele frequency observed in the endometriosis affected cases), “F_U” (the minor allele frequency observed in the control individuals), and “FlankSequence” (the DNA sequence surrounding the SNP in question). The two allelic variants observed for the SNP are indicated in square brackets in the middle of the sequence.

Tables 3-196 define the linkage disequilibrium blocks surrounding each of the Anchor and Singleton SNPs identified in Tables 1 and 2 above. The linkage disequilibrium blocks were ascertained based upon the criteria set forth by the Haploview computer algorithm under default settings (Barrett J C, Fry B, Maller J, Daly M J. “Haploview: analysis and visualization of LD and haplotype maps.” Bioinformatics, vol. 21, pp 263-265, 2005).

Each of Tables 3-196 is prefaced by one or more SNPs from the initial anchor and singleton Tables 1 or 2, and includes a list of one or more SNPs that correspond to a linkage disequilibrium block, including the anchor and singleton SNPs, which are highlighted in bold character within the table. Occasionally, an original anchor or singleton marker may not itself be present in the SNP list in which case the rsID and basepair position of the neighboring SNPs are highlighted in bold character. Also indicated in the tables is the chromosome, physical position in basepairs, minor allele frequency and observed alleles for each SNP. On rare occasions, a SNP falls outside of a linkage disequilibrium block, in which case the list is left empty.

The SNPs shown in Tables 1-196 may be useful individually, in combination with one of the other SNPs or in a haplotype involving one of the other SNPs in Tables 1-196. Linkage disequilibrium blocks can be determined from genomewide genetic population studies which results are accessible in private and public databases, and can be visualized or tabularized using, for example, the Haploview software (Barrett J C, Fry B, Maller J, Daly M J. Haploview: analysis and visualization of LD and haplotype maps. Bioinformatics. Jan. 15, 2005). The linkage disequilibrium blocks described in Tables 1-196 were identified using Haploview version 4 based on the International HapMap Consortium data release 21.

In accordance with the present invention, SNPs have been identified in a study using a whole-genome case-control approach to identify single nucleotide polymorphisms that were closely associated with the development of endometriosis, as well as SNPs found to be in linkage disequilibrium with (i.e., within the same linkage disequilibrium block as) the endometriosis-associated SNPs, which can provide haplotypes (i.e., groups of SNPs that are co-inherited) to be readily inferred. Thus, the present invention provides individual SNPs associated with endometriosis, as well as combinations of SNPs and haplotypes in genetic regions associated with endometriosis, methods of detecting these polymorphisms in a test sample, methods of determining the risk of an individual of having or developing endometriosis and for clinical sub-classification of endometriosis.

The present invention also provides SNPs associated with endometriosis, as well as SNPs that were previously known in the art, but were not previously known to be associated with endometriosis. Accordingly, the present invention provides novel compositions and methods based on the SNPs disclosed herein, and also provides novel methods of using the known but previously unassociated SNPs in methods relating to endometriosis (e.g., for diagnosing endometriosis. etc.).

Particular SNP alleles of the present invention can be associated with either an increased risk of having or developing endometriosis, or a decreased risk of having or developing endometriosis. SNP alleles that are associated with a decreased risk may be referred to as “protective” alleles, and SNP alleles that are associated with an increased risk may be referred to as “susceptibility” alleles, “risk factors”, or “high-risk” alleles. Thus, whereas certain SNPs can be assayed to determine whether an individual possesses a SNP allele that is indicative of an increased risk of having or developing endometriosis (i.e., a susceptibility allele), other SNPs can be assayed to determine whether an individual possesses a SNP allele that is indicative of a decreased risk of having or developing endometriosis (i.e., a protective allele). Similarly, particular SNP alleles of the present invention can be associated with either an increased or decreased likelihood of responding to a particular treatment. The term “altered” may be used herein to encompass either of these two possibilities (e.g., an increased or a decreased risk/likelihood).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods disclosed herein may generally target either strand. Throughout the specification, in identifying a SNP position, reference is generally made to the forward or “sense” strand, solely for the purpose of convenience. Since endogenous nucleic acid sequences exist in the form of a double helix (a duplex comprising two complementary nucleic acid strands), it is understood that the SNPs disclosed herein will have counterpart nucleic acid sequences and SNPs associated with the complementary “reverse” or “antisense” nucleic acid strand. Such complementary nucleic acid sequences, and the complementary SNPs present in those sequences, are also included within the scope of the present invention.

Isolated Nucleic Acid Molecules

The present invention provides isolated nucleic acid molecules that contain one or more SNPs disclosed Tables 1-196. Tables 1 and 2 provide context nucleic acid sequences. Tables 3-196 provide only rs identification numbers; however, the context sequences for such SNPs are known and disclosed in the art, and are not therefore shown in the tables. Isolated nucleic acid molecules contain one or more SNPs identified in Tables 1-196. Isolated nucleic acid molecules containing one or more SNPs disclosed in Tables 1-196 may be interchangeably referred to throughout the present text as “SNP-containing nucleic acid molecules.” The isolated nucleic acid molecules of the present invention also include probes and primers (which are described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” generally is one that contains a SNP of the present invention or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated.” Nucleic acid molecules present in non-human transgenic animals, which do not naturally occur in the animal, are also considered “isolated”. For example, recombinant DNA molecules contained in a vector are considered “isolated”. Further examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. The flanking sequence may be up to about 100, 60, 50, 30, 25, 20, 15, 10, 8, or 4 nucleotides (or any other length in-between) on either side of a SNP position.

For full-length genes and entire protein-coding sequences, a SNP flanking sequence can be, for example, up to, but not limited to, about 5 KB, 4 KB, 3 KB, 2 KB, 1 KB on either side of the SNP. Furthermore, in such instances, the isolated nucleic acid molecule comprises exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs disclosed herein are also encompassed by the present invention, and such fragments may be used, for example, to express any part of a protein, such as a particular functional domain or an antigenic epitope.

Thus, the present invention also encompasses fragments of the nucleic acid sequences contiguous to the SNPs disclosed in Tables 1-196, contiguous nucleotide sequence at least about 8 or more nucleotides, more preferably at least about 12 or more nucleotides, and even more preferably at least about 16 or more nucleotides. Further, a fragment could comprise at least about 18, 20, 22, 25, 30, 40, 50, 60, 100, 250 or 500 (or any other number in-between) nucleotides in length. The length of the fragment will be based on its intended use. For example, the fragment can be useful as a polynucleotide probe or primer. Such fragments can be isolated using nucleotide sequences comprising one of the SNPs in Tables 1-196 for the synthesis of a polynucleotide probe. A labeled probe can then be used, for example, to screen a cDNA library, genomic DNA library, or mRNA to isolate nucleic acid corresponding to the coding region. Further, primers can be used in amplification reactions, such as for purposes of assaying one or more SNPs sites or for cloning specific regions of a gene.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In a preferred embodiment of the invention, an amplified polynucleotide is at least about 30 nucleotides in length. In a more preferred embodiment of the invention, an amplified polynucleotide is at least about 32, 40, 45, 50, or 60 nucleotides in length. In yet another preferred embodiment of the invention, an amplified polynucleotide is at least about 100, 200, or 300 nucleotides in length. While the total length of an amplified polynucleotide of the invention can be as long as an exon, an intron or the entire gene where the SNP of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length. It is understood that irrespective of the length of an amplified polynucleotide, a SNP of interest may be located anywhere along its sequence.

In a specific embodiment of the invention, the amplified product is at least about 201 nucleotides in length, comprises one of the nucleotide sequences shown in Tables 1-196. Such a product may have additional sequences on its 5′ end or 3′ end or both. In another embodiment, the amplified product is about 101 nucleotides in length, and it contains a SNP disclosed herein. Generally, the SNP is located at the middle of the amplified product (e.g., at position 101 in an amplified product that is 201 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present invention provides isolated nucleic acid molecules that comprise, consist of, or consist essentially of one or more SNPs disclosed herein, complements thereof, and SNP-containing fragments thereof.

Accordingly, the present invention provides nucleic acid molecules that consist of any of the nucleotide sequences comprising one of the SNPs shown in Tables 1-196. A nucleic acid molecule consists of a nucleotide sequence when the nucleotide sequence is the complete nucleotide sequence of the nucleic acid molecule.

The present invention further provides nucleic acid molecules that consist essentially of any of the SNPs shown in Tables 1-196. A nucleic acid molecule consists essentially of a nucleotide sequence when such a nucleotide sequence includes only one of the SNPs disclosed in Tables 1-196, and no other SNPs associated with endometriosis, although additional nucleotide sequence may be included that does not include any additional SNPs associated with endometriosis.

The present invention further provides nucleic acid molecules that comprise any of the SNPs shown in Tables 1-196. A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated are well known to those of ordinary skill in the art (Sambrook and Russell, 2000, Molecular Cloning: A Laboratory Manual, Cold Spring Harbor Press, NY).

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition,” Trends Biotechnol. June 1997;15(6):224-9, and Hyrup et al., “Peptide nucleic acids (PNA): synthesis, properties and potential applications,” Bioorg Med Chem. January 1996; 4(1):5-23).

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in Tables 1-196. Furthermore, kits/systems (such as beads, arrays, etc.) that include these analogs are also encompassed by the present invention.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002).

Further variants of the SNPs disclosed in Tables 1-196, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence contiguous to the SNPs disclosed in Tables 1-196 (or a fragment thereof) and that includes a novel SNP allele disclosed in Tables 1-196. Thus, the present invention specifically contemplates isolated nucleic acid molecule that have a certain degree of sequence variation compared with the sequences shown in Tables 1-196, but that contain a novel SNP allele disclosed herein. In other words, as long as an isolated nucleic acid molecule contains a novel SNP allele disclosed herein, other portions of the nucleic acid molecule that flank the novel SNP allele can vary to some degree from the specific genomic and context sequences surrounding the SNPs listed in Tables 1-196.

To determine the percent identity of two nucleotide sequences of two molecules that share sequence homology, the sequences are aligned for optimal comparison purposes (e.g., gaps can be introduced in one or both of a first and a second nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The nucleotides at corresponding nucleotide positions are then compared. When a position in the first sequence is occupied by the same nucleotide as the corresponding position in the second sequence, then the molecules are identical at that position (as used herein, nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, N. J., 1994; Sequence Analysis in Molecular Biology, von Heinje, G., Academic Press, 1987; and Sequence Analysis Primer, Gribskov, M. and Devereux, J., eds., M Stockton Press, New York, 1991).

In one particular embodiment, the percent identity between two nucleotide sequences is determined using the GAP program in the GCG software package (Devereux, J., et al., Nucleic Acids Res. 12(1):387 (1984)), using a NWSgapdna, CMP matrix and a gap weight of 40, 50, 60, 70, or 80 and a length weight of 1, 2, 3, 4, 5, or 6. In another embodiment, the percent identity between two nucleotide sequences is determined using the algorithm of E. Myers and W. Miller (CABIOS, 4:11-17 (1989)) which has been incorporated into the ALIGN program (version 2.0), using a PAM120 weight residue table, a gap length penalty of 12, and a gap penalty of 4.

The nucleotide sequences of the present invention can further be used as a “query sequence” to perform a search against sequence databases to, for example, identify other family members or related sequences. Such searches can be performed using the NBLAST and XBLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g., XBLAST and NBLAST) can be used. In addition to BLAST, examples of other search and sequence comparison programs used in the art include, but are not limited to, FASTA (Pearson, Methods Mol. Biol. 25, 365-389 (1994)) and KERR (Dufresne et al., Nat Biotechnol December 2002; 20(12):1269-71). For further information regarding bioinformatics techniques, see Current Protocols in Bioinformatics, John Wiley & Sons, Inc., N.Y. Similarly, individual gene products identified by association to the SNP listed in Tables 1-196, are expected to participate together with other proteins in specific regulatory pathways that can cause or modulate the progression of endometriosis. Such genes and their products might also be candidates for diagnostic and therapeutic intervention.

The present invention further provides non-coding fragments of the nucleic acid molecules disclosed in Tables 1-196. Preferred non-coding fragments include, but are not limited to, promoter sequences, enhancer sequences, intronic sequences, 5′ untranslated regions (UTRs), 3′ untranslated regions, gene modulating sequences and gene termination sequences. Such fragments are useful, for example, in controlling heterologous gene expression and in developing screens to identify gene-modulating agents.

SNP Detection Reagents

In a specific aspect of the present invention, the SNPs disclosed herein can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a probe that hybridizes to a target nucleic acid containing one or more of the SNPs disclosed herein. In a preferred embodiment, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding to the context sequences provided in the SNPs disclosed herein. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

In one preferred embodiment of the invention, a SNP detection reagent is a synthetic polynucleotide molecule, such as an isolated or synthetic DNA or RNA polynucleotide probe or primer or PNA oligomer, or a combination of DNA, RNA and/or PNA that hybridizes to a segment of a target nucleic acid molecule containing a SNP identified herein. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer typically is a substantially purified oligonucleotide. Such oligonucleotide typically comprises a region of complementary nucleotide sequence that hybridizes under stringent conditions to at least about 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

Other preferred primer and probe sequences can readily be determined using the nucleotide sequences disclosed herein. It will be apparent to one of skill in the art that such primers and probes are directly useful as reagents for genotyping the SNPs of the present invention, and can be incorporated into any kit/system format.

In order to produce a probe or primer specific for a target SNP-containing sequence, the gene/transcript and/or context sequence surrounding the SNP of interest is typically examined using a computer algorithm which starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235,726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5× standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment which is particularly suitable for use in a oligonucleotide ligation assay (U.S. Pat. No. 4,988,617), the most 3′ nucleotide of the probe aligns with the SNP position in the target sequence.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22:1859; and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position. Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of one allelic form to which the primer exhibits perfect complementarity (Gibbs, 1989, Nucleic Acid Res. 17:2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan assay, described below.

In a specific embodiment of the invention, a primer of the invention contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment of the invention, a SNP detection reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the invention, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl. 4:357-362; Tyagi et al., 1996, Nature Biotechnology 14: 303-308; Nazarenko et al., 1997, Nucl. Acids Res. 25:2516-2521; U.S. Pat. Nos. 5,866,336 and 6,117,635).

The detection reagents of the invention may also contain other labels, including but not limited to, biotin for streptavidin binding and oligonucleotide for binding to another complementary oligonucleotide such as pairs of zipcodes.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the 3′-most end of the primer extension product, and in which the ddNTP corresponds to a SNP disclosed herein, is a composition that is encompassed by the present invention). Thus, reagents that bind to a nucleic acid molecule in a region adjacent to a SNP site, even though the bound sequences do not necessarily include the SNP site itself, are also encompassed by the present invention.

SNP Detection Kits and Systems

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting at least 1; 10; 100; 1000; 10,000; 100,000; 500,000 (or any other number in-between) or substantially all of the SNPs disclosed herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al., PCT application WO95/11995 (Chee et al.), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002;8:85-101; Sosnowski et al., “Active microelectronic array system for DNA hybridization, genotyping and pharmacogenomic applications”, Psychiatr Genet. December 2002; 12(4):181-92; Heller, “DNA microarray technology: devices, systems, and applications”, Annu Rev Biomed Eng. 2002;4:129-53. Epub Mar. 22, 2002; Kolchinsky et al., “Analysis of SNPs and other genomic variations using gel-based chips”, Hum Mutat. April 2002;19(4):343-60; and McGall et al., “High-density genechip oligonucleotide probe arrays”, Adv Biochem Eng Biotechnol. 2002;77:21-42.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of the target SNP site, sequential polynucleotides that cover the full-length sequence of a gene/transcript; or unique polynucleotides selected from particular areas along the length of a target gene/transcript sequence, particularly areas corresponding to one or more SNPs disclosed herein. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,958, 5,843,681, 5,800,999, and 5,773,628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 15-25 nucleotides in length. In further embodiments, a nucleic acid array can comprise any number of probes, in which at least one probe is capable of detecting one or more SNPs disclosed in Table 1 and/or at least one probe comprises a fragment of one of the sequences selected from the group consisting of those disclosed herein, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a SNP. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts, including DNA and/or RNA, extracts from any bodily fluids. In a preferred embodiment of the invention, the bodily fluid is blood, saliva or buccal swabs. The test samples used in the above-described methods will vary based on such factors as the assay format, nature of the detection method, and the specific tissues, cells or extracts used as the test sample to be assayed. Methods of preparing nucleic acids are well known in the art and can be readily adapted to obtain a sample that is compatible with the system utilized.

In yet another form of the kit in addition to reagents for preparation of nucleic acids and reagents for detection of one of the SNPs of this invention, the kit may include a questionnaire inquiring about non-genetic clinical factors such as age, gender, or any other non-genetic clinical factors known to be associated with endometriosis.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development”, Adv Drug Deliv Rev. Feb. 24, 2003;55(3):349-77). In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073, Dubrow et al., and U.S. Pat. No. 6,156,181, Parce et al.

For genotyping SNPs, a microfluidic system may integrate, for example, nucleic acid amplification, primer extension, capillary electrophoresis, and a detection method such as laser induced fluorescence detection.

Uses of Nucleic Acid Molecules

The nucleic acid molecules of the present invention have a variety of uses, especially in the diagnosis and treatment of endometriosis. For example, the nucleic acid molecules are useful as hybridization probes, such as for genotyping SNPs in messenger RNA, transcript, cDNA, genomic DNA, amplified DNA or other nucleic acid molecules comprising one of the SNPs disclosed in Tables 1-196, as well as their orthologs.

A probe can hybridize to any nucleotide sequence along the entire length of a nucleic acid molecule encompassing a SNP of the present invention. Preferably, a probe of the present invention hybridizes to a region of a target sequence that encompasses a SNP. More preferably, a probe hybridizes to a SNP-containing target sequence in a sequence-specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. These uses are relevant for diagnosis of disorders involving an increase or decrease in gene expression relative to normal levels. In vitro techniques for detection of mRNA include, for example, Northern blot hybridizations and in situ hybridizations. In vitro techniques for detecting DNA include Southern blot hybridizations and in situ hybridizations (Sambrook and Russell, 2000, Molecular Cloning: A Laboratory Manual, Cold Spring Harbor Press, Cold Spring Harbor, N.Y.).

Probes can be used as part of a diagnostic test kit for identifying cells or tissues in which a variant protein is expressed, such as by measuring the level of a variant protein-encoding nucleic acid (e.g., mRNA) in a sample of cells from a subject or determining if a polynucleotide contains a SNP of interest.

Thus, the nucleic acid molecules of the invention can be used as hybridization probes to detect the SNPs disclosed herein, thereby determining whether an individual with the polymorphisms is at risk for endometriosis or has developed early stage endometriosis. Detection of a SNP associated with an endometriosis phenotype provides a diagnostic and/or a prognostic tool for an active endometriosis and/or genetic predisposition to the endometriosis.

The nucleic acid molecules of the invention are also useful as primers to amplify any given region of a nucleic acid molecule, particularly a region containing a SNP of the present invention.

The nucleic acid molecules of the invention are also useful for constructing vectors containing a gene regulatory region of the nucleic acid molecules of the present invention. Further, the nucleic acid molecules of the invention also have therapeutic use in the form of siRNA (small interfering RNA).

SNP Genotyping Methods

The process of determining which specific nucleotide (i.e., allele) is present at each of one or more SNP positions, such as a SNP position in a nucleic acid molecule characterized by a SNP of the present invention, is referred to as SNP genotyping. The present invention provides methods of SNP genotyping, such as for use in screening for endometriosis or related pathologies, or determining predisposition thereto, or determining responsiveness to a form of treatment, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003;3(2):77-96; Kwok et al., “Detection of single nucleotide polymorphisms”, Curr Issues Mol. Biol. April 2003;5(2):43-60; Shi, “Technologies for individual genotyping: detection of genetic polymorphisms in drug targets and endometriosis genes”, Am J Pharmacogenomics. 2002;2(3):197-205; and Kwok, “Methods for genotyping single nucleotide polymorphisms”, Annu Rev Genomics Hum Genet 2001;2:235-58. Exemplary techniques for high-throughput SNP genotyping are described in Mamellos, “High-throughput SNP analysis for genetic association studies”, Curr Opin Drug Discov Devel. May 2003;6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (U.S. Pat. No. 6,734,294, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms include, but are not limited to, methods in which protection from cleavage agents is used to detect mismatched bases in RNA/RNA or RNA/DNA duplexes (Myers et al., Science 230:1242 (1985); Cotton et al., PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285:125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for endometriosis and related pathologies, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another preferred method for genotyping the SNPs of the present invention is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6,054,564 describe OLA strategies for performing SNP detection; WO 97/31256 and WO 00/56927 describe OLA strategies for performing SNP detection using universal arrays, wherein a zipcode sequence can be introduced into one of the hybridization probes, and the resulting product, or amplified product, hybridized to a universal zip code array; U.S. application Ser. Nos. 01/17329 (and 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

The following references provide further information describing mass spectrometry-based methods for SNP genotyping: Bocker, “SNP and mutation discovery using base-specific cleavage and MALDI-TOF mass spectrometry”, Bioinformatics. July 2003;19 Suppl 1:144-153; Storm et al., “MALDI-TOF mass spectrometry-based SNP genotyping”, Methods Mol. Biol. 2003;212:241-62; Jurinke et al., “The use of MassARRAY technology for high throughput genotyping”, Adv Biochem Eng Biotechnol. 2002;77:57-74; and Jurinke et al., “Automated genotyping using the DNA MassArray technology”, Methods Mol. Biol. 2002; 187:179-92.

An even more preferred method for genotyping the SNPs of the present invention is the use of electrospray mass spectrometry for direct analysis of an amplified nucleic acid (see, e.g., U.S. Pat. No. 6,734,294). In this method, in one aspect, an amplified nucleic acid product may be isotopically enriched in an isotope of oxygen (O), carbon (C), nitrogen (N) or any combination of those elements. In a preferred embodiment the amplified nucleic acid is isotopically enriched to a level of greater than 99.9% in the elements of O¹⁶, C^{12 and} N¹⁴ The amplified isotopically enriched product can then be analyzed by electrospray mass spectrometry to determine the nucleic acid composition and the corresponding SNP genotyping. Isotopically enriched amplified products result in a corresponding increase in sensitivity and accuracy in the mass spectrum. In another aspect of this method an amplified nucleic acid that is not isotopically enriched can also have composition and SNP genotype determined by electrospray mass spectrometry.

SNPs can also be scored by direct DNA sequencing. A variety of automated sequencing procedures can be utilized ((1995) Biotechniques 19:448), including sequencing by mass spectrometry (see, e.g., PCT International Publication No. WO94/16101; Cohen et al., Adv. Chromatogr. 36:127-162 (1996); and Griffin et al., Appl. Biochem. Biotechnol. 38:147-159 (1993)). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described below. Examples of such applications include, but are not limited to, SNP-endometriosis association analysis, endometriosis predisposition screening, endometriosis diagnosis, endometriosis prognosis, endometriosis progression monitoring, determining therapeutic strategies based on an individual's genotype, and stratifying a patient population for clinical trials for a treatment such as minimally invasive device for the treatment of endometriosis.

Analysis of Genetic Association Between SNPs and Phenotypic Traits

SNP genotyping for endometriosis diagnosis, endometriosis predisposition screening, endometriosis prognosis and endometriosis treatment and other uses described herein, typically relies on initially establishing a genetic association between one or more specific SNPs and the particular phenotypic traits of interest.

In a genetic association study, the cause of interest to be tested is a certain allele or a SNP or a combination of alleles or a haplotype from several SNPs. Thus, tissue specimens (e.g., saliva) from the sampled individuals may be collected and genomic DNA genotyped for the SNP(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in a endometriosis genetic association study, clinical information such as body mass index, age and diet may be collected. In many cases, these factors are known to be associated with diseases and/or SNP allele frequencies. There are likely gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

After all the relevant phenotypic and genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples can be summarized by descriptive statistics with tables and graphs. Data validation is preferably performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE is violated in a majority of markers, it is indicative of population substructure that should be further investigated. Moreover, Hardy-Weinberg disequilibrium in cases only can indicate genetic association of the markers with the disease of interest. (Genetic Data Analysis, Weir B., Sinauer (1990)).

To test whether an allele of a single SNP is associated with the case or control status of a phenotypic trait, one skilled in the art can compare allele frequencies in cases and controls. Standard chi-squared tests and Fisher exact tests can be carried out on a 2×2 table (2 SNP alleles×2 outcomes in the categorical trait of interest). To test whether genotypes of a SNP are associated, chi-squared tests can be carried out on a 3×2 table (3 genotypes×2 outcomes). Score tests are also carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes are calculated with the desired confidence limits, usually 95%. In the present study a software algorithm, PLINK, has been applied to automate the calculation of Hardy-Weinberg equilibrium, chi-square, p-values and odds-ratios for very large numbers of SNPs and Case-Control individuals simultaneously (Purcell et al. PLINK: a toolset for whole-genome association and population-based linkage analysis. American Journal of Human Genetics, 2007 in press).

In order to control for confounding effects and to test for interactions a stepwise multiple logistic regression analysis using statistical packages such as SAS or R may be performed. Logistic regression is a model-building technique in which the best fitting and most parsimonious model is built to describe the relation between the dichotomous outcome (for instance, getting a certain endometriosis or not) and a set of independent variables (for instance, genotypes of different associated genes, and the associated demographic and environmental factors). The most common model is one in which the logit transformation of the odds ratios is expressed as a linear combination of the variables (main effects) and their cross-product terms (interactions) (Applied Logistic Regression, Hosmer and Lemeshow, Wiley (2000)). To test whether a certain variable or interaction is significantly associated with the outcome, coefficients in the model are first estimated and then tested for statistical significance of their departure from zero.

In addition to performing association tests one marker at a time, haplotype association analysis may also be performed to study a number of markers that are closely linked together. Haplotype association tests can have better power than genotypic or allelic association tests when the tested markers are not the disease-causing mutations themselves but are in linkage disequilibrium with such mutations. The test will even be more powerful if the endometriosis is indeed caused by a combination of alleles on a haplotype. In order to perform haplotype association effectively, marker-marker linkage disequilibrium measures, both D′ and r², are typically calculated for the markers within a gene to elucidate the haplotype structure. Recent studies (Daly et al, Nature Genetics, 29, 232-235, 2001) in linkage disequilibrium indicate that SNPs within a gene are organized in block pattern, and a high degree of linkage disequilibrium exists within blocks and very little linkage disequilibrium exists between blocks. Haplotype association with the endometriosis status can be performed using such blocks once they have been elucidated.

Haplotype association tests can be carried out in a similar fashion as the allelic and genotypic association tests. Each haplotype in a gene is analogous to an allele in a multi-allelic marker. One skilled in the art can either compare the haplotype frequencies in cases and controls or test genetic association with different pairs of haplotypes. It has been proposed (Schaid et al, Am. J. Hum. Genet., 70, 425-434, 2002) that score tests can be done on haplotypes using the program “haplo.score”. In that method, haplotypes are first inferred by EM algorithm and score tests are carried out with a generalized linear model (GLM) framework that allows the adjustment of other factors.

An important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.1 (a significance level on the lenient side) may be used for generating hypotheses for significant association of a SNP with certain phenotypic characteristics of a endometriosis. It is preferred that a p-value <0.05 (a significance level traditionally used in the art) is achieved in order for a SNP to be considered to have an association with a endometriosis. It is more preferred that a p-value <0.01 (a significance level on the stringent side) is achieved for an association to be declared. Permutation tests to control for the false discovery rates, FDR, can further be employed (Benjamini and Hochberg, Journal of the Royal Statistical Society, Series B 57, 1289-1300, 1995, Resampling-based Multiple Testing, Westfall and Young, Wiley (1993)). Such methods to control for multiplicity would be preferred when the tests are dependent and controlling for false discovery rates is sufficient as opposed to controlling for the experiment-wise error rates.

In replication studies using samples from different populations after statistically significant markers have been identified in the exploratory stage, meta-analyses can then be performed by combining evidence of different studies (Modern Epidemiology, Lippincott Williams & Wilkins, 1998, 643-673). If available, association results known in the art for the same SNPs can be included in the meta-analyses.

Since both genotyping and endometriosis status classification can involve errors, sensitivity analyses may be performed to see how odds ratios and p-values would change upon various estimates on genotyping and endometriosis classification error rates.

Once individual risk factors, genetic or non-genetic, have been found for the predisposition to endometriosis, the next step is to set up a classification/prediction scheme to predict the category (for instance, endometriosis or no endometriosis) that an individual will be in depending on his genotypes of associated SNPs and other non-genetic risk factors. Logistic regression for discrete trait and linear regression for continuous trait are standard techniques for such tasks (Applied Regression Analysis, Draper and Smith, Wiley (1998)). Moreover, other techniques can also be used for setting up classification. Such techniques include, but are not limited to, MART, CART, neural network, and discriminant analyses that are suitable for use in comparing the performance of different methods (The Elements of Statistical Learning, Hastie, Tibshirani & Friedman, Springer (2002)).

Endometriosis Diagnosis and Predisposition Screening

Information on association/correlation between genotypes and endometriosis-related phenotypes can be exploited in several ways. For example, in the case of a highly statistically significant association between one or more SNPs with predisposition to a disease for which treatment is available, detection of such a genotype pattern in an individual may justify particular treatment, or at least the institution of regular monitoring of the individual. In the case of a weaker but still statistically significant association between a SNP and a human disease, immediate therapeutic intervention or monitoring may not be justified after detecting the susceptibility allele or SNP.

The SNPs of the invention may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single SNP may affect more than one phenotypic trait. Likewise, a single phenotypic trait may be affected by multiple SNPs in different genes.

The SNPs of the invention may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single SNP may affect more than one phenotypic trait. Likewise, a single phenotypic trait may be affected by multiple SNPs in different genes.

Haplotypes are particularly useful in that, for example, fewer SNPs can be genotyped to determine if a particular genomic region harbors a locus that influences a particular phenotype, such as in linkage disequilibrium-based SNP association analysis.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For diagnostic purposes, if a particular SNP site is found to be useful for diagnosing endometriosis, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For diagnostic applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., endometriosis) that is influenced by the causative SNP(s). Thus, polymorphic markers that are in LD with causative polymorphisms are useful as diagnostic markers, and are particularly useful when the actual causative polymorphism(s) is/are unknown.

Linkage disequilibrium in the human genome is reviewed in: International HapMap Consortium, “A haplotype map of the human genome” Nature Oct. 27, 2005; 437:1299-1320; Wall et al., “Haplotype blocks and linkage disequilibrium in the human genome”, Nat Rev Genet. August 2003;4(8):587-97; Garner et al., “On selecting markers for association studies: patterns of linkage disequilibrium between two and three diallelic loci”, Genet Epidemiol. January 2003;24(1):57-67; Ardlie et al., “Patterns of linkage disequilibrium in the human genome”, Nat Rev Genet. April 2002;3(4):299-309 (erratum in Nat Rev Genet July 2002;3(7):566); and Remm et al., “High-density genotyping and linkage disequilibrium in the human genome using chromosome 22 as a model”; Curr Opin Chem Biol. February 2002;6(1):24-30.

The contribution or association of particular SNPs and/or SNP haplotypes with endometriosis phenotypes, such as endometriosis, enables the SNPs of the present invention to be used to develop superior diagnostic tests capable of identifying individuals who express a detectable trait, such as endometriosis. as the result of a specific genotype, or individuals whose genotype places them at an increased or decreased risk of developing a detectable trait at a subsequent time as compared to individuals who do not have that genotype. As described herein, diagnostics may be based on a single SNP or a group of SNPs. Combined detection of a plurality of SNPs (for example, 2, 3, 4, 5, 6, 7, 8, 9, 10, 11, 12, 13, 14, 15, 16, 17, 18, 19, 20, 24, 25, 30, 32, 48, 50, 64, 96, 100, or any other number in-between, or more, of the SNPs provided in Tables 1-196 typically increases the probability of an accurate diagnosis. For example, the presence of a single SNP known to correlate with endometriosis might indicate a odds ratio of 1.5 that an individual has or is at risk of developing endometriosis, whereas detection of five SNPs, each of which correlates with endometriosis, might indicate an odds ratio of 9.5 that an individual has or is at risk of developing endometriosis. To further increase the accuracy of diagnosis or predisposition screening, analysis of the SNPs of the present invention can be combined with that of other polymorphisms or other risk factors of endometriosis, such as gender and age.

It will, of course, be understood by practitioners skilled in the treatment or diagnosis of endometriosis that the present invention generally does not intend to provide an absolute identification of individuals who are at risk (or less at risk) of developing endometriosis and/or pathologies related to endometriosis, but rather to indicate a certain increased (or decreased) degree or likelihood of developing the endometriosis based on statistically significant association results. However, this information is extremely valuable as it can be used to, for example, initiate earlier preventive treatments or to allow an individual carrying one or more significant SNPs or SNP haplotypes to regularly scheduled physical exams to monitor for the appearance or change of their endometriosis in order to identify and begin treatment of the endometriosis at an early stage.

The diagnostic techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders related to endometriosis.

Another aspect of the present invention relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more SNP positions, wherein the assayed nucleotide(s) is/are indicative of an increased or decreased risk of developing the trait or indicative that the individual expresses the trait as a result of possessing a particular trait-causing or trait-influencing allele.

The SNPs of the present invention also can be used to identify novel therapeutic targets for endometriosis. For example, genes containing the disease-associated variants (“variant genes”) or their products, as well as genes or their products that are directly or indirectly regulated by or interacting with these variant genes or their products, can be targeted for the development of therapeutics that, for example, treat the endometriosis or prevent or delay endometriosis onset. The therapeutics may be composed of, for example, small molecules, proteins, protein fragments or peptides, antibodies, nucleic acids, or their derivatives or mimetics which modulate the functions or levels of the target genes or gene products.

The SNPs/haplotypes of the present invention are also useful for improving many different aspects of the drug development process. For example, individuals can be selected for clinical trials based on their SNP genotype. Individuals with SNP genotypes that indicate that they are most likely to respond to or most likely to benefit from a device or a drug can be included in the trials and those individuals whose SNP genotypes indicate that they are less likely to or would not respond to a device or a drug, or suffer adverse reactions, can be eliminated from the clinical trials. This not only improves the safety of clinical trials, but also will enhance the chances that the trial will demonstrate statistically significant efficacy. Furthermore, the SNPs of the present invention may explain why certain previously developed devices or drugs performed poorly in clinical trials and may help identify a subset of the population that would benefit from a drug that had previously performed poorly in clinical trials, thereby “rescuing” previously developed therapeutic treatment methods or drugs, and enabling the methods or drug to be made available to a particular endometriosis patient population that can benefit from it.

Pharmaceutical Compositions

Any of the endometriosis-associated proteins, and encoding nucleic acid molecules, disclosed herein can be used as therapeutic targets (or directly used themselves as therapeutic compounds) for treating endometriosis and related pathologies, and the present disclosure enables therapeutic compounds (e.g., small molecules, antibodies, therapeutic proteins, RNAi and antisense molecules, etc.) to be developed that target (or are comprised of) any of these therapeutic targets.

Variant Proteins Encoded by SNP-Containing Nucleic Acid Molecules

The present invention provides SNP-containing nucleic acid molecules, some of which encode proteins having variant amino acid sequences as compared to the art-known (i.e., wild-type) proteins. These variants will generally be referred to herein as variant proteins/peptides/polypeptides, or polymorphic proteins/peptides/polypeptides of the present invention. The terms “protein”, “peptide”, and “polypeptide” are used herein interchangeably.

A variant protein of the present invention may be encoded by, for example, a nonsynonymous nucleotide substitution at any one of the cSNP positions disclosed herein. In addition, variant proteins may also include proteins whose expression, structure, and/or function is altered by a SNP disclosed herein, such as a SNP that creates or destroys a stop codon, a SNP that affects splicing, and a SNP in control/regulatory elements, e.g. promoters, enhancers, or transcription factor binding domains.

Uses of Variant Proteins

The variant proteins of the present invention can be used in a variety of ways, including but not limited to, in assays to determine the biological activity of a variant protein, such as in a panel of multiple proteins for high-throughput screening; to raise antibodies or to elicit another type of immune response; as a reagent (including the labeled reagent) in assays designed to quantitatively determine levels of the variant protein (or its binding partner) in biological fluids; as a marker for cells or tissues in which it is preferentially expressed (either constitutively or at a particular stage of tissue differentiation or development or in a endometriosis state); as a target for screening for a therapeutic agent; and as a direct therapeutic agent to be administered into a human subject. Any of the variant proteins disclosed herein may be developed into reagent grade or kit format for commercialization as research products. Methods for performing the uses listed above are well known to those skilled in the art (see, e.g., Molecular Cloning: A Laboratory Manual, Cold Spring Harbor Laboratory Press, Sambrook and Russell, 2000, and Methods in Enzymology: Guide to Molecular Cloning Techniques, Academic Press, Berger, S. L. and A. R. Kimmel eds., 1987).

### Computer-Related Embodiments

The SNPs provided in the present invention may be “provided” in a variety of mediums to facilitate use thereof. As used in this section, “provided” refers to a manufacture, other than an isolated nucleic acid molecule, that contains SNP information of the present invention. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present invention such as, for example, polymorphic nucleic acid and/or amino acid sequence information, information about observed SNP alleles, alternative codons, populations, allele frequencies, SNP types, and/or affected proteins, or any other information provided by the present invention in Tables 1 or 2, or in Tables 3-196.

In one application of this embodiment, the SNPs of the present invention can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the present invention. One such medium is provided with the present application, namely, the present application contains computer readable medium (CD-R) that has nucleic acid sequences (and encoded protein sequences) containing SNPs provided/recorded thereon in ASCII text format in a Sequence Listing along with accompanying Tables that contain detailed SNP and sequence information.

As used herein, “recorded” refers to a process for storing information on computer readable medium. A skilled artisan can readily adopt any of the presently known methods for recording information on computer readable medium to generate manufactures comprising the SNP information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide/amino acid sequence information of the present invention on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information of the present invention.

By providing the SNPs of the present invention in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul et at, J. Mol. Biol. 215:403-410 (1990)) and BLAZE (Brutlag et at, Comp. Chem. 17:203-207 (1993)) search algorithms.

The present invention further provides systems, particularly computer-based systems, which contain the SNP information described herein. Such systems may be designed to store and/or analyze information on, for example, a large number of SNP positions, or information on SNP genotypes from a large number of individuals. The SNP information of the present invention represents a valuable information source. The SNP information of the present invention stored/analyzed in a computer-based system may be used for such computer-intensive applications as determining or analyzing SNP allele frequencies in a population, mapping endometriosis genes, genotype-phenotype association studies, grouping SNPs into haplotypes, correlating SNP haplotypes with response to particular treatments or for various other bioinformatic, pharmacogenomic or drug development.

As used herein, “a computer-based system” refers to the hardware means, software means, and data storage means used to analyze the SNP information of the present invention. The minimum hardware means of the computer-based systems of the present invention typically comprises a central processing unit (CPU), input means, output means, and data storage means. A skilled artisan can readily appreciate that any one of the currently available computer-based systems are suitable for use in the present invention. Such a system can be changed into a system of the present invention by utilizing the SNP information provided on the CD-R, or a subset thereof, without any experimentation.

As stated above, the computer-based systems of the present invention comprise a data storage means having stored therein SNPs of the present invention and the necessary hardware means and software means for supporting and implementing a search means. As used herein, “data storage means” refers to memory which can store SNP information of the present invention, or a memory access means which can access manufactures having recorded thereon the SNP information of the present invention.

As used herein, “search means” refers to one or more programs or algorithms that are implemented on the computer-based system to identify or analyze SNPs in a target sequence based on the SNP information stored within the data storage means. Search means can be used to determine which nucleotide is present at a particular SNP position in the target sequence. As used herein, a “target sequence” can be any DNA sequence containing the SNP position(s) to be searched or queried.

As used herein, “a target structural motif,” or “target motif,” refers to any rationally selected sequence or combination of sequences containing a SNP position in which the sequence(s) is chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, hairpin structures, and inducible expression elements (protein binding sequences).

A variety of structural formats for the input and output means can be used to input and output the information in the computer-based systems of the present invention. An exemplary format for an output means is a display that depicts the presence or absence of specified nucleotides (alleles) at particular SNP positions of interest. Such presentation can provide a rapid, binary scoring system for many SNPs simultaneously.

### EXAMPLES

Overview of Association Study

Endometriosis is a debilitating disease, characterized by the presence of endometrium (glands and stroma) at sites outside of the uterus, which is estimated to affect approximately 14% of all women. Endometrioses often leads to pain, local inflammation, scarring and decreased fertility. This example identifies genetic loci in the form of SNPs associated with endometriosis.

A Genome Wide Association study was performed to identify SNPs associated with Endometriosis. The Affymetrix 500K GeneChip technology platform was employed in the study to ascertain genotypic information across a total of 500,568 individual SNPs. The 500K GeneChip system is composed of two separate assays, referred to as the Nsp and Sty chips, and designed to interrogate 262,264 and 238,304 SNPs respectively. In all, 170 individuals diagnosed with Endometriosis were tested and compared to 734 control individuals using the Nsp chip and 169 individuals diagnosed with Endometriosis were compared and tested to 738 control individuals using the Sty chip. A statistical software tool, PLINK, specifically developed to test for genetic association, was used to calculate p values for each SNP, enabling identification of a set of candidate SNPs that showed statistically significant association to Endometriosis. All members in the study (cases and controls), were collected from the same geographical region, were Caucasian and generally of Northern and Western European descent.

Scanning the Entire Genome

The Affymetrix GeneChip 500K mapping array was used to scan the whole genome. Briefly, 250 ng of genomic DNA was digested with either NspI or StyI restriction endonuclease and digested fragments were ligated to adapters that contained a universal sequence. The ligated products were then amplified using the polymerase chain reaction (PCR) to amplify fragments between 250-2000 bp in length. The PCR products were purified and diluted to a standard concentration. Furthermore, the PCR products were then fragmented with a DNase enzyme to approximately 25-150 bp in length. This fragmentation process further reduced the complexity of the genomic sample. The fragmented PCR products were then labeled with a biotin/streptavidin system and allowed to hybridize to the microarray. After hybridization the arrays were stained and non-specific binding was removed through a series of increasingly stringent washes. The genotypes were determined by fluorescent signal detection in an Affymetrix GCS 3000 scanner. Finally, genotypes were called using the BRLMM algorithm which is integrated into Affymetrix PowerTool software.

Selection of SNPs for Quality and Association

A SNP is a DNA sequence variation, occurring when a single nucleotide—adenine (A), thymine (T), cytosine (C) or guanine (G)—in the genome differs between individuals. A variation must occur in at least 1% of the population to be considered a SNP. Variations that occur in less than 1% of the population are, by definition considered to be mutations whether they cause disease or not. SNPs make up 90% of all human genetic variations, and occur every 300 to 1000 bases along the human genome. On average, two of every three SNPs substitute cytosine (C) with thymine (T). For the data to be considered valid for an individual chip, two internal quality control measures were used: SNP genotypes must have exceeded an overall call rate of >93% and the correct gender of the sample needed to be determined as based on the heterozygosity of the X chromosome SNPs. Further, a SNP that did not have at least a 96% call rate across all subjects was eliminated as having possible genotyping errors. SNPs that were monomorphic, having less than 1% apparent variation in both cases and controls, were also eliminated from analysis. In addition, SNPs that failed a Hardy-Weinberg equilibrium test in the control population only, using a p-value threshold of 0.001, were also eliminated. After removal of these SNPs, 382,851 SNPs were available for analysis. Genotypes were analyzed for significance using PLINK and Haploview software.

GeneChip microarrays consist of small DNA fragments (referred to as probes), chemically synthesized at specific locations on a coated quartz surface. The precise location where each probe is synthesized is called a feature, and millions of features can be contained on one array. The probes which represent a sequence known to contain a human SNP were selected by Affymetrix based on reliability, sensitivity and specificity. In addition to these criteria, the probes were selected to cover the human genome at approximately equal intervals.

Identification of Endometriosis Affected Individuals.

Individuals were determined to have endometriosis after medical record review by a single physician. In this study, only patients with visually confirmed disease (either by laparoscopy or other surgical intervention) were included as cases. The controls included individuals without prior history of endometriosis.

Endometriosis Associated SNPs

After sorting all remaining candidate SNPs by p-value, 610 SNPs with p-values less than or equal to 0.001 were selected as Primary SNPs. Further, 2,048 SNPs with p-values between 0.001 and 0.005 were selected as Supporting SNPs for a total of 2,658 candidate endometriosis SNPs. To select the SNPs most strongly associated with endometriosis from the 2,658 candidate SNPs, two sequential selection steps were applied. The first selection, referred to as Anchor SNPs, included any SNP with a p-value 0.001 or stronger located no more that 50 kb from any SNP in the list of 2,658 candidate SNPs. If a third SNP in turn was located within 50 kb from either of the previous SNPs, the group was expanded to include the new SNP. By repeating this approach until no additional SNPs could be added to the grouping, 108 separate Anchor Groups were established (see Table 1). In the second selection step, 88 remaining SNPs with a p-value of 0.0001 or smaller were selected regardless of the proximity and p-value of any neighboring SNPs. These SNPs are referred to as Singleton SNPs (see Table 2).

Linkage Disequilibrium Blocks

As described above, the human genome includes extensive regions of linkage disequilibrium that undergo very minimal recombination. As a result, any SNP located within the same LD blocks as any of the Anchor SNPs or Singleton SNPs listed in Table 1 and Table 2 contributes haplotype information for refined diagnostic discrimination and to the further identification of the causative mutation. Therefore, by virtue of linkage disequilibrium, a set of additional SNPs that have been determined to be in linkage disequilibrium with any of the Anchor SNPs or Singleton SNPs are listed in Tables 3-196. Specifically, by using the Haploview software package in conjunction with the Caucasian population of the HapMap data set (release 21) LD blocks were identified around all 108 Anchor Blocks and 88 Singleton SNPs listed in Table 1 and Table 2. Each of the Tables 3-196 represent SNPs located within the LD block(s) surrounding all SNPs from Table 1 and Table 2.

Tables

TABLE 001

Anchor Group SNPs

| | | | | | | | | | |
|---|---|---|---|---|---|---|---|---|---|
| Set | Tbl | Name | Chr | Position | p-value | OR | F_A | F_U | Flank Sequence |

| | | | | | | | | | |
|---|---|---|---|---|---|---|---|---|---|
| 01 | 003 | rs507667 | 1 | 37823966 | 4.6E−04 | 0.6523 | 0.4 | 0.506 | (SEQ ID No: 1) |
| | | | | | | | | | taactgcacttaattc[C/T] |
| | | | | | | | | | atgcaggtaggaaact |
| | 003 | rs661460 | 1 | 37835420 | 1.6E−03 | 0.6806 | 0.397 | 0.492 | (SEQ ID No: 2) |
| | | | | | | | | | acaccagtcaattatc[A/T] |
| | | | | | | | | | cttttgtcattgccat |
| | 003 | rs557897 | 1 | 37843278 | 2.1E−03 | 0.686 | 0.396 | 0.489 | (SEQ ID No: 3) |
| | | | | | | | | | cctaactggtgtggga[A/C] |
| | | | | | | | | | tagcacgtctgaattc |
| | 003 | rs658024 | 1 | 37849378 | 2.3E−03 | 0.6889 | 0.399 | 0.491 | (SEQ ID No: 4) |
| | | | | | | | | | ggcctgtctccaagta[C/T] |
| | | | | | | | | | ggtcaccttctgagtt |
| | 004 | rs2070929 | 1 | 47395627 | 2.2E−04 | 1.606 | 0.349 | 0.25 | (SEQ ID No: 5) |
| | | | | | | | | | cctgaaattgaatgga[C/G] |
| | | | | | | | | | agcctgagtgctacaa |
| | 004 | rs2250495 | 1 | 47405397 | 1.2E−03 | 1.512 | 0.352 | 0.264 | (SEQ ID No: 6) |
| | | | | | | | | | tccaccaaaatcataa[A/G] |
| | | | | | | | | | ctcacttccaatgaag |
| | 004 | rs2798349 | 1 | 47410723 | 4.7E−03 | 0.4276 | 0.036 | 0.079 | (SEQ ID No: 7) |
| | | | | | | | | | ctactcttcacaaaca[A/G] |
| | | | | | | | | | tcctgagaagctggat |
| | NA | rs656464 | 1 | 60313877 | 2.6E−03 | 1.473 | 0.363 | 0.279 | (SEQ ID No: 8) |
| | | | | | | | | | aaagcaaaatacgaca[A/G] |
| | | | | | | | | | aatagaacacagaaaa |
| | NA | rs11207574 | 1 | 60338189 | 1.8E−03 | 1.473 | 0.402 | 0.314 | (SEQ ID No: 9) |
| | | | | | | | | | ctaagggatttatgta[C/T] |
| | | | | | | | | | agattgctaatgccgt |
| | NA | rs12122772 | 1 | 60384564 | 1.9E−03 | 1.458 | 0.444 | 0.354 | (SEQ ID No: 10) |
| | | | | | | | | | atttataaattaaatc[G/T] |
| | | | | | | | | | catactgccaataaaa |
| 02 | 005 | rs1561168 | 1 | 64827340 | 1.5E−03 | 0.4231 | 0.044 | 0.098 | (SEQ ID No: 11) |
| | | | | | | | | | aaaaactggatctcta[C/T] |
| | | | | | | | | | gtcatcctttgtccct |
| | 005 | rs1561167 | 1 | 64827534 | 8.1E−04 | 0.4055 | 0.044 | 0.102 | (SEQ ID No: 12) |
| | | | | | | | | | gggcctgtgttatgaa[A/T] |
| | | | | | | | | | cctgtgcagtggattt |
| | 005 | rs7517300 | 1 | 64840092 | 1.2E−03 | 0.4161 | 0.044 | 0.1 | (SEQ ID No: 13) |
| | | | | | | | | | gtggggtgttggaaac[A/G] |
| | | | | | | | | | gttcccaattaggagc |
| | 005 | rs11208493 | 1 | 64840416 | 5.5E−04 | 0.3949 | 0.045 | 0.106 | (SEQ ID No: 14) |
| | | | | | | | | | agtgaatcacagtgca[A/G] |
| | | | | | | | | | aacaaaaacactaaat |
| | 005 | rs7513574 | 1 | 64847362 | 1.9E−03 | 0.4498 | 0.051 | 0.107 | (SEQ ID No: 15) |
| | | | | | | | | | ttctaagtacaacaga[A/C] |
| | | | | | | | | | caggctgtttgcaata |
| | 006 | rs1337362 | 1 | 160925801 | 5.0E−04 | 1.584 | 0.314 | 0.224 | (SEQ ID No: 16) |
| | | | | | | | | | atatattagtgaagtc[C/G] |
| | | | | | | | | | atgaaataaaggattg |
| | 006 | rs11809129 | 1 | 160961303 | 9.1E−04 | 1.507 | 0.408 | 0.314 | (SEQ ID No: 17) |
| | | | | | | | | | gagcctcttcattgcc[A/G] |
| | | | | | | | | | agatctgattgtgcaa |
| | NA | rs10489371 | 1 | 165664158 | 2.3E−03 | 0.6285 | 0.181 | 0.26 | (SEQ ID No: 18) |
| | | | | | | | | | atgctggaagtatgga[A/G] |
| | | | | | | | | | acagtcaatgaaaact |
| | NA | rs3007374 | 1 | 165664272 | 2.5E−03 | 1.443 | 0.562 | 0.471 | (SEQ ID No: 19) |
| | | | | | | | | | atttctggcatggaaa[C/G] |
| | | | | | | | | | atcattaaagaaccaa |
| | NA | rs16861817 | 1 | 165665445 | 1.2E−03 | 1.562 | 0.277 | 0.197 | (SEQ ID No: 20) |
| | | | | | | | | | ctgatgtcagaattcc[A/G] |
| | | | | | | | | | aaattccaagttaggg |
| 03 | 007 | rs12739430 | 1 | 165684746 | 9.4E−04 | 1.574 | 0.284 | 0.202 | (SEQ ID No: 21) |
| | | | | | | | | | ctcatcttcctcccca[A/G] |
| | | | | | | | | | tatggtctataaggtc |
| | NA | rs6540802 | 1 | 210359160 | 1.1E−03 | 0.35 | 0.03 | 0.08 | (SEQ ID No: 22) |
| | | | | | | | | | gagatgtctggtgcaa[C/T] |
| | | | | | | | | | acaataggcactaaat |
| | NA | rs7515439 | 1 | 210360491 | 1.6E−03 | 0.3768 | 0.033 | 0.083 | (SEQ ID No: 23) |
| | | | | | | | | | agagggcaatatttaa[A/T] |
| | | | | | | | | | gacagaattttgggtg |
| | NA | rs1578435 | 1 | 210380252 | 2.6E−03 | 0.5248 | 0.077 | 0.137 | (SEQ ID No: 24) |
| | | | | | | | | | taataccgcaataaaa[C/T] |
| | | | | | | | | | gggtcacacgaatttt |
| | 008 | rs10494966 | 1 | 210389815 | 7.2E−04 | 0.4745 | 0.071 | 0.138 | (SEQ ID No: 25) |
| | | | | | | | | | aaattcctttttgtca[C/G] |
| | | | | | | | | | aatggatcagagtcat |
| | 008 | rs9651104 | 1 | 210392113 | 7.7E−04 | 0.5449 | 0.115 | 0.193 | (SEQ ID No: 26) |
| | | | | | | | | | accttttcatcccccc[A/G] |
| | | | | | | | | | atggataaacttatta |
| | 009 | rs4520477 | 1 | 213031092 | 7.2E−04 | 1.507 | 0.456 | 0.357 | (SEQ ID No: 27) |
| | | | | | | | | | gccaaaaagccaaatg[A/G] |
| | | | | | | | | | caaatgatagatattt |
| | 009 | rs4579828 | 1 | 213031765 | 5.9E−04 | 1.518 | 0.456 | 0.356 | (SEQ ID No: 28) |
| | | | | | | | | | cctttccttctatccc[C/T] |
| | | | | | | | | | attgcaccatgctgtc |
| | 010 | rs10127631 | 1 | 216790563 | 1.3E−03 | 1.972 | 0.1 | 0.053 | (SEQ ID No: 29) |
| | | | | | | | | | tgtttgtcaagtcaca[A/G] |
| | | | | | | | | | tatgagcaaatctatc |
| | 010 | rs10863539 | 1 | 216800365 | 4.1E−04 | 2.12 | 0.1 | 0.05 | (SEQ ID No: 30) |
| | | | | | | | | | ccactctccaaataca[A/G] |
| | | | | | | | | | cattctctcttactct |
| 04 | 010 | rs10779401 | 1 | 216824394 | 3.6E−03 | 1.893 | 0.095 | 0.053 | (SEQ ID No: 31) |
| | | | | | | | | | atttctttgattttaa[C/T] |
| | | | | | | | | | ggctaaacagtattcc |
| | 012 | rs7599610 | 2 | 5871611 | 1.2E−04 | 7.059 | 0.024 | 0.003 | (SEQ ID No: 32) |
| | | | | | | | | | tttgaaaaagttccca[A/G] |
| | | | | | | | | | taacaatgacattttt |
| | NA | rs1429238 | 2 | 5909306 | 4.8E−03 | 1.434 | 0.349 | 0.272 | (SEQ ID No: 33) |
| | | | | | | | | | gagtgtatattattta[A/G] |
| | | | | | | | | | atggtcaccaaaatct |
| | NA | rs12617498 | 2 | 5961180 | 2.1E−03 | 0.6669 | 0.279 | 0.368 | (SEQ ID No: 34) |
| | | | | | | | | | gtgatctggaaagcta[C/G] |
| | | | | | | | | | agtttctcgtttctgt |
| | NA | rs2709441 | 2 | 5968216 | 4.2E−03 | 0.7028 | 0.379 | 0.465 | (SEQ ID No: 35) |
| | | | | | | | | | gttagcagacagcgga[A/G] |
| | | | | | | | | | ctatgaattagacttg |
| | NA | rs16864465 | 2 | 5971342 | 1.6E−03 | 0.6486 | 0.244 | 0.332 | (SEQ ID No: 36) |
| | | | | | | | | | tcaggtccagtgaatc[A/G] |
| | | | | | | | | | aaatctgcatcttcac |
| | NA | rs11682830 | 2 | 5979612 | 3.0E−03 | 0.6978 | 0.418 | 0.507 | (SEQ ID No: 37) |
| | | | | | | | | | tggggaggtgtgtctc[A/G] |
| | | | | | | | | | aagccaagggatgaca |
| | 013 | rs720118 | 2 | 5987961 | 5.6E−04 | 0.6365 | 0.287 | 0.387 | (SEQ ID No: 38) |
| | | | | | | | | | aaacttttgtgatcta[C/T] |
| | | | | | | | | | gtagtgcatcacaaat |
| | 014 | rs10192401 | 2 | 75313269 | 2.4E−04 | 0.619 | 0.287 | 0.394 | (SEQ ID No: 39) |
| | | | | | | | | | cctgatgaagtttcac[A/G] |
| | | | | | | | | | ctgtcccactccagcc |
| | 014 | rs10203484 | 2 | 75313393 | 2.8E−04 | 0.6218 | 0.287 | 0.393 | (SEQ ID No: 40) |
| | | | | | | | | | aactgaaactgcagaa[A/G] |
| | | | | | | | | | tggaaaccgtggataa |
| 05 | 015 | rs1192367 | 2 | 84945493 | 4.6E−03 | 1.584 | 0.178 | 0.12 | (SEQ ID No: 41) |
| | | | | | | | | | aaccatgaataatgaa[C/G] |
| | | | | | | | | | acatctaaatctccta |
| | 015 | rs1627220 | 2 | 84965114 | 1.1E−03 | 1.724 | 0.169 | 0.105 | (SEQ ID No: 42) |
| | | | | | | | | | gccagggcttaagagc[C/T] |
| | | | | | | | | | aagttaatagccctgg |
| | 015 | rs736711 | 2 | 84977903 | 7.9E−04 | 1.711 | 0.185 | 0.117 | (SEQ ID No: 43) |
| | | | | | | | | | agcctcagatttcaga[A/G] |
| | | | | | | | | | gctaatcagaggtgac |
| | NA | rs1000919 | 2 | 85008139 | 4.9E−03 | 1.574 | 0.179 | 0.122 | (SEQ ID No: 44) |
| | | | | | | | | | acttttatggaaacaa[C/T] |
| | | | | | | | | | taaaccatggcacttt |
| | 016 | rs2970924 | 2 | 88272567 | 4.7E−03 | 1.543 | 0.204 | 0.143 | (SEQ ID No: 45) |
| | | | | | | | | | ctgtgtagtataattc[A/G] |
| | | | | | | | | | acagtgcaaaccacca |
| | 016 | rs4386315 | 2 | 88275093 | 4.8E−04 | 1.73 | 0.196 | 0.124 | (SEQ ID No: 46) |
| | | | | | | | | | acttttcattacctac[C/T] |
| | | | | | | | | | gcagcaaatccaaaat |
| | 016 | rs10168005 | 2 | 88285077 | 9.8E−04 | 1.678 | 0.195 | 0.126 | (SEQ ID No: 47) |
| | | | | | | | | | ttcagacgtctaggtc[C/G] |
| | | | | | | | | | atcacagacctactga |
| | 016 | rs12463835 | 2 | 88285874 | 8.2E−04 | 1.691 | 0.195 | 0.126 | (SEQ ID No: 48) |
| | | | | | | | | | aatattctttgttttc[C/T] |
| | | | | | | | | | ccacttaggatgggat |
| | 017 | rs4666865 | 2 | 83523597 | 1.0E−03 | 0.659 | 0.328 | 0.426 | (SEQ ID No: 49) |
| | | | | | | | | | aaatggcagagaatga[A/G] |
| | | | | | | | | | actttcaagtaattgg |
| | NA | rs12473127 | 2 | 183551537 | 2.7E−03 | 0.6824 | 0.359 | 0.451 | (SEQ ID No: 50) |
| | | | | | | | | | ttcttactagctctgc[G/T] |
| | | | | | | | | | aacttggacagtaact |
| 06 | 018 | rs10206592 | 2 | 235331943 | 7.3E−04 | 1.546 | 0.357 | 0.264 | (SEQ ID No: 51) |
| | | | | | | | | | ttccctgctgcgctga[A/T] |
| | | | | | | | | | aattgactttccctga |
| | 018 | rs10167927 | 2 | 235332321 | 1.4E−03 | 1.507 | 0.349 | 0.263 | (SEQ ID No: 52) |
| | | | | | | | | | caagagcatcagcata[A/G] |
| | | | | | | | | | cagaacattaaaatgg |
| | 018 | rs10168146 | 2 | 235332486 | 1.3E−03 | 1.509 | 0.349 | 0.262 | (SEQ ID No: 53) |
| | | | | | | | | | acactgggatttctta[C/T] |
| | | | | | | | | | aggttgctccacaaag |
| | 019 | rs3772284 | 3 | 1372414 | 8.1E−04 | 1.507 | 0.461 | 0.362 | (SEQ ID No: 54) |
| | | | | | | | | | ttatcagctttactga[A/G] |
| | | | | | | | | | atttctcatttgctcc |
| | 019 | rs10510208 | 3 | 1376516 | 4.9E−03 | 1.465 | 0.29 | 0.218 | (SEQ ID No: 55) |
| | | | | | | | | | gaaataaaccttggac[A/C] |
| | | | | | | | | | gagaaaagcaggcaaa |
| | NA | rs13063912 | 3 | 1383344 | 2.2E−03 | 1.571 | 0.228 | 0.158 | (SEQ ID No: 56) |
| | | | | | | | | | gccccagcactaacat[A/G] |
| | | | | | | | | | agtaccccacagagac |
| | 020 | rs7633620 | 3 | 2950433 | 2.1E−04 | 0.5896 | 0.216 | 0.318 | (SEQ ID No: 57) |
| | | | | | | | | | tcctattggacactca[C/T] |
| | | | | | | | | | atagcacttatgctat |
| | 020 | rs6789338 | 3 | 2951094 | 1.6E−04 | 0.6195 | 0.337 | 0.451 | (SEQ ID No: 58) |
| | | | | | | | | | ttcccaaaggcttttg[A/G] |
| | | | | | | | | | aagagttgaaattcat |
| | 020 | rs4487227 | 3 | 2954275 | 2.8E−04 | 0.5911 | 0.208 | 0.308 | (SEQ ID No: 59) |
| | | | | | | | | | ttggggaaggaaagag[A/G] |
| | | | | | | | | | agacagcaatcacagg |
| | 021 | rs4640525 | 3 | 57238434 | 4.2E−03 | 1.415 | 0.465 | 0.38 | (SEQ ID No: 60) |
| | | | | | | | | | gactataaacggctca[C/G] |
| | | | | | | | | | aatggattgcacactg |
| 07 | 021 | rs1553987 | 3 | 57250185 | 2.0E−03 | 1.453 | 0.5 | 0.408 | (SEQ ID No: 61) |
| | | | | | | | | | acttcagattatgagc[A/G] |
| | | | | | | | | | aaggagaagccaatgg |
| | 021 | rs3087684 | 3 | 57281359 | 3.1E−03 | 0.6842 | 0.314 | 0.4 | (SEQ ID No: 62) |
| | | | | | | | | | acttgccaaccaggga[C/T] |
| | | | | | | | | | taaagctattatcttg |
| | 021 | rs12488822 | 3 | 57292820 | 3.4E−04 | 0.6215 | 0.287 | 0.393 | (SEQ ID No: 63) |
| | | | | | | | | | acttaattattcagta[C/T] |
| | | | | | | | | | agtcctggaaatgttt |
| | 021 | rs502442 | 3 | 57306267 | 3.0E−03 | 1.434 | 0.465 | 0.377 | (SEQ ID No: 64) |
| | | | | | | | | | aactatgtttcattca[C/T] |
| | | | | | | | | | gttaatgctcaaattg |
| | NA | rs554418 | 3 | 57308815 | 1.9E−03 | 0.6696 | 0.302 | 0.392 | (SEQ ID No: 65) |
| | | | | | | | | | aggagagctgtcacca[A/G] |
| | | | | | | | | | taacctgaccatgatg |
| | 022 | rs9830289 | 3 | 107972966 | 3.0E−04 | 1.558 | 0.446 | 0.341 | (SEQ ID No: 66) |
| | | | | | | | | | gatttctcaatggtag[A/G] |
| | | | | | | | | | atccatcttctattta |
| | 022 | rs4895039 | 3 | 108005925 | 6.6E−04 | 1.526 | 0.397 | 0.302 | (SEQ ID No: 67) |
| | | | | | | | | | taaacaaagggtaata[A/C] |
| | | | | | | | | | attggaagagcagtag |
| | 022 | rs2049118 | 3 | 108010714 | 7.0E−04 | 1.522 | 0.4 | 0.305 | (SEQ ID No: 68) |
| | | | | | | | | | acaaaacaacatttac[C/G] |
| | | | | | | | | | ctgaggaaggaacact |
| | 022 | rs1520717 | 3 | 108011334 | 1.5E−03 | 1.475 | 0.427 | 0.335 | (SEQ ID No: 69) |
| | | | | | | | | | tgctttgctttcagta[A/G] |
| | | | | | | | | | cattagctgaagtaat |
| | 022 | rs6779049 | 3 | 108018247 | 1.4E−04 | 1.606 | 0.405 | 0.298 | (SEQ ID No: 70) |
| | | | | | | | | | ttcaaaagatgacaac[A/G] |
| | | | | | | | | | aaatgacactttctaa |
| 08 | 022 | rs2399117 | 3 | 108020680 | 2.0E−03 | 0 | 0 | 0.029 | (SEQ ID No: 71) |
| | | | | | | | | | tttttctgatatttaa[C/T] |
| | | | | | | | | | gtaccaacatatgata |
| | 023 | rs2331921 | 3 | 122608097 | 5.4E−04 | 0.6315 | 0.272 | 0.372 | (SEQ ID No: 72) |
| | | | | | | | | | aaaatcacctataggc[A/G] |
| | | | | | | | | | ataatctggcatacat |
| | 023 | rs12186062 | 3 | 122626569 | 4.3E−04 | 0.6254 | 0.268 | 0.369 | (SEQ ID No: 73) |
| | | | | | | | | | aaaagttgtctttaaa[A/G] |
| | | | | | | | | | tcttccaacaaaaagt |
| | 023 | rs3772122 | 3 | 122679758 | 1.1E−03 | 0.6475 | 0.268 | 0.361 | (SEQ ID No: 74) |
| | | | | | | | | | gtcctttgatgcagag[C/T] |
| | | | | | | | | | agtgtggtacccagca |
| | 023 | rs4555467 | 3 | 122693312 | 3.4E−04 | 0.6207 | 0.269 | 0.373 | (SEQ ID No: 75) |
| | | | | | | | | | tccaagtggccagtta[A/G] |
| | | | | | | | | | aagtctaaaccagggg |
| | 023 | rs3806614 | 3 | 122747866 | 5.3E−04 | 0.6311 | 0.272 | 0.372 | (SEQ ID No: 76) |
| | | | | | | | | | tatttcagattgagaa[A/G] |
| | | | | | | | | | cattatgcaagatagc |
| | NA | rs1656376 | 3 | 159767383 | 3.2E−03 | 1.426 | 0.515 | 0.427 | (SEQ ID No: 77) |
| | | | | | | | | | gggaaaatggtgcata[C/G] |
| | | | | | | | | | cgattaagtgacctgt |
| | 024 | rs1435642 | 3 | 159782923 | 9.8E−04 | 1.498 | 0.426 | 0.331 | (SEQ ID No: 78) |
| | | | | | | | | | agcaatatttgaagaa[A/G] |
| | | | | | | | | | tgaagactaagaatta |
| | NA | rs6444482 | 3 | 192234311 | 3.6E−03 | 0.6867 | 0.303 | 0.388 | (SEQ ID No: 79) |
| | | | | | | | | | aaaaggagtgttgaca[A/G] |
| | | | | | | | | | aatcaaaatatttgtt |
| | NA | rs9290963 | 3 | 192236886 | 3.0E−03 | 0.6816 | 0.303 | 0.389 | (SEQ ID No: 80) |
| | | | | | | | | | tgtggatggaaactaa[C/G] |
| | | | | | | | | | taacttctagcaaagg |
| 09 | NA | rs6444495 | 3 | 192275264 | 2.6E−03 | 0.6778 | 0.308 | 0.396 | (SEQ ID No: 81) |
| | | | | | | | | | ttcttgtcgagcagaa[A/G] |
| | | | | | | | | | accagttgagaccact |
| | 025 | rs6444501 | 3 | 192302944 | 7.2E−04 | 1.51 | 0.438 | 0.341 | (SEQ ID No: 82) |
| | | | | | | | | | ctgtgaatgtgtggac[A/C] |
| | | | | | | | | | cttacaacattttatg |
| | NA | rs9290976 | 3 | 192343961 | 3.9E−03 | 0.7006 | 0.379 | 0.465 | (SEQ ID No: 83) |
| | | | | | | | | | gacacttagactgaaa[C/T] |
| | | | | | | | | | tattgcccaagacttg |
| | 026 | rs2365538 | 4 | 73204628 | 4.3E−03 | 0.6849 | 0.284 | 0.367 | (SEQ ID No: 84) |
| | | | | | | | | | aaattagctaagatgg[C/G] |
| | | | | | | | | | caatgtattgatctag |
| | 026 | rs7666924 | 4 | 73217392 | 3.3E−03 | 0.6614 | 0.236 | 0.319 | (SEQ ID No: 85) |
| | | | | | | | | | acctataggtcatttc[A/G] |
| | | | | | | | | | ctctaaacttaaaaaa |
| | 026 | rs6856651 | 4 | 73261492 | 7.5E−04 | 0.6209 | 0.232 | 0.327 | (SEQ ID No: 86) |
| | | | | | | | | | ctgtgtagagcaatga[A/T] |
| | | | | | | | | | tcacaattatttttgc |
| | 027 | rs3733558 | 4 | 122315696 | 4.5E−04 | 0.6372 | 0.311 | 0.414 | (SEQ ID No: 87) |
| | | | | | | | | | agaagacagtcgagct[A/C] |
| | | | | | | | | | aaagatgggaagataa |
| | 027 | SNP_A- | 4 | 122317455 | 5.4E−04 | 0.6439 | 0.318 | 0.42 | (SEQ ID No: 88) |
| | | 2218634 | | | | | | | cctggccaaatcttga[C/T] |
| | | | | | | | | | gttaatatatctgttt |
| | 027 | rs2175397 | 4 | 122320749 | 5.4E−04 | 0.6439 | 0.318 | 0.42 | (SEQ ID No: 89) |
| | | | | | | | | | ctcatactcacattta[A/G] |
| | | | | | | | | | agtcatttcttcaggg |
| | 027 | rs6832987 | 4 | 122321077 | 4.5E−04 | 0.6396 | 0.318 | 0.421 | (SEQ ID No: 90) |
| | | | | | | | | | gttcatacacctagag[A/T] |
| | | | | | | | | | ctccttctatctgaag |
| 10 | NA | rs7375286 | 4 | 165832224 | 1.8E−03 | 2.091 | 0.08 | 0.04 | (SEQ ID No: 91) |
| | | | | | | | | | ttctggtgttctgtta[C/T] |
| | | | | | | | | | acagtaggattgctat |
| | 028 | rs17621300 | 4 | 165856946 | 9.1E−05 | 1.873 | 0.189 | 0.111 | (SEQ ID No: 92) |
| | | | | | | | | | tatgtaagatgtaagg[A/C] |
| | | | | | | | | | aggactctatctgcag |
| | 028 | rs12641382 | 4 | 165868089 | 9.1E−05 | 1.869 | 0.188 | 0.11 | (SEQ ID No: 93) |
| | | | | | | | | | ttctgcaagcaacatc[A/G] |
| | | | | | | | | | aatttttcaagatctg |
| | 029 | rs1863984 | 5 | 4264882 | 1.0E−04 | 1.974 | 0.153 | 0.084 | (SEQ ID No: 94) |
| | | | | | | | | | atggctttcttgacca[A/G] |
| | | | | | | | | | taagtcttgttccttg |
| | 029 | rs1035866 | 5 | 4269069 | 3.9E−04 | 1.691 | 0.228 | 0.149 | (SEQ ID No: 95) |
| | | | | | | | | | gtaagtcccagagata[A/G] |
| | | | | | | | | | aaggcaccatttctac |
| | NA | rs12522730 | 5 | 4279713 | 1.6E−03 | 1.521 | 0.305 | 0.224 | (SEQ ID No: 96) |
| | | | | | | | | | tcatcattttattcca[C/T] |
| | | | | | | | | | tcatttgtttgagtgt |
| | NA | rs7706780 | 5 | 4280497 | 1.3E−03 | 1.533 | 0.303 | 0.221 | (SEQ ID No: 97) |
| | | | | | | | | | agattatagcctttaa[G/T] |
| | | | | | | | | | aacacagacacttata |
| | NA | rs6866903 | 5 | 4281049 | 4.8E−03 | 1.487 | 0.256 | 0.188 | (SEQ ID No: 98) |
| | | | | | | | | | acaaaccagtgagtga[G/T] |
| | | | | | | | | | aaccatcctggatgcc |
| | 030 | rs10059890 | 5 | 11199698 | 4.5E−03 | 1.41 | 0.488 | 0.404 | (SEQ ID No: 99) |
| | | | | | | | | | agtgctttgtggacaa[A/G] |
| | | | | | | | | | ataactctttaatacc |
| | 030 | rs6880938 | 5 | 11200302 | 3.5E−03 | 1.438 | 0.433 | 0.346 | (SEQ ID No: 100) |
| | | | | | | | | | gtcacttgtgaagtga[C/T] |
| | | | | | | | | | gggccttaaataccca |
| 11 | 030 | rs6859601 | 5 | 11200414 | 8.0E−04 | 1.497 | 0.503 | 0.403 | (SEQ ID No: 101) |
| | | | | | | | | | gaaggccgaatatata[C/T] |
| | | | | | | | | | ggaatgaacgtgtcaa |
| | 030 | rs12516262 | 5 | 11200575 | 9.9E−04 | 1.488 | 0.556 | 0.457 | (SEQ ID No: 102) |
| | | | | | | | | | gctagtattgcaaaca[G/T] |
| | | | | | | | | | ggtgaatgaagagcag |
| | 030 | rs1859382 | 5 | 11200672 | 8.9E−04 | 1.493 | 0.503 | 0.404 | (SEQ ID No: 103) |
| | | | | | | | | | tttattttaagcatct[C/G] |
| | | | | | | | | | agaactggtatcagcc |
| | 030 | rs886527 | 5 | 11200814 | 8.0E−04 | 1.497 | 0.503 | 0.403 | (SEQ ID No: 104) |
| | | | | | | | | | aatgaggcgacaacat[A/G] |
| | | | | | | | | | aaacgaaggtaatgtg |
| | 031 | rs31923 | 5 | 14724030 | 7.6E−04 | 1.984 | 0.109 | 0.058 | (SEQ ID No: 105) |
| | | | | | | | | | tgcacaaacatctgct[A/T] |
| | | | | | | | | | acttcatggccaccaa |
| | 031 | rs25986 | 5 | 14771036 | 2.4E−04 | 2.1 | 0.112 | 0.057 | (SEQ ID No: 106) |
| | | | | | | | | | ttttcgatttttggat[G/T] |
| | | | | | | | | | aggcatactcaatctg |
| | 032 | rs401531 | 5 | 142249012 | 5.3E−05 | 0.5102 | 0.14 | 0.242 | (SEQ ID No: 107) |
| | | | | | | | | | accgttctgagcactg[G/T] |
| | | | | | | | | | aggagaaaaatgaaaa |
| | 032 | rs153172 | 5 | 142258202 | 1.6E−03 | 0.6399 | 0.222 | 0.308 | (SEQ ID No: 108) |
| | | | | | | | | | agaaatattcaggatg[G/T] |
| | | | | | | | | | aaaactcggaggatgc |
| | 032 | rs409509 | 5 | 142269057 | 2.5E−03 | 0.6197 | 0.163 | 0.239 | (SEQ ID No: 109) |
| | | | | | | | | | gaggaccgaagtggaa[C/T] |
| | | | | | | | | | gcttgtagaggactag |
| | 032 | rs712161 | 5 | 142271912 | 1.1E−04 | 0.5572 | 0.185 | 0.289 | (SEQ ID No: 110) |
| | | | | | | | | | ttctgtgtttctgggc[A/C] |
| | | | | | | | | | ataaactagtgctatg |
| 12 | 032 | rs252234 | 5 | 142309329 | 7.9E−04 | 0.6017 | 0.185 | 0.273 | (SEQ ID No: 111) |
| | | | | | | | | | gtttagaacattgtac[C/T] |
| | | | | | | | | | gaaaagtcttgagcca |
| | 032 | rs10875598 | 5 | 142371351 | 4.2E−04 | 0.5544 | 0.139 | 0.226 | (SEQ ID No: 112) |
| | | | | | | | | | ctcatgtggctattga[A/G] |
| | | | | | | | | | gaagggcatagcggct |
| | 033 | rs4705344 | 5 | 148802311 | 2.6E−03 | 0.6888 | 0.37 | 0.46 | (SEQ ID No: 113) |
| | | | | | | | | | gttcactgatgcttga[C/T] |
| | | | | | | | | | gcttattgcaggtctg |
| | 033 | rs353249 | 5 | 148804949 | 1.0E−04 | 1.974 | 0.153 | 0.084 | (SEQ ID No: 114) |
| | | | | | | | | | tttctcatgatttcca[C/T] |
| | | | | | | | | | gtgttaagaataacgc |
| | 034 | rs7712987 | 5 | 153280199 | 2.7E−04 | 0.5925 | 0.21 | 0.31 | (SEQ ID No: 115) |
| | | | | | | | | | tccattacctccttca[A/T] |
| | | | | | | | | | ctctggcaacacaata |
| | NA | rs17617521 | 5 | 153329452 | 4.7E−03 | 0.6362 | 0.161 | 0.231 | (SEQ ID No: 116) |
| | | | | | | | | | atttgtgtggtgaata[C/T] |
| | | | | | | | | | aattctctccttctta |
| | 035 | rs2546707 | 5 | 168855250 | 4.3E−03 | 0.6915 | 0.308 | 0.391 | (SEQ ID No: 117) |
| | | | | | | | | | ggcaaatgatgctgtc[C/T] |
| | | | | | | | | | catagttgctttaatg |
| | 035 | rs29818 | 5 | 168891869 | 3.0E−03 | 0.6788 | 0.293 | 0.379 | (SEQ ID No: 118) |
| | | | | | | | | | agaaaagaggcatttg[C/T] |
| | | | | | | | | | atttctctaagaatac |
| | 035 | rs250275 | 5 | 168911599 | 4.1E−03 | 0.6874 | 0.293 | 0.376 | (SEQ ID No: 119) |
| | | | | | | | | | tgcaaatatttctgta[A/G] |
| | | | | | | | | | gatagagccctaaaaa |
| | 035 | rs29808 | 5 | 168936386 | 3.1E−03 | 0.6868 | 0.325 | 0.413 | (SEQ ID No: 120) |
| | | | | | | | | | tcaaaagtacagtcac[A/G] |
| | | | | | | | | | agaaatgttaggtgtt |
| 13 | 035 | rs29807 | 5 | 168937127 | 3.8E−04 | 0.6364 | 0.339 | 0.447 | (SEQ ID No: 121) |
| | | | | | | | | | catgtttcaggcacta[C/T] |
| | | | | | | | | | ggtacttgctgggaga |
| | 035 | rs154035 | 5 | 168941638 | 3.5E−03 | 0.6832 | 0.296 | 0.381 | (SEQ ID No: 122) |
| | | | | | | | | | agggctgtaaatgcta[C/T] |
| | | | | | | | | | gcaagtggtatgtgcc |
| | 035 | rs6912295 | 6 | 3661197 | 1.9E−03 | 0.6831 | 0.388 | 0.482 | (SEQ ID No: 123) |
| | | | | | | | | | tggcattcttttccca[C/T] |
| | | | | | | | | | cctcatcgtaggtata |
| | 036 | rs4959862 | 6 | 3662712 | 3.6E−04 | 0.633 | 0.311 | 0.416 | (SEQ ID No: 124) |
| | | | | | | | | | atcatgtcctgccaga[A/G] |
| | | | | | | | | | tatttactccctacaa |
| | 036 | rs9378813 | 6 | 3663678 | 5.8E−05 | 0.5987 | 0.321 | 0.442 | (SEQ ID No: 125) |
| | | | | | | | | | gattggtgtgcatgtg[C/T] |
| | | | | | | | | | caagcaaactagcaga |
| | 036 | rs9378381 | 6 | 3663880 | 1.8E−04 | 0.622 | 0.323 | 0.434 | (SEQ ID No: 126) |
| | | | | | | | | | ctccctatctcctccg[C/T] |
| | | | | | | | | | acagaaaccacaataa |
| | NA | rs2228220 | 6 | 12231235 | 1.3E−03 | 0.4297 | 0.047 | 0.104 | (SEQ ID No: 127) |
| | | | | | | | | | acttctlatggtaaca[C/T] |
| | | | | | | | | | tatgtttaggcaaact |
| | NA | rs3734271 | 6 | 12237922 | 2.8E−03 | 0.3437 | 0.024 | 0.066 | (SEQ ID No: 128) |
| | | | | | | | | | atttccagtgaaagat[G/T] |
| | | | | | | | | | agcatgcttgtgtttt |
| | 037 | rs9394604 | 6 | 12278647 | 7.7E−04 | 0.2072 | 0.012 | 0.055 | (SEQ ID No: 129) |
| | | | | | | | | | tccgtatttatcccta[A/C] |
| | | | | | | | | | ggcgtgccatctataa |
| | 037 | rs219963 | 6 | 12278913 | 1.2E−03 | 0.2743 | 0.018 | 0.062 | (SEQ ID No: 130) |
| | | | | | | | | | atattatggttggatt[C/T] |
| | | | | | | | | | aaggatgattagtacg |
| 14 | 038 | rs9383114 | 6 | 16207259 | 3.9E−04 | 1.536 | 0.544 | 0.438 | (SEQ ID No: 131) |
| | | | | | | | | | aatgcagagaaagtgg[A/T] |
| | | | | | | | | | aaggtagtaaactagt |
| | 038 | rs6912988 | 6 | 16208633 | 7.6E−04 | 1.5 | 0.55 | 0.449 | (SEQ ID No: 132) |
| | | | | | | | | | gcagggttggttcata[C/T] |
| | | | | | | | | | tagagattgtgaggga |
| | NA | rs1011616 | 6 | 16240388 | 4.2E−03 | 0.5283 | 0.071 | 0.126 | (SEQ ID No: 133) |
| | | | | | | | | | ttatttggtactccaa[A/G] |
| | | | | | | | | | cttctgaagatttaaa |
| | 039 | rs4510658 | 6 | 22863298 | 7.8E−04 | 0.6505 | 0.314 | 0.413 | (SEQ ID No: 134) |
| | | | | | | | | | ggaacgtagaactgta[C/T] |
| | | | | | | | | | gaagaggtcatttgag |
| | NA | rs6456506 | 6 | 22904732 | 2.0E−03 | 1.455 | 0.47 | 0.379 | (SEQ ID No: 135) |
| | | | | | | | | | tcttcaacacagtacc[A/G] |
| | | | | | | | | | aaattgctagccagag |
| | 040 | rs10484958 | 6 | 47229145 | 3.0E−04 | 1.732 | 0.213 | 0.135 | (SEQ ID No: 136) |
| | | | | | | | | | atgagttggcttaaaa[C/T] |
| | | | | | | | | | agtaattcaagtcaca |
| | 040 | rs9395236 | 6 | 47236608 | 5.8E−04 | 1.736 | 0.186 | 0.117 | (SEQ ID No: 137) |
| | | | | | | | | | gaaagaaaagttaaac[A/C] |
| | | | | | | | | | aattagcagagacacc |
| | 041 | rs10806546 | 6 | 67569403 | 2.6E−04 | 0.6387 | 0.385 | 0.495 | (SEQ ID No: 138) |
| | | | | | | | | | aggccatacctttgaa[A/T] |
| | | | | | | | | | tctcactgttcatatg |
| | 041 | rs7748379 | 6 | 67586040 | 3.2E−03 | 1.492 | 0.297 | 0.221 | (SEQ ID No: 139) |
| | | | | | | | | | tatttgaaattgaatg[C/T] |
| | | | | | | | | | aacgaatgattttata |
| | 042 | rs1014021 | 6 | 135376293 | 1.0E−03 | 0.6707 | 0.411 | 0.51 | (SEQ ID No: 140) |
| | | | | | | | | | ttacctgcttttctaa[C/T] |
| | | | | | | | | | cttatgtctcaccaca |
| 15 | 042 | rs6923765 | 6 | 135376869 | 9.3E−04 | 0.6684 | 0.406 | 0.506 | (SEQ ID No: 141) |
| | | | | | | | | | cagagccgaaacccaa[C/G] |
| | | | | | | | | | gaaacaagggacaagt |
| | 042 | rs7750300 | 6 | 135392643 | 1.2E−03 | 0.6732 | 0.408 | 0.506 | (SEQ ID No: 142) |
| | | | | | | | | | tgttaagaaagaatga[C/G] |
| | | | | | | | | | attgatctgtatatgg |
| | 042 | rs1590975 | 6 | 135393781 | 6.4E−04 | 0.6601 | 0.409 | 0.512 | (SEQ ID No: 143) |
| | | | | | | | | | atttcacagaaggtga[C/T] |
| | | | | | | | | | gtcggcctttttgata |
| | 042 | rs9321481 | 6 | 135394341 | 1.0E−03 | 0.6711 | 0.409 | 0.508 | (SEQ ID No: 144) |
| | | | | | | | | | gagtagcaggtgaaga[C/G] |
| | | | | | | | | | agatgaaccaaggatg |
| | 042 | rs11754265 | 6 | 135397909 | 6.9E−04 | 0.6612 | 0.411 | 0.514 | (SEQ ID No: 145) |
| | | | | | | | | | caaagtttcagcaatg[C/G] |
| | | | | | | | | | aggatcaatagaagct |
| | 042 | rs7741515 | 6 | 135416061 | 1.8E−04 | 0.6334 | 0.406 | 0.519 | (SEQ ID No: 146) |
| | | | | | | | | | agggatacacagatga[C/G] |
| | | | | | | | | | aacacttaattcagaa |
| | 042 | rs2150681 | 6 | 135416925 | 8.5E−04 | 0.666 | 0.408 | 0.509 | (SEQ ID No: 147) |
| | | | | | | | | | tggaaacttaggagac[A/G] |
| | | | | | | | | | gtagaaaagacacatg |
| | 042 | rs1331308 | 6 | 135446815 | 3.7E−04 | 1.551 | 0.585 | 0.477 | (SEQ ID No: 148) |
| | | | | | | | | | atttctactagtctga[G/T] |
| | | | | | | | | | aaacccattcaagcca |
| | NA | rs7786654 | 7 | 2922141 | 1.8E−03 | 0.6058 | 0.156 | 0.234 | (SEQ ID No: 149) |
| | | | | | | | | | agttaataaatccatc[A/G] |
| | | | | | | | | | aaagcccgtccccaaa |
| | NA | rs11773149 | 7 | 2922407 | 1.3E−03 | 0.5899 | 0.147 | 0.226 | (SEQ ID No: 150) |
| | | | | | | | | | caacctgttgtctcac[A/G] |
| | | | | | | | | | aagtgatctcctgcca |
| 16 | 043 | rs6958161 | 7 | 2928051 | 4.4E−04 | 0.5722 | 0.162 | 0.252 | (SEQ ID No: 151) |
| | | | | | | | | | tgggcaggtggaagac[A/G] |
| | | | | | | | | | atgggtcaaggataag |
| | 043 | rs6461856 | 7 | 2940640 | 2.1E−04 | 0.5492 | 0.15 | 0.243 | (SEQ ID No: 152) |
| | | | | | | | | | atcaattcttccttca[G/T] |
| | | | | | | | | | taatgcctcagaaaaa |
| | NA | rs10241890 | 7 | 2993500 | 2.4E−03 | 1.444 | 0.5 | 0.409 | (SEQ ID No: 153) |
| | | | | | | | | | aaagacaaatgaaata[A/G] |
| | | | | | | | | | taagtctgtggtattg |
| | NA | rs2164961 | 7 | 46934373 | 1.5E−03 | 0.5833 | 0.136 | 0.213 | (SEQ ID No: 154) |
| | | | | | | | | | cgctcatacgaaatga[A/G] |
| | | | | | | | | | acctgcctatgccttg |
| | NA | rs6978709 | 7 | 46936014 | 1.5E−03 | 0.5847 | 0.136 | 0.212 | (SEQ ID No: 155) |
| | | | | | | | | | taaaatacaattgcta[A/G] |
| | | | | | | | | | ctgaaatctgcagtag |
| | 044 | rs11770341 | 7 | 46936429 | 4.6E−04 | 1.621 | 0.277 | 0.191 | (SEQ ID No: 156) |
| | | | | | | | | | gaaggattggacaata[A/G] |
| | | | | | | | | | aagcaatttaaatgtg |
| | NA | rs12718964 | 7 | 55272626 | 1.4E−03 | 1.494 | 0.39 | 0.3 | (SEQ ID No: 157) |
| | | | | | | | | | ggtgtggggtggagaa[A/G] |
| | | | | | | | | | gtgctagtagcccacc |
| | 045 | rs10281500 | 7 | 55317099 | 4.6E−04 | 1.542 | 0.454 | 0.35 | (SEQ ID No: 158) |
| | | | | | | | | | tgtgttctccccgaaa[C/G] |
| | | | | | | | | | ttctctctgtcctgtg |
| | 045 | rs9656707 | 7 | 55327194 | 4.2E−03 | 1.417 | 0.455 | 0.371 | (SEQ ID No: 159) |
| | | | | | | | | | ccaaggctgaaaatac[C/T] |
| | | | | | | | | | gatactcaacaaacag |
| | 046 | rs10156094 | 7 | 75648618 | 6.8E−04 | 0.6579 | 0.379 | 0.481 | (SEQ ID No: 160) |
| | | | | | | | | | ccattgtcatgctcca[A/G] |
| | | | | | | | | | tctgttcaagcctttg |
| 17 | 046 | rs4728710 | 7 | 75657317 | 3.2E−03 | 0.6973 | 0.396 | 0.485 | (SEQ ID No: 161) |
| | | | | | | | | | gcctggcctgcagaag[A/G] |
| | | | | | | | | | accacggagcgttgtg |
| | 047 | rs9986802 | 7 | 141326058 | 4.4E−04 | 0.6333 | 0.302 | 0.406 | (SEQ ID No: 162) |
| | | | | | | | | | aatgtggagcaactac[A/G] |
| | | | | | | | | | gacatgcaaaatgata |
| | 047 | rs9986873 | 7 | 141326076 | 3.8E−04 | 0.6328 | 0.305 | 0.409 | (SEQ ID No: 163) |
| | | | | | | | | | agataagaaagtccaa[C/T] |
| | | | | | | | | | aaatgtggagcaacta |
| | 047 | rs4389847 | 7 | 141329303 | 3.2E−04 | 0.6303 | 0.309 | 0.415 | (SEQ ID No: 164) |
| | | | | | | | | | ggaagccacatcttga[C/T] |
| | | | | | | | | | aaatgcagaggcaatt |
| | 048 | rs7783954 | 7 | 150237027 | 3.1E−04 | 0.6355 | 0.342 | 0.45 | (SEQ ID No: 165) |
| | | | | | | | | | catggcttcaccgcag[C/T] |
| | | | | | | | | | agtgcttttattatta |
| | 048 | rs7810706 | 7 | 150268937 | 7.0E−04 | 3.099 | 0.044 | 0.015 | (SEQ ID No: 166) |
| | | | | | | | | | ctcctggcagactccc[C/T] |
| | | | | | | | | | ctagagctgctttgag |
| | 049 | rs7462266 | 8 | 6109511 | 8.2E−04 | 4.08 | 0.03 | 0.008 | (SEQ ID No: 167) |
| | | | | | | | | | gcaggcggaacacaaa[C/G] |
| | | | | | | | | | tcaggagatcgagacc |
| | 049 | rs6559135 | 8 | 6132325 | 1.8E−16 | 29.69 | 0.058 | 0.002 | (SEQ ID No: 168) |
| | | | | | | | | | acaacaagtgactaca[C/T] |
| | | | | | | | | | cgtaatgagaaataga |
| | 050 | rs1671410 | 8 | 13269142 | 3.6E−03 | 0.6885 | 0.314 | 0.399 | (SEQ ID No: 169) |
| | | | | | | | | | ttcacgtcctgagtta[C/T] |
| | | | | | | | | | aaaccctgcaggtccc |
| | 050 | rs897266 | 8 | 13269177 | 6.9E−04 | 1.506 | 0.515 | 0.413 | (SEQ ID No: 170) |
| | | | | | | | | | cagttcatgatctgga[C/T] |
| | | | | | | | | | ggattgagtgagatgc |
| 18 | 051 | rs2976427 | 8 | 24875821 | 1.5E−03 | 0.507 | 0.076 | 0.14 | (SEQ ID No: 171) |
| | | | | | | | | | aataaaaagaaaccaa[C/T] |
| | | | | | | | | | tactactacgtacaac |
| | 051 | rs2976425 | 8 | 24877700 | 4.5E−04 | 0.473 | 0.077 | 0.15 | (SEQ ID No: 172) |
| | | | | | | | | | tcactgctcaaggtcc[C/T] |
| | | | | | | | | | caagctttcccctctt |
| | 052 | rs218019 | 8 | 118776737 | 7.9E−04 | 0.6392 | 0.268 | 0.364 | (SEQ ID No: 173) |
| | | | | | | | | | ttttcaaggctgaaca[A/G] |
| | | | | | | | | | tggacttgcttttgga |
| | NA | rs218021 | 8 | 118778576 | 4.0E−03 | 0.681 | 0.268 | 0.349 | (SEQ ID No: 174) |
| | | | | | | | | | cagcaatgagttacca[A/G] |
| | | | | | | | | | tatgtgaatttccagc |
| | 053 | rs16912206 | 9 | 106942539 | 5.2E−04 | 0.383 | 0.041 | 0.101 | (SEQ ID No: 175) |
| | | | | | | | | | ataaatgagacgtgac[C/T] |
| | | | | | | | | | gtactttaaggagtgc |
| | 053 | rs16912217 | 9 | 106956465 | 1.5E−03 | 0.4883 | 0.068 | 0.13 | (SEQ ID No: 176) |
| | | | | | | | | | taatttagcagatagg[A/G] |
| | | | | | | | | | atccaaagaatatgtg |
| | 053 | rs4978605 | 9 | 106966463 | 2.1E−04 | 0.4868 | 0.095 | 0.177 | (SEQ ID No: 177) |
| | | | | | | | | | gttttaaaggtcagag[C/T] |
| | | | | | | | | | atggcgtccattcaca |
| | 053 | rs10521081 | 9 | 106968197 | 1.2E−04 | 0.4602 | 0.086 | 0.169 | (SEQ ID No: 178) |
| | | | | | | | | | attaatgctttgctct[A/G] |
| | | | | | | | | | accaactgggcaaatc |
| | 054 | rs17794822 | 9 | 109692695 | 6.0E−04 | 1.847 | 0.144 | 0.084 | (SEQ ID No: 179) |
| | | | | | | | | | gtcagtcaaggtggaa[A/G] |
| | | | | | | | | | atcactgcagggcaca |
| | 054 | rs17794870 | 9 | 109701595 | 5.4E−04 | 1.868 | 0.142 | 0.081 | (SEQ ID No: 180) |
| | | | | | | | | | atgtacccatttaacc[C/T] |
| | | | | | | | | | gcaacatcaccaggtg |
| 19 | 055 | rs10987199 | 9 | 126048541 | 2.6E−04 | 1.885 | 0.159 | 0.091 | (SEQ ID No: 181) |
| | | | | | | | | | aaggccttttctctgg[A/G] |
| | | | | | | | | | cacaggcagggagagc |
| | NA | rs2039201 | 9 | 126074416 | 2.9E−03 | 1.764 | 0.124 | 0.074 | (SEQ ID No: 182) |
| | | | | | | | | | aatgtcgtctgcccag[C/G] |
| | | | | | | | | | aatgcagagtgtagat |
| | 056 | rs947626 | 9 | 127853923 | 5.4E−04 | 1.68 | 0.219 | 0.143 | (SEQ ID No: 183) |
| | | | | | | | | | gttttcttgaaataca[G/T] |
| | | | | | | | | | tactcactcttgggat |
| | 056 | rs4837223 | 9 | 127855961 | 3.3E−03 | 1.55 | 0.219 | 0.153 | (SEQ ID No: 184) |
| | | | | | | | | | ctctgggcacctgaca[C/T] |
| | | | | | | | | | gtataaactcaccaga |
| | 056 | rs4836598 | 9 | 127856047 | 3.4E−03 | 1.548 | 0.219 | 0.153 | (SEQ ID No: 185) |
| | | | | | | | | | aggagactaagcctcg[A/G] |
| | | | | | | | | | agaagtgagttgtttg |
| | NA | rs7916062 | 10 | 19528355 | 1.6E−03 | 1.486 | 0.374 | 0.286 | (SEQ ID No: 186) |
| | | | | | | | | | gattctaacccaaacc[C/T] |
| | | | | | | | | | gatggcaaaggctatc |
| | NA | rs10508577 | 10 | 19530733 | 1.3E−03 | 1.532 | 0.313 | 0.229 | (SEQ ID No: 187) |
| | | | | | | | | | taattttgctttaccg[A/C] |
| | | | | | | | | | agatttctaaacttta |
| | NA | rs2245151 | 10 | 19539148 | 1.8E−03 | 1.62 | 0.2 | 0.134 | (SEQ ID No: 188) |
| | | | | | | | | | gcatttgagcttgcta[G/T] |
| | | | | | | | | | aataagactgactttt |
| | 057 | rs2803834 | 10 | 19540143 | 2.5E−03 | 1.466 | 0.372 | 0.288 | (SEQ ID No: 189) |
| | | | | | | | | | atcgacgtgggactaa[C/G] |
| | | | | | | | | | caaggaacactttcgt |
| | 057 | rs2782326 | 10 | 19541131 | 1.9E−04 | 1.623 | 0.372 | 0.267 | (SEQ ID No: 190) |
| | | | | | | | | | aggaaactggaggtga[A/T] |
| | | | | | | | | | gtatatttccatcacc |
| 20 | 057 | rs6481792 | 10 | 19541172 | 8.9E−04 | 1.675 | 0.204 | 0.132 | (SEQ ID No: 191) |
| | | | | | | | | | caggaatgaggggcca[A/G] |
| | | | | | | | | | agaaatgtattataaa |
| | 057 | rs2245456 | 10 | 19542000 | 1.2E−03 | 1.508 | 0.371 | 0.281 | (SEQ ID No: 192) |
| | | | | | | | | | taatagattcttttga[A/G] |
| | | | | | | | | | gagcagattctttttt |
| | 058 | rs11004685 | 10 | 56458398 | 1.4E−03 | 0.6753 | 0.377 | 0.472 | (SEQ ID No: 193) |
| | | | | | | | | | cactggaagatgaaga[C/G] |
| | | | | | | | | | gtaatgatcacatttc |
| | 058 | rs10825442 | 10 | 56459738 | 1.5E−03 | 0.6757 | 0.377 | 0.472 | (SEQ ID No: 194) |
| | | | | | | | | | accaagatttcaaaga[C/G] |
| | | | | | | | | | gtgaataaaagctcac |
| | 058 | rs11004686 | 10 | 56459901 | 4.9E−03 | 0.7007 | 0.337 | 0.421 | (SEQ ID No: 195) |
| | | | | | | | | | ttcatatttttggaaa[C/G] |
| | | | | | | | | | tgacttaatttctatg |
| | 058 | rs11004689 | 10 | 56462885 | 5.0E−04 | 0.6512 | 0.376 | 0.48 | (SEQ ID No: 196) |
| | | | | | | | | | cttcaagtgatacctc[A/G] |
| | | | | | | | | | gagagcctcaggttct |
| | 058 | rs10430541 | 10 | 56494253 | 1.8E−03 | 1.46 | 0.482 | 0.39 | (SEQ ID No: 197) |
| | | | | | | | | | aatagagattatatgc[C/T] |
| | | | | | | | | | ccaacctaatatgagt |
| | 058 | rs10825454 | 10 | 56494490 | 2.9E−03 | 0.69 | 0.38 | 0.47 | (SEQ ID No: 198) |
| | | | | | | | | | gaattagaagtaaatg[A/G] |
| | | | | | | | | | aaactcgtaactacat |
| | 059 | rs3862864 | 10 | 59582876 | 1.6E−04 | 2.022 | 0.132 | 0.07 | (SEQ ID No: 199) |
| | | | | | | | | | gatactatgaaaatca[A/G] |
| | | | | | | | | | tccaaaacatcgtgat |
| | 059 | rs1199094 | 10 | 59622636 | 8.7E−04 | 1.802 | 0.147 | 0.087 | (SEQ ID No: 200) |
| | | | | | | | | | tgtaatagaaagggta[A/T] |
| | | | | | | | | | ctgagggtacaaacat |
| 21 | NA | rs12413000 | 10 | 71988196 | 2.9E−03 | 1.431 | 0.5 | 0.411 | (SEQ ID No: 201) |
| | | | | | | | | | gtaataaccgtttgtc[A/C] |
| | | | | | | | | | aatcaatgaatgacaa |
| | 060 | rs4747053 | 10 | 71990162 | 8.9E−05 | 1.622 | 0.412 | 0.302 | (SEQ ID No: 202) |
| | | | | | | | | | tagttaacccttcaaa[C/G] |
| | | | | | | | | | agtaggcatcctgccc |
| | 061 | rs7905535 | 10 | 73200945 | 9.9E−04 | 1.499 | 0.458 | 0.36 | (SEQ ID No: 203) |
| | | | | | | | | | tgggaatgtctgtggg[A/G] |
| | | | | | | | | | aagaagaggctctgct |
| | 061 | rs7076717 | 10 | 73235195 | 8.8E−05 | 0.5173 | 0.136 | 0.233 | (SEQ ID No: 204) |
| | | | | | | | | | aagactgatacaggac[A/G] |
| | | | | | | | | | aagggtggctgtggcc |
| | NA | rs2283213 | 11 | 2707120 | 1.8E−03 | 2.158 | 0.074 | 0.036 | (SEQ ID No: 205) |
| | | | | | | | | | ggtgcccacacctata[C/T] |
| | | | | | | | | | aatgggggtaataaag |
| | 062 | rs2075870 | 11 | 2746595 | 9.4E−04 | 2.91 | 0.044 | 0.016 | (SEQ ID No: 206) |
| | | | | | | | | | cctgtgctggtcatcc[C/T] |
| | | | | | | | | | ctcactgcccctgcac |
| | 063 | rs12223197 | 11 | 19063319 | 4.0E−04 | 1.765 | 0.185 | 0.114 | (SEQ ID No: 207) |
| | | | | | | | | | gaactcctgacttcca[A/G] |
| | | | | | | | | | cctggtgctntccta |
| | 063 | rs2089051 | 11 | 19070682 | 8.7E−04 | 1.616 | 0.241 | 0.164 | (SEQ ID No: 208) |
| | | | | | | | | | gattttgattgatggg[C/T] |
| | | | | | | | | | catatattagcctggt |
| | NA | rs1255522 | 11 | 95016096 | 4.4E−03 | 0.7013 | 0.356 | 0.441 | (SEQ ID No: 209) |
| | | | | | | | | | caccctctagttgtga[A/G] |
| | | | | | | | | | attcaaaaatgtctcc |
| | 064 | rs1255523 | 11 | 95019155 | 4.4E−03 | 0.7003 | 0.352 | 0.437 | (SEQ ID No: 210) |
| | | | | | | | | | catacaccaatcagaa[A/G] |
| | | | | | | | | | gggactgttcagaaaa |
| 22 | 064 | rs1262220 | 11 | 95025261 | 7.1E−04 | 0.6629 | 0.412 | 0.514 | (SEQ ID No: 211) |
| | | | | | | | | | acctgcttgcctgcaa[C/T] |
| | | | | | | | | | atgagttttacaccat |
| | 064 | rs10831399 | 11 | 95030832 | 3.8E−04 | 1.538 | 0.571 | 0.464 | (SEQ ID No: 212) |
| | | | | | | | | | cataaagcagagatga[A/G] |
| | | | | | | | | | taaaagccagagggag |
| | 064 | rs941292 | 11 | 95040116 | 1.0E−03 | 1.487 | 0.539 | 0.44 | (SEQ ID No: 213) |
| | | | | | | | | | tctctggaacaatcac[A/T] |
| | | | | | | | | | gtctttctctcgctac |
| | 064 | rs9651554 | 11 | 95040500 | 1.8E−03 | 0.6804 | 0.41 | 0.505 | (SEQ ID No: 214) |
| | | | | | | | | | gatccatcttttagca[A/G] |
| | | | | | | | | | gaggtacatttcctta |
| | 064 | rs1939876 | 11 | 95042333 | 2.3E−04 | 1.569 | 0.578 | 0.466 | (SEQ ID No: 215) |
| | | | | | | | | | gacataaaaggttaca[C/T] |
| | | | | | | | | | attgtatgatgctact |
| | 064 | rs7932590 | 11 | 95048677 | 1.0E−04 | 1.601 | 0.577 | 0.46 | (SEQ ID No: 216) |
| | | | | | | | | | aacttaatggaataga[A/C] |
| | | | | | | | | | ggtagcaaccacacat |
| | 065 | rs473573 | 11 | 127633623 | 9.7E−04 | 0.2429 | 0.015 | 0.059 | (SEQ ID No: 217) |
| | | | | | | | | | gtgtatacttgtgagg[C/G] |
| | | | | | | | | | catgagcatgattaag |
| | 065 | rs530178 | 11 | 127637217 | 1.1E−03 | 0.2463 | 0.015 | 0.057 | (SEQ ID No: 218) |
| | | | | | | | | | agtaaagcttaagaca[A/G] |
| | | | | | | | | | ctctgcttcaaattct |
| | 065 | rs7950582 | 11 | 127684286 | 7.9E−04 | 0.1288 | 0.006 | 0.045 | (SEQ ID No: 219) |
| | | | | | | | | | agataaaaattaaaag[A/C] |
| | | | | | | | | | cttagatcctgcattc |
| | 066 | rs622513 | 12 | 3941499 | 6.7E−05 | 1.618 | 0.544 | 0.425 | (SEQ ID No: 220) |
| | | | | | | | | | agagattgatgtggta[C/T] |
| | | | | | | | | | atttagctgaattttg |
| 23 | 066 | rs609018 | 12 | 3942162 | 1.1E−04 | 1.593 | 0.527 | 0.411 | (SEQ ID No: 221) |
| | | | | | | | | | acaatttctccttcac[G/T] |
| | | | | | | | | | gccagtttgagataag |
| | NA | rs4930767 | 12 | 5467642 | 1.8E−03 | 0.6824 | 0.396 | 0.491 | (SEQ ID No: 222) |
| | | | | | | | | | aactcactggggttac[A/G] |
| | | | | | | | | | atggatccaaaaccaa |
| | 067 | rs11612899 | 12 | 5483797 | 6.0E−04 | 0.5706 | 0.148 | 0.233 | (SEQ ID No: 223) |
| | | | | | | | | | gagctttgcgattgta[C/T] |
| | | | | | | | | | agaaggggagtcaagt |
| | 068 | rs10747645 | 12 | 51416611 | 4.5E−04 | 1.559 | 0.367 | 0.271 | (SEQ ID No: 224) |
| | | | | | | | | | gaaaatcacctaatta[C/T] |
| | | | | | | | | | agcaaatgccctgatg |
| | 068 | rs4564385 | 12 | 51419279 | 2.8E−03 | 1.506 | 0.317 | 0.236 | (SEQ ID No: 225) |
| | | | | | | | | | ccaccgcgcctggcga[C/T] |
| | | | | | | | | | aaattcttttaaatat |
| | 068 | rs7956525 | 12 | 51421288 | 3.0E−03 | 1.464 | 0.348 | 0.267 | (SEQ ID No: 226) |
| | | | | | | | | | aaatataatactctaa[C/T] |
| | | | | | | | | | caccaatgacaaatac |
| | 068 | rs7398785 | 12 | 51422165 | 3.0E−04 | 1.582 | 0.371 | 0.272 | (SEQ ID No: 227) |
| | | | | | | | | | ctccctgtgctcttta[A/G] |
| | | | | | | | | | aggtccaggagtaggc |
| | 068 | rs7306051 | 12 | 51439097 | 4.1E−04 | 1.566 | 0.364 | 0.268 | (SEQ ID No: 228) |
| | | | | | | | | | gaccagtgaggcacaa[A/G] |
| | | | | | | | | | tgaaggttttctagcg |
| | 068 | rs17738862 | 12 | 51469295 | 1.1E−04 | 3.143 | 0.057 | 0.019 | (SEQ ID No: 229) |
| | | | | | | | | | aaagtgcgggctgctg[A/G] |
| | | | | | | | | | aagcccagggatggaa |
| | 069 | rs7299978 | 12 | 61538626 | 2.1E−03 | 0.4864 | 0.062 | 0.119 | (SEQ ID No: 230) |
| | | | | | | | | | aggaatggcagtttac[C/T] |
| | | | | | | | | | cacaatttctgccctg |
| 24 | NA | rs772578 | 12 | 61540544 | 1.4E−03 | 0.5459 | 0.103 | 0.174 | (SEQ ID No: 231) |
| | | | | | | | | | acaaagctctatgtca[C/T] |
| | | | | | | | | | ctaaactgataattat |
| | 069 | rs10877926 | 12 | 61580249 | 2.2E−04 | 0.4869 | 0.095 | 0.178 | (SEQ ID No: 232) |
| | | | | | | | | | tccttcttatccaaac[A/G] |
| | | | | | | | | | aggaagtcagtattca |
| | NA | rs11103862 | 12 | 76021147 | 4.0E−03 | 1.505 | 0.25 | 0.181 | (SEQ ID No: 233) |
| | | | | | | | | | catctctttgttcatc[A/C] |
| | | | | | | | | | ctgaattctctctagt |
| | NA | rs17043056 | 12 | 76063005 | 3.5E−03 | 1.494 | 0.275 | 0.203 | (SEQ ID No: 234) |
| | | | | | | | | | tccatatccaccacag[A/C] |
| | | | | | | | | | atcaaatgaaactgta |
| | 070 | rs10776994 | 12 | 76076736 | 9.6E−04 | 1.557 | 0.307 | 0.222 | (SEQ ID No: 235) |
| | | | | | | | | | caagtgttttgttcac[A/G] |
| | | | | | | | | | cacctcaagagaggag |
| | 071 | rs835481 | 12 | 103551381 | 6.0E−04 | 1.525 | 0.418 | 0.32 | (SEQ ID No: 236) |
| | | | | | | | | | tctgccatttccatca[A/G] |
| | | | | | | | | | ctattactggacatga |
| | 071 | rs1344677 | 12 | 103553434 | 2.5E−04 | 1.564 | 0.586 | 0.475 | (SEQ ID No: 237) |
| | | | | | | | | | ttgaagatgcttgcta[A/G] |
| | | | | | | | | | accaggaaatatgatt |
| | NA | rs835479 | 12 | 103577324 | 2.6E−03 | 0.6703 | 0.274 | 0.36 | (SEQ ID No: 238) |
| | | | | | | | | | agactgcaagaagaga[A/G] |
| | | | | | | | | | aacctttaaactgatc |
| | NA | rs1097245 | 12 | 103577737 | 2.5E−03 | 0.6694 | 0.274 | 0.36 | (SEQ ID No: 239) |
| | | | | | | | | | gagttgtgcaatcatc[A/G] |
| | | | | | | | | | ctgtaacctaatttta |
| | 072 | rs11058789 | 12 | 121176298 | 1.5E−04 | 1.583 | 0.571 | 0.456 | (SEQ ID No: 240) |
| | | | | | | | | | ctgtcactgaaagccc[A/G] |
| | | | | | | | | | cagttctggagatgac |
| 25 | 072 | rs7972979 | 12 | 121187736 | 4.9E−03 | 1.405 | 0.547 | 0.463 | (SEQ ID No: 241) |
| | | | | | | | | | caggagaggttgttac[A/G] |
| | | | | | | | | | agaggttgtcaagaag |
| | 072 | rs11059505 | 12 | 121190165 | 1.5E−03 | 1.468 | 0.553 | 0.458 | (SEQ ID No: 242) |
| | | | | | | | | | aatggctgatttgaca[C/G] |
| | | | | | | | | | agtagacgcccattgg |
| | 073 | rs9552597 | 13 | 21588127 | 1.5E−03 | 1.551 | 0.277 | 0.198 | (SEQ ID No: 243) |
| | | | | | | | | | ttaaacccagtaagga[C/T] |
| | | | | | | | | | cttgaacagtagacat |
| | 073 | rs12871430 | 13 | 21591188 | 2.9E−04 | 1.636 | 0.29 | 0.2 | (SEQ ID No: 244) |
| | | | | | | | | | ttgtagctttcaagac[A/G] |
| | | | | | | | | | agaacaaagaaaatgc |
| | 074 | rs636519 | 13 | 29553181 | 3.3E−04 | 1.577 | 0.364 | 0.266 | (SEQ ID No: 245) |
| | | | | | | | | | actgctgtattccgaa[A/T] |
| | | | | | | | | | ctctagaacgtctggc |
| | 074 | rs501378 | 13 | 29553961 | 3.0E−04 | 1.584 | 0.366 | 0.267 | (SEQ ID No: 246) |
| | | | | | | | | | acatcttcactctaca[C/T] |
| | | | | | | | | | ggtagcaaagtgccag |
| | 074 | rs478410 | 13 | 29554081 | 3.7E−04 | 1.571 | 0.364 | 0.267 | (SEQ ID No: 247) |
| | | | | | | | | | agccctctcactgttg[A/G] |
| | | | | | | | | | aaacatggctgtcttt |
| | NA | rs9545131 | 13 | 35190809 | 1.5E−03 | 1.469 | 0.53 | 0.434 | (SEQ ID No: 248) |
| | | | | | | | | | cctcaaacagcttaca[A/G] |
| | | | | | | | | | catgggcgaaaaggac |
| | 075 | rs2769330 | 13 | 35194460 | 7.3E−04 | 1.502 | 0.497 | 0.397 | (SEQ ID No: 249) |
| | | | | | | | | | atccacaatatttaaa[C/T] |
| | | | | | | | | | ggctgttttagtcact |
| | 075 | rs6563133 | 13 | 35216869 | 9.8E−04 | 0.6509 | 0.293 | 0.389 | (SEQ ID No: 250) |
| | | | | | | | | | tgtggataaccaaact[A/G] |
| | | | | | | | | | acatgtggaatctgac |
| 26 | 075 | rs9545255 | 13 | 35227287 | 2.9E−03 | 0.6885 | 0.344 | 0.433 | (SEQ ID No: 2551) |
| | | | | | | | | | ttgtgcacaggaaagt[C/G] |
| | | | | | | | | | aatcagctgtgatctc |
| | NA | rs1122760 | 13 | 42708524 | 3.9E−03 | 0.5982 | 0.121 | 0.188 | (SEQ ID No: 252) |
| | | | | | | | | | aacatgtgagatgcaa[A/C] |
| | | | | | | | | | ttagcgtattcttgaa |
| | NA | rs12853905 | 13 | 42714572 | 1.8E−03 | 0.5225 | 0.082 | 0.147 | (SEQ ID No: 253) |
| | | | | | | | | | atcatagctgaaccca[C/T] |
| | | | | | | | | | gagaatgaaagaaagg |
| | NA | rs7319813 | 13 | 42722441 | 2.4E−03 | 0.5868 | 0.126 | 0.197 | (SEQ ID No: 254) |
| | | | | | | | | | gcaggcttctcagtaa[A/G] |
| | | | | | | | | | gaacagacttggggtg |
| | 076 | rs17538591 | 13 | 42760855 | 8.2E−04 | 0.4895 | 0.079 | 0.15 | (SEQ ID No: 255) |
| | | | | | | | | | tattccttccttctaa[C/T] |
| | | | | | | | | | tgaacatttttgaggc |
| | 077 | rs2325197 | 13 | 68655746 | 5.0E−04 | 0.5024 | 0.091 | 0.166 | (SEQ ID No: 256) |
| | | | | | | | | | caccctccatcggtaa[C/G] |
| | | | | | | | | | aagatcaccaggcatt |
| | NA | rs12858540 | 13 | 68655983 | 3.2E−03 | 0.3274 | 0.021 | 0.061 | (SEQ ID No: 257) |
| | | | | | | | | | cctgtgcatatgctac[A/G] |
| | | | | | | | | | ccaggggtcctcaacc |
| | 078 | rs1512762 | 13 | 82708206 | 4.8E−04 | 0.6449 | 0.352 | 0.458 | (SEQ ID No: 258) |
| | | | | | | | | | ctatattaaccataga[A/T] |
| | | | | | | | | | tcatccataatagttc |
| | 078 | rs1512771 | 13 | 82715131 | 1.2E−03 | 0.6683 | 0.362 | 0.459 | (SEQ ID No: 259) |
| | | | | | | | | | agccctgctgagatta[A/G] |
| | | | | | | | | | gtgaactccagttgat |
| | 078 | rs7147585 | 14 | 27426001 | 9.9E−04 | 0.5749 | 0.142 | 0.223 | (SEQ ID No: 260) |
| | | | | | | | | | cagatgtaacactcaa[C/T] |
| | | | | | | | | | tgaagcattctcaggt |
| 27 | NA | rs1954578 | 14 | 27457754 | 4.5E−03 | 0.6193 | 0.138 | 0.206 | (SEQ ID No: 261) |
| | | | | | | | | | ttatataacttttgga[C/T] |
| | | | | | | | | | atacagcctgatttga |
| | 080 | rs8014810 | 14 | 35394781 | 6.3E−04 | 1.641 | 0.238 | 0.16 | (SEQ ID No: 262) |
| | | | | | | | | | gttgttgaaaggatta[A/C] |
| | | | | | | | | | aatgacagagtaaata |
| | 080 | rs2415306 | 14 | 35400967 | 2.6E−03 | 1.541 | 0.241 | 0.171 | (SEQ ID No: 263) |
| | | | | | | | | | acagattgaattgcta[A/C] |
| | | | | | | | | | aacatacaagactggc |
| | 081 | rs17105308 | 14 | 36324888 | 9.9E−04 | 0.5831 | 0.147 | 0.228 | (SEQ ID No: 264) |
| | | | | | | | | | tgagaccagctggaaa[C/T] |
| | | | | | | | | | agatactgaaatcttg |
| | 081 | rs17105311 | 14 | 36324910 | 1.2E−03 | 0.5874 | 0.148 | 0.228 | (SEQ ID No: 265) |
| | | | | | | | | | caaaatgcttcactta[C/T] |
| | | | | | | | | | caaagcaagatttcag |
| | NA | SNP_A- | 14 | 36328425 | 1.5E−03 | 0.5958 | 0.151 | 0.23 | (SEQ ID No: 266) |
| | | 2097394 | | | | | | | agagaaaggtgataac[A/G] |
| | | | | | | | | | aaatgaaagcataatc |
| | NA | rs2747105 | 14 | 57540518 | 1.9E−03 | 0.665 | 0.287 | 0.377 | (SEQ ID No: 267) |
| | | | | | | | | | ctatttaagacagcaa[A/G] |
| | | | | | | | | | cgtacattaagactaa |
| | 082 | rs17734691 | 14 | 57544103 | 2.8E−03 | 0.6308 | 0.181 | 0.259 | (SEQ ID No: 268) |
| | | | | | | | | | agaggagcttattttc[C/T] |
| | | | | | | | | | cttagtcagttttgaa |
| | 082 | rs8017546 | 14 | 57546972 | 7.5E−04 | 0.6041 | 0.189 | 0.279 | (SEQ ID No: 269) |
| | | | | | | | | | taagaagaaaattttg[A/C] |
| | | | | | | | | | attccaatagtgactg |
| | 082 | rs17094208 | 14 | 57547077 | 1.1E−03 | 0.5847 | 0.147 | 0.228 | (SEQ ID No: 270) |
| | | | | | | | | | aaataagccagaaaac[C/T] |
| | | | | | | | | | agcggtcacatacaaa |
| 28 | NA | rs2526901 | 14 | 72122456 | 2.8E−03 | 1.579 | 0.207 | 0.142 | (SEQ ID No: 271) |
| | | | | | | | | | aaggaagagttttaaa[C/T] |
| | | | | | | | | | ggtagagtactatgtt |
| | NA | rs2526900 | 14 | 72122784 | 2.5E−03 | 1.586 | 0.215 | 0.147 | (SEQ ID No: 272) |
| | | | | | | | | | ttgttaccattcccac[A/G] |
| | | | | | | | | | ggtttgagttagctct |
| | 083 | rs1859474 | 14 | 72125288 | 7.0E−06 | 6.044 | 0.036 | 0.006 | (SEQ ID No: 273) |
| | | | | | | | | | cctttcttcacccagg[A/C] |
| | | | | | | | | | caacgagtttgtggaa |
| | 084 | rs12431457 | 14 | 73384916 | 1.6E−03 | 1.463 | 0.506 | 0.412 | (SEQ ID No: 274) |
| | | | | | | | | | gttgcactaagtgtca[C/T] |
| | | | | | | | | | ataccatgcaggttgg |
| | 084 | rs2159179 | 14 | 73386601 | 1.6E−03 | 1.466 | 0.562 | 0.467 | (SEQ ID No: 275) |
| | | | | | | | | | gtaactaataagtaaa[A/G] |
| | | | | | | | | | gagcaagttatcctgc |
| | 084 | rs8013446 | 14 | 73429247 | 4.2E−03 | 0.5555 | 0.085 | 0.144 | (SEQ ID No: 276) |
| | | | | | | | | | cctcagctgtaaaata[C/T] |
| | | | | | | | | | gccttaagtttgggtg |
| | 084 | rs2336547 | 14 | 73469044 | 2.3E−03 | 0.5489 | 0.094 | 0.159 | (SEQ ID No: 277) |
| | | | | | | | | | ggcactcttgctatca[A/G] |
| | | | | | | | | | tcatcctgtctccaca |
| | 084 | rs17094182 | 14 | 73490193 | 4.0E−05 | 2.561 | 0.089 | 0.037 | (SEQ ID No: 278) |
| | | | | | | | | | agttgaagatgtggac[A/C] |
| | | | | | | | | | cctgcagttgctactg |
| | NA | rs4646855 | 14 | 73619509 | 4.5E−03 | 1.517 | 0.232 | 0.166 | (SEQ ID No: 279) |
| | | | | | | | | | gatactagaacagaac[A/G] |
| | | | | | | | | | aggatcgcagcaagaa |
| | NA | rs7160897 | 14 | 73642440 | 2.7E−03 | 1.548 | 0.234 | 0.165 | (SEQ ID No: 280) |
| | | | | | | | | | aacccttttggtgtgc[A/C] |
| | | | | | | | | | aattttggaatgcagt |
| 29 | NA | rs8013357 | 14 | 73643916 | 3.7E−03 | 1.526 | 0.232 | 0.166 | (SEQ ID No: 281) |
| | | | | | | | | | aaagctgatagaagga[C/T] |
| | | | | | | | | | agcttaccgagaaaaa |
| | NA | rs2300192 | 14 | 73649222 | 2.8E−03 | 1.545 | 0.234 | 0.165 | (SEQ ID No: 282) |
| | | | | | | | | | ttcatacaaggctatg[C/T] |
| | | | | | | | | | aggggataggaagata |
| | NA | rs2300189 | 14 | 73651915 | 4.0E−03 | 1.532 | 0.228 | 0.161 | (SEQ ID No: 283) |
| | | | | | | | | | gccagagaacttgatt[C/G] |
| | | | | | | | | | aaaagcaccccaaaga |
| | NA | rs11159062 | 14 | 73681769 | 1.1E−03 | 1.612 | 0.241 | 0.165 | (SEQ ID No: 284) |
| | | | | | | | | | tggagggcacacataa[A/G] |
| | | | | | | | | | agtaatatggaagaac |
| | NA | rs2358626 | 14 | 73682764 | 3.4E−03 | 1.54 | 0.232 | 0.164 | (SEQ ID No: 285) |
| | | | | | | | | | aggcatatttttctta[C/T] |
| | | | | | | | | | ggttggcacagatata |
| | NA | rs4903199 | 14 | 73703039 | 4.8E−03 | 1.514 | 0.229 | 0.164 | (SEQ ID No: 286) |
| | | | | | | | | | aatctgtgtgcccaaa[A/C] |
| | | | | | | | | | aatccaaaatatataa |
| | NA | rs1029891 | 14 | 73703618 | 4.2E−03 | 1.524 | 0.229 | 0.163 | (SEQ ID No: 287) |
| | | | | | | | | | aatgacatttgctgca[C/G] |
| | | | | | | | | | gttgataattagtttt |
| | NA | rs1548769 | 14 | 73716352 | 2.7E−03 | 1.548 | 0.234 | 0.165 | (SEQ ID No: 288) |
| | | | | | | | | | tcatgctaaagtctga[A/G] |
| | | | | | | | | | aaccattagcctatat |
| | 085 | rs887595 | 14 | 73736394 | 4.4E−04 | 1.664 | 0.24 | 0.159 | (SEQ ID No: 289) |
| | | | | | | | | | acgcacacacacgaaa[A/G] |
| | | | | | | | | | agaatcttctgactta |
| | 086 | rs10139335 | 14 | 90637635 | 2.8E−04 | 0.4227 | 0.06 | 0.132 | (SEQ ID No: 290) |
| | | | | | | | | | agtgcacgacttccaa[C/T] |
| | | | | | | | | | gtcctggtggaaatgt |
| 30 | NA | rs17224404 | 14 | 90683393 | 2.3E−03 | 0.4893 | 0.062 | 0.119 | (SEQ ID No: 291) |
| | | | | | | | | | gattaccaaatgttca[A/G] |
| | | | | | | | | | aactcagctcctgccc |
| | 087 | rs12324783 | 15 | 27384612 | 5.0E−04 | 0.3135 | 0.027 | 0.081 | (SEQ ID No: 292) |
| | | | | | | | | | ttaacactgttttgag[C/G] |
| | | | | | | | | | atttgagatagatcag |
| | 087 | rs12441963 | 15 | 27414002 | 1.2E−04 | 0.5301 | 0.141 | 0.237 | (SEQ ID No: 293) |
| | | | | | | | | | ggattctaagggctac[A/G] |
| | | | | | | | | | atgaaagtctctgcta |
| | 087 | rs8032547 | 15 | 27428127 | 1.4E−05 | 0.1453 | 0.012 | 0.076 | (SEQ ID No: 294) |
| | | | | | | | | | tgaaaggttcgcaaaa[C/G] |
| | | | | | | | | | tagctctgaagaagtg |
| | 087 | rs8043428 | 15 | 27457583 | 7.8E−04 | 0.586 | 0.162 | 0.248 | (SEQ ID No: 295) |
| | | | | | | | | | ctacagttggctggaa[A/C] |
| | | | | | | | | | aaaggaggatttggga |
| | 087 | rs16955403 | 15 | 27462960 | 3.4E−04 | 0.1912 | 0.012 | 0.059 | (SEQ ID No: 296) |
| | | | | | | | | | cagcatgtgagaaggc[C/T] |
| | | | | | | | | | gaattaaaaggaaggt |
| | 087 | rs8041543 | 15 | 27466210 | 3.9E−04 | 0.2698 | 0.021 | 0.073 | (SEQ ID No: 297) |
| | | | | | | | | | aggatgacgccttcac[A/G] |
| | | | | | | | | | atgggactggtagctc |
| | 087 | rs17749882 | 15 | 27469798 | 7.6E−05 | 0.5828 | 0.244 | 0.357 | (SEQ ID No: 298) |
| | | | | | | | | | tagccatgaaattacc[A/G] |
| | | | | | | | | | catcatgaaaccttca |
| | 088 | rs2899412 | 15 | 45445823 | 9.7E−04 | 1.682 | 0.192 | 0.124 | (SEQ ID No: 299) |
| | | | | | | | | | caggttcacactgtac[A/G] |
| | | | | | | | | | actccagaaggtgcca |
| | 088 | rs16959379 | 15 | 45465635 | 9.9E−04 | 1.78 | 0.151 | 0.091 | (SEQ ID No: 300) |
| | | | | | | | | | tctaccagatgttgag[C/T] |
| | | | | | | | | | cccttgaacctaatgg |
| 31 | 089 | rs1453862 | 15 | 45928633 | 1.4E−04 | 1.581 | 0.535 | 0.422 | (SEQ ID No: 301) |
| | | | | | | | | | aagacttctatcagac[A/G] |
| | | | | | | | | | atccctgaagttcctt |
| | 089 | rs1453861 | 15 | 45928750 | 1.0E−03 | 0.6659 | 0.362 | 0.46 | (SEQ ID No: 202) |
| | | | | | | | | | catttgtttcctgaaa[G/T] |
| | | | | | | | | | ttctgattgtttaaac |
| | 089 | rs11070617 | 15 | 45932546 | 1.6E−03 | 0.6675 | 0.309 | 0.401 | (SEQ ID No: 303) |
| | | | | | | | | | gaagttttaacatagc[A/G] |
| | | | | | | | | | caggaaagaagggcat |
| | 089 | rs649496 | 15 | 45936550 | 6.8E−04 | 0.6492 | 0.355 | 0.459 | (SEQ ID No: 304) |
| | | | | | | | | | ctctaaaagttacata[C/T] |
| | | | | | | | | | tatagccttaactcac |
| | 089 | rs649122 | 15 | 45936614 | 8.8E−04 | 0.6623 | 0.362 | 0.461 | (SEQ ID No: 305) |
| | | | | | | | | | gtcattacgcccaaac[A/G] |
| | | | | | | | | | agtcaatttcaaggat |
| | 090 | rs2444043 | 15 | 53321634 | 6.5E−04 | 0.6573 | 0.377 | 0.479 | (SEQ ID No: 306) |
| | | | | | | | | | ggaaaatttggctttc[A/G] |
| | | | | | | | | | gaaaatggctgaccta |
| | NA | rs12910930 | 15 | 53332998 | 1.4E−03 | 0.6753 | 0.377 | 0.472 | (SEQ ID No: 307) |
| | | | | | | | | | tgcctaggtaggaaaa[C/G] |
| | | | | | | | | | gaaacttagtgaagga |
| | NA | rs12916842 | 15 | 59799003 | 4.8E−03 | 0.6917 | 0.291 | 0.373 | (SEQ ID No: 308) |
| | | | | | | | | | ggtgtccctgtctgga[C/T] |
| | | | | | | | | | gggtttagtcagcatc |
| | NA | rs7180530 | 15 | 59799616 | 1.6E−03 | 0.4976 | 0.071 | 0.133 | (SEQ ID No: 309) |
| | | | | | | | | | gttcaagggctgaacc[A/T] |
| | | | | | | | | | aaggacaaaagaaggg |
| | NA | rs7183554 | 15 | 59803632 | 2.8E−03 | 0.6717 | 0.285 | 0.372 | (SEQ ID No: 310) |
| | | | | | | | | | tatttagcatttatta[A/G] |
| | | | | | | | | | gaacctcctgtgggct |
| 32 | 090 | rs2414743 | 15 | 59808360 | 4.8E−04 | 0.6349 | 0.293 | 0.395 | (SEQ ID No: 311) |
| | | | | | | | | | tgttacctttcctgga[C/T] |
| | | | | | | | | | acatgagggtttcttt |
| | 091 | rs7168148 | 15 | 59813412 | 3.0E−03 | 0.5221 | 0.074 | 0.132 | (SEQ ID No: 312) |
| | | | | | | | | | ttttaattttagcaca[A/G] |
| | | | | | | | | | gctgttacttctacaa |
| | 091 | rs17303705 | 15 | 59814607 | 2.4E−03 | 0.5456 | 0.091 | 0.155 | (SEQ ID No: 313) |
| | | | | | | | | | ttctagaaagatggcc[A/G] |
| | | | | | | | | | actaaagacacgctca |
| | 091 | rs8039022 | 15 | 59820338 | 1.7E−04 | 0.6031 | 0.262 | 0.37 | (SEQ ID No: 314) |
| | | | | | | | | | gacttagagaatttca[C/G] |
| | | | | | | | | | aagagtagtagtacaa |
| | 091 | rs8039554 | 15 | 59827295 | 1.2E−03 | 0.6533 | 0.286 | 0.38 | (SEQ ID No: 315) |
| | | | | | | | | | aagtgggaaaaactaa[C/T] |
| | | | | | | | | | caacgtgcttgaacat |
| | 091 | rs11853350 | 15 | 59834186 | 3.8E−04 | 0.6309 | 0.299 | 0.403 | (SEQ ID No: 316) |
| | | | | | | | | | taacggatgggaaaaa[A/T] |
| | | | | | | | | | tggtttgtagatggag |
| | NA | rs7178749 | 15 | 59841189 | 2.0E−03 | 0.6756 | 0.324 | 0.415 | (SEQ ID No: 317) |
| | | | | | | | | | gcaaaatctgtgttca[G/T] |
| | | | | | | | | | gttgagttaggttatg |
| | NA | rs7342610 | 15 | 59863973 | 2.1E−03 | 0.6744 | 0.337 | 0.43 | (SEQ ID No: 318) |
| | | | | | | | | | tgtcaatgttatttaa[C/T] |
| | | | | | | | | | ccgaataagaacatta |
| | NA | rs7178911 | 15 | 59880533 | 2.1E−03 | 1.459 | 0.426 | 0.337 | (SEQ ID No: 319) |
| | | | | | | | | | cattactctcaaagta[C/T] |
| | | | | | | | | | gccagccctcccagat |
| | 092 | rs3883011 | 15 | 82889398 | 3.5E−04 | 0 | 0 | 0.037 | (SEQ ID No: 320) |
| | | | | | | | | | ttggagggaatacatt[C/G] |
| | | | | | | | | | aaacagtagcaaattt |
| 33 | 092 | rs3883013 | 15 | 82889661 | 3.1E−04 | 0 | 0 | 0.037 | (SEQ ID No: 321) |
| | | | | | | | | | actgtcagaaaataaa[C/T] |
| | | | | | | | | | gactgctcttcataaa |
| | 092 | rs3883014 | 15 | 82889733 | 3.4E−04 | 0 | 0 | 0.037 | (SEQ ID No: 322) |
| | | | | | | | | | tgaaaaatcattcaaa[C/G] |
| | | | | | | | | | aaatttgtcttagtct |
| | 093 | rs11074160 | 15 | 91631564 | 1.0E−04 | 0.2785 | 0.027 | 0.089 | (SEQ ID No: 323) |
| | | | | | | | | | tattagttatccatcc[A/G] |
| | | | | | | | | | aagaaactaagcagtg |
| | NA | rs936679 | 15 | 91631621 | 1.8E−03 | 0.144 | 0.006 | 0.04 | (SEQ ID No: 324) |
| | | | | | | | | | atgggttcataaatgc[C/T] |
| | | | | | | | | | agtcctagggtatggc |
| | 094 | rs7202780 | 16 | 3500902 | 7.6E−04 | 1.506 | 0.542 | 0.44 | (SEQ ID No: 325) |
| | | | | | | | | | ggtagctggtatgtga[A/G] |
| | | | | | | | | | actagagcccagggaa |
| | NA | rs13332500 | 16 | 3515271 | 3.4E−03 | 0.6706 | 0.252 | 0.334 | (SEQ ID No: 326) |
| | | | | | | | | | gccccagataatacaa[A/C] |
| | | | | | | | | | aaaatggccaacgttt |
| | 095 | rs17312836 | 16 | 49298963 | 2.5E−04 | 1.562 | 0.47 | 0.362 | (SEQ ID No: 327) |
| | | | | | | | | | ttctgcaggctgtaaa[G/T] |
| | | | | | | | | | catttggaagagaagt |
| | 095 | rs748855 | 16 | 49308899 | 4.2E−04 | 1.535 | 0.465 | 0.361 | (SEQ ID No: 328) |
| | | | | | | | | | ccagctagaggggcaa[C/T] |
| | | | | | | | | | ttaaaggcatcaggta |
| | 095 | rs1861758 | 16 | 49309288 | 4.8E−03 | 1.412 | 0.444 | 0.361 | (SEQ ID No: 329) |
| | | | | | | | | | tctctcctgcacatac[C/T] |
| | | | | | | | | | ggaggactactgtgtt |
| | 095 | rs3135499 | 16 | 49323628 | 1.2E−03 | 1.48 | 0.482 | 0.386 | (SEQ ID No: 330) |
| | | | | | | | | | ttcatcagagcagtgg[G/T] |
| | | | | | | | | | aaggggtcttttgtga |
| 34 | 095 | rs8060598 | 16 | 49339303 | 2.5E−03 | 1.443 | 0.491 | 0.401 | (SEQ ID No: 331) |
| | | | | | | | | | agccaagacaggatac[A/G] |
| | | | | | | | | | ggttaggcacctcctt |
| | 095 | rs3135503 | 16 | 49348751 | 3.9E−04 | 1.535 | 0.485 | 0.381 | (SEQ ID No: 332) |
| | | | | | | | | | tataggattcaaatga[G/T] |
| | | | | | | | | | agctttatttcaattt |
| | 095 | rs4785450 | 16 | 49349769 | 1.1E−03 | 1.49 | 0.459 | 0.362 | (SEQ ID No: 333) |
| | | | | | | | | | agaaagttgtaggagc[C/T] |
| | | | | | | | | | gcagtgggagctggaa |
| | 095 | rs11076540 | 16 | 49358148 | 3.9E−04 | 1.541 | 0.482 | 0.377 | (SEQ ID No: 334) |
| | | | | | | | | | tattttttccactgaa[A/C] |
| | | | | | | | | | tcttcagtccctcaaa |
| | 096 | rs254353 | 16 | 64469211 | 3.3E−04 | 1.569 | 0.385 | 0.285 | (SEQ ID No: 335) |
| | | | | | | | | | aaatgtaaccaggaaa[A/T] |
| | | | | | | | | | gtaatgttgattacaa |
| | 096 | rs1094921 | 16 | 64499283 | 4.3E−04 | 1.528 | 0.518 | 0.413 | (SEQ ID No: 336) |
| | | | | | | | | | atcccagccattctta[A/C] |
| | | | | | | | | | cagcgggagttatcaa |
| | 096 | rs37162 | 16 | 64503998 | 3.4E−03 | 1.424 | 0.506 | 0.418 | (SEQ ID No: 337) |
| | | | | | | | | | ctcagccgtgatggga[G/T] |
| | | | | | | | | | accatggttgtggaga |
| | NA | rs254363 | 16 | 64542877 | 4.8E−03 | 1.419 | 0.396 | 0.316 | (SEQ ID No: 338) |
| | | | | | | | | | gacataaaaggcagta[A/G] |
| | | | | | | | | | agaccgggcacagtgg |
| | NA | rs16969590 | 16 | 64545873 | 4.8E−03 | 1.419 | 0.396 | 0.316 | (SEQ ID No: 339) |
| | | | | | | | | | gagaaccactggtcca[C/G] |
| | | | | | | | | | ggaagaacagtcttat |
| | 097 | rs4488477 | 16 | 73343602 | 8.2E−06 | 1.815 | 0.313 | 0.2 | (SEQ ID No: 340) |
| | | | | | | | | | tcaagagtggaagcag[C/T] |
| | | | | | | | | | aagagagttaagaggc |
| 35 | 097 | rs4570874 | 16 | 73343663 | 3.8E−04 | 1.597 | 0.317 | 0.225 | (SEQ ID No: 341) |
| | | | | | | | | | atggcagccagagcta[A/T] |
| | | | | | | | | | ctgatcaaatgtagag |
| | 098 | rs12450330 | 17 | 478479 | 1.4E−03 | 1.76 | 0.147 | 0.089 | (SEQ ID No: 342) |
| | | | | | | | | | gggaaatgggcagtaa[C/T] |
| | | | | | | | | | gatcacgggaaatttg |
| | 098 | rs16954363 | 17 | 522254 | 4.4E−04 | 1.845 | 0.154 | 0.09 | (SEQ ID No: 343) |
| | | | | | | | | | caagcccagctgagac[A/G] |
| | | | | | | | | | ctatccttctggcggt |
| | 098 | rs7207469 | 17 | 532225 | 1.4E−03 | 1.76 | 0.147 | 0.089 | (SEQ ID No: 344) |
| | | | | | | | | | tcattaattcaatttc[A/C] |
| | | | | | | | | | aatcccacactgcagc |
| | NA | rs394061 | 17 | 579175 | 1.3E−03 | 1.775 | 0.144 | 0.087 | (SEQ ID No: 345) |
| | | | | | | | | | gtgtaacatggaaata[A/C] |
| | | | | | | | | | caggtttagccaacct |
| | 099 | rs8068990 | 17 | 9892826 | 2.5E−04 | 1.782 | 0.195 | 0.12 | (SEQ ID No: 346) |
| | | | | | | | | | aggcactgagagagaa[A/T] |
| | | | | | | | | | attaggaagacaacac |
| | 099 | rs8072311 | 17 | 9903752 | 3.0E−04 | 1.825 | 0.172 | 0.102 | (SEQ ID No: 347) |
| | | | | | | | | | gtcagatttgcataag[G/T] |
| | | | | | | | | | acacaccacaaggctg |
| | 099 | rs17810635 | 17 | 9905657 | 4.7E−04 | 1.793 | 0.169 | 0.102 | (SEQ ID No: 348) |
| | | | | | | | | | tgcatctttggacgaa[A/G] |
| | | | | | | | | | ctagaaaagaagctga |
| | NA | rs12604208 | 18 | 8666626 | 4.9E−03 | 1.431 | 0.383 | 0.303 | (SEQ ID No: 349) |
| | | | | | | | | | atgggcccttttcaga[A/G] |
| | | | | | | | | | taaaacatatagactt |
| | 100 | rs489570 | 18 | 8703965 | 3.7E−05 | 1.678 | 0.393 | 0.278 | (SEQ ID No: 350) |
| | | | | | | | | | aagcggggaaggggca[C/T] |
| | | | | | | | | | ggatatctttaccgca |
| 36 | 101 | rs2244081 | 18 | 61675524 | 1.0E−03 | 2.214 | 0.077 | 0.037 | (SEQ ID No: 351) |
| | | | | | | | | | gaggaatatagcaaga[A/C] |
| | | | | | | | | | cctgcatctataataa |
| | 101 | rs17075338 | 18 | 61687357 | 2.2E−03 | 2.115 | 0.077 | 0.038 | (SEQ ID No: 352) |
| | | | | | | | | | catggtcaattagtcc[A/G] |
| | | | | | | | | | cacatactgttaactt |
| | 101 | rs9960373 | 18 | 61689748 | 1.1E−03 | 2.194 | 0.077 | 0.037 | (SEQ ID No: 353) |
| | | | | | | | | | tacagtgagtaggaag[A/T] |
| | | | | | | | | | attgtttgagtggagc |
| | 101 | rs9950064 | 18 | 61691931 | 5.2E−04 | 2.325 | 0.076 | 0.034 | (SEQ ID No: 354) |
| | | | | | | | | | attttatggctaatta[A/G] |
| | | | | | | | | | aaatgatgattgttca |
| | 101 | rs9953248 | 18 | 61693003 | 2.1E−03 | 2.127 | 0.076 | 0.037 | (SEQ ID No: 355) |
| | | | | | | | | | atgtggccttatttag[C/T] |
| | | | | | | | | | aatggtaccattttgt |
| | 101 | rs17075420 | 18 | 61700461 | 7.9E−04 | 2.335 | 0.071 | 0.032 | (SEQ ID No: 356) |
| | | | | | | | | | aactcaaacttaaaca[C/G] |
| | | | | | | | | | atgatgacttgcaatt |
| | NA | rs1564815 | 18 | 61715545 | 1.9E−03 | 2.184 | 0.071 | 0.034 | (SEQ ID No: 357) |
| | | | | | | | | | tgggatagatgcctaa[A/G] |
| | | | | | | | | | ccatgatgcctgccca |
| | NA | rs2587438 | 18 | 61716853 | 1.3E−03 | 2.247 | 0.071 | 0.033 | (SEQ ID No: 358) |
| | | | | | | | | | aataaaggcttgaaca[C/T] |
| | | | | | | | | | tatttgtctgtttctg |
| | NA | rs2628223 | 18 | 61719665 | 1.6E−03 | 2.251 | 0.07 | 0.032 | (SEQ ID No: 359) |
| | | | | | | | | | ccttcacaggagttaa[G/T] |
| | | | | | | | | | agagtcctccaaaacc |
| | NA | rs205084 | 18 | 61736852 | 2.8E−03 | 2.146 | 0.068 | 0.033 | (SEQ ID No: 360) |
| | | | | | | | | | aactgcttttattgac[A/G] |
| | | | | | | | | | accacataaaatcacc |
| 37 | 102 | rs1725472 | 19 | 43279885 | 5.0E−03 | 0.7108 | 0.42 | 0.505 | (SEQ ID No: 361) |
| | | | | | | | | | ttcaaaaatagacaga[A/T] |
| | | | | | | | | | ataaatgatggtgttg |
| | 102 | rs3852911 | 19 | 43297276 | 4.7E−03 | 0.7008 | 0.345 | 0.429 | (SEQ ID No: 362) |
| | | | | | | | | | gttgactggctagtga[C/T] |
| | | | | | | | | | ccttcatgccccagga |
| | 102 | rs855614 | 19 | 43311582 | 8.6E−04 | 1.494 | 0.503 | 0.404 | (SEQ ID No: 363) |
| | | | | | | | | | ataaaaacaagatgaa[C/T] |
| | | | | | | | | | gtaaccagccatggct |
| | 103 | rs6140121 | 20 | 6860028 | 5.7E−04 | 0.5057 | 0.091 | 0.166 | (SEQ ID No: 364) |
| | | | | | | | | | atagcaacccaaagcg[A/T] |
| | | | | | | | | | catatgttcaagttaa |
| | 103 | rs6054616 | 20 | 6868028 | 4.3E−03 | 0.5697 | 0.095 | 0.155 | (SEQ ID No: 365) |
| | | | | | | | | | tgtgtggcttagaaaa[C/G] |
| | | | | | | | | | cattagcctctaggga |
| | 103 | rs2057039 | 20 | 6870810 | 4.7E−03 | 0.5724 | 0.095 | 0.155 | (SEQ ID No: 366) |
| | | | | | | | | | caggctgatgatttgc[A/G] |
| | | | | | | | | | actagtaaaaataata |
| | 103 | rs6085753 | 20 | 6873667 | 5.5E−04 | 0.5049 | 0.091 | 0.166 | (SEQ ID No: 367) |
| | | | | | | | | | gaccatcaaatactta[C/T] |
| | | | | | | | | | ggctagatgctccagt |
| | 104 | rs819144 | 20 | 32325217 | 3.7E−03 | 0.5119 | 0.065 | 0.12 | (SEQ ID No: 368) |
| | | | | | | | | | gtagacgagttaccct[G/T] |
| | | | | | | | | | tcttcttccatgtacc |
| | 104 | rs819159 | 20 | 32343381 | 6.2E−04 | 0.4573 | 0.066 | 0.134 | (SEQ ID No: 369) |
| | | | | | | | | | ttccccttgctctgca[A/T] |
| | | | | | | | | | atcaggtcaaacactt |
| | 105 | rs2827566 | 21 | 22848288 | 8.2E−04 | 1.519 | 0.393 | 0.299 | (SEQ ID No: 370) |
| | | | | | | | | | attaagtgcttagtga[C/T] |
| | | | | | | | | | ctctatgacagaaaag |
| 38 | 105 | rs2827573 | 21 | 22854664 | 5.5E−04 | 1.54 | 0.393 | 0.296 | (SEQ ID No: 371) |
| | | | | | | | | | caattagttggagtca[C/T] |
| | | | | | | | | | tctagattttattttg |
| | 105 | rs1392922 | 21 | 22864584 | 7.4E−04 | 1.521 | 0.394 | 0.3 | (SEQ ID No: 372) |
| | | | | | | | | | aaattaatgaaggaga[C/T] |
| | | | | | | | | | gatttcaggataacat |
| | 105 | rs2827604 | 21 | 22865197 | 1.8E−03 | 1.476 | 0.388 | 0.3 | (SEQ ID No: 373) |
| | | | | | | | | | gtgttctgggtcatta[C/T] |
| | | | | | | | | | atattatgttactatt |
| | 105 | rs2827606 | 21 | 22865549 | 4.4E−04 | 1.554 | 0.398 | 0.299 | (SEQ ID No: 374) |
| | | | | | | | | | atcatcataatttaga[A/C] |
| | | | | | | | | | cttttttgtgtgtgtt |
| | 106 | rs2828494 | 21 | 24038001 | 1.4E−03 | 0.3728 | 0.033 | 0.083 | (SEQ ID No: 375) |
| | | | | | | | | | atcccatgcagcaaaa[C/T] |
| | | | | | | | | | cactgttccttcttgt |
| | 106 | rs2828495 | 21 | 24038081 | 3.1E−04 | 0.5665 | 0.163 | 0.255 | (SEQ ID No: 376) |
| | | | | | | | | | gttggatatgagagca[C/T] |
| | | | | | | | | | tgaattgtggagctta |
| | 106 | rs2828506 | 21 | 24048609 | 7.3E−04 | 0.6621 | 0.397 | 0.499 | (SEQ ID No: 377) |
| | | | | | | | | | tttactgacaattaca[A/C] |
| | | | | | | | | | ggatgcaaacaaacaa |
| | 106 | rs2828512 | 21 | 24051399 | 2.4E−04 | 0.2784 | 0.024 | 0.08 | (SEQ ID No: 378) |
| | | | | | | | | | cattttagaactgcac[A/T] |
| | | | | | | | | | ccacttacaaaggtaa |
| | 106 | rs2828516 | 21 | 24057342 | 8.4E−04 | 0.343 | 0.03 | 0.082 | (SEQ ID No: 379) |
| | | | | | | | | | gtatccattacaaaaa[A/C] |
| | | | | | | | | | gtcaattgcaaatggt |
| | NA | rs2040256 | 21 | 24070123 | 1.9E−03 | 0.664 | 0.284 | 0.374 | (SEQ ID No: 380) |
| | | | | | | | | | ttagagcttgaaacta[C/T] |
| | | | | | | | | | gaattttgagtggatg |
| 39 | NA | rs2252653 | 21 | 24070703 | 2.3E−03 | 0.6764 | 0.312 | 0.401 | (SEQ ID No: 381) |
| | | | | | | | | | agagattaccgtggcc[A/G] |
| | | | | | | | | | atcttctatgagtgca |
| | 106 | rs1157277 | 21 | 24084426 | 9.5E−04 | 0.6156 | 0.204 | 0.293 | (SEQ ID No: 382) |
| | | | | | | | | | ggcctagtgcaggtag[A/C] |
| | | | | | | | | | agctgggctaaagttt |
| | 106 | rs207473 | 21 | 24093800 | 1.3E−03 | 0.6658 | 0.332 | 0.428 | (SEQ ID No: 383) |
| | | | | | | | | | tgcttatttttgttgc[A/T] |
| | | | | | | | | | atcagataatgcgtta |
| | NA | rs746064 | 21 | 41503318 | 5.0E−03 | 0.691 | 0.292 | 0.373 | (SEQ ID No: 384) |
| | | | | | | | | | ggctgtttctcaaata[C/T] |
| | | | | | | | | | atcactctgcagaaaa |
| | NA | rs6517656 | 21 | 41505608 | 2.5E−03 | 0.5847 | 0.121 | 0.191 | (SEQ ID No: 385) |
| | | | | | | | | | actaactagatgaatc[A/G] |
| | | | | | | | | | cccagtttttcccttt |
| | 107 | rs2837979 | 21 | 41532183 | 3.0E−11 | | 0.03 | 0 | (SEQ ID No: 386) |
| | | | | | | | | | ctacccaaaggaaata[C/T] |
| | | | | | | | | | gtctgtatgcaggagg |
| | 108 | rs6007450 | 22 | 43823566 | 1.6E−04 | 1.844 | 0.19 | 0.113 | (SEQ ID No: 387) |
| | | | | | | | | | tcagctcaggctgtta[C/T] |
| | | | | | | | | | gacaaaaataccacag |
| | 108 | rs132873 | 22 | 43887239 | 2.3E−03 | 0.5611 | 0.104 | 0.171 | (SEQ ID No: 388) |
| | | | | | | | | | ccattcgaacatatgc[C/T] |
| | | | | | | | | | aatgaaatgtgtggaa |
| | 108 | rs132875 | 22 | 43887449 | 2.3E−03 | 0.5611 | 0.104 | 0.171 | (SEQ ID No: 389) |
| | | | | | | | | | atgtccacgtcagaaa[G/T] |
| | | | | | | | | | ctgctgaatatcttcg |
| | 108 | rs8135904 | 22 | 43888956 | 7.6E−04 | 0.6634 | 0.405 | 0.507 | (SEQ ID No: 390) |
| | | | | | | | | | gtaagtatttccgctc[C/T] |
| | | | | | | | | | caggaagtctgcatta |
| 40 | 109 | rs6529723 | 23 | 4857004 | 5.9E−04 | 2.617 | 0.059 | 0.023 | (SEQ ID No: 391) |
| | | | | | | | | | atcacaatatcaatac[A/G] |
| | | | | | | | | | gaggcttattgtgttg |
| | NA | rs5915934 | 23 | 4857364 | 1.2E−03 | 2.533 | 0.056 | 0.023 | (SEQ ID No: 392) |
| | | | | | | | | | gtagacagtacatccc[A/G] |
| | | | | | | | | | agaaagagaggattca |
| | NA | rs5915482 | 23 | 4858842 | 1.8E−03 | 2.382 | 0.06 | 0.026 | (SEQ ID No: 393) |
| | | | | | | | | | tttgcttcccatcctc[G/T] |
| | | | | | | | | | aggtgcacagtgtcca |
| | NA | rs1900762 | 23 | 861000 | 1.5E−03 | 2.416 | 0.059 | 0.025 | (SEQ ID No: 394) |
| | | | | | | | | | gtcatgtgacctactc[C/T] |
| | | | | | | | | | gaaaaaacacataatt |
| | 109 | rs1900761 | 23 | 4863509 | 3.4E−04 | 2.75 | 0.059 | 0.022 | (SEQ ID No: 395) |
| | | | | | | | | | tgacaattttcacttg[A/G] |
| | | | | | | | | | caaacatttattcctt |
| | 109 | rs5961617 | 23 | 4871915 | 1.6E−03 | 2.398 | 0.059 | 0.025 | (SEQ ID No: 396) |
| | | | | | | | | | gctatggcagtcctag[A/C] |
| | | | | | | | | | acagtttcaattcttc |
| | 109 | rs6529732 | 23 | 4872539 | 3.7E−03 | 2.275 | 0.056 | 0.026 | (SEQ ID No: 397) |
| | | | | | | | | | aggaggaagagagaga[G/T] |
| | | | | | | | | | atacctcttgttttct |
| | 109 | rs5961620 | 23 | 4875328 | 5.7E−04 | 2.625 | 0.059 | 0.023 | (SEQ ID No: 398) |
| | | | | | | | | | ctttaatgcactaaac[C/T] |
| | | | | | | | | | gactcagtgaaaattt |
| | 109 | rs6413640 | 23 | 4877578 | 4.3E−03 | 2.195 | 0.06 | 0.028 | (SEQ ID No: 399) |
| | | | | | | | | | atgaacatatgagtac[A/C] |
| | | | | | | | | | ggtatgtttttgatgt |
| | 109 | rs5915487 | 23 | 4880410 | 1.6E−03 | 2.398 | 0.059 | 0.025 | (SEQ ID No: 400) |
| | | | | | | | | | ctgagaagtcgtgtgg[A/T] |
| | | | | | | | | | ccaacctttgctcatt |
| 41 | 110 | rs2041633 | 23 | 116825004 | 3.5E−04 | 1.638 | 0.277 | 0.189 | (SEQ ID No: 401) |
| | | | | | | | | | ttctgagaaaatattg[A/G] |
| | | | | | | | | | agaccatctttatgcc |
| | 110 | rs5956813 | 23 | 116826923 | 2.7E−03 | 0.4195 | 0.038 | 0.087 | (SEQ ID No: 402) |
| | | | | | | | | | acaaaaacaagcctac[A/C] |
| | | | | | | | | | ataaaaaatgccagtg |
| | NA | rs5955990 | 23 | 116832359 | 4.2E−03 | 0.4572 | 0.044 | 0.092 | (SEQ ID No: 403) |
| | | | | | | | | | aggttggctcttgttc[C/T] |
| | | | | | | | | | attaacactgatgctc |
| | 110 | rs5956817 | 23 | 116854460 | 2.9E−03 | 0.4439 | 0.044 | 0.095 | (SEQ ID No: 404) |
| | | | | | | | | | aaatttactagaaacc[A/G] |
| | | | | | | | | | aacaatgattgccact |

TABLE 002

Singleton SNPs

| | | | | | | | | | |
|---|---|---|---|---|---|---|---|---|---|
| Set | Tbl | Name | Chr | Position | p-value | OR | F_A | F_U | Flank Sequence |

| | | | | | | | | | |
|---|---|---|---|---|---|---|---|---|---|
| 42 | 111 | rs475929 | 1 | 74479600 | 2.7E−34 | 61.78 | 0.113 | 0.002 | (SEQ ID No: 405) |
| | | | | | | | | | cagcagtgacttctaa[A/G] |
| | | | | | | | | | tgactatgtggaggag |
| | 112 | rs11164386 | 1 | 102290118 | 2.1E−13 | 34.01 | 0.045 | 0.001 | (SEQ ID No: 406) |
| | | | | | | | | | atgaactaaattttgg[A/C] |
| | | | | | | | | | atgacaaagacagaat |
| | 011 | rs4427395 | 1 | 234188628 | 1.6E−05 | 0.5473 | 0.224 | 0.345 | (SEQ ID No: 407) |
| | | | | | | | | | actggtaatgtatcac[A/G] |
| | | | | | | | | | caggaaaaatggtaat |
| | 011 | rs10925498 | 1 | 234189251 | 4.2E−05 | 0.5629 | 0.224 | 0.338 | (SEQ ID No: 408) |
| | | | | | | | | | gttttgttggatttcg[G/T] |
| | | | | | | | | | attaactatactcact |
| | 113 | rs17012999 | 2 | 119032452 | 2.5E−14 | 13.94 | 0.063 | 0.005 | (SEQ ID No: 409) |
| | | | | | | | | | acactttaaattttca[C/T] |
| | | | | | | | | | aattgccttggaccat |
| | 114 | rs16844553 | 2 | 160633928 | 1.6E−06 | 17.02 | 0.023 | 0.001 | (SEQ ID No: 410) |
| | | | | | | | | | gtttcactccactaac[G/T] |
| | | | | | | | | | acacttggcttaatcc |
| | 115 | rs2198731 | 2 | 182195451 | 1.4E−10 | 27.04 | 0.036 | 0.001 | (SEQ ID No: 411) |
| | | | | | | | | | gactttcagttttgta[A/T] |
| | | | | | | | | | ccctagagctgccttg |
| | 116 | rs7562389 | 2 | 231268001 | 6.3E−05 | 0.5819 | 0.273 | 0.393 | (SEQ ID No: 412) |
| | | | | | | | | | cccctatgccacatca[A/G] |
| | | | | | | | | | ccatcgagcccggtgc |
| | 117 | rs17042155 | 3 | 16346233 | 1.8E−25 | 13.05 | 0.122 | 0.011 | (SEQ ID No: 413) |
| | | | | | | | | | aagtaatcataacctc[A/G] |
| | | | | | | | | | caaagcttgagagtgt |
| | 118 | rs17005751 | 3 | 19217997 | 2.7E−06 | 16.35 | 0.022 | 0.001 | (SEQ ID No: 414) |
| | | | | | | | | | gaaaactgtggtttaa[C/G] |
| | | | | | | | | | agtgagaaaggatcat |
| 43 | 119 | rs17006139 | 3 | 19691079 | 1.4E−05 | 14.76 | 0.02 | 0.001 | (SEQ ID No: 415) |
| | | | | | | | | | ctaatccagcacccac[A/G] |
| | | | | | | | | | attaacataaaggaaa |
| | 120 | rs2033776 | 3 | 29439472 | 2.7E−05 | 13.86 | 0.019 | 0.001 | (SEQ ID No: 416) |
| | | | | | | | | | ctaataaagcaacaaa[A/T] |
| | | | | | | | | | ctattttatgtcatgg |
| | 121 | rs17054509 | 3 | 54893785 | 1.7E−07 | 8.278 | 0.039 | 0.005 | (SEQ ID No: 417) |
| | | | | | | | | | ttacaggatcctaagt[C/G] |
| | | | | | | | | | aaaacggtcatccgcc |
| | 122 | rs1880214 | 3 | 61195849 | 9.4E−09 | 6.548 | 0.056 | 0.009 | (SEQ ID No: 418) |
| | | | | | | | | | ttgtccttatccatta[C/G] |
| | | | | | | | | | acttcattttacagaa |
| | 123 | rs1444477 | 3 | 78679108 | 3.2E−05 | 13.66 | 0.019 | 0.001 | (SEQ ID No: 419) |
| | | | | | | | | | atcaacaaatggataa[A/C] |
| | | | | | | | | | cattataccacatgaa |
| | 124 | rs6815823 | 4 | 13515096 | 6.4E−06 | 7.683 | 0.03 | 0.004 | (SEQ ID No: 420) |
| | | | | | | | | | gtggtacaagcaggca[C/T] |
| | | | | | | | | | tagtttcatgggggtc |
| | 125 | SNP_A- | 4 | 48491108 | 1.8E−08 | 10.11 | 0.041 | 0.004 | (SEQ ID No: 421) |
| | | 2081938 | | | | | | | acctttatctcacata[C/T] |
| | | | | | | | | | agtctctttcaatagc |
| | 126 | rs17090112 | 4 | 61222559 | 6.6E−25 | 63.98 | 0.081 | 0.001 | (SEQ ID No: 422) |
| | | | | | | | | | ggttgaggtactatga[A/G] |
| | | | | | | | | | ctcacatttaggattt |
| | 127 | rs10012057 | 4 | 112318188 | 2.6E−05 | 13.9 | 0.019 | 0.001 | (SEQ ID No: 423) |
| | | | | | | | | | tttttccaatccatta[A/G] |
| | | | | | | | | | aaatcattctgtatta |
| | 128 | rs17046129 | 4 | 114789761 | 8.8E−11 | 20.1 | 0.04 | 0.002 | (SEQ ID No: 424) |
| | | | | | | | | | agcaaactcagaataa[C/T] |
| | | | | | | | | | tgaaattgcaatagca |
| 44 | 129 | SNP_A- | 4 | 153149584 | 4.1E−23 | 41.15 | 0.079 | 0.002 | (SEQ ID No: 425) |
| | | 2177362 | | | | | | | caccttcatgtggaaa[C/G] |
| | | | | | | | | | tgtacaggcccctttc |
| | 130 | rs16891296 | 5 | 33573443 | 4.9E−14 | 15.04 | 0.06 | 0.004 | (SEQ ID No: 426) |
| | | | | | | | | | cagcaactatccaaga[A/G] |
| | | | | | | | | | tacaacttcatttgct |
| | 131 | rs11953095 | 5 | 96425437 | 9.7E−06 | 4.599 | 0.046 | 0.01 | (SEQ ID No: 427) |
| | | | | | | | | | tgccaagattaaaaaa[C/T] |
| | | | | | | | | | ggtgcattcttgttta |
| | 132 | rs1422010 | 5 | 143812852 | 2.8E−08 | 21.25 | 0.028 | 0.001 | (SEQ ID No: 428) |
| | | | | | | | | | ttggcaacatcttggg[A/G] |
| | | | | | | | | | catggttagggccatg |
| | 133 | rs10041351 | 5 | 174633402 | 8.5E−05 | 1.606 | 0.488 | 0.373 | (SEQ ID No: 429) |
| | | | | | | | | | cccagggtgtcagcta[C/T] |
| | | | | | | | | | atccaaggctagaaaa |
| | 134 | rs2875970 | 6 | 4807112 | 6.5E−16 | 40.68 | 0.052 | 0.001 | (SEQ ID No: 430) |
| | | | | | | | | | ggttatgatgcgcatg[G/T] |
| | | | | | | | | | atctggttaagttaca |
| | 135 | rs259920 | 6 | 30133738 | 2.9E−05 | 0 | 0 | 0.054 | (SEQ ID No: 431) |
| | | | | | | | | | tacccagctatatcca[C/T] |
| | | | | | | | | | agttgcaaaacaaagt |
| | 136 | rs6926440 | 6 | 53511839 | 1.1E−14 | 11.15 | 0.072 | 0.007 | (SEQ ID No: 432) |
| | | | | | | | | | ctatgatgtaaatgta[A/G] |
| | | | | | | | | | caaatatgacttgatt |
| | 137 | rs4119563 | 6 | 56229665 | 7.3E−05 | 0.5335 | 0.161 | 0.264 | (SEQ ID No: 433) |
| | | | | | | | | | tatccataatcaatca[C/T] |
| | | | | | | | | | acaaaatacaagtaaa |
| | 138 | rs6941591 | 6 | 113833795 | 1.1E−08 | 13.42 | 0.036 | 0.003 | (SEQ ID No: 434) |
| | | | | | | | | | gggcctgggtggatcc[A/C] |
| | | | | | | | | | aaataagatattactg |
| 45 | 139 | rs9496804 | 6 | 144260788 | 7.6E−06 | 11.65 | 0.024 | 0.002 | (SEQ ID No: 435) |
| | | | | | | | | | cttaagaacttctcaa[C/T] |
| | | | | | | | | | gaaatgaattatttag |
| | 140 | rs17076363 | 6 | 147206578 | 1.9E−21 | 30.36 | 0.077 | 0.003 | (SEQ ID No: 436) |
| | | | | | | | | | ataacaaaaaaccaat[A/C] |
| | | | | | | | | | agtctgtagaaaaatg |
| | 141 | rs2079495 | 7 | 25276568 | 6.5E−05 | 4.419 | 0.039 | 0.009 | (SEQ ID No: 437) |
| | | | | | | | | | accatcaaatgttatt[A/T] |
| | | | | | | | | | aagagagctataaaaa |
| | 142 | rs2366858 | 7 | 79985273 | 3.5E−09 | 8.356 | 0.049 | 0.006 | (SEQ ID No: 438) |
| | | | | | | | | | aacaaatgcctagaaa[A/C] |
| | | | | | | | | | tattggagaattagaa |
| | 143 | rs1238058 | 8 | 4265211 | 4.0E−07 | 9.829 | 0.03 | 3 0.003 | (SEQ ID No: 439) |
| | | | | | | | | | tttaaataacttgaga[C/G] |
| | | | | | | | | | aatttcttcttgaaaa |
| | 144 | rs1107050 | 8 | 34056049 | 1.5E−08 | 13.18 | 0.035 | 0.003 | (SEQ ID No: 440) |
| | | | | | | | | | cctaatattttctaga[A/G] |
| | | | | | | | | | ccctctccaaaaggtc |
| | 145 | rs6472980 | 8 | 77732821 | 2.1E−05 | 0.5061 | 0.154 | 0.264 | (SEQ ID No: 441) |
| | | | | | | | | | ggagggagagagagca[C/G] |
| | | | | | | | | | acctaaatcttctacc |
| | 146 | rs7817645 | 8 | 85212373 | 7.3E−09 | 16.86 | 0.034 | 0.002 | (SEQ ID No: 442) |
| | | | | | | | | | tcaacacggaagcccc[A/G] |
| | | | | | | | | | aaagaatcttcagaca |
| | 147 | rs10963149 | 9 | 1756892 | 5.6E−21 | 53.53 | 0.068 | 0.001 | (SEQ ID No: 443) |
| | | | | | | | | | ctgcttcttcctggtc[A/G] |
| | | | | | | | | | cctattctcaatcctt |
| | 148 | rs1413262 | 9 | 20127211 | 2.1E−06 | 7.5 | 0.035 | 0.005 | (SEQ ID No: 444) |
| | | | | | | | | | ataaaagtctataata[A/T] |
| | | | | | | | | | gaggcacctatgcaaa |
| 46 | 149 | rs10124385 | 9 | 70986459 | 7.6E−05 | 2.82 | 0.068 | 0.025 | (SEQ ID No: 445) |
| | | | | | | | | | ttcgggcattatatac[C/T] |
| | | | | | | | | | ggtgttttggacaaag |
| | 150 | rs17787401 | 9 | 76630755 | 9.4E−07 | 5.28 | 0.049 | 0.01 | (SEQ ID No: 446) |
| | | | | | | | | | tgctcataggagagaa[C/T] |
| | | | | | | | | | ataagaactctgagtg |
| | 151 | rs10978782 | 9 | 96588232 | 1.8E−07 | 19.34 | 0.026 | 0.001 | (SEQ ID No: 447) |
| | | | | | | | | | agcagaataatgtgac[C/G] |
| | | | | | | | | | caaaatcaagagggaa |
| | 152 | rs16934355 | 9 | 116997213 | 1.4E−07 | 14.54 | 0.029 | 0.002 | (SEQ ID No: 448) |
| | | | | | | | | | aaataatagagatcca[A/T] |
| | | | | | | | | | cctagggagccatttg |
| | 153 | SNP_A- | 9 | 120455105 | 4.7E−07 | 13.83 | 0.028 | 0.002 | (SEQ ID No: 449) |
| | | 2281124 | | | | | | | gagtacactaaccaga[C/T] |
| | | | | | | | | | ggatataaacatgagg |
| | 154 | rs2156323 | 9 | 133750375 | 9.6E−05 | 1.813 | 0.243 | 0.151 | (SEQ ID No: 450) |
| | | | | | | | | | agccataaggaaatgc[A/G] |
| | | | | | | | | | aggtcactgcttccag |
| | 155 | rs17369334 | 10 | 15563073 | 3.2E−05 | 2.519 | 0.094 | 0.04 | (SEQ ID No: 451) |
| | | | | | | | | | gtatttagtttggtgc[A/C] |
| | | | | | | | | | gaagtaacgcacaatt |
| | 156 | rs1887218 | 10 | 18287669 | 1.4E−21 | 25.61 | 0.081 | 0.003 | (SEQ ID No: 452) |
| | | | | | | | | | tactgcaaagaacaca[G/T] |
| | | | | | | | | | gaattacacctttctt |
| | 157 | rs12268320 | 10 | 19402289 | 4.3E−09 | 23.21 | 0.031 | 0.001 | (SEQ ID No: 453) |
| | | | | | | | | | tacacactggaattaa[C/T] |
| | | | | | | | | | aaaagcttttgcatta |
| | 158 | rs1904756 | 10 | 60464000 | 7.9E−09 | 10.54 | 0.042 | 0.004 | (SEQ ID No: 454) |
| | | | | | | | | | atcacaagagggtaaa[A/C] |
| | | | | | | | | | tcagtgtcacagcaga |
| 47 | 159 | rs16925318 | 10 | 69756962 | 7.2E−11 | 20.27 | 0.041 | 0.002 | (SEQ ID No: 455) |
| | | | | | | | | | tttggtaaactttgaa[A/G] |
| | | | | | | | | | cactactttatctact |
| | 160 | rs12254170 | 10 | 91287697 | 7.5E−05 | 1.614 | 0.479 | 0.363 | t(SEQ ID No: 456) |
| | | | | | | | | | ttacgagttcagtaa[C/T] |
| | | | | | | | | | tgtgggttgatgatgg |
| | 161 | rs1965595 | 10 | 105758261 | 4.0E−07 | 13.61 | 0.027 | 0.002 | (SEQ ID No: 457) |
| | | | | | | | | | taatgattgaccttat[G/T] |
| | | | | | | | | | acaactctatatgact |
| | 162 | rs7117749 | 11 | 5453501 | 1.5E−05 | | 0.013 | 0 | (SEQ ID No: 458) |
| | | | | | | | | | agatggtcatgtttca[C/T] |
| | | | | | | | | | ggaatttacataataa |
| | 163 | rs11246628 | 11 | 51394606 | 2.1E−05 | 8.81 | 0.024 | 0.003 | (SEQ ID No: 459) |
| | | | | | | | | | ggatttatcctacaaa[C/G] |
| | | | | | | | | | gcatataaatgggaaa |
| | 164 | rs17610514 | 11 | 55652374 | 3.6E−05 | 0.1566 | 0.012 | 0.074 | (SEQ ID No: 460) |
| | | | | | | | | | aaaagtatacaagaac[C/T] |
| | | | | | | | | | ctatggcaatgtcaag |
| | 165 | rs558073 | 11 | 63959003 | 3.7E−05 | 6.273 | 0.029 | 0.005 | (SEQ ID No: 461) |
| | | | | | | | | | ttctgttctcttccca[A/G] |
| | | | | | | | | | ctgacaaggaaacttc |
| | 166 | rs597227 | 11 | 120899984 | 1.0E−27 | 24.75 | 0.107 | 0.005 | (SEQ ID No: 462) |
| | | | | | | | | | acacatatttgcgttc[A/G] |
| | | | | | | | | | ccacatacagtacaca |
| | 167 | rs11220034 | 11 | 124741629 | 1.7E−06 | | 0.016 | 0 | (SEQ ID No: 463) |
| | | | | | | | | | tttctcttcactccca[A/G] |
| | | | | | | | | | ctaaaacgtaagctca |
| | 168 | rs12227654 | 12 | 23580022 | 9.1E−05 | 1.712 | 0.281 | 0.186 | (SEQ ID No: 464) |
| | | | | | | | | | gaattctctcaaaaac[C/T] |
| | | | | | | | | | caaaataaagttgttc |
| 48 | 169 | rs11114962 | 12 | 80548120 | 2.0E−05 | 2.031 | 0.175 | 0.094 | (SEQ ID No: 465) |
| | | | | | | | | | atgctatcttcaaaag[C/G] |
| | | | | | | | | | caataataccaaagga |
| | 170 | rs7489074 | 12 | 130429948 | 7.2E−05 | 2.006 | 0.154 | 0.083 | (SEQ ID No: 466) |
| | | | | | | | | | aagagcacctggatac[C/T] |
| | | | | | | | | | ggggtcatgttagcgc |
| | 171 | rs17088665 | 13 | 63132563 | 1.7E−16 | 15.37 | 0.069 | 0.005 | (SEQ ID No: 467) |
| | | | | | | | | | atgtccagagaatagc[A/G] |
| | | | | | | | | | aggaagagaattgtag |
| | 172 | rs17062387 | 13 | 73697866 | 5.1E−05 | 7.316 | 0.025 | 0.003 | (SEQ ID No: 468) |
| | | | | | | | | | taaatgcaaaaataaa[C/G] |
| | | | | | | | | | gtagaaacaatgtgct |
| | 173 | rs16953855 | 13 | 96562206 | 4.2E−18 | 14.15 | 0.082 | 0.006 | (SEQ ID No: 469) |
| | | | | | | | | | taagtgcaattaggaa[C/T] |
| | | | | | | | | | gagtggaaacaatgag |
| | 174 | rs1961605 | 14 | 58535847 | 5.5E−05 | 0.1621 | 0.013 | 0.072 | (SEQ ID No: 470) |
| | | | | | | | | | cccaaaactcgaagtc[C/T] |
| | | | | | | | | | aaagtcatatccaaga |
| | 175 | rs8010814 | 14 | 62261229 | 4.8E−10 | 15.31 | 0.04 | 0.003 | (SEQ ID No: 471) |
| | | | | | | | | | tcaacactacagcaac[A/C] |
| | | | | | | | | | aagttattagctagta |
| | 176 | SNP_A- | 14 | 75059876 | 7.2E−06 | 0.2507 | 0.033 | 0.12 | (SEQ ID No: 472) |
| | | 2099420 | | | | | | | agaggcttaggatata[C/T] |
| | | | | | | | | | gccctgttcaatattt |
| | 177 | rs2767531 | 14 | 87648625 | 2.0E−09 | 24.16 | 0.032 | 0.001 | (SEQ ID No: 473) |
| | | | | | | | | | tttgctaacgaggcta[A/T] |
| | | | | | | | | | gtggtagatttttttt |
| | 178 | rs1877250 | 15 | 23673136 | 5.6E−05 | 1.633 | 0.589 | 0.467 | (SEQ ID No: 474) |
| | | | | | | | | | caaagggcagaacgtg[A/G] |
| | | | | | | | | | agcagcatttgaggct |
| 49 | 179 | rs8026608 | 15 | 38274294 | 1.8E−05 | 10.84 | 0.022 | 0.002 | (SEQ ID No: 475) |
| | | | | | | | | | ctaattcttttggata[C/G] |
| | | | | | | | | | taaaccatgaaaaatg |
| | 180 | rs341369 | 15 | 58894143 | 2.9E−34 | 61.39 | 0.113 | 0.002 | (SEQ ID No: 476) |
| | | | | | | | | | gagcttcctcttctga[C/T] |
| | | | | | | | | | ggcaggagtttgaagc |
| | 181 | rs16942965 | 15 | 87634875 | 5.5E−06 | 9.822 | 0.027 | 0.003 | (SEQ ID No: 477) |
| | | | | | | | | | atgtatccattaccca[C/G] |
| | | | | | | | | | atttaagaaataatat |
| | 182 | rs7190922 | 16 | 12725421 | 3.3E−06 | 0.4262 | 0.119 | 0.241 | (SEQ ID No: 478) |
| | | | | | | | | | gtttgcatacatcaca[A/G] |
| | | | | | | | | | gttatttggggaagta |
| | 183 | rs1925499 | 16 | 19967712 | 3.6E−07 | 8.135 | 0.037 | 0.005 | (SEQ ID No: 479) |
| | | | | | | | | | tgtgttgcacgttcag[A/G] |
| | | | | | | | | | caatggctcctaaaat |
| | 184 | rs16970520 | 16 | 69353341 | 5.1E−07 | 9.682 | 0.033 | 0.003 | (SEQ ID No: 480) |
| | | | | | | | | | ctgccctcattgaaca[A/G] |
| | | | | | | | | | aggaaaactgagatgc |
| | 185 | rs6564907 | 16 | 80263472 | 3.5E−05 | 7.563 | 0.026 | 0.003 | (SEQ ID No: 481) |
| | | | | | | | | | caccactggagaggta[C/T] |
| | | | | | | | | | gacacactggtgtgtg |
| | 186 | rs9908463 | 17 | 42507913 | 5.8E−19 | 47.88 | 0.062 | 0.001 | (SEQ ID No: 482) |
| | | | | | | | | | tcacttggatgtataa[A/C] |
| | | | | | | | | | cgtgaacaaactactt |
| | 187 | rs347546 | 18 | 48231042 | 1.5E−05 | 2.38 | 0.12 | 0.054 | (SEQ ID No: 483) |
| | | | | | | | | | tgaacaaaatggatga[A/G] |
| | | | | | | | | | gttctatcttcctgag |
| | 188 | rs714448 | 18 | 65799732 | 4.2E−09 | 10.63 | 0.043 | 0.004 | (SEQ ID No: 484) |
| | | | | | | | | | ccaaatataacaaaaa[A/C] |
| | | | | | | | | | cagatcataaacctca |
| 50 | 189 | SNP_A- | 18 | 67249199 | 1.2E−17 | 25.38 | 0.066 | 0.003 | (SEQ ID No: 485) |
| | | 4205076 | | | | | | | cctgtgtggagactgc[A/G] |
| | | | | | | | | | aaatacagacttttta |
| | 190 | rs6096732 | 20 | 50115618 | 6.3E−15 | 27.43 | 0.053 | 0.002 | (SEQ ID No: 486) |
| | | | | | | | | | aggatgctgagcttcc[A/G] |
| | | | | | | | | | agaaattgatggacct |
| | 191 | SNP_A- | 20 | 52465343 | 7.0E−06 | 11.73 | 0.024 | 0.002 | (SEQ ID No: 487) |
| | | 4235645 | | | | | | | ctggtgtatcttggta[A/G] |
| | | | | | | | | | aagctttagaaagttt |
| | 192 | rs4578944 | 21 | 18684634 | 1.6E−06 | 10.44 | 0.028 | 0.003 | (SEQ ID No: 488) |
| | | | | | | | | | ttagcaaatgaatgaa[A/G] |
| | | | | | | | | | aattatttagcagctg |
| | 193 | rs16991923 | 22 | 31889795 | 7.9E−10 | 14.96 | 0.039 | 0.003 | (SEQ ID No: 489) |
| | | | | | | | | | actacctcattatacg[A/T] |
| | | | | | | | | | aagacctaattcaatc |
| | 194 | SNP_A- | 22 | 38602840 | 1.9E−07 | 14.28 | 0.029 | 0.002 | (SEQ ID No: 490) |
| | | 1904522 | | | | | | | tggggatagagggaaa[A/C] |
| | | | | | | | | | aggattctgagctagg |
| | 195 | rs6608322 | 23 | 148809819 | 1.1E−06 | 9.219 | 0.032 | 0.004 | (SEQ ID No: 491) |
| | | | | | | | | | tttggctttagggaat[G/T] |
| | | | | | | | | | aaattatttctggctg |
| | 196 | rs4535928 | 23 | 152349432 | 1.6E−06 | 5.526 | 0.046 | 0.009 | (SEQ ID No: 492) |
| | | | | | | | | | aaaaaaaggaaaagcc[A/G] |
| | | | | | | | | | gaatgctatggactga |

TABLE 3
Anchor Tag
Block 001
Chromosome 1
Rs507667 (BP 37823966) LD block SNPs

| | | Minor | |
|---|---|---|---|
| | Base | Allele | |
| SNP ID (rs) | Position | Freq | Alleles |
| rs510208 | 37816259 | 0.275 | T:C |
| rs14307 | 37816699 | 0.225 | T:A |
| rs539585 | 37817216 | 0.275 | T:G |
| rs684260 | 37817858 | 0.225 | G:A |
| rs686083 | 37818286 | 0.275 | C:T |
| rs591567 | 37818940 | 0.275 | A:G |
| rs525379 | 37820798 | 0.275 | T:C |
| rs501252 | 37823263 | 0.275 | A:G |
| rs507667 | 37823966 | 0.492 | G:A |
| rs678511 | 37825520 | 0.275 | T:C |
| rs3762352 | 37825995 | 0.233 | C:T |
| rs565316 | 37826717 | 0.275 | T:C |
| rs768659 | 37831800 | 0.233 | A:C |
| rs497705 | 37831853 | 0.267 | C:G |
| rs10908369 | 37832432 | 0.192 | G:A |
| rs11264087 | 37832480 | 0.225 | C:T |
| rs4653320 | 37833919 | 0.075 | G:A |
| rs11264089 | 37834750 | 0.017 | C:T |
| rs661460 | 37835420 | 0.492 | A:T |
| rs12119598 | 37837897 | 0.142 | C:T |
| rs12139097 | 37838139 | 0.042 | T:G |
| rs3790623 | 37838671 | 0.258 | G:A |
| rs589927 | 37839091 | 0.492 | G:A |
| rs3790624 | 37839800 | 0.233 | G:A |
| rs11264090 | 37842475 | 0.233 | C:T |
| rs531454 | 37842690 | 0.492 | A:G |
| rs3748679 | 37842787 | 0.233 | A:G |
| rs557897 | 37843278 | 0.492 | G:T |
| rs546522 | 37845814 | 0.492 | C:G |
| rs548352 | 37846017 | 0.492 | G:C |
| rs571153 | 37846137 | 0.492 | A:G |
| rs631102 | 37847929 | 0.258 | C:T |
| rs658024 | 37849378 | 0.492 | A:G |
| rs502776 | 37851257 | 0.258 | T:A |
| rs609437 | 37853409 | 0.267 | T:C |
| rs610213 | 37853563 | 0.208 | C:G |
| rs7533921 | 37853974 | 0.233 | A:C |
| rs6670599 | 37854816 | 0.067 | C:T |
| rs12124242 | 37854907 | 0.192 | G:A |
| rs12131931 | 37854908 | 0.033 | C:A |
| rs6671088 | 37855282 | 0.192 | C:T |
| rs12726343 | 37855299 | 0.067 | C:G |
| rs1008879 | 37855387 | 0.192 | C:T |
| rs12729055 | 37855403 | 0.067 | G:A |
| rs17511088 | 37856266 | 0.067 | G:A |
| rs17511136 | 37856972 | 0.067 | C:T |
| rs12756715 | 37857122 | 0.067 | A:G |
| rs6426030 | 37857288 | 0.258 | A:G |
| rs12126510 | 37857315 | 0.192 | G:A |

TABLE 4
Anchor Tag
Block 002
Chromosome 1
Rs2070929 (BP 47,395,627) LD block SNPs

| | | Minor | |
|---|---|---|---|
| | Base | Allele | |
| SNP ID (rs) | Position | Freq | Alleles |
| rs741959 | 47388253 | 0.475 | G:A |
| rs2758743 | 47390375 | 0.025 | G:T |
| rs2070930 | 47395484 | 0.108 | G:A |
| rs2070929 | 47395627 | 0.292 | G:C |
| rs977747 | 47396697 | 0.467 | G:T |
| rs2249636 | 47398553 | 0.292 | A:C |
| rs2249665 | 47399104 | 0.108 | G:A |
| rs2984618 | 47402458 | 0.467 | G:T |
| rs2250495 | 47405397 | 0.292 | T:C |
| rs7534271 | 47406001 | 0.467 | G:C |
| rs2845 | 47406100 | 0.033 | G:A |
| rs11211481 | 47406187 | 0.467 | G:A |
| rs2798349 | 47410723 | 0.092 | A:G |
| rs6700838 | 47412047 | 0.467 | T:C |
| rs1015890 | 47414249 | 0.475 | A:G |
| rs12083555 | 47415420 | 0.408 | T:G |
| rs911910 | 47418990 | 0.475 | A:G |
| rs11211483 | 47419455 | 0.108 | G:C |
| rs10890472 | 47420132 | 0.442 | T:G |
| rs2057393 | 47420696 | 0.150 | T:A |
| rs765861 | 47421221 | 0.425 | T:A |

TABLE 5
Anchor Tag
Block 004
Chromosome 1
Rs1561167 (BP 64827534) and Rs11208493 (BP 64840416)
LD block SNPs

| | | Minor | |
|---|---|---|---|
| | Base | Allele | |
| SNP ID (rs) | Position | Freq | Alleles |
| rs12061466 | 64824609 | 0.092 | G:A |
| rs1561168 | 64827340 | 0.092 | G:A |
| rs1561167 | 64827534 | 0.092 | T:A |
| rs10493361 | 64827833 | 0.208 | A:C |
| rs10493362 | 64828353 | 0.092 | A:G |
| rs12742304 | 64830930 | 0.117 | C:T |
| rs7552864 | 64831167 | 0.092 | T:C |
| rs7518616 | 64831418 | 0.092 | A:G |
| rs12565437 | 64833582 | 0.092 | A:T |
| rs11208490 | 64836283 | 0.092 | C:T |
| rs11208491 | 64836720 | 0.033 | G:T |
| rs12565594 | 64837188 | 0.092 | T:C |
| rs6673348 | 64839925 | 0.058 | A:T |
| rs7517300 | 64840092 | 0.092 | T:C |
| rs11208492 | 64840396 | 0.092 | G:A |
| rs11208493 | 64840416 | 0.092 | C:T |
| rs4915996 | 64840944 | 0.092 | T:A |
| rs4578253 | 64842080 | 0.092 | A:T |
| rs11208496 | 64842622 | 0.092 | A:C |
| rs12136735 | 64844887 | 0.008 | A:G |
| rs12039471 | 64846725 | 0.092 | T:G |
| rs11208499 | 64847089 | 0.092 | C:A |
| rs7513574 | 64847362 | 0.092 | G:T |

TABLE 6
Anchor Tag
Block 005
Chromosome 1
Rs1337362 (BP 160925801) and Rs11809129 (BP 160961303)
LD block SNPs. These tags are in separate LD blocks.

| | | Minor | |
|---|---|---|---|
| | Base | Allele | |
| SNP ID (rs) | Position | Freq | Alleles |
| rs4258194 | 160912388 | 0.317 | G:A |
| rs803441 | 160915489 | 0.442 | G:A |
| rs782851 | 160925222 | 0.367 | C:T |
| rs1337362 | 160925801 | 0.267 | C:G |
| rs6672097 | 160925836 | 0.267 | C:T |
| rs6672382 | 160925957 | 0.267 | G:A |
| rs12060048 | 160926784 | 0.367 | A:G |
| rs12075422 | 160926939 | 0.367 | C:T |
| rs16826577 | 160927211 | 0.050 | T:C |
| rs1361066 | 160927773 | 0.050 | C:T |
| rs782854 | 160927831 | 0.275 | A:G |
| rs10800011 | 160932814 | 0.383 | A:G |
| rs16826634 | 160933236 | 0.017 | A:C |
| rs16826651 | 160934094 | 0.017 | G:T |
| rs7549902 | 160934431 | 0.050 | T:G |
| rs2153184 | 160935655 | 0.333 | G:A |
| rs7544769 | 160936605 | 0.050 | G:T |
| rs16826732 | 160936798 | 0.017 | G:A |
| rs16826743 | 160936910 | 0.017 | A:G |
| rs16826800 | 160938280 | 0.017 | G:C |
| rs12137386 | 160939201 | 0.425 | C:A |
| rs12126442 | 160939276 | 0.350 | A:G |
| rs16826820 | 160939381 | 0.083 | G:A |
| rs12129943 | 160939514 | 0.333 | G:C |
| rs16826843 | 160941459 | 0.017 | T:C |
| rs10494411 | 160942537 | 0.367 | T:C |
| rs6697445 | 160943572 | 0.050 | G:C |
| rs2881506 | 160944114 | 0.317 | A:G |
| rs10494412 | 160944138 | 0.050 | T:C |
| rs10494413 | 160945310 | 0.050 | G:A |
| rs1556867 | 160945344 | 0.275 | C:T |
| rs12040357 | 160946010 | 0.017 | G:A |
| rs12133767 | 160948436 | 0.308 | G:C |
| rs7520925 | 160948454 | 0.025 | G:T |
| rs713338 | 160948937 | 0.367 | T:G |
| rs1338988 | 160949090 | 0.308 | C:A |
| rs10917963 | 160950230 | 0.308 | C:T |
| rs1338991 | 160951167 | 0.367 | T:C |
| rs12049205 | 160952758 | 0.017 | C:A |
| rs7520430 | 160953966 | 0.350 | G:T |
| rs7532341 | 160954057 | 0.367 | T:C |
| rs12756531 | 160957469 | 0.042 | C:T |
| rs2881507 | 160957910 | 0.367 | G:A |
| rs10917965 | 160957934 | 0.367 | A:C |
| rs6682660 | 160959095 | 0.333 | C:A |
| rs11807906 | 160959721 | 0.133 | A:C |
| rs10917966 | 160959874 | 0.325 | C:G |
| rs1416430 | 160960255 | 0.017 | G:A |
| rs1416431 | 160960526 | 0.350 | G:C |
| rs11809129 | 160961303 | 0.308 | T:C |
| rs10917967 | 160962041 | 0.325 | A:G |
| rs7516405 | 160962899 | 0.308 | G:T |
| rs1416432 | 160963200 | 0.308 | T:C |
| rs2346885 | 160963607 | 0.333 | G:C |
| rs1338994 | 160965841 | 0.350 | T:C |
| rs1338995 | 160965993 | 0.350 | A:G |
| rs7512727 | 160968850 | 0.050 | A:G |
| rs9645323 | 160969260 | 0.133 | T:C |
| rs1416433 | 160970463 | 0.308 | C:A |
| rs1338996 | 160971413 | 0.308 | G:A |
| rs1338997 | 160971809 | 0.083 | G:A |
| rs1591821 | 160973951 | 0.308 | C:T |
| rs1953819 | 160974023 | 0.383 | C:T |
| rs1934038 | 160974384 | 0.383 | C:T |
| rs1934040 | 160975540 | 0.358 | C:T |
| rs1578087 | 160975754 | 0.383 | G:T |
| rs4656414 | 160976687 | 0.333 | A:G |
| rs1578090 | 160979060 | 0.308 | A:G |

TABLE 7
Anchor Tag
Block 006
Chromosome 1
Rs12739430 (BP 165684746) LD block SNPs

| | | Minor | |
|---|---|---|---|
| | Base | Allele | |
| SNP ID (rs) | Position | Freq | Alleles |
| rs2104189 | 165667434 | 0.175 | G:A |
| rs2104190 | 165667446 | 0.175 | G:T |
| rs10489373 | 165671447 | 0.192 | G:A |
| rs6427168 | 165672674 | 0.492 | G:A |
| rs2419688 | 165673284 | 0.175 | C:T |
| rs10919026 | 165673548 | 0.483 | C:T |
| rs10800398 | 165674487 | 0.492 | T:C |
| rs2181210 | 165675428 | 0.492 | C:T |
| rs12067790 | 165675478 | 0.092 | T:G |
| rs12026831 | 165676552 | 0.192 | G:A |
| rs3007378 | 165676883 | 0.333 | G:T |
| rs10919028 | 165677084 | 0.100 | G:A |
| rs7529999 | 165677827 | 0.192 | T:C |
| rs10458393 | 165680880 | 0.192 | T:C |
| rs3007379 | 165681159 | 0.175 | G:T |
| rs3007381 | 165682166 | 0.333 | T:G |
| rs12030460 | 165682799 | 0.150 | T:C |
| rs1033806 | 165684137 | 0.175 | C:T |
| rs1033805 | 165684192 | 0.342 | A:G |
| rs12739430 | 165684746 | 0.192 | A:G |
| rs4142001 | 165684982 | 0.175 | T:C |
| rs7538643 | 165685892 | 0.017 | T:C |
| rs2146194 | 165686272 | 0.192 | C:T |
| rs2146195 | 165686371 | 0.175 | C:T |
| rs4656634 | 165687953 | 0.408 | T:C |
| rs2984594 | 165688313 | 0.175 | C:T |
| rs10800399 | 165688442 | 0.175 | C:A |
| rs10489374 | 165688646 | 0.125 | T:C |
| rs992325 | 165688734 | 0.042 | G:A |
| rs10489375 | 165689281 | 0.192 | T:A |
| rs12747138 | 165690669 | 0.192 | T:C |
| rs1322486 | 165691752 | 0.175 | T:C |

TABLE 8
Anchor Tag
Block 007
Chromosome 1
Rs10494966 (BP 210389815) and Rs9651104
(BP 210392113) LD block SNPs

| | | Minor | |
|---|---|---|---|
| | Base | Allele | |
| SNP ID (rs) | Position | Freq | Alleles |
| rs12036965 | 210387043 | 0.142 | T:G |
| rs1341152 | 210387829 | 0.133 | G:A |
| rs12084751 | 210388137 | 0.042 | G:T |
| rs10779610 | 210389623 | 0.183 | T:A |
| rs10494966 | 210389815 | 0.133 | G:C |
| rs10494967 | 210389989 | 0.183 | G:A |
| rs6693625 | 210390655 | 0.050 | C:T |
| rs17021510 | 210392000 | 0.008 | C:G |
| rs9651104 | 210392113 | 0.183 | A:G |
| rs7553890 | 210394300 | 0.183 | T:C |

TABLE 9
Anchor Tag
Block 008
Chromosome 1
Rs4520477 (BP 213031092) and Rs4579828
(BP 213031765) LD block SNPs

| | | Minor | |
|---|---|---|---|
| | Base | Allele | |
| SNP ID (rs) | Position | Freq | Alleles |
| rs4486507 | 213026494 | 0.350 | C:G |
| rs12142867 | 213026644 | 0.042 | G:A |
| rs12725653 | 213026668 | 0.325 | T:C |
| rs6422637 | 213028375 | 0.008 | A:G |
| rs6604601 | 213028593 | 0.008 | G:A |
| rs17042581 | 213029308 | 0.008 | C:T |
| rs7533687 | 213030289 | 0.008 | A:G |
| rs4520477 | 213031092 | 0.350 | A:G |
| rs4579828 | 213031765 | 0.350 | G:A |
| rs13376678 | 213034867 | 0.008 | G:A |
| rs4338426 | 213038062 | 0.358 | T:C |
| rs7411184 | 213040199 | 0.367 | C:T |
| rs11117589 | 213040241 | 0.350 | G:T |
| rs6660708 | 213043390 | 0.042 | T:C |
| rs6691953 | 213043517 | 0.008 | G:A |
| rs6604611 | 213043646 | 0.050 | C:A |
| rs7554439 | 213044094 | 0.375 | C:T |
| rs12067849 | 213044365 | 0.008 | T:C |
| rs7548662 | 213045041 | 0.358 | G:A |
| rs4240923 | 213045654 | 0.333 | G:T |
| rs4348785 | 213046243 | 0.333 | G:A |
| rs6604612 | 213046504 | 0.350 | C:T |
| rs6671250 | 213046703 | 0.008 | T:C |
| rs12031665 | 213053850 | 0.342 | A:G |
| rs10863244 | 213055116 | 0.008 | A:G |
| rs6681237 | 213055259 | 0.350 | T:C |

TABLE 10
Anchor Tag
Block 009
Chromosome 1
Rs10863539 (BP 216800365) LD block SNPs

| | | Minor | |
|---|---|---|---|
| | Base | Allele | |
| SNP ID (rs) | Position | Freq | Alleles |
| rs12404566 | 216761874 | 0.183 | T:A |
| rs12129903 | 216762442 | 0.150 | A:C |
| rs12026681 | 216763535 | 0.300 | T:C |
| rs7541290 | 216764348 | 0.192 | A:T |
| rs7551804 | 216764788 | 0.192 | G:C |
| rs11118501 | 216765068 | 0.025 | G:C |
| rs12025702 | 216765825 | 0.025 | G:A |
| rs4142940 | 216767898 | 0.292 | G:C |
| rs4142941 | 216767911 | 0.008 | T:G |
| rs12035921 | 216769214 | 0.442 | A:C |
| rs4492628 | 216771742 | 0.008 | T:C |
| rs4511114 | 216771844 | 0.292 | C:G |
| rs4351641 | 216773838 | 0.192 | C:T |
| rs17565134 | 216780807 | 0.183 | A:C |
| rs12040095 | 216781809 | 0.025 | T:C |
| rs17007186 | 216783404 | 0.025 | G:A |
| rs6541126 | 216784676 | 0.025 | A:G |
| rs17007187 | 216788303 | 0.008 | T:G |
| rs11118506 | 216788995 | 0.025 | C:T |
| rs11585179 | 216789211 | 0.017 | G:A |
| rs6541127 | 216789404 | 0.292 | T:C |
| rs10127631 | 216790563 | 0.025 | G:A |
| rs10863537 | 216790798 | 0.217 | T:C |
| rs7535619 | 216793106 | 0.292 | A:G |
| rs12086760 | 216794449 | 0.025 | T:C |
| rs11118507 | 216795750 | 0.292 | A:G |
| rs11118508 | 216798601 | 0.192 | A:C |
| rs10495145 | 216798841 | 0.008 | A:T |
| rs11118509 | 216799210 | 0.025 | G:A |
| rs10863539 | 216800365 | 0.025 | A:G |
| rs10863540 | 216801952 | 0.025 | T:C |
| rs2221833 | 216803267 | 0.300 | A:G |
| rs11118510 | 216805132 | 0.025 | C:T |
| rs2808009 | 216808819 | 0.308 | T:C |
| rs7537417 | 216818213 | 0.283 | C:G |
| rs10779400 | 216821276 | 0.283 | T:C |
| rs10779401 | 216824394 | 0.025 | T:C |
| rs2808016 | 216825337 | 0.283 | G:A |
| rs12404532 | 216830764 | 0.175 | G:C |
| rs2808019 | 216833320 | 0.283 | G:A |
| rs6690426 | 216835204 | 0.025 | T:C |
| rs2667961 | 216835997 | 0.008 | A:C |
| rs1511695 | 216836725 | 0.183 | G:A |
| rs2814495 | 216840552 | 0.217 | C:G |

TABLE 11
Anchor Tag
Block 010
Chromosome 1
Rs4427395 (BP 234188628) and Rs10925498 (BP 234189251)
LD block SNPs. These tags are in separate LD blocks.

| | | Minor | |
|---|---|---|---|
| | Base | Allele | |
| SNP ID (rs) | Position | Freq | Alleles |
| rs4427395 | 234188628 | 0.317 | A:G |
| rs10925498 | 234189251 | 0.308 | T:G |

TABLE 12
Anchor Tag
Block 001
Chromosome 2
Rs7599610 (BP 5871611) LD block SNPs

| | | Minor | |
|---|---|---|---|
| | Base | Allele | |
| SNP ID (rs) | Position | Freq | Alleles |
| rs2722596 | 5852524 | 0.392 | G:A |
| rs16864155 | 5853338 | 0.075 | C:A |
| rs1560243 | 5853783 | 0.033 | G:A |
| rs1429244 | 5854295 | 0.392 | T:C |
| rs1429245 | 5854474 | 0.233 | G:A |
| rs16864162 | 5856852 | 0.075 | T:A |
| rs16864163 | 5857115 | 0.100 | T:C |
| rs1366745 | 5857821 | 0.392 | G:A |
| rs1366746 | 5857861 | 0.233 | T:C |
| rs1366747 | 5857892 | 0.333 | A:G |
| rs6710207 | 5858109 | 0.033 | C:T |
| rs16864170 | 5858478 | 0.075 | T:C |
| rs1560244 | 5858542 | 0.392 | T:C |
| rs1560245 | 5858717 | 0.033 | G:A |
| rs1560246 | 5858749 | 0.492 | A:G |
| rs1030494 | 5860845 | 0.392 | C:A |
| rs16864175 | 5861984 | 0.033 | T:C |
| rs2163030 | 5862170 | 0.392 | C:T |
| rs16864179 | 5863087 | 0.033 | C:T |
| rs2564068 | 5863300 | 0.392 | A:C |
| rs16864181 | 5863695 | 0.033 | T:A |
| rs17363208 | 5864206 | 0.017 | G:A |
| rs2564070 | 5865008 | 0.392 | C:T |
| rs2722598 | 5865919 | 0.392 | G:A |
| rs7571516 | 5865956 | 0.392 | G:C |
| rs6432295 | 5866512 | 0.033 | T:A |
| rs4613249 | 5866636 | 0.392 | T:C |
| rs2163024 | 5867185 | 0.392 | T:C |
| rs10929818 | 5867846 | 0.342 | G:A |
| rs1366735 | 5868807 | 0.233 | T:C |
| rs1429224 | 5868921 | 0.392 | A:G |
| rs16864190 | 5869014 | 0.017 | C:T |
| rs1429223 | 5869077 | 0.342 | C:T |
| rs4668769 | 5870774 | 0.267 | G:A |
| rs1429222 | 5871300 | 0.392 | T:G |
| rs16864193 | 5871693 | 0.075 | C:T |
| rs16864200 | 5876754 | 0.258 | C:T |
| rs2564077 | 5876786 | 0.275 | A:G |
| rs1346608 | 5877517 | 0.375 | G:T |
| rs16864209 | 5878691 | 0.258 | T:C |
| rs17359248 | 5878969 | 0.033 | A:G |
| rs2722608 | 5880921 | 0.450 | T:C |
| rs1429220 | 5881639 | 0.275 | G:A |
| rs10495524 | 5881900 | 0.258 | G:A |
| rs7425362 | 5882495 | 0.258 | G:A |
| rs2722606 | 5884092 | 0.275 | A:G |
| rs10803749 | 5884443 | 0.258 | C:T |

TABLE 13
Anchor Tag
Block 002
Chromosome 2
Rs720118 (BP 5987961) LD block SNPs

| | | Minor | |
|---|---|---|---|
| | Base | Allele | |
| SNP ID (rs) | Position | Freq | Alleles |
| rs6760889 | 5979682 | 0.442 | T:C |
| rs16864471 | 5985162 | 0.042 | C:T |
| rs2882450 | 5987356 | 0.042 | C:A |
| rs720118 | 5987961 | 0.442 | T:C |
| rs2564041 | 5988912 | 0.083 | G:A |
| rs2464989 | 5989204 | 0.083 | A:G |
| rs7606978 | 5989441 | 0.083 | A:G |
| rs12475323 | 5989534 | 0.042 | T:C |
| rs16864476 | 5989662 | 0.042 | G:T |
| rs7569997 | 5989739 | 0.083 | T:A |
| rs16864477 | 5989775 | 0.042 | G:A |
| rs12618928 | 5989789 | 0.442 | C:T |

TABLE 14
Anchor Tag
Block 003
Chromosome 2
Rs10192401 (BP 75313269) and Rs10203484
(BP 75313393) LD block SNPs

| | | Minor | |
|---|---|---|---|
| | Base | Allele | |
| SNP ID (rs) | Position | Freq | Alleles |
| rs10192401 | 75313269 | 0.392 | C:T |
| rs10203484 | 75313393 | 0.392 | G:A |
| rs12477554 | 75313720 | 0.458 | G:A |

TABLE 15
Anchor Tag
Block 004
Chromosome 2
Rs736711 (BP 84977903) LD block SNPs

| | | Minor | |
|---|---|---|---|
| | Base | Allele | |
| SNP ID (rs) | Position | Freq | Alleles |
| rs13406208 | 84910304 | 0.150 | T:C |
| rs11681478 | 84912388 | 0.150 | C:T |
| rs13425682 | 84912640 | 0.083 | A:G |
| rs13407222 | 84914248 | 0.083 | G:A |
| rs1192378 | 84917869 | 0.150 | C:T |
| rs7566739 | 84918368 | 0.017 | C:T |
| rs1192377 | 84918816 | 0.150 | G:T |
| rs1192376 | 84919142 | 0.025 | A:G |
| rs11690152 | 84919493 | 0.025 | T:G |
| rs11685534 | 84919809 | 0.025 | G:A |
| rs11685490 | 84919839 | 0.025 | C:G |
| rs1192375 | 84920031 | 0.108 | C:T |
| rs1192374 | 84920682 | 0.067 | G:A |
| rs1192372 | 84923204 | 0.125 | T:C |
| rs1192316 | 84924860 | 0.108 | G:A |
| rs1192311 | 84930989 | 0.108 | G:A |
| rs17025551 | 84931564 | 0.017 | G:T |
| rs17608438 | 84931630 | 0.075 | A:G |
| rs1192309 | 84931802 | 0.108 | A:G |
| rs17025553 | 84932007 | 0.125 | C:T |
| rs17025556 | 84932360 | 0.125 | G:A |
| rs1192399 | 84934173 | 0.150 | C:G |
| rs754273 | 84935212 | 0.125 | A:G |
| rs754274 | 84935311 | 0.125 | G:C |
| rs1192396 | 84936012 | 0.125 | A:G |
| rs1192395 | 84936389 | 0.108 | C:T |
| rs1192394 | 84938458 | 0.150 | T:G |
| rs1007696 | 84938895 | 0.125 | C:T |
| rs1192388 | 84941284 | 0.150 | G:A |
| rs1192368 | 84944997 | 0.150 | A:C |
| rs1192367 | 84945493 | 0.108 | C:G |
| rs11692245 | 84945704 | 0.025 | T:A |
| rs11679655 | 84946624 | 0.025 | G:C |
| rs1192366 | 84946714 | 0.108 | T:C |
| rs11686936 | 84947598 | 0.025 | T:A |
| rs1192365 | 84948658 | 0.108 | A:G |
| rs1192364 | 84949187 | 0.125 | C:A |
| rs2276610 | 84951221 | 0.017 | C:T |
| rs11687426 | 84951595 | 0.042 | G:A |
| rs1192362 | 84952051 | 0.150 | G:C |
| rs2276609 | 84952063 | 0.042 | A:G |
| rs1192361 | 84952076 | 0.108 | A:G |
| rs1192360 | 84952953 | 0.108 | T:C |
| rs1192359 | 84952974 | 0.150 | T:C |
| rs17025592 | 84953294 | 0.017 | A:G |
| rs1192358 | 84953994 | 0.108 | T:G |
| rs17025602 | 84955011 | 0.008 | G:A |
| rs11693821 | 84955489 | 0.025 | G:C |
| rs1192356 | 84955762 | 0.150 | T:C |
| rs10490704 | 84957867 | 0.017 | A:G |
| rs7584703 | 84958576 | 0.017 | G:T |
| rs1192353 | 84959002 | 0.150 | T:C |
| rs17025617 | 84959825 | 0.008 | A:G |
| rs11674022 | 84962146 | 0.050 | G:A |
| rs1627220 | 84965114 | 0.108 | C:T |
| rs1192280 | 84966758 | 0.108 | C:G |
| rs1077440 | 84966860 | 0.150 | A:G |
| rs1192277 | 84967480 | 0.108 | A:G |
| rs1192275 | 84969212 | 0.108 | A:T |
| rs1192273 | 84970721 | 0.125 | G:A |
| rs7584746 | 84973733 | 0.142 | T:G |
| rs736711 | 84977903 | 0.100 | G:A |

TABLE 16
Anchor Tag
Block 005
Chromosome 2
Rs4386315 (BP 88275093), Rs10168005 (BP 88285077),
and Rs12463835 (BP 88285874) LD block SNPs

| | | Minor | |
|---|---|---|---|
| | Base | Allele | |
| SNP ID (rs) | Position | Freq | Alleles |
| rs2919872 | 88267833 | 0.392 | C:T |
| rs10199430 | 88268016 | 0.008 | A:T |
| rs2970901 | 88268096 | 0.392 | G:T |
| rs2970902 | 88268445 | 0.300 | G:C |
| rs2970903 | 88268853 | 0.092 | C:T |
| rs2860391 | 88269522 | 0.267 | T:A |
| rs13009478 | 88271590 | 0.325 | T:C |
| rs12470985 | 88271656 | 0.292 | G:T |
| rs2860399 | 88272243 | 0.283 | A:G |
| rs2970924 | 88272567 | 0.108 | C:T |
| rs7581571 | 88274497 | 0.175 | G:A |
| rs4386315 | 88275093 | 0.092 | G:A |
| rs6735524 | 88275263 | 0.408 | G:A |
| rs7420937 | 88276430 | 0.100 | G:C |
| rs10200591 | 88276841 | 0.175 | T:C |
| rs4246598 | 88277312 | 0.408 | C:A |
| rs4281907 | 88277488 | 0.408 | C:A |
| rs13386415 | 88278518 | 0.092 | G:A |
| rs4618056 | 88279275 | 0.283 | C:T |
| rs6737877 | 88280894 | 0.092 | C:G |
| rs17350803 | 88283140 | 0.308 | T:C |
| rs12478496 | 88283462 | 0.025 | G:A |
| rs4325755 | 88283742 | 0.317 | C:T |
| rs7572594 | 88284635 | 0.017 | G:A |
| rs10168005 | 88285077 | 0.083 | C:G |
| rs12463835 | 88285874 | 0.092 | C:T |
| rs10865489 | 88286154 | 0.208 | T:G |
| rs6547752 | 88286699 | 0.208 | A:G |
| rs7582011 | 88292002 | 0.208 | T:A |
| rs11896492 | 88294221 | 0.175 | T:C |
| rs10210295 | 88299497 | 0.008 | C:T |
| rs13010480 | 88299661 | 0.308 | T:C |
| rs7425197 | 88300251 | 0.308 | C:T |
| rs6727043 | 88300774 | 0.208 | T:A |
| rs10173112 | 88302417 | 0.208 | G:C |
| rs10185660 | 88302499 | 0.175 | T:C |
| rs4490184 | 88302640 | 0.092 | A:G |
| rs4359651 | 88304102 | 0.208 | T:G |
| rs4575723 | 88304153 | 0.092 | T:C |
| rs4246599 | 88304188 | 0.092 | G:A |
| rs9636487 | 88304225 | 0.167 | C:G |
| rs4633926 | 88304738 | 0.083 | C:A |
| rs10469977 | 88306741 | 0.092 | T:C |
| rs10469888 | 88306838 | 0.208 | G:T |

TABLE 17
Anchor Tag
Block 004
Chromosome 2
Rs4666865 (BP 183523597) LD block SNPs.
No defined LD in Haploview for this marker.

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

| | | | |
|---|---|---|---|
| rs4666865 | 183523597 | N/A | N/A |

TABLE 18
Anchor Tag
Block 007
Chromosome 2
Rs10206592 (BP 235331943) LD block SNPs

| | | Minor | |
|---|---|---|---|
| | Base | Allele | |
| SNP ID (rs) | Position | Freq | Alleles |
| rs12692146 | 235317223 | 0.342 | G:T |
| rs2890384 | 235319746 | 0.083 | T:C |
| rs4663449 | 235320902 | 0.325 | C:A |
| rs6431278 | 235321546 | 0.242 | G:A |
| rs6431279 | 235321687 | 0.242 | C:A |
| rs4663152 | 235323104 | 0.242 | T:C |
| rs4257436 | 235323490 | 0.242 | T:G |
| rs4519586 | 235324138 | 0.242 | T:C |
| rs4606985 | 235324389 | 0.242 | C:T |
| rs13402575 | 235324430 | 0.158 | C:T |
| rs4408776 | 235324492 | 0.242 | C:T |
| rs4074893 | 235324860 | 0.492 | T:C |
| rs7580207 | 235325299 | 0.242 | G:A |
| rs4603827 | 235325316 | 0.258 | C:T |
| rs2386510 | 235327521 | 0.325 | T:C |
| rs2091347 | 235327812 | 0.242 | G:T |
| rs10929046 | 235329067 | 0.242 | C:T |
| rs1405139 | 235329395 | 0.242 | T:C |
| rs1405140 | 235329435 | 0.242 | G:C |
| rs1526114 | 235330352 | 0.250 | A:T |
| rs1526117 | 235331882 | 0.492 | T:C |
| rs10206592 | 235331943 | 0.242 | A:T |
| rs10206795 | 235332136 | 0.242 | C:T |
| rs10206814 | 235332191 | 0.242 | C:T |
| rs10167927 | 235332321 | 0.242 | A:G |
| rs10168146 | 235332486 | 0.242 | A:G |
| rs11694116 | 235333074 | 0.242 | C:A |
| rs11695467 | 235333608 | 0.242 | T:A |
| rs13033817 | 235333799 | 0.242 | T:A |
| rs13383923 | 235333977 | 0.158 | T:C |
| rs1358068 | 235335280 | 0.242 | A:T |
| rs4663452 | 235335920 | 0.242 | T:A |

TABLE 19
Anchor Tag
Block 001
Chromosome 3
Rs3772284 (BP 1372414) LD block SNPs

| | | Minor | |
|---|---|---|---|
| | Base | Allele | |
| SNP ID (rs) | Position | Freq | Alleles |
| rs3796174 | 1367751 | 0.475 | T:C |
| rs2036686 | 1368163 | 0.375 | A:G |
| rs7651808 | 1368675 | 0.400 | G:C |
| rs7648067 | 1370906 | 0.333 | A:G |
| rs2133452 | 1370966 | 0.342 | A:G |
| rs7653048 | 1371176 | 0.083 | C:T |
| rs1499215 | 1372069 | 0.333 | T:C |
| rs17193334 | 1372373 | 0.058 | G:A |
| rs3772284 | 1372414 | 0.375 | C:T |
| rs17038138 | 1372582 | 0.050 | G:A |
| rs3772283 | 1373272 | 0.033 | T:C |
| rs1351272 | 1373511 | 0.325 | G:T |
| rs269672 | 1373990 | 0.450 | G:A |
| rs1479529 | 1375391 | 0.375 | G:A |
| rs1479530 | 1375419 | 0.375 | C:G |
| rs155895 | 1376122 | 0.342 | T:C |
| rs10510208 | 1376516 | 0.258 | T:G |
| rs155896 | 1376541 | 0.150 | A:C |
| rs9837911 | 1376808 | 0.375 | T:C |
| rs13093486 | 1377904 | 0.325 | A:G |
| rs155900 | 1378076 | 0.458 | C:T |
| rs9829288 | 1379038 | 0.333 | C:T |
| rs9848880 | 1379179 | 0.333 | A:G |

TABLE 20
Anchor Tag
Block 002
Chromosome 3
Rs7633620 (BP 2950433), Rs6789338 (BP 2951094), and
Rs4487227 (BP 2954275) LD block SNPs

| | | Minor | |
|---|---|---|---|
| | Base | Allele | |
| SNP ID (rs) | Position | Freq | Alleles |
| rs7633620 | 2950433 | 0.325 | A:G |
| rs6442766 | 2950573 | 0.325 | G:T |
| rs6789338 | 2951094 | 0.442 | A:G |
| rs12493308 | 2952837 | 0.125 | C:T |
| rs4487227 | 2954275 | 0.308 | G:A |
| rs1499137 | 2954471 | 0.308 | G:A |
| rs4684371 | 2955545 | 0.258 | A:G |
| rs4685582 | 2956587 | 0.258 | G:A |
| rs6786174 | 2957630 | 0.125 | T:G |
| rs12493948 | 2958799 | 0.158 | C:G |
| rs6787320 | 2958948 | 0.175 | G:A |
| rs9310890 | 2959356 | 0.392 | T:G |
| rs6805012 | 2960839 | 0.158 | G:T |
| rs12496487 | 2961176 | 0.067 | C:A |
| rs2874550 | 2961817 | 0.067 | T:A |
| rs876254 | 2964603 | 0.092 | A:C |
| rs12495321 | 2965078 | 0.092 | C:T |
| rs1909396 | 2965400 | 0.092 | G:A |
| rs6442770 | 2965978 | 0.258 | A:C |

TABLE 21
Anchor Tag
Block 003
Chromosome 3
Rs12488822 (BP 57292820) LD block SNPs

| | | Minor | |
|---|---|---|---|
| | Base | Allele | |
| SNP ID (rs) | Position | Freq | Alleles |
| rs6445860 | 57184237 | 0.475 | G:T |
| rs4493401 | 57188764 | 0.408 | T:C |
| rs4493402 | 57188891 | 0.433 | T:C |
| rs6791499 | 57194562 | 0.025 | T:C |
| rs7433481 | 57201631 | 0.417 | T:G |
| rs11710045 | 57212261 | 0.442 | T:C |
| rs975418 | 57213644 | 0.392 | G:T |
| rs1028040 | 57215650 | 0.392 | C:T |
| rs6445863 | 57219937 | 0.033 | T:C |
| rs6774584 | 57220362 | 0.433 | A:G |
| rs6794393 | 57231161 | 0.392 | T:C |
| rs6794708 | 57231676 | 0.392 | A:T |
| rs6445864 | 57233519 | 0.392 | T:A |
| rs3806622 | 57235870 | 0.417 | C:G |
| rs4640525 | 57238434 | 0.408 | G:C |
| rs6784630 | 57242608 | 0.392 | C:G |
| rs7643473 | 57248175 | 0.017 | C:T |
| rs6762354 | 57248965 | 0.392 | G:A |
| rs1553987 | 57250185 | 0.433 | A:G |
| rs7615217 | 57253713 | 0.433 | A:G |
| rs4681966 | 57264324 | 0.417 | T:C |
| rs925566 | 57268337 | 0.408 | T:C |
| rs10510791 | 57269125 | 0.392 | C:G |
| rs1533272 | 57269335 | 0.392 | T:C |
| rs2102628 | 57271792 | 0.392 | C:T |
| rs3087684 | 57281359 | 0.458 | C:T |
| rs17791685 | 57281651 | 0.125 | C:A |
| rs1913302 | 57282001 | 0.458 | A:G |
| rs17735309 | 57283243 | 0.125 | T:C |
| rs1355797 | 57290597 | 0.092 | A:G |
| rs951339 | 57290937 | 0.450 | T:A |
| rs11130579 | 57292626 | 0.400 | C:T |
| rs13059664 | 57292759 | 0.008 | C:G |
| rs12488822 | 57292820 | 0.442 | C:T |
| rs9840663 | 57295044 | 0.442 | C:T |
| rs12487501 | 57295522 | 0.092 | G:A |
| rs528035 | 57298210 | 0.392 | A:G |
| rs536036 | 57301923 | 0.458 | A:G |
| rs693378 | 57302485 | 0.017 | T:G |
| rs4681968 | 57303907 | 0.125 | G:T |
| rs502442 | 57306267 | 0.392 | G:A |
| rs6445868 | 57306715 | 0.125 | C:A |
| rs547505 | 57308767 | 0.392 | G:T |

TABLE 22
Anchor Tag
Block 004
Chromosome 3
Rs9830289 (BP 107972966), Rs4895039 (BP 108005925), Rs2049118
(BP 108010714), and rs6779049 (BP 108,018,247) LD block SNPs

| | | Minor | |
|---|---|---|---|
| | Base | Allele | |
| SNP ID (rs) | Position | Freq | Alleles |
| rs4895025 | 107952140 | 0.167 | C:T |
| rs1520720 | 107953485 | 0.167 | C:T |
| rs6781971 | 107953819 | 0.333 | A:C |
| rs13325649 | 107956326 | 0.167 | C:T |
| rs7610979 | 107958917 | 0.042 | T:C |
| rs4895026 | 107962231 | 0.333 | G:C |
| rs7631620 | 107970710 | 0.033 | T:C |
| rs7620084 | 107970868 | 0.008 | C:T |
| rs11917560 | 107971579 | 0.033 | G:A |
| rs11921595 | 107971603 | 0.033 | T:C |
| rs9830289 | 107972966 | 0.333 | T:C |
| rs6785279 | 107975580 | 0.017 | C:T |
| rs1520710 | 107976035 | 0.017 | T:C |
| rs1520711 | 107976056 | 0.167 | C:T |
| rs16852952 | 107976676 | 0.292 | T:C |
| rs1356933 | 107976694 | 0.017 | A:G |
| rs9852248 | 107977375 | 0.017 | T:C |
| rs12492041 | 107977767 | 0.333 | C:T |
| rs11713942 | 107977980 | 0.117 | T:C |
| rs1402304 | 107978014 | 0.217 | G:A |
| rs1520712 | 107978525 | 0.017 | G:A |
| rs4894858 | 107978614 | 0.200 | G:A |
| rs10804451 | 107979696 | 0.117 | G:A |
| rs6765217 | 107980700 | 0.017 | G:A |
| rs1402305 | 107981349 | 0.017 | A:T |
| rs2139710 | 107981907 | 0.017 | G:C |
| rs2139709 | 107982049 | 0.017 | G:A |
| rs6796767 | 107982786 | 0.033 | C:T |
| rs9865847 | 107984940 | 0.333 | A:C |
| rs4895028 | 107986238 | 0.167 | T:C |
| rs4894860 | 107987393 | 0.367 | C:T |
| rs4894861 | 107987467 | 0.017 | G:A |
| rs4895030 | 107987742 | 0.017 | A:G |
| rs4895031 | 107987748 | 0.017 | G:A |
| rs1607793 | 107991884 | 0.167 | A:C |
| rs9917651 | 107998724 | 0.117 | G:A |
| rs4895039 | 108005925 | 0.300 | T:G |
| rs1915330 | 108008292 | 0.308 | T:C |
| rs2049118 | 108010714 | 0.308 | G:C |
| rs10511259 | 108010853 | 0.133 | C:T |
| rs1520717 | 108011334 | 0.342 | T:C |
| rs939452 | 108012332 | 0.308 | G:A |
| rs16852987 | 108013014 | 0.167 | A:G |
| rs9875891 | 108015024 | 0.333 | A:C |
| rs6779049 | 108018247 | 0.308 | T:C |
| rs4894863 | 108018368 | 0.342 | C:G |
| rs2399117 | 108020680 | 0.033 | C:T |
| rs6804867 | 108021613 | 0.042 | T:C |
| rs17189858 | 108021718 | 0.117 | C:T |
| rs4895042 | 108021965 | 0.392 | A:C |
| rs7652022 | 108024742 | 0.375 | G:A |
| rs1467261 | 108025178 | 0.033 | T:C |
| rs9830673 | 108025656 | 0.008 | G:A |
| rs13083414 | 108025660 | 0.008 | G:A |
| rs6787966 | 108025914 | 0.150 | C:T |
| rs9831080 | 108026134 | 0.008 | C:G |
| rs13089375 | 108026873 | 0.033 | G:A |
| rs954995 | 108028687 | 0.375 | T:C |
| rs4602430 | 108028767 | 0.017 | G:C |
| rs2176898 | 108030375 | 0.017 | T:G |
| rs796467 | 108031811 | 0.008 | C:G |
| rs773234 | 108032104 | 0.008 | C:G |
| rs951940 | 108034203 | 0.150 | G:A |
| rs12637775 | 108035061 | 0.150 | T:C |

TABLE 23
Anchor Tag
Block 005
Chromosome 30
Rs2331921 (BP 122608097), Rs12186062 (BP 122626569), Rs4555467
(BP 122693312), and Rs3806614 (BP 122747866) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs11707293 | 122567157 | 0.450 | T:C | rs6782033 | 122620705 | 0.250 | T:C |
| rs12494335 | 122577282 | 0.467 | T:C | rs10212489 | 122622744 | 0.158 | T:A |
| rs4346498 | 122580925 | 0.250 | G:T | rs6787048 | 122623726 | 0.250 | G:T |
| rs17740066 | 122582973 | 0.092 | G:A | rs17678695 | 122623786 | 0.050 | A:G |
| rs17740072 | 122583104 | 0.158 | A:G | rs6798699 | 122623898 | 0.408 | G:A |
| rs12107515 | 122584542 | 0.125 | G:C | rs7632907 | 122624408 | 0.250 | C:T |
| rs3914164 | 122586192 | 0.125 | G:A | rs12186062 | 122626569 | 0.408 | T:C |
| rs4080279 | 122597266 | 0.467 | C:T | rs9289170 | 122627199 | 0.092 | T:C |
| rs4107753 | 122597484 | 0.175 | T:A | rs2126140 | 122627958 | 0.408 | C:G |
| rs6796068 | 122600457 | 0.408 | A:G | rs2169302 | 122628751 | 0.175 | T:C |
| rs6766105 | 122601791 | 0.308 | T:C | rs9862879 | 122631699 | 0.158 | C:T |
| rs9878946 | 122604648 | 0.100 | C:T | rs716688 | 122632246 | 0.100 | A:T |
| rs1986730 | 122604974 | 0.408 | C:A | rs9868609 | 122632861 | 0.100 | G:A |
| rs11914436 | 122605913 | 0.092 | G:A | rs2306211 | 122634474 | 0.008 | G:A |
| rs7644928 | 122606733 | 0.408 | C:G | rs12629829 | 122636880 | 0.008 | G:T |
| rs1987097 | 122606848 | 0.408 | C:T | rs6800901 | 122636883 | 0.283 | C:T |
| rs4440066 | 122607087 | 0.100 | G:C | rs1381057 | 122637664 | 0.350 | C:T |
| rs1987098 | 122607217 | 0.250 | A:G | rs2030531 | 122637888 | 0.425 | T:C |
| rs9828910 | 122607697 | 0.100 | A:C | rs16832303 | 122638449 | 0.125 | A:G |
| rs2331921 | 122608097 | 0.408 | G:A | rs1522357 | 122638633 | 0.308 | G:A |
| rs6438625 | 122610411 | 0.408 | C:T | rs9883968 | 122641358 | 0.100 | G:A |
| rs2127024 | 122610787 | 0.250 | G:T | rs4676677 | 122643059 | 0.350 | A:G |
| rs7627978 | 122613827 | 0.250 | T:A | rs6806191 | 122643568 | 0.258 | T:C |
| rs7650658 | 122614066 | 0.250 | A:G | rs7621376 | 122645479 | 0.408 | A:T |
| rs9849118 | 122614958 | 0.250 | A:T | rs6438630 | 122649466 | 0.350 | C:A |
| rs13081748 | 122615053 | 0.408 | G:A | rs11709726 | 122650322 | 0.408 | A:G |
| rs9829579 | 122615090 | 0.167 | G:A | rs3218650 | 122651034 | 0.050 | G:A |
| rs7609781 | 122615507 | 0.250 | C:A | rs650469 | 122660437 | 0.100 | A:G |
| rs13086811 | 122615596 | 0.408 | G:A | rs669277 | 122662360 | 0.100 | T:C |
| rs6438626 | 122616238 | 0.408 | A:G | rs6438633 | 122664513 | 0.408 | A:C |
| rs3732404 | 122617681 | 0.250 | G:A | rs6797376 | 122665700 | 0.408 | A:G |
| rs2169300 | 122619313 | 0.400 | G:A | rs7645027 | 122666965 | 0.408 | A:G |
| rs11709797 | 122620353 | 0.133 | G:C | rs532411 | 122669112 | 0.100 | G:A |
| rs6782025 | 122620666 | 0.250 | T:C | rs1381058 | 122669233 | 0.408 | G:A |
| rs3218647 | 122675040 | 0.058 | G:A | rs4676727 | 122715191 | 0.125 | G:A |
| rs7615529 | 122677016 | 0.408 | C:G | rs12488196 | 122719825 | 0.350 | C:G |
| rs3772122 | 122679758 | 0.408 | C:T | rs13059229 | 122723748 | 0.408 | G:C |
| rs3821367 | 122685766 | 0.317 | A:G | rs494140 | 122726189 | 0.100 | G:A |
| rs3218642 | 122690327 | 0.017 | G:T | rs9863608 | 122727046 | 0.158 | G:A |
| rs3218651 | 122690866 | 0.142 | T:C | rs502643 | 122727731 | 0.250 | A:G |
| rs3218649 | 122691523 | 0.408 | C:G | rs11717597 | 122729375 | 0.142 | T:C |
| rs4555467 | 122693312 | 0.408 | G:A | rs7622867 | 122732168 | 0.408 | T:A |
| rs13097659 | 122693459 | 0.408 | C:G | rs697019 | 122733401 | 0.100 | T:C |
| rs11719937 | 122697642 | 0.133 | A:G | rs501099 | 122736588 | 0.100 | T:C |
| rs10934551 | 122704642 | 0.408 | A:G | rs7612018 | 122740670 | 0.408 | T:C |
| rs3911713 | 122706143 | 0.408 | G:A | rs13065220 | 122747733 | 0.350 | T:C |
| rs693403 | 122710082 | 0.250 | T:C | rs3806614 | 122747866 | 0.408 | A:G |
| rs3732406 | 122711523 | 0.408 | C:A | rs587553 | 122748603 | 0.250 | C:T |
| rs487848 | 122711650 | 0.100 | G:A | | | | |

TABLE 24
Anchor Tag
Block 006
Chromosome 3
Rs1435642 (BP 159782923) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs7638518 | 159769444 | 0.192 | A:C | rs7617983 | 159806418 | 0.183 | T:A |
| rs7638606 | 159769541 | 0.192 | A:G | rs2731114 | 159808096 | 0.133 | T:G |
| rs16829116 | 159770503 | 0.192 | T:C | rs16829135 | 159808540 | 0.192 | A:G |
| rs1714515 | 159770620 | 0.133 | G:A | rs6792976 | 159809272 | 0.192 | A:G |
| rs1656379 | 159771475 | 0.133 | A:G | rs13315529 | 159810976 | 0.192 | G:A |
| rs16829118 | 159771524 | 0.192 | C:A | rs2573182 | 159811528 | 0.133 | G:T |
| rs3817373 | 159771737 | 0.192 | T:A | rs3851362 | 159811671 | 0.392 | T:A |
| rs2731112 | 159772930 | 0.283 | G:C | rs6803047 | 159812231 | 0.392 | G:A |
| rs17629390 | 159773309 | 0.417 | G:T | rs7612250 | 159813838 | 0.417 | G:T |
| rs4368546 | 159774852 | 0.192 | G:A | rs10154981 | 159814962 | 0.192 | T:C |
| rs7631310 | 159779618 | 0.417 | T:C | rs6774895 | 159816584 | 0.133 | T:G |
| rs6441208 | 159782257 | 0.208 | T:A | rs6788046 | 159816956 | 0.192 | T:C |
| rs1435642 | 159782923 | 0.283 | A:G | rs6799605 | 159817042 | 0.192 | A:G |
| rs10513527 | 159784384 | 0.192 | C:A | rs7624771 | 159818111 | 0.233 | C:T |
| rs2082838 | 159788596 | 0.192 | G:A | rs9857678 | 159820267 | 0.417 | T:C |
| rs2566339 | 159791577 | 0.133 | C:T | rs12636691 | 159821343 | 0.392 | C:T |
| rs3765025 | 159793548 | 0.417 | G:A | rs1809752 | 159822568 | 0.133 | A:T |
| rs1435644 | 159794856 | 0.133 | G:C | rs6784320 | 159824661 | 0.133 | A:G |
| rs16829122 | 159795369 | 0.183 | T:C | rs2566334 | 159825206 | 0.258 | G:A |
| rs1435643 | 159795705 | 0.392 | C:T | rs2566333 | 159825536 | 0.133 | G:A |
| rs13318109 | 159795908 | 0.192 | A:G | rs2566332 | 159825630 | 0.258 | C:T |
| rs6804230 | 159796890 | 0.208 | T:C | rs1845138 | 159826609 | 0.417 | C:T |
| rs12107742 | 159799770 | 0.192 | C:T | rs6801874 | 159830098 | 0.183 | C:G |
| rs2639656 | 159800717 | 0.133 | T:C | rs6768636 | 159830267 | 0.183 | T:C |
| rs4875 | 159803299 | 0.392 | C:T | rs4680449 | 159832820 | 0.392 | C:T |
| rs15967 | 159803405 | 0.417 | C:A | rs6441213 | 159841368 | 0.183 | C:G |
| rs2304408 | 159803582 | 0.233 | G:C | rs2082159 | 159842369 | 0.183 | C:T |
| rs2566343 | 159803677 | 0.133 | T:C | rs6776901 | 159843294 | 0.233 | T:C |
| rs4679831 | 159803690 | 0.183 | C:T | rs6777231 | 159843646 | 0.233 | T:C |
| rs12629635 | 159804771 | 0.183 | C:T | rs3806642 | 159844740 | 0.192 | G:T |
| rs6800914 | 159804876 | 0.400 | A:G | rs1864505 | 159845290 | 0.425 | C:G |
| rs2731113 | 159805124 | 0.033 | G:A | rs2303910 | 159847011 | 0.142 | C:T |
| rs6804259 | 159805914 | 0.400 | A:G | rs966146 | 159847935 | 0.425 | A:T |
| rs7628293 | 159806367 | 0.417 | A:G | rs2303909 | 159849602 | 0.392 | A:G |
| rs12633674 | 159850919 | 0.183 | G:T | rs6441219 | 159876424 | 0.425 | T:G |
| rs12639052 | 159851226 | 0.392 | C:A | rs9842500 | 159880982 | 0.425 | G:T |
| rs2060017 | 159851862 | 0.425 | T:C | rs9873186 | 159883201 | 0.425 | T:C |
| rs9867957 | 159852154 | 0.425 | T:C | rs17630980 | 159884256 | 0.208 | A:T |
| rs9884039 | 159855204 | 0.425 | T:C | rs10936158 | 159886969 | 0.392 | T:C |
| rs6763018 | 159855632 | 0.425 | A:G | rs12638363 | 159887460 | 0.192 | G:C |
| rs9813980 | 159856161 | 0.425 | T:C | rs17631078 | 159888407 | 0.192 | G:A |
| rs9819567 | 159857411 | 0.392 | C:T | rs17698494 | 159888575 | 0.233 | G:A |
| rs17697458 | 159859056 | 0.233 | C:G | rs13324770 | 159888601 | 0.392 | G:A |
| rs7648050 | 159860172 | 0.183 | G:A | rs12496174 | 159889617 | 0.392 | G:A |
| rs6779780 | 159860193 | 0.425 | A:G | rs1047355 | 159891964 | 0.425 | C:T |
| rs4680454 | 159860615 | 0.183 | C:T | rs1047366 | 159892184 | 0.392 | T:C |
| rs2291594 | 159861538 | 0.192 | A:G | rs17642107 | 159894383 | 0.425 | C:T |
| rs17630223 | 159861874 | 0.192 | G:C | rs17698754 | 159894406 | 0.233 | G:A |
| rs9873667 | 159862351 | 0.425 | G:A | rs10276 | 159897868 | 0.183 | C:T |
| rs891464 | 159864795 | 0.392 | A:G | rs1051109 | 159898046 | 0.183 | T:C |
| rs9814061 | 159865224 | 0.425 | G:A | rs3867391 | 159898542 | 0.233 | T:G |
| rs16829273 | 159865322 | 0.192 | A:G | rs6772483 | 159898701 | 0.392 | G:A |
| rs17630394 | 159868498 | 0.175 | G:C | rs16829314 | 159899111 | 0.183 | G:A |
| rs1492908 | 159869209 | 0.425 | A:G | rs7621897 | 159899348 | 0.392 | C:A |
| rs9844619 | 159869291 | 0.075 | A:G | rs9822456 | 159899675 | 0.017 | T:A |
| rs6441215 | 159870189 | 0.425 | T:C | rs6768290 | 159900959 | 0.425 | T:C |
| rs8455 | 159871482 | 0.417 | C:T | rs17699103 | 159902491 | 0.233 | A:G |
| rs16829283 | 159871830 | 0.192 | G:A | rs7610009 | 159903071 | 0.233 | C:A |
| rs7624934 | 159872474 | 0.425 | C:T | rs7640040 | 159903178 | 0.425 | G:A |
| rs17630607 | 159872562 | 0.233 | C:T | rs6763232 | 159904351 | 0.425 | T:C |
| rs9832803 | 159873826 | 0.425 | C:G | rs6763412 | 159904511 | 0.425 | T:G |
| rs9833067 | 159873840 | 0.425 | G:T | rs12488447 | 159904526 | 0.183 | T:C |
| rs6767792 | 159874547 | 0.425 | G:A | rs16847130 | 159906276 | 0.192 | T:A |
| rs17630785 | 159874928 | 0.192 | C:T | rs16847135 | 159906555 | 0.192 | T:C |

TABLE 25
Anchor Tag
Block 007
Chromosome 3
Rs6444501 (BP 192302944) LD block SNPs

| | | Minor | |
|---|---|---|---|
| | Base | Allele | |
| SNP ID (rs) | Position | Freq | Alleles |
| rs13079057 | 192287260 | 0.125 | C:T |
| rs7634909 | 192289418 | 0.133 | A:G |
| rs7613062 | 192289470 | 0.125 | C:T |
| rs13077441 | 192289978 | 0.125 | A:G |
| rs9290973 | 192293952 | 0.125 | T:C |
| rs9814786 | 192294992 | 0.408 | G:A |
| rs6799860 | 192297907 | 0.125 | A:G |
| rs10513863 | 192300602 | 0.242 | G:A |
| rs1195356 | 192301158 | 0.375 | C:T |
| rs1195357 | 192301264 | 0.183 | T:C |
| rs1195358 | 192301796 | 0.183 | A:T |
| rs6444501 | 192302944 | 0.367 | G:T |
| rs1195359 | 192303107 | 0.192 | G:A |
| rs7624125 | 192303960 | 0.158 | C:G |
| rs1195361 | 192304009 | 0.375 | T:C |
| rs13093019 | 192304753 | 0.125 | A:G |
| rs1201618 | 192306000 | 0.375 | A:T |
| rs1195362 | 192306503 | 0.033 | A:G |
| rs6791655 | 192307502 | 0.125 | G:A |
| rs7372356 | 192308267 | 0.433 | A:C |
| rs13085049 | 192308777 | 0.158 | A:C |
| rs2666367 | 192309681 | 0.192 | C:T |
| rs9837642 | 192309736 | 0.125 | G:A |
| rs9844117 | 192311332 | 0.158 | G:A |

TABLE 26
Anchor Tag
Block 001
Chromosome 4
Rs6856651 (BP 73261492) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs4694443 | 73169513 | 0.442 | T:A | rs11936367 | 73250013 | 0.358 | A:G |
| rs2139639 | 73171986 | 0.383 | A:G | rs7699080 | 73250934 | 0.392 | C:T |
| rs6823008 | 73172519 | 0.383 | A:G | rs10028416 | 73256502 | 0.367 | A:G |
| rs7673356 | 73173074 | 0.383 | T:C | rs6810525 | 73258168 | 0.008 | C:T |
| rs2365530 | 73173299 | 0.383 | T:C | rs7699403 | 73258359 | 0.367 | G:C |
| rs10937987 | 73174035 | 0.383 | A:G | rs6856651 | 73261492 | 0.367 | A:T |
| rs10518096 | 73174597 | 0.383 | C:T | rs7654531 | 73265656 | 0.367 | C:T |
| rs1520502 | 73175834 | 0.383 | T:C | rs6824342 | 73268217 | 0.375 | T:C |
| rs6446772 | 73176558 | 0.383 | T:C | rs6824703 | 73268353 | 0.375 | T:C |
| rs1356878 | 73177269 | 0.383 | C:T | rs12503480 | 73268827 | 0.367 | C:T |
| rs1520501 | 73178193 | 0.383 | C:A | rs12512773 | 73270124 | 0.375 | G:A |
| rs11726886 | 73187634 | 0.300 | C:A | rs13107758 | 73271841 | 0.017 | G:C |
| rs1949573 | 73188475 | 0.383 | G:A | rs7695255 | 73273144 | 0.017 | G:A |
| rs12503466 | 73189093 | 0.383 | G:A | rs7670209 | 73273242 | 0.017 | C:G |
| rs17712557 | 73190080 | 0.242 | A:T | rs7670216 | 73273253 | 0.017 | C:G |
| rs1402162 | 73191117 | 0.383 | G:A | rs13121468 | 73273465 | 0.017 | T:C |
| rs6446775 | 73192247 | 0.383 | G:A | rs13122374 | 73273827 | 0.017 | T:C |
| rs1915188 | 73199267 | 0.367 | C:T | rs7679137 | 73274771 | 0.017 | T:C |
| rs11733593 | 73199348 | 0.383 | C:T | rs6841203 | 73275422 | 0.017 | A:G |
| rs10518094 | 73199925 | 0.383 | G:C | rs6843941 | 73275979 | 0.017 | G:C |
| rs10518093 | 73200067 | 0.317 | A:T | rs10937991 | 73276159 | 0.017 | A:C |
| rs950110 | 73200567 | 0.383 | T:C | rs13435787 | 73276333 | 0.358 | C:A |
| rs1520503 | 73200988 | 0.383 | G:A | rs11727771 | 73276518 | 0.375 | A:G |
| rs2365538 | 73204628 | 0.383 | C:G | rs9637554 | 73278192 | 0.017 | C:G |
| rs12504627 | 73214243 | 0.350 | G:T | rs4533728 | 73278715 | 0.017 | G:C |
| rs7666924 | 73217392 | 0.350 | T:C | rs7658272 | 73279116 | 0.017 | C:T |
| rs12643653 | 73217516 | 0.008 | C:T | rs5004264 | 73279292 | 0.017 | G:A |
| rs13103331 | 73218158 | 0.350 | C:T | rs4295230 | 73279440 | 0.017 | C:T |
| rs4694445 | 73220628 | 0.275 | T:A | rs6811187 | 73279780 | 0.017 | G:A |
| rs10032596 | 73224760 | 0.350 | G:A | rs6816572 | 73280107 | 0.017 | G:A |
| rs11722798 | 73226913 | 0.350 | A:G | rs11722579 | 73280602 | 0.017 | G:A |
| rs4263362 | 73234038 | 0.350 | A:C | rs11722657 | 73280755 | 0.017 | T:A |
| rs4694113 | 73234688 | 0.350 | T:A | rs7661544 | 73280969 | 0.017 | G:A |
| rs11723796 | 73240632 | 0.350 | C:T | rs9994192 | 73281018 | 0.017 | A:G |
| rs7687603 | 73281031 | 0.358 | G:T | rs6446807 | 73286948 | 0.017 | T:A |
| rs7688022 | 73281307 | 0.017 | A:G | rs6446808 | 73287051 | 0.017 | G:A |
| rs7439017 | 73283378 | 0.017 | G:A | rs7435387 | 73287354 | 0.017 | G:A |
| rs6844604 | 73284354 | 0.017 | G:A | rs4398493 | 73287640 | 0.017 | G:A |
| rs4560375 | 73285435 | 0.017 | A:G | rs4566627 | 73287683 | 0.017 | T:G |
| rs10012432 | 73285866 | 0.017 | G:T | rs11726556 | 73287710 | 0.383 | T:C |
| rs7675387 | 73286753 | 0.300 | G:T | | | | |

TABLE 27
Anchor Tag
Block 002
Chromosome 4
Rs3733558 (BP 122315696), SNP_A-2218634 (BP 122317455),
Rs2175397 (BP 122320749), and Rs6832987 (BP 122321077)
LD block SNPs

| | | Minor | |
|---|---|---|---|
| | Base | Allele | |
| SNP ID (rs) | Position | Freq | Alleles |
| rs6840113 | 122294112 | 0.458 | G:A |
| rs4323133 | 122294400 | 0.442 | G:A |
| rs13108974 | 122294511 | 0.175 | A:G |
| rs4532258 | 122294684 | 0.208 | T:C |
| rs1807071 | 122295901 | 0.208 | A:C |
| rs4337753 | 122297230 | 0.208 | G:A |
| rs6534243 | 122297248 | 0.050 | C:T |
| rs6818106 | 122297591 | 0.050 | A:G |
| rs7440273 | 122298078 | 0.175 | T:C |
| rs4524412 | 122298504 | 0.208 | C:T |
| rs11931955 | 122298603 | 0.175 | T:C |
| rs11945593 | 122298885 | 0.175 | G:A |
| rs11721617 | 122301133 | 0.042 | G:A |
| rs12509928 | 122302537 | 0.258 | T:C |
| rs6848952 | 122303691 | 0.258 | C:T |
| rs11946567 | 122305140 | 0.175 | G:A |
| rs1135508 | 122308293 | 0.208 | T:C |
| rs12501078 | 122308728 | 0.458 | G:C |
| rs1329 | 122310385 | 0.433 | C:T |
| rs4604073 | 122311038 | 0.208 | C:G |
| rs1397645 | 122315339 | 0.175 | C:T |
| rs3733558 | 122315696 | 0.433 | G:T |
| rs11939546 | 122319347 | 0.050 | C:T |
| rs7690446 | 122319833 | 0.175 | G:C |
| rs2175397 | 122320749 | 0.433 | A:G |
| rs2136949 | 122320836 | 0.175 | T:C |
| rs6832987 | 122321077 | 0.433 | A:T |
| rs1355395 | 122321180 | 0.225 | A:T |
| rs6832603 | 122322776 | 0.433 | T:C |
| rs17051283 | 122322874 | 0.050 | C:T |
| rs2276959 | 122324412 | 0.225 | T:C |
| rs6534244 | 122324928 | 0.433 | A:G |
| rs6534245 | 122325242 | 0.225 | G:C |
| rs6837474 | 122326914 | 0.433 | C:T |
| rs4446338 | 122326969 | 0.225 | C:A |
| rs1912680 | 122328106 | 0.225 | C:G |
| rs11098598 | 122329267 | 0.458 | C:A |
| rs1397646 | 122329389 | 0.175 | G:A |
| rs11098599 | 122329660 | 0.433 | G:A |
| rs17051284 | 122329907 | 0.233 | T:C |
| rs11098600 | 122329957 | 0.442 | A:C |
| rs6833075 | 122330108 | 0.233 | A:G |
| rs13103315 | 122331407 | 0.458 | A:G |
| rs4833704 | 122331469 | 0.208 | T:C |

TABLE 28
Anchor Tag
Block 003
Chromosome 4
Rs17621300 (BP 165856946) and Rs12641382 (BP 165868089)
LD block SNPs

| | | Minor | |
|---|---|---|---|
| | Base | Allele | |
| SNP ID (rs) | Position | Freq | Alleles |
| rs1126336 | 165854326 | 0.075 | G:A |
| rs4691996 | 165854471 | 0.075 | C:T |
| rs10517808 | 165854578 | 0.058 | G:A |
| rs4145327 | 165854988 | 0.017 | G:A |
| rs4691997 | 165855272 | 0.017 | A:G |
| rs17045152 | 165855807 | 0.050 | C:T |
| rs16999502 | 165856320 | 0.017 | A:T |
| rs17677822 | 165856558 | 0.067 | C:T |
| rs10517809 | 165856883 | 0.017 | T:C |
| rs17621300 | 165856946 | 0.067 | A:C |
| rs7669898 | 165857281 | 0.017 | T:C |
| rs7695224 | 165857649 | 0.017 | G:T |
| rs17045154 | 165858394 | 0.058 | C:T |
| rs11731849 | 165860611 | 0.017 | A:C |
| rs2141423 | 165863836 | 0.083 | T:C |
| rs12641382 | 165868089 | 0.075 | A:G |
| rs1980394 | 165870656 | 0.067 | G:C |
| rs1534577 | 165871603 | 0.067 | G:T |
| rs13114228 | 165873008 | 0.075 | A:G |
| rs13114410 | 165873051 | 0.075 | A:G |
| rs13114673 | 165873209 | 0.083 | T:G |

TABLE 29
Anchor Tag
Block 001
Chromosome 5
Rs1863984 (BP 4264882) and Rs1035866 (BP 4269069)
LD block SNPs

| | | Minor | |
|---|---|---|---|
| | Base | Allele | |
| SNP ID (rs) | Position | Freq | Alleles |
| rs7734785 | 4257909 | 0.383 | A:C |
| rs7734004 | 4258074 | 0.392 | T:G |
| rs13359718 | 4261475 | 0.017 | T:A |
| rs13359738 | 4261572 | 0.017 | T:C |
| rs16872922 | 4262095 | 0.017 | T:C |
| rs890784 | 4262189 | 0.017 | C:T |
| rs16872924 | 4262492 | 0.017 | A:G |
| rs16872926 | 4262763 | 0.017 | T:C |
| rs16872927 | 4263030 | 0.017 | C:G |
| rs16872928 | 4264418 | 0.017 | T:G |
| rs1863984 | 4264882 | 0.067 | G:A |
| rs7711612 | 4266021 | 0.017 | C:T |
| rs7715735 | 4266360 | 0.017 | C:T |
| rs7735751 | 4266582 | 0.017 | A:G |
| rs16872940 | 4266646 | 0.017 | C:T |
| rs919256 | 4266940 | 0.017 | C:G |
| rs2081860 | 4267217 | 0.017 | G:T |
| rs16872946 | 4267540 | 0.017 | C:T |
| rs16872947 | 4267947 | 0.017 | T:C |
| rs17722784 | 4268700 | 0.017 | T:C |
| rs4479812 | 4269057 | 0.017 | G:T |
| rs1035866 | 4269069 | 0.150 | G:A |

TABLE 30
Anchor Tag
Block 002
Chromosome 5
Rs6859601 (BP 11200414), Rs12516262 (BP 11200575), Rs1859382
(BP 11200672), and Rs886527 (BP 11200814) LD block SNPs

| | | Minor | |
|---|---|---|---|
| | Base | Allele | |
| SNP ID (rs) | Position | Freq | Alleles |
| rs1547940 | 11180359 | 0.117 | T:C |
| rs11743332 | 11181377 | 0.092 | G:A |
| rs17788598 | 11183879 | 0.092 | C:A |
| rs6864900 | 11184057 | 0.017 | G:C |
| rs6865035 | 11184114 | 0.017 | G:A |
| rs2158444 | 11185342 | 0.383 | T:G |
| rs6876115 | 11186153 | 0.383 | G:C |
| rs6881497 | 11186905 | 0.292 | G:C |
| rs10513073 | 11187772 | 0.292 | T:C |
| rs13165911 | 11188377 | 0.092 | T:G |
| rs6892380 | 11188615 | 0.292 | T:C |
| rs7732720 | 11189164 | 0.083 | G:A |
| rs7714658 | 11189251 | 0.292 | C:T |
| rs2214599 | 11189893 | 0.400 | T:C |
| rs6873490 | 11190438 | 0.292 | A:G |
| rs6873671 | 11190695 | 0.292 | G:C |
| rs10513074 | 11190855 | 0.092 | T:C |
| rs6874039 | 11190921 | 0.292 | T:C |
| rs10513075 | 11191759 | 0.025 | A:C |
| rs730610 | 11192051 | 0.292 | T:A |
| rs13188516 | 11195037 | 0.092 | C:T |
| rs13176246 | 11196181 | 0.092 | T:C |
| rs2158445 | 11198076 | 0.267 | A:T |
| rs10073056 | 11199253 | 0.258 | C:A |
| rs10059890 | 11199698 | 0.275 | T:C |
| rs10041627 | 11200082 | 0.258 | G:T |
| rs6885587 | 11200114 | 0.258 | C:G |
| rs6880938 | 11200302 | 0.258 | C:T |
| rs6859601 | 11200414 | 0.275 | T:C |
| rs12516262 | 11200575 | 0.358 | G:T |
| rs1859382 | 11200672 | 0.275 | C:G |
| rs886527 | 11200814 | 0.275 | A:G |
| rs11948241 | 11201013 | 0.008 | C:T |
| rs886526 | 11201027 | 0.275 | C:A |
| rs886525 | 11201111 | 0.358 | A:C |
| rs2400029 | 11201257 | 0.358 | T:C |
| rs12514212 | 11201537 | 0.442 | T:C |
| rs757459 | 11201693 | 0.425 | C:T |
| rs757458 | 11201837 | 0.425 | C:A |
| rs10036188 | 11202203 | 0.467 | G:A |
| rs10055024 | 11202808 | 0.467 | C:T |
| rs10059397 | 11204919 | 0.375 | G:C |
| rs11953748 | 11206927 | 0.008 | G:A |
| rs13358276 | 11207447 | 0.442 | T:C |
| rs2057795 | 11207597 | 0.275 | C:T |
| rs13158585 | 11208378 | 0.083 | G:A |

TABLE 31
Anchor Tag
Block 003
Chromosome 5
Rs31923 (BP 14724030) and Rs25986 (BP 14771036) LD block SNPs

| | | Minor | |
|---|---|---|---|
| | Base | Allele | |
| SNP ID (rs) | Position | Freq | Alleles |
| rs1875513 | 14710062 | 0.250 | A:G |
| rs7380063 | 14714675 | 0.200 | T:C |
| rs250878 | 14716362 | 0.042 | T:C |
| rs2455458 | 14718615 | 0.467 | G:A |
| rs17297276 | 14720143 | 0.283 | C:T |
| rs31920 | 14720484 | 0.050 | T:A |
| rs17363158 | 14721165 | 0.100 | G:A |
| rs31922 | 14721915 | 0.192 | G:T |
| rs6884420 | 14723698 | 0.100 | T:C |
| rs31923 | 14724030 | 0.050 | T:A |
| rs16903631 | 14726696 | 0.083 | C:T |
| rs31925 | 14727999 | 0.192 | G:A |
| rs31926 | 14728600 | 0.050 | A:T |
| rs6892588 | 14729218 | 0.100 | C:T |
| rs992737 | 14730305 | 0.217 | A:G |
| rs31928 | 14730638 | 0.050 | G:A |
| rs42358 | 14730868 | 0.050 | C:G |
| rs31930 | 14731996 | 0.050 | A:G |
| rs25941 | 14733451 | 0.042 | C:T |
| rs28004 | 14734227 | 0.050 | A:G |
| rs250440 | 14738548 | 0.192 | G:A |
| rs258374 | 14740362 | 0.192 | G:C |
| rs250438 | 14741417 | 0.100 | G:A |
| rs152788 | 14742707 | 0.192 | A:G |
| rs31919 | 14744586 | 0.042 | T:C |
| rs40578 | 14745579 | 0.050 | G:T |
| rs17363829 | 14746398 | 0.092 | C:T |
| rs25951 | 14746480 | 0.050 | A:G |
| rs25952 | 14748247 | 0.050 | C:A |
| rs6886163 | 14750849 | 0.075 | A:G |
| rs39609 | 14750914 | 0.042 | C:T |
| rs31995 | 14751233 | 0.042 | T:C |
| rs31994 | 14752582 | 0.025 | C:T |
| rs10065032 | 14753035 | 0.100 | G:A |
| rs149617 | 14753853 | 0.192 | T:A |
| rs258290 | 14755375 | 0.192 | A:G |
| rs27911 | 14760378 | 0.050 | A:G |
| rs25992 | 14760600 | 0.050 | T:C |
| rs17364066 | 14763796 | 0.100 | G:A |
| rs3045 | 14764275 | 0.100 | T:C |
| rs25988 | 14765514 | 0.050 | A:G |
| rs25987 | 14765709 | 0.042 | T:A |
| rs10491477 | 14768140 | 0.042 | A:G |
| rs25986 | 14771036 | 0.042 | T:G |
| rs27356 | 14775441 | 0.175 | T:C |
| rs10060885 | 14777089 | 0.025 | T:C |
| rs25983 | 14779958 | 0.183 | G:C |
| rs13189930 | 14780882 | 0.183 | C:A |

TABLE 32
Anchor Tag
Block 004
Chromosome 5
Rs401531 (BP 142249012), Rs712161 (BP 142271912),
Rs252234 (BP 142309329), and
Rs10875598 (BP 142371351) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs707177 | 142232076 | 0.333 | G:A | rs728465 | 142364353 | 0.225 | T:C |
| rs447923 | 142232441 | 0.317 | C:T | rs728463 | 142364563 | 0.242 | T:G |
| rs10039856 | 142232733 | 0.058 | C:T | rs4912888 | 142367296 | 0.225 | A:G |
| rs26707 | 142233857 | 0.267 | C:G | rs3822398 | 142368154 | 0.225 | T:C |
| rs27958 | 142233870 | 0.267 | G:A | rs11167798 | 142368418 | 0.258 | G:A |
| rs185200 | 142234863 | 0.283 | A:G | rs245850 | 142370766 | 0.017 | C:T |
| rs26706 | 142237044 | 0.275 | G:C | rs10875598 | 142371351 | 0.225 | T:C |
| rs17208943 | 142237302 | 0.025 | T:C | rs3822397 | 142373755 | 0.225 | A:G |
| rs187552 | 142239766 | 0.217 | A:G | rs245852 | 142374573 | 0.208 | A:G |
| rs17208950 | 142242909 | 0.025 | G:A | rs245853 | 142375143 | 0.025 | C:T |
| rs17099647 | 142243163 | 0.017 | A:G | rs7723504 | 142375209 | 0.033 | T:C |
| rs17099651 | 142243319 | 0.017 | G:T | rs245813 | 142377218 | 0.208 | A:T |
| rs12658615 | 142243357 | 0.017 | C:T | rs28304 | 142262043 | 0.292 | T:C |
| rs13176749 | 142243632 | 0.017 | G:A | rs26692 | 142262752 | 0.017 | C:A |
| rs6580260 | 142244590 | 0.017 | G:T | rs35292 | 142263239 | 0.033 | A:G |
| rs26696 | 142244786 | 0.242 | A:T | rs27899 | 142263779 | 0.258 | G:C |
| rs6886351 | 142245753 | 0.017 | A:G | rs26691 | 142263926 | 0.283 | G:A |
| rs27546 | 142245929 | 0.267 | A:T | rs434456 | 142265935 | 0.242 | G:A |
| rs412738 | 142246597 | 0.283 | G:A | rs415235 | 142266140 | 0.258 | T:C |
| rs401531 | 142249012 | 0.242 | G:T | rs3776409 | 142267058 | 0.017 | G:A |
| rs389008 | 142249384 | 0.292 | G:C | rs364211 | 142268502 | 0.225 | A:T |
| rs23282 | 142250485 | 0.258 | G:A | rs7705560 | 142268711 | 0.258 | G:A |
| rs2304041 | 142254341 | 0.017 | A:G | rs13187932 | 142268729 | 0.017 | G:A |
| rs37215 | 142254642 | 0.017 | T:A | rs377269 | 142268968 | 0.258 | G:A |
| rs3850572 | 142257207 | 0.017 | G:T | rs409509 | 142269057 | 0.242 | A:G |
| rs153169 | 142257316 | 0.242 | G:T | rs830532 | 142269725 | 0.283 | C:T |
| rs153170 | 142257435 | 0.283 | G:A | rs17099693 | 142270946 | 0.017 | A:G |
| rs17208964 | 142258147 | 0.025 | A:C | rs712161 | 142271912 | 0.258 | A:C |
| rs153172 | 142258202 | 0.258 | C:A | rs7723139 | 142272054 | 0.017 | C:G |
| rs37214 | 142258820 | 0.225 | C:T | rs153167 | 142273386 | 0.300 | G:C |
| rs10056531 | 142259278 | 0.017 | C:T | rs3776408 | 142273913 | 0.017 | A:G |
| rs28052 | 142260054 | 0.258 | C:G | rs17099698 | 142273951 | 0.025 | G:C |
| rs26693 | 142260450 | 0.242 | G:A | rs17208999 | 142275678 | 0.025 | C:T |
| rs17287483 | 142260851 | 0.025 | G:A | rs37192 | 142278327 | 0.225 | G:A |
| rs712169 | 142289330 | 0.283 | T:C | rs712165 | 142278349 | 0.300 | T:C |
| rs712172 | 142290533 | 0.283 | A:G | rs712166 | 142278537 | 0.300 | A:G |
| rs712175 | 142290681 | 0.283 | C:T | rs37191 | 142280677 | 0.033 | C:T |
| rs37185 | 142291083 | 0.250 | C:T | rs153165 | 142281849 | 0.300 | G:A |
| rs712176 | 142291626 | 0.283 | A:G | rs3797081 | 142282544 | 0.017 | A:G |
| rs712177 | 142291693 | 0.283 | T:C | rs153166 | 142282801 | 0.300 | G:A |
| rs37184 | 142292154 | 0.225 | C:T | rs37189 | 142284369 | 0.033 | C:T |
| rs245711 | 142293305 | 0.225 | T:A | rs698287 | 142285615 | 0.283 | G:A |
| rs42466 | 142294414 | 0.225 | G:A | rs712167 | 142285858 | 0.283 | G:C |
| rs37224 | 142295455 | 0.225 | A:G | rs10062088 | 142287997 | 0.292 | T:A |
| rs830301 | 142295707 | 0.258 | T:C | rs712168 | 142288120 | 0.283 | C:T |
| rs42465 | 142296801 | 0.033 | G:C | rs37187 | 142288374 | 0.217 | C:G |
| rs153164 | 142297112 | 0.283 | T:C | rs712182 | 142315778 | 0.308 | C:T |
| rs37222 | 142297839 | 0.250 | A:T | rs40360 | 142315793 | 0.033 | G:A |
| rs13188405 | 142298359 | 0.025 | C:T | rs37210 | 142316372 | 0.225 | A:G |
| rs37221 | 142298961 | 0.225 | G:C | rs712184 | 142316658 | 0.283 | A:G |
| rs712178 | 142299485 | 0.283 | T:C | rs37209 | 142318497 | 0.058 | T:G |
| rs245779 | 142300534 | 0.308 | T:A | rs37208 | 142318517 | 0.033 | C:G |
| rs26697 | 142304596 | 0.283 | G:T | rs712186 | 142319854 | 0.283 | G:A |
| rs17287545 | 142304735 | 0.017 | G:A | rs37207 | 142320378 | 0.033 | T:C |
| rs26698 | 142305738 | 0.283 | T:C | rs37206 | 142320959 | 0.225 | G:T |
| rs28053 | 142306423 | 0.033 | G:T | rs17287559 | 142321363 | 0.025 | A:G |
| rs11954679 | 142306447 | 0.017 | G:A | rs37205 | 142322309 | 0.225 | A:G |
| rs252232 | 142308871 | 0.283 | G:A | rs252223 | 142324054 | 0.283 | A:C |
| rs252233 | 142308914 | 0.283 | C:G | rs37203 | 142324484 | 0.033 | C:T |
| rs181865 | 142309150 | 0.283 | C:T | rs173750 | 142324494 | 0.283 | T:C |
| rs252234 | 142309329 | 0.258 | A:G | rs698288 | 142325638 | 0.283 | T:C |
| rs712180 | 142310091 | 0.283 | T:C | rs712187 | 142325711 | 0.283 | C:T |
| rs245780 | 142311697 | 0.383 | C:T | rs830286 | 142327078 | 0.283 | A:G |
| rs42464 | 142311818 | 0.225 | G:A | rs35291 | 142329249 | 0.283 | T:C |
| rs37213 | 142313059 | 0.225 | A:C | rs37200 | 142330860 | 0.033 | T:C |
| rs37212 | 142313187 | 0.225 | T:C | rs43158 | 142331258 | 0.033 | C:T |
| rs37211 | 142315350 | 0.225 | G:T | rs1837261 | 142332249 | 0.025 | A:G |
| rs712181 | 142315519 | 0.283 | T:C | rs17096377 | 142333053 | 0.025 | T:C |
| rs10054462 | 142344701 | 0.017 | A:G | rs37199 | 142333281 | 0.058 | A:C |
| rs1197509 | 142350612 | 0.250 | C:G | rs245854 | 142334640 | 0.017 | T:G |
| rs11167795 | 142351660 | 0.258 | T:G | rs17099768 | 142335655 | 0.017 | G:T |
| rs6888562 | 142351711 | 0.017 | A:C | rs37196 | 142337161 | 0.033 | C:T |
| rs13172733 | 142351873 | 0.258 | G:A | rs35295 | 142337300 | 0.242 | T:G |
| rs7712624 | 142352603 | 0.017 | G:A | rs37195 | 142337942 | 0.017 | G:A |
| rs6898510 | 142352628 | 0.017 | T:C | rs37194 | 142338086 | 0.033 | G:T |
| rs151973 | 142353733 | 0.225 | T:A | rs187396 | 142338113 | 0.008 | A:T |
| rs245713 | 142357605 | 0.017 | G:A | rs37193 | 142338930 | 0.025 | C:T |
| rs245712 | 142357709 | 0.017 | A:G | rs35294 | 142339220 | 0.258 | A:C |
| rs17099782 | 142358157 | 0.017 | T:C | rs3776372 | 142342212 | 0.017 | G:A |
| rs6877111 | 142358443 | 0.017 | C:T | rs245729 | 142343370 | 0.033 | T:C |
| rs13161500 | 142359054 | 0.017 | G:A | rs245814 | 142377649 | 0.025 | C:A |
| rs17099783 | 142359281 | 0.017 | A:G | rs10056505 | 142377880 | 0.017 | G:A |
| rs6580264 | 142359528 | 0.017 | A:G | rs245815 | 142378134 | 0.025 | G:T |
| rs7721905 | 142359819 | 0.225 | A:G | rs9324900 | 142378530 | 0.017 | C:A |
| rs168667 | 142360708 | 0.017 | C:T | rs245816 | 142378638 | 0.208 | T:C |
| rs1370982 | 142361277 | 0.275 | A:G | rs3776362 | 142379050 | 0.175 | A:C |
| rs17099784 | 142361962 | 0.017 | C:T | rs1347813 | 142379487 | 0.233 | C:T |
| rs3776370 | 142363196 | 0.017 | C:T | rs7721749 | 142380312 | 0.017 | A:G |
| rs3776369 | 142363302 | 0.242 | G:A | rs245818 | 142381140 | 0.208 | A:G |

TABLE 33
Anchor Tag
Block 005
Chromosome 5
Rs353249 (BP 148804949) LD block SNPs

| | | Minor | |
|---|---|---|---|
| | Base | Allele | |
| SNP ID (rs) | Position | Freq | Alleles |
| rs403084 | 148799907 | 0.192 | C:T |
| rs9325142 | 148801090 | 0.100 | G:A |
| rs2454104 | 148802183 | 0.450 | A:G |
| rs4705344 | 148802311 | 0.375 | A:G |
| rs2431723 | 148802697 | 0.442 | T:C |
| rs734510 | 148803200 | 0.458 | T:C |
| rs2254161 | 148803855 | 0.458 | G:T |
| rs353249 | 148804949 | 0.050 | T:C |
| rs353248 | 148805034 | 0.050 | A:G |
| rs10062536 | 148805862 | 0.108 | C:T |
| rs353244 | 148807108 | 0.442 | C:A |
| rs13436354 | 148807272 | 0.108 | A:G |
| rs353243 | 148807387 | 0.042 | A:C |
| rs353242 | 148808047 | 0.475 | C:T |
| rs353241 | 148808671 | 0.150 | C:T |
| rs17653932 | 148811326 | 0.467 | C:T |
| rs11743818 | 148811774 | 0.483 | C:T |

TABLE 34
Anchor Tag
Block 006
Chromosome 5
Rs7712987 (BP 153280199) LD block SNPs

| | | Minor | |
|---|---|---|---|
| | Base | Allele | |
| SNP ID (rs) | Position | Freq | Alleles |
| rs2614124 | 153246504 | 0.467 | C:G |
| rs2614125 | 153247946 | 0.467 | G:A |
| rs2560036 | 153248813 | 0.325 | T:C |
| rs10061978 | 153250036 | 0.192 | G:T |
| rs2614126 | 153250909 | 0.325 | A:G |
| rs919221 | 153251269 | 0.483 | C:T |
| rs919222 | 153251349 | 0.150 | G:A |
| rs919223 | 153251423 | 0.325 | C:T |
| rs1366095 | 153251949 | 0.483 | A:G |
| rs2560037 | 153252166 | 0.483 | A:G |
| rs4958352 | 153252925 | 0.150 | C:T |
| rs6873055 | 153253361 | 0.150 | G:T |
| rs10515702 | 153253787 | 0.150 | C:T |
| rs2434220 | 153257952 | 0.325 | G:A |
| rs2560055 | 153258125 | 0.325 | G:A |
| rs10054136 | 153258278 | 0.050 | A:C |
| rs2964001 | 153259877 | 0.325 | A:C |
| rs1593827 | 153261086 | 0.317 | G:A |
| rs2560030 | 153261599 | 0.333 | C:T |
| rs1461243 | 153263155 | 0.150 | T:C |
| rs1903442 | 153266052 | 0.150 | G:C |
| rs898709 | 153267157 | 0.317 | A:G |
| rs1870738 | 153267742 | 0.317 | T:A |
| rs2614119 | 153269618 | 0.483 | A:G |
| rs1599408 | 153276210 | 0.358 | C:T |
| rs4590221 | 153278407 | 0.183 | C:T |
| rs7712987 | 153280199 | 0.317 | A:T |
| rs2614123 | 153280408 | 0.317 | C:T |
| rs2434221 | 153280765 | 0.325 | G:C |

TABLE 35
Anchor Tag
Block 007
Chromosome 5
Rs29807 (BP 168937127) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs255738 | 168833277 | 0.350 | C:G | rs3892760 | 168946565 | 0.025 | C:T |
| rs185330 | 168834318 | 0.442 | G:C | rs255824 | 168946879 | 0.242 | T:C |
| rs2545384 | 168844883 | 0.450 | T:G | rs2288383 | 168947791 | 0.025 | A:G |
| rs13355764 | 168845424 | 0.450 | T:A | rs17668965 | 168949808 | 0.067 | A:G |
| rs12716247 | 168846290 | 0.450 | G:A | rs2254334 | 168951470 | 0.317 | T:C |
| rs12187314 | 168846694 | 0.450 | T:C | rs10516067 | 168951725 | 0.033 | C:G |
| rs2546707 | 168855250 | 0.392 | G:A | rs4493692 | 168954188 | 0.317 | T:C |
| rs2559978 | 168856007 | 0.442 | T:C | rs17558484 | 168954280 | 0.033 | A:G |
| rs2546705 | 168856444 | 0.442 | C:T | rs7730272 | 168957158 | 0.025 | A:G |
| rs2546704 | 168857051 | 0.450 | A:T | rs29787 | 168885235 | 0.458 | C:T |
| rs2559976 | 168857063 | 0.442 | C:A | rs3849731 | 168888377 | 0.167 | C:T |
| rs2559975 | 168857116 | 0.458 | T:C | rs29821 | 168889633 | 0.458 | T:C |
| rs185334 | 168857177 | 0.442 | C:G | rs29820 | 168890891 | 0.342 | G:A |
| rs255736 | 168857630 | 0.450 | T:A | rs29819 | 168891060 | 0.342 | G:T |
| rs255735 | 168857942 | 0.442 | G:A | rs29818 | 168891869 | 0.342 | T:C |
| rs255733 | 168858930 | 0.458 | G:T | rs17643975 | 168892466 | 0.200 | G:C |
| rs17071326 | 168860022 | 0.008 | C:T | rs185335 | 168893445 | 0.458 | G:A |
| rs29810 | 168862698 | 0.433 | T:C | rs29816 | 168894136 | 0.458 | C:T |
| rs29809 | 168863243 | 0.350 | C:T | rs4507506 | 168895065 | 0.025 | G:A |
| rs2546703 | 168864685 | 0.450 | C:A | rs10516064 | 168895486 | 0.108 | G:C |
| rs17643822 | 168865062 | 0.167 | C:T | rs17644016 | 168895935 | 0.167 | G:A |
| rs10068843 | 168865171 | 0.033 | C:A | rs29815 | 168896494 | 0.342 | T:C |
| rs3849728 | 168865444 | 0.167 | A:G | rs29814 | 168898178 | 0.342 | C:T |
| rs2546702 | 168865520 | 0.458 | T:C | rs187647 | 168898214 | 0.458 | T:G |
| rs2559974 | 168870881 | 0.308 | C:T | rs29812 | 168899670 | 0.458 | G:C |
| rs2546700 | 168875805 | 0.317 | T:C | rs29811 | 168900357 | 0.100 | G:A |
| rs1833732 | 168876075 | 0.317 | C:A | rs255829 | 168900814 | 0.458 | C:T |
| rs255834 | 168879053 | 0.342 | C:G | rs255827 | 168903083 | 0.342 | C:A |
| rs255833 | 168879441 | 0.458 | T:C | rs39798 | 168904091 | 0.342 | A:G |
| rs12654922 | 168881456 | 0.200 | G:A | rs187646 | 168905820 | 0.375 | T:C |
| rs17071343 | 168882123 | 0.033 | G:A | rs255816 | 168907125 | 0.350 | T:G |
| rs17643909 | 168882152 | 0.033 | T:G | rs17071366 | 168907410 | 0.033 | A:G |
| rs29790 | 168883917 | 0.458 | T:C | rs3893837 | 168907770 | 0.167 | A:G |
| rs29788 | 168884881 | 0.433 | G:T | rs17071370 | 168908009 | 0.033 | C:T |
| rs6859112 | 168918641 | 0.350 | A:G | rs29793 | 168908334 | 0.458 | G:A |
| rs6899115 | 168918655 | 0.317 | G:A | rs25747 | 168908807 | 0.375 | G:C |
| rs2249080 | 168919049 | 0.350 | T:C | rs29792 | 168910726 | 0.317 | T:A |
| rs2431713 | 168919982 | 0.350 | A:T | rs29791 | 168911378 | 0.342 | T:C |
| rs2249184 | 168920267 | 0.375 | T:C | rs250275 | 168911599 | 0.342 | C:T |
| rs2546691 | 168922919 | 0.375 | T:C | rs17071396 | 168914407 | 0.033 | T:C |
| rs10516065 | 168923995 | 0.033 | G:A | rs173892 | 168915132 | 0.375 | G:A |
| rs10516066 | 168924241 | 0.033 | G:A | rs255815 | 168917416 | 0.317 | A:T |
| rs255825 | 168928262 | 0.317 | G:A | rs2263844 | 168918357 | 0.375 | T:C |
| rs27403 | 168929904 | 0.375 | T:C | rs3815729 | 168958367 | 0.375 | A:G |
| rs25746 | 168930683 | 0.017 | T:C | rs918512 | 168958933 | 0.350 | A:C |
| rs27674 | 168930725 | 0.350 | G:A | rs11958514 | 168959560 | 0.025 | T:C |
| rs25745 | 168930838 | 0.350 | A:G | rs6868672 | 168960388 | 0.317 | A:G |
| rs27871 | 168931108 | 0.342 | G:A | rs3797712 | 168960724 | 0.350 | C:G |
| rs17071455 | 168931314 | 0.025 | A:C | rs3797713 | 168961059 | 0.317 | C:T |
| rs25748 | 168933735 | 0.317 | T:C | rs13167686 | 168961664 | 0.350 | T:G |
| rs27974 | 168934181 | 0.375 | G:A | rs3822664 | 168961981 | 0.350 | G:A |
| rs29808 | 168936386 | 0.367 | T:C | rs3777084 | 168963728 | 0.317 | C:T |
| rs39800 | 168937049 | 0.317 | G:T | rs2042248 | 168963796 | 0.317 | T:G |
| rs29807 | 168937127 | 0.375 | C:T | rs6555868 | 168967552 | 0.375 | G:T |
| rs10061474 | 168939039 | 0.033 | A:G | rs10475538 | 168976470 | 0.317 | C:A |
| rs154035 | 168941638 | 0.350 | G:A | rs264858 | 168986819 | 0.317 | T:A |
| rs154034 | 168943691 | 0.308 | G:C | rs264863 | 168992855 | 0.317 | C:A |
| rs154033 | 168944118 | 0.317 | A:C | | | | |

TABLE 36
Anchor Tag
Block 001
Chromosome 6
Rs4959862 (BP 3662712), Rs9378813 (BP 3663678), and
Rs9378381 (BP 3663880) LD block
SNPs

| | | Minor | |
|---|---|---|---|
| | Base | Allele | |
| SNP ID (rs) | Position | Freq | Alleles |
| rs4959862 | 3662712 | 0.417 | T:C |
| rs4959863 | 3662770 | 0.417 | A:C |
| rs13194192 | 3663243 | 0.417 | T:G |
| rs190129 | 3663360 | 0.042 | A:G |
| rs9405663 | 3663529 | 0.417 | A:G |
| rs9378813 | 3663678 | 0.417 | G:A |
| rs9378381 | 3663880 | 0.417 | T:C |

TABLE 37
Anchor Tag
Block 002
Chromosome 6
Rs9394604 (BP 12278647) LD block SNPs

| | | Minor | |
|---|---|---|---|
| | Base | Allele | |
| SNP ID (rs) | Position | Freq | Alleles |
| rs3777755 | 12267685 | 0.300 | C:T |
| rs3777754 | 12268533 | 0.075 | T:C |
| rs16872341 | 12270982 | 0.042 | A:T |
| rs1042054 | 12272598 | 0.042 | C:G |
| rs219950 | 12273349 | 0.308 | C:T |
| rs17650437 | 12273505 | 0.042 | C:T |
| rs16872348 | 12274485 | 0.275 | T:C |
| rs9394604 | 12278647 | 0.075 | C:A |
| rs17590854 | 12278885 | 0.042 | G:A |
| rs219963 | 12278913 | 0.075 | T:C |
| rs9394605 | 12279143 | 0.292 | G:A |

TABLE 38
Anchor Tag
Block 003
Chromosome 6
Rs9383114 (BP 16207259) and Rs6912988 (BP 16208633)
LD block SNPs

| | | Minor | |
|---|---|---|---|
| | Base | Allele | |
| SNP ID (rs) | Position | Freq | Alleles |
| rs7768105 | 16199373 | 0.467 | T:C |
| rs7744733 | 16199621 | 0.133 | C:A |
| rs9358083 | 16200051 | 0.467 | G:A |
| rs9358084 | 16200504 | 0.467 | G:A |
| rs16877619 | 16200923 | 0.142 | G:C |
| rs9396643 | 16200972 | 0.467 | T:G |
| rs16877620 | 16201404 | 0.142 | G:T |
| rs12192563 | 16201441 | 0.467 | A:T |
| rs12524068 | 16204525 | 0.125 | C:T |
| rs4716045 | 16204575 | 0.433 | C:T |
| rs6459447 | 16205265 | 0.425 | C:T |
| rs12201671 | 16205941 | 0.433 | A:C |
| rs6459448 | 16206269 | 0.433 | G:A |
| rs12208024 | 16206367 | 0.008 | G:A |
| rs6921677 | 16206923 | 0.267 | T:C |
| rs11966052 | 16206969 | 0.142 | A:G |
| rs9383114 | 16207259 | 0.433 | T:A |
| rs6928267 | 16208132 | 0.433 | C:G |
| rs6912988 | 16208633 | 0.433 | T:C |
| rs10949343 | 16209236 | 0.442 | T:C |
| rs11751891 | 16209734 | 0.050 | C:A |
| rs12192575 | 16210281 | 0.433 | C:T |
| rs7745965 | 16210721 | 0.433 | G:A |
| rs7749877 | 16210781 | 0.433 | G:A |

TABLE 39
Anchor Tag
Block 004
Chromosome 6
Rs4510658 (BP 22863298) LD block SNPs

| | | Minor | |
|---|---|---|---|
| | Base | Allele | |
| SNP ID (rs) | Position | Freq | Alleles |
| rs4275050 | 22858623 | 0.108 | C:G |
| rs10498710 | 22859927 | 0.142 | T:C |
| rs10806962 | 22860201 | 0.033 | T:C |
| rs4305713 | 22860585 | 0.142 | T:C |
| rs7356884 | 22861026 | 0.033 | A:G |
| rs6917263 | 22862025 | 0.108 | C:G |
| rs12196870 | 22862925 | 0.033 | A:G |
| rs4466239 | 22863118 | 0.450 | G:A |
| rs4510658 | 22863298 | 0.450 | G:A |
| rs12154136 | 22863558 | 0.033 | G:A |
| rs9356835 | 22863789 | 0.108 | G:C |
| rs4562131 | 22864796 | 0.017 | G:A |
| rs4607423 | 22865239 | 0.108 | C:T |

TABLE 40
Anchor Tag
Block 005
Chromosome 6
Rs10484958 (BP 47229145) and Rrs9395236
(BP 47236608) LD block SNPs

| | | Minor | |
|---|---|---|---|
| | Base | Allele | |
| SNP ID (rs) | Position | Freq | Alleles |
| rs10484958 | 47229145 | 0.100 | G:A |
| rs926774 | 47229234 | 0.133 | A:G |
| rs12193269 | 47230204 | 0.050 | C:T |
| rs9381514 | 47232015 | 0.442 | C:A |
| rs7769886 | 47232643 | 0.367 | T:G |
| rs7774223 | 47232946 | 0.175 | G:A |
| rs6919892 | 47233282 | 0.400 | T:C |
| rs11755368 | 47233294 | 0.025 | A:G |
| rs6458543 | 47233395 | 0.017 | T:C |
| rs11760005 | 47233420 | 0.025 | C:T |
| rs9473020 | 47233768 | 0.400 | G:A |
| rs6458544 | 47234328 | 0.183 | A:G |
| rs7762257 | 47234520 | 0.142 | T:A |
| rs9473021 | 47235282 | 0.400 | G:A |
| rs4714996 | 47235748 | 0.133 | G:A |
| rs9296533 | 47236556 | 0.025 | C:G |
| rs9395236 | 47236608 | 0.075 | A:C |
| rs9296534 | 47236688 | 0.442 | G:A |
| rs9473022 | 47236852 | 0.400 | C:T |
| rs2179921 | 47237878 | 0.192 | C:A |
| rs9296535 | 47238869 | 0.408 | G:A |
| rs9296536 | 47238947 | 0.158 | A:G |

TABLE 41
Anchor Tag
Block 006
Chromosome 6
Rs10806546 (BP 67569403) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs4710626 | 67554588 | 0.492 | A:G | rs9360234 | 67577822 | 0.217 | G:A |
| rs7450297 | 67558018 | 0.233 | G:A | rs7743143 | 67577875 | 0.358 | T:A |
| rs4546468 | 67558222 | 0.250 | C:T | rs7743165 | 67577943 | 0.500 | G:G |
| rs9354475 | 67559766 | 0.242 | C:A | rs7756661 | 67577980 | 0.142 | G:A |
| rs9354476 | 67559892 | 0.258 | G:C | rs6900851 | 67578148 | 0.108 | A:G |
| rs9354477 | 67559943 | 0.242 | T:G | rs13206385 | 67578273 | 0.142 | C:T |
| rs13204310 | 67560505 | 0.133 | G:T | rs12211205 | 67582438 | 0.358 | A:C |
| rs13207643 | 67560742 | 0.133 | G:A | rs12214943 | 67584179 | 0.358 | A:G |
| rs9345890 | 67560989 | 0.250 | G:A | rs12208036 | 67584323 | 0.358 | C:T |
| rs7742967 | 67561124 | 0.500 | A:A | rs10944927 | 67584390 | 0.500 | A:A |
| rs7761600 | 67561164 | 0.233 | G:C | rs13205957 | 67585197 | 0.142 | C:T |
| rs7751803 | 67561703 | 0.233 | A:T | rs7748379 | 67586040 | 0.233 | C:T |
| rs12212990 | 67561753 | 0.150 | C:T | rs9351600 | 67586556 | 0.250 | G:A |
| rs12214955 | 67562931 | 0.367 | C:A | rs10455620 | 67587076 | 0.358 | T:A |
| rs12214995 | 67562974 | 0.367 | C:T | rs4391249 | 67588711 | 0.358 | C:T |
| rs12201273 | 67563266 | 0.367 | G:A | rs9345896 | 67590725 | 0.250 | C:A |
| rs12203012 | 67564026 | 0.367 | G:T | rs3924536 | 67592330 | 0.358 | C:G |
| rs9453891 | 67564680 | 0.200 | C:T | rs4394199 | 67593121 | 0.142 | A:G |
| rs11755246 | 67565704 | 0.217 | T:A | rs12530164 | 67593524 | 0.142 | C:A |
| rs9345891 | 67567217 | 0.267 | T:C | rs12527023 | 67593685 | 0.133 | G:A |
| rs12526882 | 67568683 | 0.133 | G:A | rs13214258 | 67594819 | 0.142 | T:C |
| rs10806546 | 67569403 | 0.492 | T:A | rs4269356 | 67595800 | 0.200 | A:G |
| rs9363639 | 67569490 | 0.258 | G:C | rs12200870 | 67596378 | 0.367 | A:G |
| rs7758200 | 67572963 | 0.358 | G:A | rs13199500 | 67596401 | 0.142 | G:C |
| rs7758357 | 67573055 | 0.358 | G:A | rs9345898 | 67596908 | 0.208 | G:A |
| rs12528149 | 67574053 | 0.142 | T:A | rs12212434 | 67596947 | 0.367 | C:T |
| rs12209119 | 67574926 | 0.500 | C:C | rs12212564 | 67597138 | 0.367 | C:T |
| rs12209528 | 67575835 | 0.358 | A:G | rs4523069 | 67597865 | 0.492 | T:C |
| rs12215749 | 67575922 | 0.358 | G:A | rs4307154 | 67597979 | 0.208 | T:A |
| rs12530311 | 67575994 | 0.142 | G:C | rs7752177 | 67598152 | 0.142 | A:C |
| rs12529103 | 67576063 | 0.500 | C:C | rs11753204 | 67598300 | 0.367 | T:C |
| rs9445781 | 67576740 | 0.017 | G:A | rs12216145 | 67598652 | 0.367 | C:A |
| rs10944924 | 67576860 | 0.500 | G:G | rs12197720 | 67599044 | 0.367 | T:C |
| rs4298331 | 67577683 | 0.358 | T:A | rs12191227 | 67599773 | 0.367 | C:T |
| rs4526179 | 67600582 | 0.367 | T:C | rs4519994 | 67604995 | 0.150 | T:A |
| rs4377773 | 67600701 | 0.367 | A:G | rs11754855 | 67605192 | 0.367 | C:G |
| rs12193333 | 67601245 | 0.033 | C:T | rs13218643 | 67605593 | 0.133 | G:A |
| rs4318854 | 67601325 | 0.367 | G:A | rs12209519 | 67605861 | 0.367 | A:G |
| rs12200302 | 67601382 | 0.142 | A:G | rs7757041 | 67606051 | 0.492 | G:A |
| rs12201841 | 67601779 | 0.142 | A:T | rs12202718 | 67606133 | 0.367 | C:T |
| rs10944928 | 67602337 | 0.483 | T:C | rs12211126 | 67606172 | 0.367 | T:C |
| rs12205538 | 67603263 | 0.367 | T:C | rs9342581 | 67610962 | 0.242 | C:T |
| rs4395708 | 67603627 | 0.242 | A:G | rs9351602 | 67611947 | 0.242 | C:T |
| rs3931506 | 67604891 | 0.483 | A:G | | | | |

TABLE 42
Anchor Tag
Block 007
Chromosome 6
Rs6923765 (BP 135376869), Rs1590975 (BP 135393781),
Rs11754265 (BP 135397909), Rs7741515 (BP 135416061),
Rs2150681 (BP 135416925), and Rs1331308 (BP 135446815) LD
block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs4646868 | 135313639 | 0.417 | A:G | rs12526072 | 135345049 | 0.108 | A:G |
| rs4346862 | 135314049 | 0.042 | A:T | rs9373119 | 135346702 | 0.067 | G:C |
| rs4267967 | 135314170 | 0.417 | T:G | rs12525425 | 135346997 | 0.108 | T:C |
| rs4472368 | 135315925 | 0.417 | A:G | rs4376364 | 135349178 | 0.108 | T:G |
| rs7768278 | 135316650 | 0.375 | T:C | rs11754021 | 135350068 | 0.375 | A:G |
| rs4289677 | 135317178 | 0.375 | T:C | rs9399130 | 135350824 | 0.108 | T:C |
| rs4896118 | 135317758 | 0.375 | G:A | rs4445070 | 135350905 | 0.483 | C:T |
| rs4440481 | 135318389 | 0.417 | T:C | rs13218642 | 135351633 | 0.483 | A:G |
| rs7767201 | 135321955 | 0.042 | T:C | rs4451151 | 135352239 | 0.483 | C:T |
| rs11753985 | 135322131 | 0.375 | C:T | rs12110789 | 135353786 | 0.008 | T:C |
| rs1135205 | 135323488 | 0.425 | C:T | rs13197750 | 135354073 | 0.375 | G:A |
| rs7739722 | 135323918 | 0.375 | T:C | rs6902954 | 135355687 | 0.483 | C:A |
| rs9389248 | 135324349 | 0.417 | T:C | rs9376079 | 135355976 | 0.108 | C:T |
| rs4475342 | 135325041 | 0.492 | T:C | rs9376080 | 135357091 | 0.108 | C:T |
| rs13196486 | 135325567 | 0.375 | C:G | rs6909975 | 135357263 | 0.492 | C:T |
| rs9399129 | 135326841 | 0.483 | C:A | rs12663447 | 135358719 | 0.375 | A:C |
| rs4594969 | 135327279 | 0.375 | A:T | rs4895438 | 135359358 | 0.483 | G:T |
| rs13199205 | 135328198 | 0.483 | T:C | rs9402671 | 135359482 | 0.117 | T:C |
| rs9402669 | 135329106 | 0.108 | A:T | rs7773126 | 135360083 | 0.492 | A:G |
| rs12663810 | 135331475 | 0.383 | G:C | rs4895439 | 135360547 | 0.483 | A:T |
| rs9376074 | 135333811 | 0.317 | T:C | rs11754504 | 135363202 | 0.483 | C:T |
| rs13208464 | 135333948 | 0.025 | A:G | rs9373120 | 135364022 | 0.108 | A:C |
| rs4895434 | 135335058 | 0.492 | G:A | rs9402674 | 135364437 | 0.108 | C:T |
| rs4896119 | 135335084 | 0.483 | C:T | rs4559100 | 135365778 | 0.483 | T:C |
| rs4895435 | 135335156 | 0.492 | C:A | rs12527707 | 135366204 | 0.492 | G:A |
| rs4896120 | 135335205 | 0.492 | C:G | rs12526055 | 135367611 | 0.483 | C:T |
| rs7383710 | 135335338 | 0.483 | T:C | rs6929661 | 135370485 | 0.483 | G:A |
| rs7755680 | 135335952 | 0.483 | A:G | rs9389253 | 135371448 | 0.483 | T:C |
| rs4895436 | 135336694 | 0.483 | G:A | rs2327578 | 135371662 | 0.483 | C:T |
| rs7381584 | 135337103 | 0.492 | T:G | rs9389256 | 135372947 | 0.483 | A:C |
| rs9373118 | 135338528 | 0.125 | T:C | rs1014021 | 135376293 | 0.492 | A:G |
| rs9376075 | 135338656 | 0.483 | A:G | rs6923765 | 135376869 | 0.483 | G:C |
| rs10484496 | 135341375 | 0.375 | T:C | rs6923512 | 135376961 | 0.375 | C:T |
| rs13214669 | 135341652 | 0.375 | T:C | rs6923827 | 135377075 | 0.492 | C:T |
| rs6902438 | 135378280 | 0.483 | C:T | rs9402677 | 135412401 | 0.483 | A:T |
| rs6908681 | 135379426 | 0.483 | A:G | rs9389261 | 135412841 | 0.483 | C:G |
| rs3756799 | 135382704 | 0.483 | A:G | rs9376085 | 135412917 | 0.483 | T:C |
| rs4896125 | 135384262 | 0.483 | C:T | rs9376086 | 135413022 | 0.483 | T:C |
| rs949547 | 135385192 | 0.483 | C:G | rs1041480 | 135414769 | 0.483 | G:T |
| rs13209780 | 135386488 | 0.375 | G:C | rs9373122 | 135415227 | 0.483 | G:A |
| rs9385714 | 135387640 | 0.108 | C:T | rs7741515 | 135416061 | 0.483 | C:G |
| rs13192235 | 135388044 | 0.100 | G:A | rs2150681 | 135416925 | 0.483 | C:T |
| rs9376082 | 135388082 | 0.483 | T:A | rs2297338 | 135417455 | 0.483 | G:A |
| rs11756988 | 135389288 | 0.483 | A:G | rs9389262 | 135418102 | 0.483 | G:T |
| rs12661423 | 135391263 | 0.483 | C:T | rs13208043 | 135418168 | 0.017 | T:C |
| rs4896128 | 135391449 | 0.375 | G:A | rs2183709 | 135418784 | 0.483 | C:T |
| rs4896129 | 135391538 | 0.375 | C:T | rs4142299 | 135418879 | 0.483 | G:T |
| rs987690 | 135392072 | 0.475 | A:G | rs11154791 | 135420841 | 0.375 | A:G |
| rs7750574 | 135392625 | 0.375 | G:C | rs11759062 | 135422941 | 0.375 | A:C |
| rs7750300 | 135392643 | 0.483 | C:G | rs11759077 | 135423044 | 0.375 | A:G |
| rs9376083 | 135393413 | 0.108 | G:A | rs7755121 | 135423457 | 0.008 | A:G |
| rs1590975 | 135393781 | 0.483 | C:T | rs6904897 | 135424673 | 0.375 | T:G |
| rs6569988 | 135393996 | 0.492 | C:A | rs11758774 | 135425482 | 0.375 | T:G |
| rs6919862 | 135394136 | 0.483 | C:A | rs2327582 | 135425688 | 0.325 | G:A |
| rs9321481 | 135394341 | 0.483 | G:C | rs7748365 | 135427300 | 0.008 | G:A |
| rs10872427 | 135395145 | 0.483 | T:C | rs1547247 | 135432529 | 0.283 | G:A |
| rs7766189 | 135395544 | 0.483 | A:C | rs11757577 | 135433158 | 0.483 | G:A |
| rs4134030 | 135395787 | 0.500 | G:G | rs11752907 | 135433188 | 0.108 | A:G |
| rs4896130 | 135396811 | 0.492 | C:T | rs13220662 | 135437241 | 0.333 | G:A |
| rs11754265 | 135397909 | 0.492 | G:C | rs7763587 | 135440758 | 0.125 | C:G |
| rs7742542 | 135400260 | 0.483 | T:G | rs9483783 | 135442733 | 0.008 | T:C |
| rs9373121 | 135400609 | 0.108 | G:A | rs9389264 | 135442805 | 0.125 | C:G |
| rs9376084 | 135401739 | 0.108 | C:T | rs6914564 | 135445317 | 0.125 | C:T |
| rs6569990 | 135407511 | 0.483 | C:T | rs1331308 | 135446815 | 0.467 | A:C |
| rs6940258 | 135407825 | 0.375 | C:T | rs949895 | 135447052 | 0.467 | C:T |
| rs9399135 | 135410007 | 0.492 | G:A | rs9402683 | 135448491 | 0.125 | T:A |
| rs9389260 | 135410024 | 0.108 | G:A | rs914346 | 135450731 | 0.050 | C:T |
| rs6915770 | 135410907 | 0.483 | G:A | rs6913541 | 135450809 | 0.125 | A:G |
| rs11755229 | 135411606 | 0.483 | A:G | rs9376090 | 135452921 | 0.217 | T:C |

TABLE 43
Anchor Tag
Block 001
Chromosome 7
Rs6958161 (BP 2928051) and Rs6461856 (BP 2940640) LD block SNPs

| | | Minor | |
|---|---|---|---|
| | Base | Allele | |
| SNP ID (rs) | Position | Freq | Alleles |
| rs6958161 | 2928051 | 0.250 | C:T |
| rs10951052 | 2928542 | 0.350 | A:G |
| rs4722438 | 2929054 | 0.408 | G:A |
| rs4722440 | 2929142 | 0.392 | T:C |
| rs17253464 | 2929404 | 0.025 | A:G |
| rs12531881 | 2930083 | 0.367 | C:T |
| rs6972403 | 2930717 | 0.025 | A:G |
| rs12700596 | 2930884 | 0.025 | T:C |
| rs6955702 | 2930943 | 0.425 | A:G |
| rs10499319 | 2931157 | 0.333 | G:A |
| rs12155183 | 2931866 | 0.025 | A:G |
| rs11770525 | 2932229 | 0.025 | T:G |
| rs6975452 | 2933752 | 0.417 | A:T |
| rs6461856 | 2940640 | 0.217 | G:T |
| rs6942973 | 2940992 | 0.083 | A:G |
| rs10270815 | 2941511 | 0.217 | T:A |
| rs17133056 | 2941770 | 0.100 | G:A |
| rs12674252 | 2942735 | 0.283 | C:T |
| rs12532214 | 2943131 | 0.125 | G:C |
| rs4722453 | 2943510 | 0.275 | A:C |

TABLE 44
Anchor Tag
Block 002
Chromosome 7
Rs11770341 (BP 46936429) LD block SNPs

| | | Minor | |
|---|---|---|---|
| | Base | Allele | |
| SNP ID (rs) | Position | Freq | Alleles |
| rs6978453 | 46936076 | 0.150 | A:G |
| rs11770341 | 46936429 | 0.158 | G:A |
| rs4724541 | 46936589 | 0.233 | T:A |
| rs1898663 | 46936996 | 0.383 | G:A |
| rs2118665 | 46937385 | 0.233 | A:G |
| rs1036587 | 46937636 | 0.217 | C:A |
| rs10229059 | 46938024 | 0.308 | T:C |
| rs4724542 | 46938213 | 0.317 | G:C |
| rs12702305 | 46938972 | 0.058 | G:T |
| rs9792026 | 46939074 | 0.317 | C:T |
| rs876922 | 46939514 | 0.317 | C:T |
| rs873278 | 46939971 | 0.317 | C:A |
| rs873277 | 46939989 | 0.317 | A:G |
| rs920313 | 46940634 | 0.317 | T:C |
| rs2053314 | 46941069 | 0.317 | A:G |
| rs2053315 | 46941162 | 0.317 | A:G |
| rs17172823 | 46941205 | 0.008 | T:C |
| rs12674402 | 46941786 | 0.308 | T:C |
| rs6969764 | 46943060 | 0.317 | T:A |
| rs4724543 | 46944838 | 0.317 | G:T |
| rs13239282 | 46946489 | 0.092 | T:C |
| rs10807944 | 46946996 | 0.317 | G:A |
| rs12534724 | 46947312 | 0.317 | T:C |
| rs10951893 | 46948782 | 0.317 | T:A |
| rs10235993 | 46953836 | 0.417 | G:A |
| rs7456147 | 46955416 | 0.075 | A:G |
| rs6957177 | 46956299 | 0.317 | A:G |
| rs2083206 | 46957290 | 0.317 | C:T |
| rs17172825 | 46957348 | 0.233 | A:G |
| rs6973264 | 46959126 | 0.317 | G:A |
| rs4144076 | 46959192 | 0.233 | G:A |
| rs4144077 | 46959386 | 0.450 | G:C |
| rs2918194 | 46959652 | 0.450 | A:C |
| rs7810580 | 46960304 | 0.317 | T:A |
| rs959856 | 46961214 | 0.450 | A:C |
| rs1438603 | 46961304 | 0.317 | G:A |
| rs768904 | 46961362 | 0.067 | G:A |
| rs12671659 | 46962063 | 0.317 | T:A |
| rs2728475 | 46962447 | 0.317 | T:C |
| rs1438604 | 46962759 | 0.233 | C:G |
| rs2053313 | 46962990 | 0.292 | A:G |
| rs2728478 | 46963618 | 0.317 | G:A |
| rs2728479 | 46963672 | 0.317 | G:A |
| rs2728480 | 46963835 | 0.317 | G:C |
| rs960217 | 46964196 | 0.317 | C:T |
| rs2728481 | 46964590 | 0.317 | G:T |
| rs2728483 | 46966592 | 0.317 | G:A |
| rs10499657 | 46966949 | 0.133 | G:A |
| rs2578430 | 46968079 | 0.450 | C:A |
| rs2728484 | 46968293 | 0.317 | G:T |
| rs7800065 | 46970986 | 0.308 | G:T |
| rs2578435 | 46971846 | 0.208 | A:G |
| rs2578436 | 46972287 | 0.317 | G:A |
| rs6968935 | 46972641 | 0.208 | T:C |
| rs7787540 | 46973719 | 0.450 | G:A |
| rs10241085 | 46979861 | 0.125 | C:A |

TABLE 45
Anchor Tag
Block 003
Chromosome 7
Rs10281500 (BP 55317099) LD block SNPs

| | | Minor | |
|---|---|---|---|
| | Base | Allele | |
| SNP ID (rs) | Position | Freq | Alleles |
| rs6972560 | 55290080 | 0.325 | G:A |
| rs10265664 | 55292879 | 0.008 | T:C |
| rs6961353 | 55308056 | 0.475 | C:T |
| rs10229613 | 55309073 | 0.350 | T:C |
| rs712820 | 55309247 | 0.350 | T:C |
| rs6973769 | 55311381 | 0.342 | A:T |
| rs940633 | 55311706 | 0.300 | A:C |
| rs815957 | 55314916 | 0.342 | G:A |
| rs712819 | 55315000 | 0.117 | C:G |
| rs10281500 | 55317099 | 0.342 | C:G |
| rs815959 | 55317472 | 0.308 | T:C |
| rs815961 | 55318933 | 0.350 | C:T |
| rs815962 | 55320604 | 0.317 | G:A |
| rs815964 | 55321903 | 0.342 | C:T |
| rs12718967 | 55324596 | 0.350 | G:A |
| rs6957301 | 55324803 | 0.100 | G:A |
| rs6957545 | 55324902 | 0.342 | T:C |
| rs815965 | 55326041 | 0.342 | T:C |
| rs11770090 | 55326137 | 0.192 | G:C |
| rs9656707 | 55327194 | 0.350 | T:C |
| rs13225043 | 55327479 | 0.342 | A:C |
| rs2692607 | 55328424 | 0.108 | G:C |
| rs815966 | 55330837 | 0.350 | T:C |
| rs815967 | 55330900 | 0.117 | G:A |
| rs10226502 | 55332987 | 0.325 | T:G |
| rs12718968 | 55334604 | 0.333 | G:A |

TABLE 46
Anchor Tag
Block 004
Chromosome 7
Rs10156094 (BP 75648618) LD block SNPs

| | | Minor | |
|---|---|---|---|
| | Base | Allele | |
| SNP ID (rs) | Position | Freq | Alleles |
| rs962627 | 75640141 | 0.258 | C:T |
| rs4728696 | 75645385 | 0.233 | G:A |
| rs10156094 | 75648618 | 0.500 | C:C |
| rs10224066 | 75649360 | 0.500 | A:A |
| rs10231308 | 75650347 | 0.242 | T:C |
| rs10272095 | 75651173 | 0.017 | T:C |
| rs1860148 | 75651465 | 0.258 | T:A |
| rs868269 | 75652460 | 0.017 | T:C |
| rs17149195 | 75654152 | 0.017 | C:T |
| rs10270398 | 75655107 | 0.300 | C:G |
| rs4728710 | 75657317 | 0.492 | T:C |
| rs11767818 | 75659232 | 0.200 | G:A |
| rs11771651 | 75660873 | 0.475 | C:A |
| rs4728712 | 75674346 | 0.267 | C:T |
| rs10228441 | 75676942 | 0.483 | G:C |
| rs6958115 | 75680817 | 0.458 | G:C |
| rs17149199 | 75682234 | 0.042 | A:G |
| rs17149201 | 75682710 | 0.008 | G:A |
| rs11486951 | 75682804 | 0.458 | A:T |
| rs12540545 | 75683499 | 0.042 | A:G |
| rs12540573 | 75683664 | 0.042 | A:C |
| rs7799638 | 75685178 | 0.033 | T:C |
| rs7777294 | 75685214 | 0.033 | C:T |
| rs10278706 | 75687169 | 0.008 | C:T |
| rs1019096 | 75690975 | 0.475 | G:A |
| rs11983026 | 75691549 | 0.042 | C:A |
| rs750103 | 75693186 | 0.025 | T:G |
| rs10241243 | 75693327 | 0.017 | T:C |
| rs2868366 | 75694783 | 0.492 | G:A |

TABLE 47
Anchor Tag
Block 005
Chromosome 7
Rs9986802 (BP 141326058), Rs9986873 (BP 141326076) and
Rs4389847 (BP 141329303) LD block SNPs

| | | Minor | |
|---|---|---|---|
| | Base | Allele | |
| SNP ID (rs) | Position | Freq | Alleles |
| rs4726493 | 141318229 | 0.267 | C:T |
| rs7794533 | 141319686 | 0.092 | T:G |
| rs7779634 | 141324435 | 0.342 | A:T |
| rs9986802 | 141326058 | 0.442 | T:C |
| rs9986873 | 141326076 | 0.442 | G:A |
| rs12532545 | 141328451 | 0.250 | C:A |
| rs6958066 | 141328778 | 0.250 | C:A |
| rs6958102 | 141328855 | 0.250 | C:G |
| rs4389847 | 141329303 | 0.442 | C:T |
| rs4285402 | 141335639 | 0.250 | A:G |
| rs4406326 | 141335822 | 0.242 | T:C |
| rs10952518 | 141338387 | 0.250 | A:G |

TABLE 48
Anchor Tag
Block 006
Chromosome 7
Rs7783954 (BP 150237027) and Rs7810706 (BP 150268937)
LD block SNPs.
These tags are in separate LD blocks.

| | | Minor | |
|---|---|---|---|
| | Base | Allele | |
| SNP ID (rs) | Position | Freq | Alleles |
| rs11769150 | 150223401 | 0.167 | C:T |
| rs6957724 | 150225952 | 0.208 | C:T |
| rs6464127 | 150227204 | 0.250 | G:C |
| rs10279252 | 150227439 | 0.017 | C:G |
| rs4725988 | 150229327 | 0.217 | C:T |
| rs4517031 | 150234575 | 0.025 | T:A |
| rs1036062 | 150234594 | 0.217 | C:T |
| rs1036061 | 150234737 | 0.217 | C:T |
| rs4236428 | 150234900 | 0.217 | C:T |
| rs1978655 | 150235229 | 0.492 | A:G |
| rs7783954 | 150237027 | 0.492 | C:T |
| rs10244332 | 150238471 | 0.217 | T:G |
| rs4725991 | 150238886 | 0.025 | A:G |
| rs4725391 | 150242785 | 0.008 | A:G |
| rs6951528 | 150246812 | 0.233 | A:C |
| rs6968877 | 150267663 | 0.408 | A:G |
| rs7810706 | 150268937 | 0.017 | C:T |
| rs10952303 | 150270561 | 0.283 | G:A |

TABLE 49
Anchor Tag
Block 001
Chromosome 8
Rs7462266 (BP 6109511) and Rs6559135 (BP 6132325)
LD block SNPs. These tags are in separate LD blocks.

| | | Minor | |
|---|---|---|---|
| | Base | Allele | |
| SNP ID (rs) | Position | Freq | Alleles |
| rs17381669 | 6107334 | 0.342 | G:A |
| rs17466189 | 6107715 | 0.133 | A:G |
| rs4570185 | 6108358 | 0.350 | C:T |
| rs17466210 | 6110545 | 0.342 | A:G |
| rs17063413 | 6110801 | 0.258 | A:G |
| rs6995192 | 6111533 | 0.292 | A:T |
| rs17466259 | 6113325 | 0.058 | A:G |
| rs17381755 | 6113403 | 0.325 | C:T |
| rs12234944 | 6130460 | 0.142 | T:C |
| rs6994029 | 6133731 | 0.067 | G:A |

TABLE 50
Anchor Tag
Block 002
Chromosome 8
Rs897266 (BP 13269177) LD block SNPs

| | | Minor | |
|---|---|---|---|
| | Base | Allele | |
| SNP ID (rs) | Position | Freq | Alleles |
| rs897265 | 13269043 | 0.442 | G:A |
| rs1671410 | 13269142 | 0.442 | G:A |
| rs897266 | 13269177 | 0.350 | A:G |
| rs1671411 | 13269203 | 0.425 | G:C |
| rs4102144 | 13269236 | 0.158 | C:T |
| rs7833624 | 13269651 | 0.158 | C:T |
| rs7834479 | 13270183 | 0.158 | C:T |

TABLE 51
Anchor Tag
Block 003
Chromosome 8
Rs2976425 (BP 24877700) LD block SNPs

| | | Minor | |
|---|---|---|---|
| | Base | Allele | |
| SNP ID (rs) | Position | Freq | Alleles |
| rs196819 | 24851878 | 0.150 | A:G |
| rs2205169 | 24853187 | 0.125 | A:T |
| rs2975179 | 24854667 | 0.158 | G:C |
| rs6557786 | 24855657 | 0.158 | A:G |
| rs6998940 | 24855915 | 0.158 | G:A |
| rs10100189 | 24856105 | 0.125 | G:A |
| rs11994760 | 24858152 | 0.158 | C:T |
| rs6557789 | 24859196 | 0.008 | T:C |
| rs2976441 | 24863702 | 0.317 | A:G |
| rs17052849 | 24865862 | 0.033 | T:C |
| rs2976438 | 24870664 | 0.300 | T:G |
| rs17052863 | 24872182 | 0.033 | C:T |
| rs3757985 | 24872354 | 0.500 | A:A |
| rs4546670 | 24872489 | 0.025 | C:T |
| rs2979685 | 24872522 | 0.317 | C:A |
| rs17052871 | 24873029 | 0.025 | C:G |
| rs17830286 | 24873062 | 0.158 | C:T |
| rs2979684 | 24873358 | 0.317 | A:G |
| rs2976433 | 24873412 | 0.317 | T:C |
| rs11995677 | 24873749 | 0.317 | A:C |
| rs11988344 | 24873818 | 0.317 | A:G |
| rs13254844 | 24874430 | 0.050 | C:T |
| rs17830392 | 24874818 | 0.092 | A:G |
| rs2976428 | 24874922 | 0.317 | G:A |
| rs2979682 | 24875033 | 0.317 | A:G |
| rs11135835 | 24875360 | 0.317 | A:G |
| rs17052895 | 24875401 | 0.008 | A:T |
| rs2976427 | 24875821 | 0.133 | C:T |
| rs2979681 | 24876081 | 0.283 | T:A |
| rs11984639 | 24877494 | 0.025 | C:T |
| rs12545967 | 24877564 | 0.158 | T:G |
| rs2976425 | 24877700 | 0.133 | A:G |
| rs2976424 | 24877787 | 0.317 | T:C |
| rs2976423 | 24877933 | 0.167 | A:C |
| rs2979689 | 24879804 | 0.008 | A:G |
| rs17052916 | 24890962 | 0.025 | G:A |
| rs7829603 | 24891033 | 0.150 | A:C |
| rs17052921 | 24891123 | 0.025 | C:T |
| rs4498549 | 24891783 | 0.325 | G:A |
| rs4242416 | 24894385 | 0.125 | C:T |
| rs11779789 | 24894532 | 0.033 | T:C |
| rs11779795 | 24894579 | 0.167 | T:G |
| rs4242418 | 24895987 | 0.125 | A:G |
| rs4242419 | 24896045 | 0.325 | A:T |
| rs17052934 | 24896236 | 0.125 | A:C |

TABLE 52
Anchor Tag
Block 004
Chromosome 8
Rs218019 (BP 118776737) LD block SNPs

| | | Minor | |
|---|---|---|---|
| | Base | Allele | |
| SNP ID (rs) | Position | Freq | Alleles |
| rs218017 | 118775957 | 0.342 | T:G |
| rs2136106 | 118776405 | 0.008 | A:G |
| rs218018 | 118776669 | 0.325 | A:T |
| rs218019 | 118776737 | 0.392 | C:T |

TABLE 53
Anchor Tag
Block 001
Chromosome 9
Rs16912206 (BP 106942539), Rs4978605 (BP 106966463), and
Rs10521081 (BP 106968197) LD block SNPs

| | | Minor | |
|---|---|---|---|
| | Base | Allele | |
| SNP ID (rs) | Position | Freq | Alleles |
| rs2030235 | 106922898 | 0.092 | T:C |
| rs4443716 | 106923157 | 0.092 | A:G |
| rs4978917 | 106923184 | 0.167 | T:C |
| rs4978920 | 106923479 | 0.042 | G:T |
| rs2056258 | 106923839 | 0.092 | G:A |
| rs2056257 | 106923855 | 0.092 | T:A |
| rs7043490 | 106923869 | 0.008 | A:T |
| rs7047518 | 106924631 | 0.175 | C:T |
| rs4351470 | 106925106 | 0.092 | C:T |
| rs7853704 | 106926884 | 0.108 | A:G |
| rs7861402 | 106928364 | 0.092 | C:T |
| rs7861530 | 106928469 | 0.092 | C:T |
| rs7038796 | 106929756 | 0.092 | C:G |
| rs7869632 | 106930375 | 0.125 | C:A |
| rs7870580 | 106931117 | 0.275 | C:T |
| rs7873660 | 106931152 | 0.275 | C:A |
| rs12338560 | 106932151 | 0.067 | T:C |
| rs12006494 | 106933459 | 0.008 | G:A |
| rs7865188 | 106935117 | 0.167 | C:T |
| rs7869137 | 106935765 | 0.283 | G:A |
| rs12000257 | 106936390 | 0.008 | C:G |
| rs16912204 | 106937734 | 0.092 | T:C |
| rs1713660 | 106938129 | 0.008 | G:T |
| rs17785871 | 106939643 | 0.108 | C:T |
| rs17785883 | 106941128 | 0.042 | G:A |
| rs16912206 | 106942539 | 0.092 | C:T |
| rs784661 | 106943248 | 0.300 | G:A |
| rs784663 | 106943691 | 0.300 | T:A |
| rs16912209 | 106944586 | 0.092 | G:C |
| rs7030936 | 106945391 | 0.092 | A:G |
| rs698471 | 106947324 | 0.017 | A:G |
| rs7029669 | 106948413 | 0.292 | T:C |
| rs4117408 | 106948898 | 0.158 | G:A |
| rs7044846 | 106949087 | 0.275 | A:G |
| rs17785925 | 106951503 | 0.042 | C:G |
| rs7855383 | 106952086 | 0.008 | G:A |
| rs1878735 | 106953548 | 0.183 | C:A |
| rs13300739 | 106954299 | 0.008 | C:G |
| rs7853997 | 106954662 | 0.283 | C:T |
| rs16912217 | 106956465 | 0.108 | C:T |
| rs17785985 | 106956695 | 0.075 | C:G |
| rs1878929 | 106957145 | 0.125 | A:T |
| rs4979300 | 106958874 | 0.042 | T:G |
| rs7875051 | 106959810 | 0.008 | G:A |
| rs784660 | 106960540 | 0.400 | C:T |
| rs796753 | 106961771 | 0.308 | G:A |
| rs1007061 | 106961887 | 0.125 | G:A |
| rs17785997 | 106962282 | 0.042 | C:T |
| rs10978720 | 106964287 | 0.117 | C:A |
| rs7874076 | 106964949 | 0.008 | G:A |
| rs7848294 | 106965452 | 0.017 | G:A |
| rs4978605 | 106966463 | 0.158 | G:A |
| rs4246906 | 106968019 | 0.283 | C:T |
| rs10521081 | 106968197 | 0.150 | C:T |
| rs4979480 | 106969414 | 0.292 | G:C |

TABLE 54
Anchor Tag
Block 002
Chromosome 9
Rs17794822 (BP 109692695) and Rs17794870 (BP 1097001595)
LD block SNPs. These tags are in separate LD blocks.

| | | Minor | |
|---|---|---|---|
| | Base | Allele | |
| SNP ID (rs) | Position | Freq | Alleles |
| rs2769151 | 109688265 | 0.258 | G:A |
| rs10816895 | 109689077 | 0.183 | A:C |
| rs10816896 | 109689167 | 0.050 | G:A |
| rs10816897 | 109690385 | 0.300 | A:C |
| rs913360 | 109690934 | 0.233 | T:G |
| rs2252649 | 109690984 | 0.317 | T:C |
| rs913361 | 109691080 | 0.192 | G:A |
| rs2252637 | 109691194 | 0.342 | G:C |
| rs1077760 | 109692108 | 0.375 | G:T |
| rs913362 | 109692244 | 0.192 | C:T |
| rs1077761 | 109692337 | 0.442 | C:G |
| rs17794822 | 109692695 | 0.067 | C:T |
| rs2418059 | 109693232 | 0.008 | G:T |
| rs10980086 | 109693663 | 0.050 | A:C |
| rs2769149 | 109694887 | 0.208 | C:G |
| rs2769145 | 109701316 | 0.208 | A:G |
| rs16914551 | 109701534 | 0.042 | A:G |
| rs17794870 | 109701595 | 0.067 | G:A |
| rs2769146 | 109702164 | 0.200 | C:T |
| rs7040108 | 109702198 | 0.133 | G:A |
| rs9299174 | 109703186 | 0.133 | G:A |
| rs9299175 | 109703199 | 0.133 | T:C |
| rs9299176 | 109703217 | 0.133 | A:G |
| rs9299178 | 109703356 | 0.133 | T:C |
| rs10453216 | 109703718 | 0.133 | C:T |
| rs2479311 | 109703967 | 0.383 | T:A |
| rs10980094 | 109704699 | 0.067 | G:A |
| rs10816901 | 109704742 | 0.117 | A:T |
| rs10980095 | 109704892 | 0.133 | C:G |
| rs10816902 | 109705503 | 0.133 | A:G |
| rs13298370 | 109707385 | 0.192 | G:C |
| rs2479315 | 109707723 | 0.200 | C:G |

TABLE 55
Anchor Tag
Block 003
Chromosome 9
Rs10987199 (BP 126048541) LD block SNPs

| | | Minor | |
|---|---|---|---|
| | Base | Allele | |
| SNP ID (rs) | Position | Freq | Alleles |
| rs10987197 | 126047746 | 0.058 | A:G |
| rs10987198 | 126048085 | 0.250 | G:T |
| rs10987199 | 126048541 | 0.058 | C:T |
| rs2773395 | 126049019 | 0.292 | G:T |
| rs2417008 | 126049773 | 0.058 | G:A |
| rs10987202 | 126050360 | 0.042 | G:A |
| rs11788140 | 126053723 | 0.058 | G:A |

TABLE 56
Anchor Tag
Block 004
Chromosome 9
Rs947626 (BP 127853923) LD block SNPs

| | | Minor | |
|---|---|---|---|
| | Base | Allele | |
| SNP ID (rs) | Position | Freq | Alleles |
| rs947627 | 127853490 | 0.142 | A:T |
| rs947626 | 127853923 | 0.142 | C:A |
| rs4837221 | 127854561 | 0.142 | C:G |
| rs4131493 | 127854954 | 0.142 | G:A |
| rs4837223 | 127855961 | 0.142 | T:C |
| rs4836598 | 127856047 | 0.142 | A:G |
| rs10760523 | 127856451 | 0.142 | C:T |
| rs10819333 | 127857022 | 0.125 | A:G |
| rs10819334 | 127857703 | 0.142 | G:A |

TABLE 57
Anchor Tag
Block 001
Chromosome 10
Rs2782326 (BP 19541131) and Rs6481792 (BP 19541172)
LD block SNPs

| | | Minor | |
|---|---|---|---|
| | Base | Allele | |
| SNP ID (rs) | Position | Freq | Alleles |
| rs2245223 | 19539322 | 0.242 | T:C |
| rs2245235 | 19539478 | 0.242 | G:A |
| rs2583647 | 19539770 | 0.242 | T:C |
| rs2803835 | 19539999 | 0.242 | G:C |
| rs2803834 | 19540143 | 0.242 | G:C |
| rs12250334 | 19540158 | 0.333 | G:A |
| rs2782326 | 19541131 | 0.242 | A:T |
| rs6481792 | 19541172 | 0.133 | C:T |
| rs7393104 | 19541401 | 0.333 | G:A |
| rs2358357 | 19541704 | 0.242 | T:C |
| rs2245456 | 19542000 | 0.233 | G:A |

TABLE 58
Anchor Tag
Block 002
Chromosome 10
Rs11004689 (BP 56462885) LD block SNPs

| | | Minor | |
|---|---|---|---|
| | Base | Allele | |
| SNP ID (rs) | Position | Freq | Alleles |
| rs4558080 | 56429694 | 0.275 | G:A |
| rs6481134 | 56433592 | 0.267 | G:T |
| rs1950140 | 56433646 | 0.283 | T:A |
| rs7090160 | 56435409 | 0.258 | T:C |
| rs2141841 | 56438107 | 0.275 | G:A |
| rs10825430 | 56444532 | 0.067 | T:C |
| rs16906812 | 56445112 | 0.233 | A:G |
| rs2204866 | 56445312 | 0.200 | C:T |
| rs1922149 | 56445592 | 0.483 | G:A |
| rs1922150 | 56445788 | 0.275 | C:A |
| rs12240687 | 56446374 | 0.008 | C:T |
| rs10763170 | 56447030 | 0.483 | C:T |
| rs4363501 | 56447660 | 0.408 | A:G |
| rs1922152 | 56449204 | 0.483 | T:A |
| rs1922153 | 56449507 | 0.483 | A:G |
| rs10825431 | 56449775 | 0.067 | T:C |
| rs10825433 | 56450312 | 0.067 | A:G |
| rs10825436 | 56450470 | 0.067 | A:G |
| rs1922154 | 56451214 | 0.483 | A:T |
| rs2891554 | 56454036 | 0.483 | T:G |
| rs2384610 | 56454308 | 0.450 | T:C |
| rs11004683 | 56454702 | 0.483 | T:G |
| rs10825440 | 56454788 | 0.017 | T:C |
| rs7924112 | 56455398 | 0.483 | A:C |
| rs7923704 | 56455781 | 0.483 | A:G |
| rs16906827 | 56456372 | 0.067 | G:A |
| rs9299555 | 56456440 | 0.475 | G:A |
| rs12769204 | 56456604 | 0.067 | G:T |
| rs4326703 | 56456959 | 0.450 | T:C |
| rs4636569 | 56457410 | 0.450 | C:T |
| rs1922155 | 56458141 | 0.483 | T:C |
| rs2384611 | 56458367 | 0.483 | T:C |
| rs11004685 | 56458398 | 0.450 | C:G |
| rs12412292 | 56459016 | 0.483 | T:C |
| rs12761443 | 56485206 | 0.067 | G:C |
| rs11004708 | 56485272 | 0.067 | G:A |
| rs10825451 | 56485785 | 0.483 | A:T |
| rs11004710 | 56487409 | 0.067 | C:T |
| rs11004712 | 56487502 | 0.067 | T:C |
| rs17528196 | 56487539 | 0.408 | A:G |
| rs11004713 | 56487560 | 0.067 | A:G |
| rs1922159 | 56489938 | 0.042 | T:C |
| rs11004717 | 56493183 | 0.050 | G:T |
| rs10740611 | 56493447 | 0.483 | T:C |
| rs10430540 | 56493843 | 0.283 | T:C |
| rs11004719 | 56494150 | 0.067 | C:T |
| rs7081992 | 56459190 | 0.475 | C:T |
| rs10825442 | 56459738 | 0.450 | G:C |
| rs11004686 | 56459901 | 0.483 | C:G |
| rs10740609 | 56460975 | 0.258 | A:T |
| rs12260813 | 56461965 | 0.008 | T:G |
| rs4421665 | 56462086 | 0.483 | T:C |
| rs11004689 | 56462885 | 0.450 | A:G |
| rs12416394 | 56462921 | 0.067 | G:A |
| rs1922161 | 56463088 | 0.450 | A:C |
| rs1922165 | 56465030 | 0.483 | C:T |
| rs1922166 | 56465084 | 0.483 | A:C |
| rs10763175 | 56465487 | 0.483 | T:A |
| rs11004690 | 56465654 | 0.042 | C:T |
| rs10825445 | 56465681 | 0.483 | T:C |
| rs10825446 | 56465702 | 0.483 | G:A |
| rs11004691 | 56467351 | 0.067 | A:T |
| rs10825447 | 56467446 | 0.408 | T:C |
| rs12264342 | 56468321 | 0.008 | C:T |
| rs11004693 | 56468324 | 0.067 | G:C |
| rs11004694 | 56468646 | 0.483 | T:C |
| rs17599730 | 56468873 | 0.450 | C:A |
| rs2178646 | 56469340 | 0.208 | G:A |
| rs11004697 | 56470488 | 0.067 | A:G |
| rs11004700 | 56470779 | 0.067 | A:G |
| rs12244680 | 56471769 | 0.008 | G:T |
| rs7081117 | 56471854 | 0.483 | G:T |
| rs1922160 | 56474157 | 0.275 | A:G |
| rs11593351 | 56475179 | 0.450 | T:A |
| rs11004703 | 56476104 | 0.008 | T:C |
| rs10763176 | 56478812 | 0.483 | C:T |
| rs7896003 | 56478999 | 0.475 | A:G |
| rs10825450 | 56480046 | 0.275 | A:T |
| rs7915118 | 56483313 | 0.258 | T:C |
| rs11004706 | 56483675 | 0.067 | T:C |
| rs10825453 | 56494234 | 0.067 | G:T |
| rs10430541 | 56494253 | 0.367 | G:A |
| rs10825454 | 56494490 | 0.442 | T:C |
| rs1922158 | 56494637 | 0.492 | C:T |
| rs3858273 | 56494895 | 0.083 | A:G |
| rs11004721 | 56494915 | 0.425 | C:G |
| rs4472838 | 56495160 | 0.083 | G:A |
| rs3905359 | 56495364 | 0.042 | G:C |
| rs4007192 | 56495946 | 0.017 | G:A |
| rs10825455 | 56496449 | 0.083 | A:G |
| rs11004722 | 56496614 | 0.425 | G:A |
| rs11004723 | 56497436 | 0.492 | T:C |

TABLE 59
Anchor Tag
Block 003
Chromosome 10
Rs3862864 (BP 59582876) and Rs1199094
(BP 59622636) LD block SNPs

| | | Minor | |
|---|---|---|---|
| | Base | Allele | |
| SNP ID (rs) | Position | Freq | Alleles |
| rs2247911 | 59563936 | 0.317 | C:T |
| rs7918059 | 59565799 | 0.075 | T:C |
| rs4315014 | 59566432 | 0.025 | A:G |
| rs1199093 | 59566800 | 0.325 | G:C |
| rs17635621 | 59567539 | 0.117 | C:A |
| rs12359064 | 59567591 | 0.117 | A:G |
| rs11006065 | 59568644 | 0.108 | C:T |
| rs16911833 | 59570269 | 0.017 | G:A |
| rs1199091 | 59571565 | 0.217 | C:T |
| rs2653510 | 59575066 | 0.083 | T:C |
| rs1199086 | 59575821 | 0.225 | T:A |
| rs10509086 | 59575855 | 0.058 | C:G |
| rs1875592 | 59576047 | 0.017 | G:C |
| rs17635794 | 59577503 | 0.083 | C:T |
| rs11006067 | 59578851 | 0.117 | G:A |
| rs1199088 | 59579210 | 0.292 | C:A |
| rs16911842 | 59580636 | 0.017 | G:C |
| rs3862864 | 59582876 | 0.067 | A:G |
| rs1819658 | 59583157 | 0.200 | C:T |
| rs7082709 | 59585106 | 0.133 | G:A |
| rs11815860 | 59588839 | 0.058 | A:G |
| rs1199071 | 59591338 | 0.008 | C:T |
| rs1199072 | 59594588 | 0.200 | C:T |
| rs7072206 | 59595054 | 0.067 | C:T |
| rs16911872 | 59596041 | 0.017 | C:T |
| rs1199075 | 59596342 | 0.200 | G:A |
| rs1199076 | 59596457 | 0.200 | A:T |
| rs1199077 | 59596492 | 0.200 | A:G |
| rs1199078 | 59596541 | 0.200 | T:C |
| rs1199079 | 59596561 | 0.200 | A:G |
| rs1199083 | 59596841 | 0.200 | T:A |
| rs1199085 | 59597283 | 0.200 | A:G |
| rs1308356 | 59597321 | 0.200 | G:A |
| rs1308358 | 59597765 | 0.200 | T:C |
| rs7073090 | 59613240 | 0.083 | G:A |
| rs1199107 | 59613715 | 0.192 | T:G |
| rs7903198 | 59614660 | 0.083 | G:T |
| rs7071333 | 59615459 | 0.083 | T:A |
| rs7087498 | 59616224 | 0.017 | G:A |
| rs7088095 | 59616602 | 0.017 | G:A |
| rs1199105 | 59616678 | 0.133 | T:C |
| rs1199103 | 59617237 | 0.192 | A:G |
| rs12252749 | 59617549 | 0.058 | A:T |
| rs1199101 | 59617907 | 0.192 | C:T |
| rs1199100 | 59618050 | 0.192 | G:A |
| rs1199099 | 59618341 | 0.192 | C:T |
| rs1199098 | 59619742 | 0.192 | C:T |
| rs1199097 | 59620016 | 0.192 | G:A |
| rs1199096 | 59620240 | 0.192 | G:A |
| rs1199094 | 59622636 | 0.075 | A:T |
| rs17636964 | 59623052 | 0.158 | G:C |
| rs17710296 | 59625184 | 0.025 | C:T |
| rs2790149 | 59626562 | 0.192 | C:T |
| rs1769044 | 59628962 | 0.192 | C:A |
| rs1630731 | 59629536 | 0.192 | T:G |
| rs1769045 | 59630528 | 0.192 | C:T |
| rs1698473 | 59630868 | 0.075 | T:C |
| rs9416674 | 59631596 | 0.192 | A:G |
| rs9416675 | 59633483 | 0.192 | A:G |
| rs1619597 | 59633564 | 0.008 | T:C |
| rs2653505 | 59634148 | 0.192 | C:T |
| rs2455577 | 59634713 | 0.192 | A:T |
| rs2440855 | 59634750 | 0.192 | T:A |
| rs7475387 | 59635896 | 0.075 | G:A |
| rs9415543 | 59638096 | 0.192 | A:T |
| rs12247282 | 59639006 | 0.192 | C:T |
| rs12261547 | 59639117 | 0.058 | G:C |
| rs2486491 | 59640397 | 0.192 | G:A |
| rs9651352 | 59678470 | 0.192 | C:T |
| rs2590339 | 59682237 | 0.192 | T:C |
| rs7077184 | 59683979 | 0.025 | C:A |
| rs2590349 | 59684652 | 0.192 | G:A |
| rs2590350 | 59684874 | 0.008 | A:C |
| rs2790233 | 59684935 | 0.075 | T:C |
| rs2790241 | 59685319 | 0.192 | G:T |
| rs7895739 | 59686081 | 0.192 | A:G |
| rs2590357 | 59689970 | 0.192 | C:T |
| rs2590358 | 59690988 | 0.192 | G:C |
| rs2590359 | 59691253 | 0.192 | A:T |
| rs2095892 | 59693725 | 0.192 | A:C |
| rs2019022 | 59694058 | 0.192 | G:A |
| rs2590362 | 59694118 | 0.008 | A:G |
| rs2790167 | 59694476 | 0.192 | T:C |
| rs2590363 | 59694718 | 0.192 | C:G |
| rs7088198 | 59694838 | 0.042 | T:G |
| rs2790168 | 59695454 | 0.192 | C:T |
| rs2790169 | 59695660 | 0.192 | T:C |
| rs7899961 | 59696431 | 0.075 | G:T |
| rs946555 | 59696505 | 0.008 | G:A |
| rs1416763 | 59699169 | 0.192 | C:T |
| rs2790178 | 59704089 | 0.192 | A:G |
| rs2275442 | 59705158 | 0.058 | C:T |
| rs2790179 | 59705378 | 0.192 | T:C |
| rs2254775 | 59706821 | 0.192 | A:G |
| rs7073539 | 59707902 | 0.017 | C:T |
| rs2590364 | 59708412 | 0.192 | C:T |
| rs1867571 | 59708856 | 0.192 | A:G |
| rs1769025 | 59598172 | 0.200 | G:A |
| rs1698382 | 59598288 | 0.200 | G:A |
| rs1769024 | 59598321 | 0.200 | C:T |
| rs1769023 | 59598343 | 0.200 | C:T |
| rs1698386 | 59598974 | 0.075 | A:C |
| rs1416759 | 59599518 | 0.200 | T:C |
| rs1769016 | 59599999 | 0.200 | G:A |
| rs11006071 | 59600398 | 0.200 | C:T |
| rs12220092 | 59600855 | 0.150 | T:C |
| rs1416761 | 59600984 | 0.200 | A:C |
| rs7068355 | 59602608 | 0.017 | T:C |
| rs1698408 | 59603542 | 0.200 | G:C |
| rs1698407 | 59603737 | 0.200 | A:C |
| rs954951 | 59604107 | 0.200 | T:C |
| rs1111821 | 59605227 | 0.192 | C:G |
| rs1769007 | 59605345 | 0.008 | A:G |
| rs1698404 | 59605466 | 0.192 | A:G |
| rs1769006 | 59605553 | 0.192 | T:C |
| rs1698403 | 59605847 | 0.192 | A:T |
| rs1769005 | 59605946 | 0.192 | A:G |
| rs1698402 | 59606450 | 0.192 | C:T |
| rs1698396 | 59607314 | 0.192 | T:A |
| rs1769003 | 59607596 | 0.192 | A:C |
| rs10509087 | 59607666 | 0.017 | C:T |
| rs12570088 | 59608342 | 0.058 | A:G |
| rs17709956 | 59608348 | 0.025 | G:A |
| rs10826162 | 59608674 | 0.125 | T:A |
| rs1624017 | 59608964 | 0.192 | T:C |
| rs3956816 | 59609876 | 0.017 | G:A |
| rs2174021 | 59610865 | 0.083 | C:T |
| rs11006074 | 59611458 | 0.125 | A:G |
| rs16911916 | 59611793 | 0.083 | A:C |
| rs10509088 | 59611797 | 0.058 | C:T |
| rs16911920 | 59612011 | 0.083 | G:A |
| rs12251970 | 59641824 | 0.058 | C:T |
| rs2440845 | 59641890 | 0.192 | C:T |
| rs2153283 | 59642305 | 0.192 | C:A |
| rs2153282 | 59642422 | 0.192 | T:A |
| rs2153281 | 59642459 | 0.192 | G:T |
| rs11818436 | 59643038 | 0.067 | T:C |
| rs2590305 | 59643850 | 0.192 | C:T |
| rs2790156 | 59644727 | 0.192 | C:T |
| rs2790155 | 59644750 | 0.192 | C:T |
| rs2790154 | 59644794 | 0.192 | A:C |
| rs11006083 | 59645174 | 0.125 | G:A |
| rs2590306 | 59646972 | 0.192 | T:G |
| rs2590307 | 59647167 | 0.192 | C:T |
| rs1339587 | 59648911 | 0.008 | A:G |
| rs2790244 | 59649251 | 0.192 | G:C |
| rs16911967 | 59650200 | 0.050 | G:C |
| rs2790243 | 59650474 | 0.008 | T:C |
| rs2790242 | 59650658 | 0.192 | G:A |
| rs7068428 | 59652531 | 0.125 | C:A |
| rs2790236 | 59655108 | 0.192 | T:C |
| rs2790234 | 59655393 | 0.075 | C:G |
| rs2790232 | 59655677 | 0.008 | C:T |
| rs2590314 | 59656453 | 0.008 | G:A |
| rs2590315 | 59656498 | 0.192 | C:T |
| rs2590316 | 59657445 | 0.192 | T:A |
| rs2590317 | 59657664 | 0.133 | T:C |
| rs11006086 | 59658082 | 0.117 | T:C |
| rs2790218 | 59667307 | 0.192 | C:T |
| rs2790216 | 59667932 | 0.192 | G:A |
| rs2253192 | 59669329 | 0.192 | A:G |
| rs2590322 | 59669937 | 0.192 | C:T |
| rs6481383 | 59672351 | 0.317 | C:T |
| rs2486483 | 59673006 | 0.192 | A:G |
| rs1988577 | 59673156 | 0.192 | T:G |
| rs1867572 | 59708946 | 0.192 | C:A |
| rs2590366 | 59709299 | 0.308 | C:A |
| rs2790182 | 59709883 | 0.192 | G:A |
| rs1416764 | 59710357 | 0.192 | A:G |
| rs2590367 | 59711580 | 0.192 | C:T |
| rs16912053 | 59711670 | 0.017 | A:G |
| rs2790188 | 59712192 | 0.192 | G:A |
| rs2790189 | 59712582 | 0.192 | A:G |
| rs11812882 | 59712631 | 0.042 | T:C |
| rs1867573 | 59712978 | 0.192 | A:G |
| rs2590368 | 59713488 | 0.192 | C:T |
| rs1550773 | 59714447 | 0.192 | T:A |
| rs2790190 | 59715034 | 0.092 | A:G |
| rs2486486 | 59716660 | 0.192 | G:A |
| rs2590370 | 59719026 | 0.192 | G:A |
| rs16912085 | 59719530 | 0.042 | G:A |
| rs12241783 | 59719796 | 0.058 | G:T |
| rs16912088 | 59719824 | 0.067 | G:T |
| rs17620218 | 59720459 | 0.150 | A:G |
| rs1856565 | 59721905 | 0.008 | A:G |
| rs16912090 | 59721979 | 0.067 | A:G |
| rs11006105 | 59722207 | 0.058 | C:T |
| rs12264744 | 59723005 | 0.058 | C:T |
| rs2590289 | 59729647 | 0.092 | T:C |
| rs2790196 | 59729969 | 0.092 | G:T |
| rs7078335 | 59729989 | 0.125 | G:A |
| rs965867 | 59731252 | 0.117 | A:G |

TABLE 060
Anchor Tag
Block 004
Chromosome 10
Rs4747053 (BP 71990162) LD block SNPs

| | | Minor | |
|---|---|---|---|
| | Base | Allele | |
| SNP ID (rs) | Position | Freq | Alleles |
| rs10762392 | 71989336 | 0.342 | T:C |
| rs16927685 | 71989866 | 0.175 | T:G |
| rs4747053 | 71990162 | 0.342 | G:C |
| rs2394730 | 71990510 | 0.442 | G:T |

TABLE 61
Anchor Tag
Block 005
Chromosome 10
Rs7905535 (BP 73200945) and Rs7076717 (BP 73235195)
LD block SNPs. These tags are in separate LD blocks.

| | | Minor | |
|---|---|---|---|
| | Base | Allele | |
| SNP ID (rs) | Position | Freq | Alleles |
| rs9415041 | 73194190 | 0.383 | A:G |
| rs9415996 | 73194374 | 0.333 | G:A |
| rs7919533 | 73195531 | 0.442 | T:C |
| rs2121538 | 73196208 | 0.383 | A:G |
| rs1867982 | 73197053 | 0.092 | G:A |
| rs7905535 | 73200945 | 0.367 | G:A |
| rs10762477 | 73201075 | 0.175 | A:G |
| rs12569990 | 73204826 | 0.192 | C:G |
| rs10466026 | 73220975 | 0.367 | G:A |
| rs10823847 | 73221264 | 0.358 | T:C |
| rs4747192 | 73225932 | 0.383 | T:C |
| rs4747194 | 73228892 | 0.367 | G:A |
| rs6480557 | 73231404 | 0.367 | G:C |
| rs2121534 | 73234459 | 0.233 | G:A |
| rs7076717 | 73235195 | 0.233 | A:G |
| rs4747197 | 73236209 | 0.367 | T:A |
| rs1992823 | 73236768 | 0.117 | G:A |
| rs4747199 | 73239324 | 0.358 | C:T |
| rs2394840 | 73243371 | 0.117 | G:T |
| rs2290022 | 73245245 | 0.117 | A:G |
| rs1054635 | 73245551 | 0.117 | G:A |
| rs9415053 | 73245916 | 0.383 | G:C |
| rs4747202 | 73247273 | 0.117 | G:A |
| rs885828 | 73248509 | 0.367 | G:A |
| rs4747203 | 73248888 | 0.367 | T:C |
| rs11000016 | 73249223 | 0.242 | C:T |
| rs12769743 | 73249793 | 0.033 | A:T |
| rs2070968 | 73251566 | 0.250 | A:T |
| rs9415055 | 73252040 | 0.033 | G:A |
| rs2249814 | 73253526 | 0.233 | C:G |
| rs11000017 | 73254251 | 0.217 | G:C |
| rs1015193 | 73254432 | 0.392 | T:C |
| rs2121535 | 73254479 | 0.242 | C:A |
| rs2854992 | 73256591 | 0.242 | G:A |
| rs231890 | 2732635 | 0.142 | A:G |

TABLE 62
Anchor Tag
Block 001
Chromosome 11
Rs2075870 (BP 2746595) LD block SNPs

| | | Minor | |
|---|---|---|---|
| | Base | Allele | |
| SNP ID (rs) | Position | Freq | Alleles |
| rs7948952 | 2732816 | 0.125 | G:A |
| rs11023996 | 2733024 | 0.167 | A:G |
| rs231889 | 2733768 | 0.133 | T:C |
| rs11023999 | 2734659 | 0.325 | G:A |
| rs6578296 | 2736546 | 0.142 | T:C |
| rs6578297 | 2736929 | 0.142 | T:C |
| rs179785 | 2738095 | 0.375 | A:G |
| rs463535 | 2738246 | 0.125 | C:T |
| rs548566 | 2739224 | 0.142 | T:C |
| rs231875 | 2741407 | 0.125 | A:G |
| rs11024017 | 2741721 | 0.200 | A:G |
| rs4430486 | 2741967 | 0.200 | C:G |
| rs231873 | 2742118 | 0.142 | C:G |
| rs4255520 | 2742199 | 0.200 | A:G |
| rs151288 | 2742878 | 0.383 | G:A |
| rs12795571 | 2745532 | 0.025 | G:A |
| rs63934 | 2745638 | 0.142 | A:G |
| rs163160 | 2746531 | 0.125 | A:G |
| rs11024034 | 2746739 | 0.150 | T:C |
| rs163158 | 2747312 | 0.117 | C:A |

TABLE 63
Anchor Tag
Block 002
Chromosome 11
Rs12223197 (BP 19063319) and Rs2089051 (BP 19070682)
LD block SNPs. These tags are in separate LD blocks.

| | | Minor | |
|---|---|---|---|
| | Base | Allele | |
| SNP ID (rs) | Position | Freq | Alleles |
| rs16936091 | 19051299 | 0.083 | A:G |
| rs9633837 | 19051866 | 0.083 | C:T |
| rs905552 | 19052735 | 0.275 | T:C |
| rs905551 | 19052875 | 0.150 | C:G |
| rs1011179 | 19053875 | 0.267 | G:C |
| rs2062819 | 19054261 | 0.150 | A:G |
| rs16936098 | 19054488 | 0.117 | T:G |
| rs4756986 | 19056814 | 0.075 | T:C |
| rs10741758 | 19057054 | 0.108 | C:T |
| rs10766526 | 19057129 | 0.108 | A:G |
| rs10766527 | 19057312 | 0.108 | A:G |
| rs868337 | 19058067 | 0.075 | T:C |
| rs3893309 | 19058481 | 0.075 | G:A |
| rs10833055 | 19058867 | 0.033 | C:T |
| rs12802650 | 19059343 | 0.283 | T:C |
| rs16936105 | 19059496 | 0.075 | G:A |
| rs10833056 | 19061908 | 0.158 | T:C |
| rs11024979 | 19062621 | 0.108 | C:T |
| rs11024980 | 19062843 | 0.108 | A:G |
| rs11024981 | 19062858 | 0.108 | T:C |
| rs12223197 | 19063319 | 0.108 | G:A |
| rs16936113 | 19064719 | 0.033 | C:T |
| rs16936115 | 19065259 | 0.033 | T:C |
| rs11024984 | 19065546 | 0.108 | G:C |
| rs7125292 | 19065817 | 0.092 | A:G |
| rs7125667 | 19066084 | 0.075 | A:G |
| rs2089051 | 19070682 | 0.167 | G:A |
| rs11024992 | 19070777 | 0.100 | C:T |
| rs11024993 | 19071489 | 0.100 | T:A |
| rs11024994 | 19071718 | 0.100 | G:C |
| rs11024995 | 19071755 | 0.100 | C:T |
| rs11024996 | 19071810 | 0.100 | C:G |
| rs11024999 | 19071984 | 0.083 | G:T |
| rs16936140 | 19072642 | 0.100 | G:A |
| rs11025018 | 19093482 | 0.175 | C:A |
| rs10833063 | 19094149 | 0.175 | A:G |
| rs10444252 | 19073229 | 0.100 | C:T |
| rs11025000 | 19074354 | 0.067 | C:T |
| rs10833059 | 19074415 | 0.167 | T:C |
| rs11025001 | 19074893 | 0.067 | T:C |
| rs9299952 | 19075037 | 0.100 | G:A |
| rs4757753 | 19075279 | 0.067 | T:C |
| rs11025002 | 19075351 | 0.100 | T:A |
| rs1125217 | 19076735 | 0.267 | C:G |
| rs1962511 | 19077252 | 0.067 | C:T |
| rs11025004 | 19077402 | 0.100 | T:C |
| rs11025006 | 19077634 | 0.092 | C:G |
| rs11025007 | 19077807 | 0.075 | G:A |
| rs11025008 | 19077899 | 0.092 | A:G |
| rs7945401 | 19078036 | 0.258 | G:A |
| rs12223284 | 19079166 | 0.075 | T:G |
| rs11606080 | 19081419 | 0.158 | A:G |
| rs10833061 | 19083434 | 0.150 | G:A |
| rs10833062 | 19084628 | 0.175 | C:T |
| rs7945127 | 19084800 | 0.075 | G:A |
| rs11025012 | 19085518 | 0.067 | C:G |
| rs11025013 | 19087434 | 0.167 | C:T |
| rs4756989 | 19087569 | 0.167 | A:T |
| rs4757754 | 19088777 | 0.183 | T:C |
| rs4757755 | 19088864 | 0.183 | G:A |
| rs1875724 | 19088955 | 0.092 | C:T |
| rs11025014 | 19089452 | 0.183 | C:T |
| rs11606222 | 19089778 | 0.183 | T:C |
| rs11025015 | 19089988 | 0.017 | G:C |
| rs7123033 | 19090293 | 0.183 | G:C |
| rs11608243 | 19090485 | 0.183 | A:G |
| rs7934179 | 19090619 | 0.075 | T:A |
| rs16936167 | 19091398 | 0.192 | A:C |
| rs924751 | 19092214 | 0.108 | C:T |
| rs4267049 | 19092380 | 0.017 | G:A |
| rs11025020 | 19094200 | 0.167 | C:T |
| rs1546329 | 19094523 | 0.267 | A:C |

TABLE 64
Anchor Tag
Block 003
Chromosome 11
Rs1262220 (BP 95025261), Rs10831399 (BP 95030832), Rs1939876
(BP 95042333), and Rs7932590 (BP 95048677) LD block SNPs

| | | Minor | |
|---|---|---|---|
| | Base | Allele | |
| SNP ID (rs) | Position | Freq | Alleles |
| rs1405732 | 95018143 | 0.183 | G:A |
| rs6483450 | 95018885 | 0.025 | A:G |
| rs1255523 | 95019155 | 0.458 | A:G |
| rs16922328 | 95020816 | 0.183 | C:T |
| rs16922332 | 95022862 | 0.008 | C:T |
| rs1150347 | 95023218 | 0.125 | G:A |
| rs1255524 | 95024706 | 0.375 | G:T |
| rs1262220 | 95025261 | 0.392 | C:T |
| rs17799724 | 95026826 | 0.142 | A:G |
| rs7103583 | 95029039 | 0.367 | A:G |
| rs7942321 | 95029409 | 0.367 | G:A |
| rs7927909 | 95029525 | 0.375 | T:C |
| rs7945592 | 95029586 | 0.367 | A:T |
| rs7942546 | 95029600 | 0.367 | T:A |
| rs2141165 | 95030328 | 0.367 | A:G |
| rs10831396 | 95030503 | 0.367 | T:A |
| rs10831399 | 95030832 | 0.367 | A:G |
| rs11021269 | 95031262 | 0.375 | G:A |
| rs7102632 | 95031870 | 0.367 | C:T |
| rs10765757 | 95032909 | 0.350 | C:T |
| rs12272718 | 95033681 | 0.367 | T:G |
| rs12294525 | 95033816 | 0.367 | A:T |
| rs10765758 | 95035255 | 0.367 | G:A |
| rs7926745 | 95036493 | 0.367 | T:G |
| rs11021274 | 95037054 | 0.367 | C:T |
| rs10831407 | 95037269 | 0.367 | C:T |
| rs10437578 | 95037914 | 0.392 | A:G |
| rs10437579 | 95038000 | 0.358 | G:T |
| rs10741515 | 95038129 | 0.375 | A:G |
| rs10741516 | 95038141 | 0.367 | C:A |
| rs9651561 | 95038990 | 0.392 | A:G |
| rs941292 | 95040116 | 0.358 | T:A |
| rs9651554 | 95040500 | 0.392 | A:G |
| rs873219 | 95040782 | 0.358 | A:G |
| rs873603 | 95041658 | 0.392 | G:T |
| rs1939876 | 95042333 | 0.367 | G:A |
| rs10831409 | 95046267 | 0.358 | A:C |
| rs7946150 | 95046985 | 0.358 | T:G |
| rs7939277 | 95047191 | 0.383 | C:T |
| rs7949315 | 95047252 | 0.367 | A:G |
| rs7939398 | 95047274 | 0.392 | C:T |
| rs7932590 | 95048677 | 0.367 | T:G |
| rs6483452 | 95049274 | 0.400 | T:A |
| rs7931238 | 95051248 | 0.425 | A:G |
| rs7944958 | 95051289 | 0.425 | C:T |
| rs765722 | 95052766 | 0.425 | T:A |
| rs998938 | 95054232 | 0.242 | G:A |

TABLE 65
Anchor Tag
Block 004
Chromosome 11
Rs473573 (BP 127633623) and Rs7950582 (BP 127684286)
LD block SNPs. These tags are in separate LD blocks.

| | | Minor | |
|---|---|---|---|
| | Base | Allele | |
| SNP ID (rs) | Position | Freq | Alleles |
| rs528684 | 127629301 | 0.05 | A:C |
| rs508974 | 127629837 | 0.05 | A:G |
| rs471893 | 127630829 | 0.05 | C:T |
| rs535908 | 127631502 | 0.258 | C:T |
| rs12805818 | 127631683 | 0.058 | C:G |
| rs6590316 | 127632018 | 0.058 | G:A |
| rs473573 | 127633623 | 0.058 | G:C |
| rs11221227 | 127634397 | 0.067 | T:C |
| rs553763 | 127634678 | 0.05 | A:G |
| rs12363345 | 127635869 | 0.017 | C:A |
| rs498282 | 127636048 | 0.05 | A:G |
| rs1940378 | 127636223 | 0.175 | G:A |
| rs580446 | 127636888 | 0.05 | A:C |
| rs475360 | 127637071 | 0.05 | G:T |
| rs530178 | 127637217 | 0.05 | A:G |
| rs10893833 | 127638633 | 0.017 | G:A |
| rs489719 | 127638723 | 0.05 | C:G |
| rs498826 | 127639686 | 0.05 | C:T |
| rs11221228 | 127640053 | 0.017 | T:C |
| rs11602305 | 127640120 | 0.025 | A:G |
| rs565536 | 127641599 | 0.358 | G:A |
| rs10893834 | 127641670 | 0.242 | T:A |
| rs541057 | 127641924 | 0.017 | C:T |
| rs542082 | 127642411 | 0.042 | T:C |
| rs540260 | 127642610 | 0.05 | T:C |
| rs10893835 | 127643114 | 0.067 | C:G |
| rs10893837 | 127643914 | 0.067 | A:C |
| rs1216063 | 127644754 | 0.358 | T:A |
| rs531538 | 127646377 | 0.258 | A:G |
| rs12280315 | 127646746 | 0.017 | A:G |
| rs10893840 | 127647302 | 0.067 | A:G |
| rs10893841 | 127648067 | 0.017 | T:C |
| rs554906 | 127648314 | 0.05 | G:C |
| rs518199 | 127649277 | 0.358 | T:G |
| rs11221232 | 127650074 | 0.067 | C:A |
| rs505875 | 127650189 | 0.05 | C:T |
| rs485119 | 127650592 | 0.05 | A:C |
| rs11221247 | 127682538 | 0.442 | G:A |
| rs12226163 | 127682722 | 0.058 | T:A |
| rs1522434 | 127683003 | 0.05 | C:G |
| rs7950582 | 127684286 | 0.05 | G:A |
| rs1522435 | 127684677 | 0.442 | G:A |
| rs12360982 | 127686020 | 0.017 | T:G |
| rs4144897 | 127686401 | 0.05 | G:A |
| rs11823333 | 127686911 | 0.267 | A:G |

TABLE 66
Anchor Tag
Block 001
Chromosome 12
Rs622513 (BP 3941499) and Rs609018 (BP 3942162) LD block SNPs

| | | Minor | |
|---|---|---|---|
| | Base | Allele | |
| SNP ID (rs) | Position | Freq | Alleles |
| rs622513 | 3941499 | 0.408 | A:G |
| rs2532524 | 3941530 | 0.083 | C:T |
| rs609018 | 3942162 | 0.375 | T:G |

TABLE 67
Anchor Tag
Block 002
Chromosome 12
Rs11612899 (BP 5483797) LD block SNPs

| | | Minor | |
|---|---|---|---|
| | Base | Allele | |
| SNP ID (rs) | Position | Freq | Alleles |
| rs4075788 | 5480873 | 0.233 | A:G |
| rs11063717 | 5481108 | 0.008 | G:A |
| rs10774334 | 5482060 | 0.100 | C:T |
| rs11614714 | 5482357 | 0.025 | T:A |
| rs10849279 | 5483255 | 0.358 | G:A |
| rs11612899 | 5483797 | 0.250 | C:T |
| rs11063718 | 5484705 | 0.025 | G:A |
| rs11063721 | 5487569 | 0.400 | C:T |
| rs7133664 | 5488193 | 0.108 | A:G |
| rs11063722 | 5488521 | 0.008 | A:G |
| rs11063723 | 5489075 | 0.408 | T:C |

TABLE 68
Anchor Tag
Block 003
Chromosome 12
Rs10747645 (BP 51416611), Rs7398785 (BP 51422165), Rs7306051 (BP 51439097),
and Rs17738862 (BP 51469295) LD block SNPs.
These tags are in separate LD blocks.

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs10876331 | 51414298 | 0.242 | T:G | rs10876344 | 51465749 | 0.017 | G:A |
| rs10876333 | 51414711 | 0.050 | C:T | rs4600258 | 51434130 | 0.050 | T:C |
| rs12820919 | 51415425 | 0.025 | A:C | rs4761959 | 51434533 | 0.225 | T:A |
| rs17118511 | 51416552 | 0.008 | C:T | rs11170266 | 51434688 | 0.017 | G:A |
| rs10747645 | 51416611 | 0.242 | A:G | rs10128773 | 51435197 | 0.225 | T:A |
| rs1473860 | 51418734 | 0.242 | A:G | rs1521348 | 51435450 | 0.150 | G:A |
| rs4564385 | 51419279 | 0.200 | C:T | rs17118733 | 51435916 | 0.017 | G:T |
| rs11170256 | 51419584 | 0.017 | G:C | rs1402592 | 51436257 | 0.158 | C:G |
| rs10747650 | 51419741 | 0.250 | C:T | rs10506312 | 51436632 | 0.100 | G:T |
| rs10747651 | 51420691 | 0.242 | C:T | rs10876335 | 51436807 | 0.233 | G:C |
| rs7969242 | 51420962 | 0.242 | C:A | rs10876336 | 51436817 | 0.225 | G:T |
| rs7956525 | 51421288 | 0.242 | C:T | rs10876337 | 51436833 | 0.225 | G:A |
| rs7398785 | 51422165 | 0.242 | A:G | rs10876338 | 51436863 | 0.225 | G:A |
| rs7398818 | 51422809 | 0.042 | G:A | rs6580900 | 51438297 | 0.042 | A:G |
| rs7974402 | 51422817 | 0.192 | C:T | rs1402594 | 51438591 | 0.042 | G:C |
| rs12297836 | 51423198 | 0.008 | C:T | rs17118767 | 51439068 | 0.075 | G:A |
| rs1521338 | 51424897 | 0.108 | G:A | rs7306051 | 51439097 | 0.225 | T:C |
| rs11170263 | 51425669 | 0.017 | A:G | rs11170268 | 51439395 | 0.017 | G:A |
| rs17737740 | 51426635 | 0.158 | G:A | rs2037432 | 51439547 | 0.225 | A:G |
| rs17118620 | 51426650 | 0.017 | G:A | rs1554448 | 51440672 | 0.042 | C:T |
| rs7304418 | 51426930 | 0.242 | A:C | rs1818762 | 51441336 | 0.042 | G:A |
| rs884337 | 51428342 | 0.242 | A:C | rs1818763 | 51441612 | 0.042 | C:T |
| rs673077 | 51428489 | 0.042 | T:C | rs7979912 | 51441864 | 0.042 | T:C |
| rs17118672 | 51429458 | 0.008 | T:C | rs4499050 | 51442331 | 0.150 | G:T |
| rs688435 | 51429557 | 0.008 | A:G | rs1533753 | 51443028 | 0.217 | T:C |
| rs17118679 | 51430140 | 0.008 | C:T | rs6580901 | 51443337 | 0.158 | G:A |
| rs17118681 | 51430184 | 0.017 | A:T | rs1533755 | 51443376 | 0.042 | G:A |
| rs17118684 | 51430272 | 0.017 | A:T | rs1357094 | 51443404 | 0.017 | G:A |
| rs12815838 | 51430659 | 0.008 | G:A | rs1879559 | 51443793 | 0.158 | C:T |
| rs4761958 | 51431003 | 0.050 | T:C | rs7313996 | 51443998 | 0.008 | G:A |
| rs1521346 | 51431986 | 0.050 | C:T | rs1521350 | 51444127 | 0.042 | A:G |
| rs11170264 | 51432916 | 0.017 | T:G | rs17118856 | 51444235 | 0.158 | T:C |
| rs7488334 | 51432999 | 0.158 | G:T | rs12829541 | 51444732 | 0.008 | G:A |
| rs11170265 | 51433496 | 0.050 | C:A | rs950305 | 51445535 | 0.008 | A:G |
| rs7954921 | 51445732 | 0.158 | G:A | rs10506311 | 51445539 | 0.150 | C:T |
| | | | | rs11170276 | 51465940 | 0.175 | C:T |
| rs3923832 | 51457993 | 0.358 | A:G | rs17119082 | 51466819 | 0.050 | G:A |
| rs6580908 | 51458209 | 0.183 | G:T | rs17738791 | 51466949 | 0.025 | G:A |
| rs6421174 | 51458700 | 0.017 | T:A | rs17738815 | 51467309 | 0.025 | G:A |
| rs7397684 | 51459340 | 0.275 | A:G | rs4919753 | 51467737 | 0.225 | T:G |
| rs12578793 | 51459380 | 0.142 | C:T | rs10876346 | 51467906 | 0.217 | G:A |
| rs12829797 | 51460534 | 0.175 | G:A | rs1552756 | 51468260 | 0.225 | G:A |
| rs11612631 | 51461427 | 0.175 | T:C | rs4267154 | 51468382 | 0.367 | C:T |
| rs4919756 | 51463589 | 0.217 | G:A | rs17738862 | 51469295 | 0.025 | C:T |
| rs17119043 | 51463627 | 0.050 | C:T | rs11170279 | 51470758 | 0.183 | A:G |
| rs7309662 | 51464071 | 0.358 | C:T | | | | |

TABLE 69
Anchor Tag
Block 004
Chromosome 12
Rs10877926 (BP 61580249) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

| | | | |
|---|---|---|---|
| rs10877926 | 61580249 | 0.208 | C:T |

TABLE 70
Anchor Tag
Block 005
Chromosome 12
Rs10776994 (BP 76076736) LD block SNPs

| | | Minor | |
|---|---|---|---|
| | Base | Allele | |
| SNP ID (rs) | Position | Freq | Alleles |
| rs771620 | 76068578 | 0.225 | T:C |
| rs12317454 | 76076675 | 0.042 | T:C |
| rs10776994 | 76076736 | 0.275 | T:C |
| rs12819839 | 76077504 | 0.158 | T:C |
| rs12821026 | 76078079 | 0.158 | T:C |
| rs11104042 | 76078093 | 0.192 | G:A |
| rs11104044 | 76078152 | 0.033 | G:T |
| rs12319645 | 76078210 | 0.042 | T:C |
| rs1851095 | 76079570 | 0.225 | G:A |
| rs2133314 | 76080348 | 0.042 | A:G |
| rs11104055 | 76080832 | 0.033 | A:G |
| rs11104056 | 76080880 | 0.025 | G:A |
| rs10506734 | 76081026 | 0.033 | A:G |
| rs12824568 | 76081253 | 0.158 | A:C |
| rs771641 | 76081373 | 0.433 | G:A |
| rs17043087 | 76081488 | 0.042 | A:G |
| rs12829904 | 76081542 | 0.158 | A:G |
| rs12830758 | 76081903 | 0.158 | A:G |
| rs17043088 | 76082055 | 0.042 | T:C |
| rs771640 | 76082098 | 0.433 | C:A |
| rs771639 | 76082277 | 0.225 | C:G |
| rs771638 | 76082938 | 0.233 | T:C |
| rs771633 | 76086380 | 0.275 | T:A |
| rs1915356 | 76089124 | 0.275 | G:A |
| rs10858504 | 76093884 | 0.275 | T:G |
| rs884221 | 76096201 | 0.225 | T:A |
| rs869117 | 76097269 | 0.275 | A:T |
| rs10745436 | 76098327 | 0.225 | G:C |

TABLE 71
Anchor Tag
Block 006
Chromosome 12
Rs835481 (BP 103551381) and Rs1344677 (BP 103553434)
LD block SNPs

| | | Minor | |
|---|---|---|---|
| | Base | Allele | |
| SNP ID (rs) | Position | Freq | Alleles |
| rs2694414 | 103544131 | 0.292 | G:A |
| rs2696002 | 103544714 | 0.142 | A:G |
| rs2696000 | 103546290 | 0.158 | A:G |
| rs2695999 | 103546390 | 0.142 | A:G |
| rs10507181 | 103546419 | 0.492 | T:A |
| rs2695998 | 103546756 | 0.142 | A:G |
| rs2694410 | 103547192 | 0.083 | G:C |
| rs2695997 | 103547720 | 0.133 | A:G |
| rs2694409 | 103547800 | 0.142 | C:T |
| rs10861261 | 103548190 | 0.150 | C:G |
| rs1863879 | 103548717 | 0.142 | G:A |
| rs11112143 | 103549155 | 0.150 | C:T |
| rs2695996 | 103549358 | 0.275 | G:A |
| rs12300833 | 103549685 | 0.033 | A:T |
| rs12582801 | 103549978 | 0.117 | T:C |
| rs7974223 | 103550927 | 0.417 | G:A |
| rs835481 | 103551381 | 0.275 | T:C |
| rs703659 | 103552046 | 0.008 | A:G |
| rs703660 | 103552919 | 0.025 | C:T |
| rs1344677 | 103553434 | 0.417 | C:T |
| rs11112146 | 103554236 | 0.317 | C:T |
| rs12300231 | 103554717 | 0.417 | C:A |
| rs7962537 | 103555252 | 0.417 | A:G |
| rs7965164 | 103555280 | 0.317 | A:G |

TABLE 72
Anchor Tag
Block 007
Chromosome 12
Rs11058789 (BP 121176298) LD block SNPs

| | | Minor | |
|---|---|---|---|
| | Base | Allele | |
| SNP ID (rs) | Position | Freq | Alleles |
| rs7964127 | 121165445 | 0.400 | C:T |
| rs11611988 | 121171615 | 0.450 | C:T |
| rs11058587 | 121171751 | 0.450 | A:G |
| rs11834745 | 121174055 | 0.008 | C:A |
| rs7296522 | 121176055 | 0.017 | A:G |
| rs11058785 | 121176135 | 0.133 | G:A |
| rs11058789 | 121176298 | 0.450 | C:T |
| rs7310689 | 121176504 | 0.133 | G:A |
| rs4372487 | 121177232 | 0.450 | A:G |
| rs4758680 | 121180232 | 0.375 | G:T |
| rs7977932 | 121182263 | 0.158 | C:G |
| rs10847385 | 121184230 | 0.167 | A:C |
| rs7972875 | 121187655 | 0.458 | C:T |
| rs7972979 | 121187736 | 0.458 | C:T |
| rs11059505 | 121190165 | 0.458 | C:G |
| rs11059508 | 121190202 | 0.167 | C:T |
| rs2292443 | 121192971 | 0.375 | C:T |

TABLE 73
Anchor Tag
Block 001
Chromosome 13
Rs12871430 (BP 21591188) LD block SNPs

| | | Minor | |
|---|---|---|---|
| | Base | Allele | |
| SNP ID (rs) | Position | Freq | Alleles |
| rs9552597 | 21588127 | 0.217 | G:A |
| rs1570459 | 21589988 | 0.017 | T:G |
| rs9316766 | 21590292 | 0.225 | A:G |
| rs17076054 | 21590432 | 0.042 | C:A |
| rs1125958 | 21590850 | 0.017 | A:G |
| rs1125957 | 21591171 | 0.017 | C:A |
| rs12871430 | 21591188 | 0.225 | C:T |
| rs7139808 | 21591228 | 0.017 | T:C |
| rs17377549 | 21591910 | 0.067 | A:G |
| rs11838655 | 21593311 | 0.275 | T:C |

TABLE 74
Anchor Tag
Block 002
Chromosome 13
Rs636519 (BP 29553181), Rs501378 (BP 29553961),
and Rs478410 (BP 29554081) LD block SNPs

| | | Minor | |
|---|---|---|---|
| | Base | Allele | |
| SNP ID (rs) | Position | Freq | Alleles |
| rs486253 | 29551802 | 0.075 | C:G |
| rs621099 | 29551981 | 0.075 | A:C |
| rs636519 | 29553181 | 0.175 | T:A |
| rs501378 | 29553961 | 0.175 | C:T |
| rs478410 | 29554081 | 0.175 | A:G |
| rs574428 | 29558554 | 0.083 | A:G |
| rs9551822 | 29559096 | 0.050 | A:T |
| rs846721 | 29559593 | 0.183 | T:C |
| rs9551823 | 29560049 | 0.050 | T:C |
| rs9550523 | 29560249 | 0.050 | C:T |
| rs846722 | 29561698 | 0.183 | T:C |
| rs676495 | 29561754 | 0.192 | C:T |
| rs9506261 | 29562703 | 0.200 | G:A |
| rs584444 | 29562982 | 0.133 | A:C |
| rs504508 | 29562999 | 0.133 | A:G |
| rs500717 | 29563459 | 0.133 | G:A |

TABLE 75
Anchor Tag
Block 003
Chromosome 13
Rs2769330 (BP 35194460) and Rs6563133 (BP 35216869)
LD block SNPs. These tags are in separate LD blocks.

| | | Minor | |
|---|---|---|---|
| | Base | Allele | |
| SNP ID (rs) | Position | Freq | Alleles |
| rs9574435 | 35192750 | 0.183 | A:T |
| rs2147674 | 35193038 | 0.025 | A:C |
| rs2769330 | 35194460 | 0.392 | G:A |
| rs9574437 | 35195517 | 0.067 | T:G |
| rs7318433 | 35196299 | 0.183 | G:A |
| rs9574438 | 35196390 | 0.225 | A:C |
| rs275972 | 35197154 | 0.408 | G:T |
| rs17177245 | 35197388 | 0.025 | G:A |
| rs1998697 | 35198045 | 0.200 | G:A |
| rs9574443 | 35198318 | 0.183 | G:T |
| rs2477458 | 35199684 | 0.433 | A:G |
| rs9545157 | 35200201 | 0.208 | T:G |
| rs17052708 | 35201712 | 0.200 | C:T |
| rs9545161 | 35202717 | 0.208 | A:G |
| rs9545162 | 35202734 | 0.433 | A:C |
| rs9574453 | 35204060 | 0.183 | T:C |
| rs17330812 | 35205417 | 0.200 | C:T |
| rs9530945 | 35206757 | 0.158 | C:T |
| rs11618526 | 35207492 | 0.183 | C:A |
| rs4943326 | 35207538 | 0.200 | C:T |
| rs4941815 | 35207635 | 0.408 | T:C |
| rs4635204 | 35208309 | 0.208 | A:C |
| rs9593411 | 35208416 | 0.433 | G:T |
| rs7323895 | 35208925 | 0.408 | A:G |
| rs4943327 | 35209062 | 0.408 | C:T |
| rs9574471 | 35214940 | 0.200 | T:C |
| rs9574474 | 35215379 | 0.200 | T:C |
| rs7317552 | 35216804 | 0.192 | C:T |
| rs6563133 | 35216869 | 0.392 | G:A |
| rs9574483 | 35217579 | 0.200 | C:T |
| rs7330084 | 35218565 | 0.050 | C:T |
| rs7331603 | 35218987 | 0.042 | A:C |
| rs9545221 | 35221749 | 0.192 | G:T |
| rs6563142 | 35224783 | 0.167 | A:G |
| rs7328278 | 35249766 | 0.017 | A:C |
| rs9545328 | 35250139 | 0.175 | C:T |
| rs7992754 | 35250224 | 0.217 | A:G |
| rs7999483 | 35251437 | 0.133 | A:C |
| rs2068335 | 35252243 | 0.200 | G:A |
| rs4614590 | 35252658 | 0.042 | C:T |
| rs9545332 | 35253680 | 0.217 | C:T |
| rs2874868 | 35254679 | 0.317 | T:G |
| rs6563147 | 35227029 | 0.192 | C:T |
| rs9545255 | 35227287 | 0.433 | C:G |
| rs7992892 | 35228674 | 0.192 | A:C |
| rs7997939 | 35228761 | 0.192 | T:C |
| rs4941816 | 35231519 | 0.233 | A:G |
| rs9574508 | 35232125 | 0.192 | A:G |
| rs9574510 | 35232809 | 0.183 | G:C |
| rs9574511 | 35232881 | 0.183 | G:A |
| rs9574512 | 35232954 | 0.183 | G:A |
| rs9574513 | 35233136 | 0.183 | C:T |
| rs9574515 | 35233203 | 0.183 | A:C |
| rs9574516 | 35233335 | 0.183 | C:T |
| rs9565538 | 35236045 | 0.183 | C:T |
| rs9574527 | 35236151 | 0.183 | A:T |
| rs9574528 | 35236195 | 0.183 | C:T |
| rs9574529 | 35236432 | 0.183 | C:G |
| rs9574530 | 35236459 | 0.183 | G:C |
| rs9565540 | 35236503 | 0.183 | C:T |
| rs872060 | 35236917 | 0.183 | T:C |
| rs1886549 | 35237275 | 0.183 | G:C |
| rs11843245 | 35238137 | 0.142 | C:G |
| rs9545294 | 35238767 | 0.217 | C:T |
| rs7320361 | 35239322 | 0.042 | T:C |
| rs11843701 | 35239438 | 0.175 | G:A |
| rs7318335 | 35239548 | 0.042 | G:A |
| rs9545297 | 35239668 | 0.175 | T:G |
| rs12865680 | 35241088 | 0.025 | G:A |
| rs7981115 | 35242931 | 0.042 | C:T |
| rs1045696 | 35243816 | 0.158 | C:T |
| rs3764062 | 35244211 | 0.317 | T:C |
| rs17052784 | 35244962 | 0.017 | T:C |
| rs9574551 | 35245006 | 0.467 | C:T |
| rs9645963 | 35247503 | 0.042 | A:G |
| rs9574568 | 35249355 | 0.175 | C:T |
| rs4591003 | 35256050 | 0.467 | C:T |
| rs9574593 | 35257732 | 0.158 | A:C |
| rs12430800 | 35257974 | 0.200 | C:T |
| rs6563159 | 35258574 | 0.200 | C:A |
| rs9646082 | 35260830 | 0.133 | T:G |
| rs7326177 | 35261908 | 0.158 | A:T |
| rs7995214 | 35262756 | 0.317 | C:T |

TABLE 76
Anchor Tag
Block 004
Chromosome 13
Rs17538591 (BP 42760855) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

| | | | |
|---|---|---|---|
| rs17538591 | 42760855 | 0.167 | T:C |

TABLE 77
Anchor Tag
Block 005
Chromosome 13
Rs2325197 (BP 68655746) LD blocks SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

| | | | |
|---|---|---|---|
| rs2325197 | 68655746 | 0.150 | C:G |

TABLE 78
Anchor Tag
Block 006
Chromosome 13
Rs1512762 (BP 82708206) LD block SNPs

| | | Minor | |
|---|---|---|---|
| | Base | Allele | |
| SNP ID (rs) | Position | Freq | Alleles |
| rs1512757 | 82703400 | 0.450 | C:A |
| rs1935712 | 82704165 | 0.433 | T:C |
| rs7987748 | 82704429 | 0.042 | C:A |
| rs1858450 | 82705571 | 0.375 | A:G |
| rs1858451 | 82705614 | 0.342 | C:A |
| rs1512759 | 82705915 | 0.400 | T:G |
| rs1849391 | 82706006 | 0.342 | C:T |
| rs2137436 | 82706285 | 0.342 | T:A |
| rs9546333 | 82706588 | 0.425 | A:G |
| rs1858452 | 82706997 | 0.342 | C:T |
| rs2669191 | 82707358 | 0.333 | T:C |
| rs2669192 | 82707692 | 0.467 | G:A |
| rs12865675 | 82707700 | 0.375 | C:T |
| rs1512762 | 82708206 | 0.433 | A:T |
| rs2796871 | 82708549 | 0.400 | T:G |
| rs2796872 | 82708837 | 0.400 | T:A |
| rs2819229 | 82709037 | 0.433 | G:A |
| rs9602132 | 82709649 | 0.058 | C:A |
| rs2669193 | 82709938 | 0.400 | C:A |
| rs2669194 | 82711857 | 0.342 | A:G |
| rs2819231 | 82712083 | 0.442 | A:C |
| rs1512770 | 82715032 | 0.342 | G:A |
| rs1512771 | 82715131 | 0.433 | T:C |
| rs9546336 | 82715912 | 0.375 | T:A |
| rs978221 | 82718664 | 0.342 | G:T |

TABLE 79
Anchor Tag
Block 001
Chromosome 14
Rs7147585 (BP 27426001) LD block SNPs

| | | Minor | |
|---|---|---|---|
| | Base | Allele | |
| SNP ID (rs) | Position | Freq | Alleles |
| rs2333185 | 27387295 | 0.250 | A:G |
| rs7145469 | 27387322 | 0.200 | G:A |
| rs2333186 | 27387525 | 0.258 | G:A |
| rs2022835 | 27388352 | 0.200 | T:A |
| rs4127326 | 27394680 | 0.275 | A:G |
| rs8007727 | 27399213 | 0.267 | G:A |
| rs8007650 | 27399538 | 0.258 | T:C |
| rs10498297 | 27401752 | 0.458 | C:T |
| rs10498298 | 27401813 | 0.458 | T:C |
| rs2022836 | 27402804 | 0.400 | G:A |
| rs4143693 | 27403258 | 0.458 | A:G |
| rs12586282 | 27404365 | 0.458 | G:A |
| rs10130737 | 27404534 | 0.250 | T:C |
| rs10147655 | 27404898 | 0.400 | T:A |
| rs11626891 | 27405080 | 0.092 | A:C |
| rs1954596 | 27405193 | 0.450 | T:G |
| rs9972229 | 27405514 | 0.250 | G:A |
| rs12587753 | 27406001 | 0.458 | C:A |
| rs8005944 | 27407759 | 0.367 | G:T |
| rs8013650 | 27408556 | 0.325 | T:G |
| rs8012193 | 27408968 | 0.292 | G:A |
| rs7152910 | 27410676 | 0.292 | T:C |
| rs8010147 | 27413789 | 0.200 | G:A |
| rs2022813 | 27414900 | 0.258 | G:A |
| rs8008404 | 27416885 | 0.058 | C:T |
| rs11847425 | 27418799 | 0.250 | T:C |
| rs11626153 | 27422270 | 0.092 | A:G |
| rs4559841 | 27422655 | 0.275 | G:A |
| rs12147172 | 27422971 | 0.067 | G:A |
| rs2333189 | 27424100 | 0.100 | T:C |
| rs1954572 | 27424438 | 0.183 | T:C |
| rs8019309 | 27424594 | 0.017 | C:G |
| rs7147585 | 27426001 | 0.250 | G:A |

TABLE 80
Anchor Tag
Block 002
Chromosome 14
Rs8014810 (BP 35394781) LD block SNPs

| | | Minor | |
|---|---|---|---|
| | Base | Allele | |
| SNP ID (rs) | Position | Freq | Alleles |
| rs10149613 | 35362494 | 0.142 | A:G |
| rs4982310 | 35362682 | 0.008 | G:A |
| rs7144448 | 35366822 | 0.142 | T:G |
| rs10146494 | 35368683 | 0.125 | G:T |
| rs10134750 | 35369853 | 0.075 | A:G |
| rs10141289 | 35371747 | 0.125 | C:T |
| rs1888135 | 35373573 | 0.125 | C:G |
| rs2899840 | 35373604 | 0.125 | C:G |
| rs4981309 | 35374119 | 0.125 | T:G |
| rs4982314 | 35384218 | 0.125 | A:G |
| rs7140602 | 35384440 | 0.008 | G:C |
| rs8014810 | 35394781 | 0.142 | G:T |
| rs1958075 | 35397181 | 0.125 | T:G |
| rs2415306 | 35400967 | 0.142 | G:T |
| rs11624286 | 35401392 | 0.033 | C:A |
| rs12890607 | 35403781 | 0.375 | C:G |
| rs1958074 | 35405129 | 0.008 | A:G |
| rs4981314 | 35408241 | 0.142 | A:C |
| rs8003661 | 35408995 | 0.142 | A:G |
| rs2148410 | 35411654 | 0.142 | T:A |

TABLE 81
Anchor Tag
Block 003
Chromosome 14
rs17105308 (BP 36324888) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs2764962 | 36297971 | 0.467 | C:T | rs848113 | 36323927 | 0.142 | G:C |
| rs2253012 | 36298255 | 0.467 | C:T | rs17105308 | 36324888 | 0.233 | T:C |
| rs1749927 | 36299568 | 0.133 | G:T | rs17105311 | 36324910 | 0.233 | A:G |
| rs1749928 | 36300034 | 0.133 | A:G | rs848116 | 36326520 | 0.133 | G:C |
| rs1325535 | 36300212 | 0.133 | C:G | rs848117 | 36326610 | 0.117 | G:A |
| rs1325536 | 36300329 | 0.167 | C:T | rs17105314 | 36327422 | 0.233 | C:T |
| rs1325537 | 36300439 | 0.133 | C:T | rs1853812 | 36327543 | 0.142 | T:A |
| rs1749929 | 36300860 | 0.133 | T:C | rs2774036 | 36328455 | 0.375 | T:C |
| rs2774039 | 36301274 | 0.133 | A:C | rs848099 | 36331053 | 0.142 | C:A |
| rs2764953 | 36301654 | 0.133 | A:G | rs848097 | 36331233 | 0.375 | T:C |
| rs2774038 | 36301835 | 0.133 | A:T | rs848096 | 36331855 | 0.142 | T:A |
| rs17105233 | 36303643 | 0.233 | T:G | rs17105347 | 36335202 | 0.233 | T:C |
| rs17105237 | 36303889 | 0.167 | G:C | rs848094 | 36335453 | 0.142 | T:G |
| rs848110 | 36304213 | 0.133 | C:T | rs848093 | 36335481 | 0.142 | C:T |
| rs712324 | 36304444 | 0.133 | G:C | rs712332 | 36336400 | 0.167 | T:C |
| rs712325 | 36304714 | 0.467 | C:G | rs712333 | 36336561 | 0.142 | T:C |
| rs17105249 | 36304891 | 0.167 | C:G | rs1325528 | 36337811 | 0.233 | A:G |
| rs848108 | 36306880 | 0.133 | T:C | rs17105367 | 36338361 | 0.233 | A:G |
| rs17105268 | 36308326 | 0.167 | G:A | rs848092 | 36338548 | 0.308 | G:A |
| rs712326 | 36308576 | 0.467 | C:G | rs848091 | 36339103 | 0.467 | T:C |
| rs712327 | 36308756 | 0.467 | A:T | rs1594268 | 36339404 | 0.392 | T:C |
| rs730643 | 36308898 | 0.400 | G:A | rs1749930 | 36339567 | 0.392 | G:A |
| rs1806899 | 36308939 | 0.400 | G:A | rs1429847 | 36340197 | 0.200 | A:T |
| rs2415363 | 36309605 | 0.400 | A:C | rs1408965 | 36340385 | 0.392 | G:A |
| rs712328 | 36310276 | 0.133 | G:T | rs8008462 | 36341562 | 0.392 | A:T |
| rs712329 | 36310342 | 0.467 | A:G | rs1429849 | 36342755 | 0.225 | T:A |
| rs912814 | 36311812 | 0.400 | A:C | rs1367035 | 36343386 | 0.233 | G:A |
| rs17105301 | 36312837 | 0.167 | G:A | rs848090 | 36344416 | 0.142 | C:T |
| rs848119 | 36312889 | 0.133 | G:T | rs848089 | 36345429 | 0.450 | A:C |
| rs8004848 | 36314123 | 0.400 | C:A | rs848088 | 36346249 | 0.450 | C:T |
| rs848123 | 36315238 | 0.458 | A:G | rs1429845 | 36347066 | 0.175 | A:C |
| rs8022501 | 36317227 | 0.408 | G:A | rs848087 | 36347394 | 0.458 | G:A |
| rs8022435 | 36317400 | 0.400 | C:T | rs1749935 | 36347898 | 0.408 | C:T |
| rs848111 | 36323277 | 0.142 | T:G | rs7144852 | 36348073 | 0.408 | G:A |
| rs1325541 | 36348098 | 0.408 | G:T | rs2774035 | 36348898 | 0.458 | G:A |
| rs1652275 | 36348564 | 0.408 | G:C | rs1749936 | 36349080 | 0.400 | T:G |
| rs1631933 | 36348715 | 0.408 | A:G | | | | |

TABLE 82
Anchor Tag
Block 004
Chromosome 14
Rs8017546 (BP 57546972) LD block SNPs

| | | Minor | |
|---|---|---|---|
| | Base | Allele | |
| SNP ID (rs) | Position | Freq | Alleles |
| rs727203 | 57541452 | 0.408 | T:C |
| rs11158190 | 57542594 | 0.283 | A:C |
| rs11846103 | 57542886 | 0.017 | A:G |
| rs12889802 | 57543711 | 0.283 | C:T |
| rs17734691 | 57544103 | 0.267 | G:A |
| rs8004714 | 57544781 | 0.283 | G:A |
| rs11626754 | 57546143 | 0.283 | A:G |
| rs11622790 | 57546588 | 0.183 | T:C |
| rs8017546 | 57546972 | 0.283 | T:C |
| rs17094208 | 57547077 | 0.183 | T:C |
| rs8016701 | 57547287 | 0.308 | C:A |
| rs11629246 | 57547923 | 0.200 | G:A |
| rs3783687 | 57548694 | 0.017 | A:C |
| rs3783689 | 57548743 | 0.217 | T:A |
| rs3783690 | 57548908 | 0.200 | C:T |
| rs12884342 | 57549452 | 0.200 | T:C |
| rs11621819 | 57549606 | 0.200 | C:T |
| rs12892041 | 57550225 | 0.183 | T:C |
| rs4901832 | 57551094 | 0.075 | T:C |
| rs17734750 | 57551196 | 0.267 | T:C |
| rs17094212 | 57551545 | 0.200 | G:A |
| rs1955633 | 57552970 | 0.283 | G:T |
| rs1955634 | 57555964 | 0.267 | C:T |
| rs17093271 | 57556410 | 0.183 | A:G |
| rs9323323 | 57556664 | 0.300 | T:C |
| rs10136424 | 57556772 | 0.433 | G:T |
| rs1955637 | 57557126 | 0.267 | G:C |
| rs11621309 | 57559026 | 0.283 | A:T |
| rs10130984 | 57559046 | 0.267 | C:T |
| rs10143224 | 57559058 | 0.333 | T:C |
| rs1955639 | 57559419 | 0.300 | G:A |
| rs1955641 | 57560660 | 0.267 | A:G |
| rs12884758 | 57561655 | 0.017 | T:C |
| rs8014603 | 57562663 | 0.450 | T:C |
| rs12895361 | 57563099 | 0.017 | T:C |
| rs2223934 | 57563317 | 0.450 | C:A |
| rs11847792 | 57563697 | 0.275 | T:C |
| rs11620985 | 57563937 | 0.183 | G:T |
| rs17094248 | 57563968 | 0.450 | C:T |
| rs4901834 | 57564898 | 0.267 | G:C |
| rs17094254 | 57565004 | 0.200 | G:A |
| rs4901836 | 57565100 | 0.267 | T:C |
| rs4901839 | 57565270 | 0.267 | A:G |
| rs11629444 | 57567192 | 0.183 | T:G |

TABLE 83
Anchor Tag
Block 005
Chromosome 14
Rs1859474 (BP 72125288) LD block SNPs

| | | Minor | |
|---|---|---|---|
| | Base | Allele | |
| SNP ID (rs) | Position | Freq | Alleles |
| rs2526897 | 72124704 | 0.150 | C:A |
| rs2526896 | 72124860 | 0.458 | A:G |
| rs1859473 | 72125357 | 0.392 | G:A |
| rs2526947 | 72127012 | 0.458 | G:T |
| rs8007165 | 72127149 | 0.458 | G:A |
| rs12050113 | 72127566 | 0.267 | G:C |
| rs12587303 | 72128367 | 0.050 | C:A |
| rs8019145 | 72129004 | 0.008 | T:A |
| rs2803947 | 72129058 | 0.467 | G:A |
| rs2526944 | 72129581 | 0.467 | A:G |
| rs714149 | 72130610 | 0.458 | T:C |
| rs2526942 | 72131272 | 0.467 | A:C |
| rs17780651 | 72131479 | 0.033 | C:G |
| rs17118212 | 72131508 | 0.467 | A:G |
| rs7492545 | 72132181 | 0.458 | C:T |
| rs17118221 | 72132295 | 0.092 | C:A |
| rs2526940 | 72132453 | 0.075 | C:T |
| rs9671286 | 72132591 | 0.450 | G:A |
| rs12885159 | 72132903 | 0.458 | C:T |
| rs6574086 | 72133383 | 0.458 | C:T |
| rs6574087 | 72133414 | 0.458 | C:T |
| rs2877813 | 72133756 | 0.458 | G:A |
| rs2252463 | 72133782 | 0.083 | T:A |
| rs2877814 | 72133811 | 0.458 | G:A |
| rs17118280 | 72135231 | 0.092 | G:A |
| rs2526937 | 72138382 | 0.083 | C:T |
| rs2107689 | 72138928 | 0.083 | A:G |
| rs717517 | 72140228 | 0.033 | G:A |
| rs7160623 | 72140408 | 0.450 | T:C |
| rs2803952 | 72140587 | 0.075 | G:A |
| rs2803953 | 72141256 | 0.050 | C:T |
| rs11625926 | 72141606 | 0.475 | C:A |

TABLE 84
Anchor Tag
Block 006
Chromosome 14
Rs17094182 (BP 73490193) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs8017668 | 73384341 | 0.442 | C:G | rs8013446 | 73429247 | 0.150 | C:T |
| rs12431457 | 73384916 | 0.400 | T:C | rs1468516 | 73430673 | 0.192 | G:A |
| rs2159179 | 73386601 | 0.442 | C:T | rs2302139 | 73432688 | 0.050 | A:C |
| rs17092737 | 73386753 | 0.042 | C:T | rs1476755 | 73433145 | 0.392 | G:A |
| rs8020091 | 73388300 | 0.008 | C:G | rs2041188 | 73434133 | 0.392 | C:G |
| rs2270074 | 73388398 | 0.442 | G:C | rs10483856 | 73435048 | 0.192 | A:G |
| rs8020630 | 73388426 | 0.400 | G:A | rs10483857 | 73435964 | 0.217 | G:A |
| rs8020267 | 73388467 | 0.117 | G:A | rs4622451 | 73436000 | 0.367 | G:A |
| rs8020523 | 73388539 | 0.150 | G:C | rs1013644 | 73437317 | 0.042 | C:T |
| rs17182621 | 73390469 | 0.400 | C:A | rs17093882 | 73438494 | 0.042 | C:T |
| rs17092878 | 73391075 | 0.042 | G:A | rs2240632 | 73440919 | 0.050 | T:C |
| rs11626950 | 73391806 | 0.442 | A:C | rs917419 | 73445043 | 0.442 | A:G |
| rs8019583 | 73393226 | 0.442 | T:G | rs17782269 | 73447514 | 0.192 | T:G |
| rs9646165 | 73394560 | 0.217 | G:A | rs727523 | 73447888 | 0.150 | T:A |
| rs1008701 | 73397374 | 0.042 | A:C | rs7157730 | 73447955 | 0.008 | G:A |
| rs11159040 | 73398552 | 0.042 | G:T | rs17182675 | 73448558 | 0.192 | A:G |
| rs1985773 | 73400216 | 0.442 | C:T | rs7156534 | 73450836 | 0.050 | G:A |
| rs12588108 | 73402231 | 0.217 | C:T | rs11626866 | 73453233 | 0.442 | T:C |
| rs17782222 | 73408302 | 0.392 | T:A | rs2302121 | 73457128 | 0.433 | A:T |
| rs1029715 | 73408535 | 0.442 | C:G | rs2302120 | 73457203 | 0.208 | T:A |
| rs11159042 | 73410550 | 0.442 | A:T | rs758244 | 73457920 | 0.442 | A:G |
| rs12050434 | 73411191 | 0.042 | G:A | rs11620776 | 73458971 | 0.442 | G:A |
| rs17093474 | 73412166 | 0.050 | T:C | rs1548667 | 73459620 | 0.367 | G:A |
| rs8022626 | 73415124 | 0.008 | A:G | rs8013123 | 73462703 | 0.367 | C:T |
| rs17093520 | 73416048 | 0.042 | A:G | rs8015935 | 73462809 | 0.367 | T:C |
| rs17782239 | 73417192 | 0.392 | C:T | rs17094045 | 73466004 | 0.042 | G:A |
| rs6574151 | 73418572 | 0.367 | G:A | rs17094056 | 73467783 | 0.042 | C:T |
| rs17182656 | 73421066 | 0.192 | C:T | rs8004396 | 73467818 | 0.008 | G:C |
| rs17093546 | 73422773 | 0.042 | G:A | rs6574155 | 73468965 | 0.367 | T:C |
| rs2286426 | 73422957 | 0.367 | T:G | rs2336547 | 73469044 | 0.150 | A:G |
| rs2159177 | 73423079 | 0.392 | C:T | rs17094076 | 73470245 | 0.050 | A:G |
| rs8008877 | 73424973 | 0.392 | A:C | rs4903157 | 73472230 | 0.033 | C:T |
| rs11626813 | 73425431 | 0.392 | T:C | rs17182699 | 73472446 | 0.067 | C:T |
| rs11628074 | 73426592 | 0.442 | T:C | rs7157725 | 73475784 | 0.367 | T:C |
| rs11628813 | 73476063 | 0.442 | G:T | rs3784041 | 73505236 | 0.192 | A:G |
| rs17094107 | 73476135 | 0.042 | G:A | rs3784040 | 73505368 | 0.008 | G:C |
| rs11624780 | 73476364 | 0.433 | T:C | rs2336742 | 73506255 | 0.433 | G:A |
| rs12895829 | 73476553 | 0.192 | C:G | rs17094448 | 73509872 | 0.042 | A:G |
| rs7159394 | 73476664 | 0.008 | C:G | rs2072292 | 73509914 | 0.425 | C:G |
| rs12881125 | 73476803 | 0.192 | T:C | rs11623041 | 73510194 | 0.058 | T:C |
| rs7142206 | 73476815 | 0.367 | C:A | rs7157377 | 73510929 | 0.225 | T:C |
| rs740507 | 73477976 | 0.392 | G:A | rs758240 | 73511098 | 0.383 | T:G |
| rs1005564 | 73480158 | 0.367 | C:T | rs2074933 | 73511290 | 0.375 | C:T |
| rs758241 | 73480416 | 0.433 | C:T | rs2074932 | 73511437 | 0.383 | A:G |
| rs12434704 | 73481787 | 0.433 | T:C | rs12895057 | 73511506 | 0.017 | T:C |
| rs7148485 | 73483173 | 0.433 | T:C | rs17782328 | 73512610 | 0.192 | G:A |
| rs11624578 | 73484826 | 0.392 | G:A | rs2215134 | 73514378 | 0.375 | A:C |
| rs10459545 | 73488962 | 0.367 | G:A | rs17782344 | 73514531 | 0.350 | G:T |
| rs4903159 | 73489578 | 0.367 | A:G | rs11159051 | 73515143 | 0.383 | G:C |
| rs17094182 | 73490193 | 0.008 | C:A | rs12431489 | 73519417 | 0.383 | A:G |
| rs2079632 | 73490965 | 0.150 | G:C | rs4903163 | 73520924 | 0.150 | C:T |
| rs2302118 | 73492082 | 0.225 | G:C | rs4903164 | 73521068 | 0.192 | C:T |
| rs3180946 | 73494691 | 0.383 | T:C | rs17782351 | 73522182 | 0.083 | G:A |
| rs3764859 | 73494809 | 0.425 | C:A | rs7148202 | 73522218 | 0.383 | C:T |
| rs740505 | 73496607 | 0.192 | T:C | rs2302117 | 73524566 | 0.225 | C:A |
| rs740504 | 73496683 | 0.192 | A:G | rs11159052 | 73525142 | 0.425 | C:T |
| rs10873263 | 73496695 | 0.383 | A:C | rs8019765 | 73527697 | 0.425 | C:T |
| rs2074930 | 73497753 | 0.017 | A:T | rs11621114 | 73529848 | 0.425 | C:T |
| rs7141392 | 73498039 | 0.150 | G:A | rs11621118 | 73529879 | 0.383 | T:C |
| rs8500 | 73498198 | 0.425 | G:A | rs1468505 | 73530241 | 0.425 | A:T |
| rs1029704 | 73500200 | 0.425 | C:T | rs9323594 | 73532443 | 0.300 | C:G |
| rs3742815 | 73500944 | 0.383 | G:A | rs8015927 | 73535509 | 0.042 | T:A |
| rs4140924 | 73502183 | 0.383 | A:G | rs4903168 | 73538366 | 0.342 | T:C |
| rs3742813 | 73503612 | 0.425 | T:C | rs10132807 | 73543106 | 0.300 | A:G |
| rs12432463 | 73504376 | 0.417 | T:A | | | | |

TABLE 85
Anchor Tag
Block 007
Chromosome 14
Rs887595 (BP 73736394) LD block SNPs

| | | Minor | |
|---|---|---|---|
| | Base | Allele | |
| SNP ID (rs) | Position | Freq | Alleles |
| rs887595 | 73736394 | 0.133 | G:A |
| rs1972565 | 73736577 | 0.133 | G:A |

TABLE 86
Anchor Tag
Block 008
Chromosome 14
Rs10139335 (BP 90637635) LD block SNPs

| | | Minor | |
|---|---|---|---|
| | Base | Allele | |
| SNP ID (rs) | Position | Freq | Alleles |
| rs10139335 | 90637635 | 0.117 | G:A |
| rs17224369 | 90641113 | 0.217 | A:G |
| rs17261134 | 90641721 | 0.042 | A:G |
| rs11159992 | 90642124 | 0.208 | A:G |
| rs11159993 | 90644374 | 0.033 | G:A |
| rs10137942 | 90649699 | 0.117 | C:G |
| rs2295529 | 90650796 | 0.117 | G:T |

TABLE 87
Anchor Tag
Block 001
Chromosome 15
Rs12324783 (BP 27384612), Rs12441963 (BP 27414002), Rs8032547
(BP 27428127), Rs8043428 (BP 27457583), Rs16955403 (BP 27462960),
Rs8041543 (BP 27466210), and Rs17749882 (BP 27469798) LD block SNPs.
These tags are in separate LD blocks.

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs7164004 | 27377093 | 0.125 | A:C | rs9972472 | 27418298 | 0.267 | A:T |
| rs935313 | 27377574 | 0.425 | C:A | rs12909680 | 27418512 | 0.250 | C:T |
| rs8042826 | 27377698 | 0.125 | G:A | rs817963 | 27418711 | 0.242 | C:T |
| rs8042708 | 27377785 | 0.125 | C:T | rs2004657 | 27421806 | 0.267 | C:T |
| rs8043070 | 27377950 | 0.125 | C:T | rs11855710 | 27421867 | 0.008 | C:T |
| rs4436748 | 27378171 | 0.117 | G:A | rs1464389 | 27423772 | 0.267 | G:A |
| rs8036406 | 27378240 | 0.125 | T:C | rs6494955 | 27424160 | 0.258 | G:A |
| rs966993 | 27378426 | 0.125 | C:G | rs11635198 | 27425481 | 0.267 | G:C |
| rs10519645 | 27381829 | 0.233 | A:G | rs11631080 | 27425517 | 0.100 | A:G |
| rs12324783 | 27384612 | 0.050 | C:G | rs11854062 | 27425782 | 0.167 | A:C |
| rs10163008 | 27387140 | 0.417 | T:C | rs12441809 | 27426653 | 0.008 | G:A |
| rs4780054 | 27387295 | 0.408 | C:T | rs16955362 | 27427599 | 0.100 | A:C |
| rs10519646 | 27390295 | 0.008 | C:T | rs8032547 | 27428127 | 0.100 | G:C |
| rs1025989 | 27391874 | 0.125 | T:C | rs7176689 | 27428369 | 0.017 | A:T |
| rs4341711 | 27392389 | 0.408 | G:C | rs16955366 | 27428661 | 0.017 | A:G |
| rs8042498 | 27393223 | 0.017 | G:A | rs11852981 | 27429475 | 0.100 | T:C |
| rs7174417 | 27395906 | 0.008 | C:T | rs12907598 | 27430473 | 0.275 | T:A |
| rs1373891 | 27395970 | 0.117 | C:T | rs7169173 | 27431427 | 0.125 | G:A |
| rs1664326 | 27397708 | 0.133 | T:A | rs16955368 | 27432392 | 0.017 | C:T |
| rs4779597 | 27398520 | 0.050 | C:G | rs8039737 | 27432555 | 0.275 | A:G |
| rs7165196 | 27398760 | 0.008 | T:A | rs1996709 | 27432765 | 0.150 | G:A |
| rs1445492 | 27399245 | 0.008 | C:A | rs1520940 | 27434516 | 0.150 | C:G |
| rs1716928 | 27399416 | 0.008 | G:A | rs8042027 | 27436369 | 0.283 | A:G |
| rs1584158 | 27399829 | 0.008 | A:G | rs2176922 | 27440122 | 0.350 | C:G |
| rs1445491 | 27400620 | 0.417 | T:C | rs1520941 | 27440581 | 0.350 | T:C |
| rs817959 | 27413231 | 0.133 | G:A | rs17670130 | 27456486 | 0.408 | G:T |
| rs11072207 | 27413741 | 0.267 | C:G | rs8030832 | 27457361 | 0.283 | A:T |
| rs7176361 | 27413773 | 0.017 | A:G | rs8043428 | 27457583 | 0.292 | C:A |
| rs12441963 | 27414002 | 0.267 | A:G | rs17682359 | 27457606 | 0.200 | G:A |
| rs920972 | 27414515 | 0.250 | C:T | rs11858589 | 27458061 | 0.292 | T:C |
| rs934521 | 27414654 | 0.242 | T:C | rs16955401 | 27458373 | 0.292 | A:T |
| rs817961 | 27415730 | 0.242 | C:A | rs11858668 | 27458395 | 0.292 | T:C |
| rs9972308 | 27416165 | 0.267 | A:G | rs12911188 | 27458511 | 0.058 | A:G |
| rs8043513 | 27416803 | 0.250 | G:A | rs8036951 | 27458587 | 0.292 | A:G |
| rs8041618 | 27458992 | 0.292 | A:G | rs8041543 | 27466210 | 0.100 | A:G |
| rs8041779 | 27459066 | 0.292 | A:G | rs17749882 | 27469798 | 0.408 | A:G |
| rs11858800 | 27459434 | 0.292 | A:G | rs521260 | 27469886 | 0.033 | T:C |
| rs8039422 | 27462516 | 0.092 | A:G | rs1870237 | 27470259 | 0.058 | C:T |
| rs8039581 | 27462566 | 0.083 | A:C | rs607503 | 27471950 | 0.142 | T:C |
| rs8031615 | 27462586 | 0.058 | C:T | rs9920343 | 27472244 | 0.425 | C:T |
| rs4779623 | 27462723 | 0.200 | G:A | rs1655656 | 27472426 | 0.067 | T:A |
| rs16955403 | 27462960 | 0.058 | C:T | rs533554 | 27472632 | 0.067 | G:T |
| rs17569233 | 27462976 | 0.083 | T:C | rs16955419 | 27473056 | 0.092 | T:G |
| rs16955406 | 27463819 | 0.058 | C:T | rs1195501 | 27474153 | 0.242 | T:C |
| rs17569247 | 27463936 | 0.033 | T:A | | | | |
| rs1520935 | 27465374 | 0.092 | T:C | | | | |

TABLE 88
Anchor Tag
Block 002
Chromosome 15
Rs2899412 (BP 45445823) and Rs16959379 (BP 45465635)
LD block SNPs. These tags are in separate LD blocks.

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs1496922 | 45436100 | 0.267 | G:A | rs7174964 | 45463520 | 0.283 | A:C |
| rs1496923 | 45436202 | 0.267 | G:A | rs6493274 | 45463781 | 0.292 | A:C |
| rs735999 | 45436399 | 0.267 | A:G | rs419055 | 45463811 | 0.375 | A:T |
| rs16959332 | 45436538 | 0.100 | G:A | rs6493275 | 45463871 | 0.283 | A:T |
| rs2413876 | 45437078 | 0.275 | T:A | rs8040191 | 45464008 | 0.283 | C:T |
| rs1807237 | 45437087 | 0.267 | C:A | rs8040226 | 45464122 | 0.283 | A:G |
| rs3371 | 45437398 | 0.267 | C:T | rs8041603 | 45464147 | 0.283 | T:C |
| rs737520 | 45437429 | 0.267 | C:A | rs12917367 | 45464379 | 0.283 | G:A |
| rs495918 | 45438219 | 0.100 | T:C | rs2433018 | 45464888 | 0.375 | A:G |
| rs4774494 | 45438453 | 0.158 | T:C | rs4368110 | 45465245 | 0.083 | C:T |
| rs4774495 | 45438654 | 0.158 | T:C | rs7166534 | 45465424 | 0.283 | T:C |
| rs680246 | 45439773 | 0.100 | T:C | rs4528506 | 45465528 | 0.083 | G:A |
| rs17289269 | 45440108 | 0.250 | G:T | rs16959379 | 45465635 | 0.092 | G:A |
| rs281267 | 45441740 | 0.267 | G:C | rs4485292 | 45466190 | 0.283 | G:A |
| rs488695 | 45444025 | 0.100 | A:C | rs281282 | 45466549 | 0.108 | G:A |
| rs420095 | 45445506 | 0.075 | A:C | rs16959381 | 45466648 | 0.008 | G:A |
| rs2899412 | 45445823 | 0.100 | T:C | rs2132661 | 45467018 | 0.283 | A:G |
| rs7163407 | 45446956 | 0.133 | T:C | rs2132660 | 45467099 | 0.283 | A:G |
| rs13329568 | 45447314 | 0.133 | A:T | rs281284 | 45467699 | 0.108 | A:C |
| rs281281 | 45448723 | 0.358 | A:G | rs8032333 | 45467859 | 0.283 | A:G |
| rs281244 | 45450652 | 0.275 | T:C | rs281285 | 45468263 | 0.392 | G:C |
| rs12913596 | 45451162 | 0.133 | G:A | rs281286 | 45468542 | 0.392 | T:C |
| rs281247 | 45453215 | 0.267 | G:A | rs281287 | 45468844 | 0.400 | A:G |
| rs281248 | 45454125 | 0.267 | G:A | rs281290 | 45469449 | 0.008 | T:C |
| rs281251 | 45456834 | 0.267 | G:A | rs281291 | 45469720 | 0.392 | A:G |
| rs4624112 | 45456974 | 0.133 | C:T | rs281292 | 45470364 | 0.050 | A:G |
| rs166837 | 45458066 | 0.267 | G:A | rs12901436 | 45470387 | 0.283 | C:G |
| rs281253 | 45458404 | 0.267 | T:C | rs281294 | 45470938 | 0.108 | A:G |
| rs281254 | 45458441 | 0.267 | A:G | rs4359384 | 45471756 | 0.092 | C:T |
| rs281255 | 45458666 | 0.267 | C:T | rs4497626 | 45471800 | 0.392 | A:G |
| rs656813 | 45458779 | 0.100 | T:C | rs4338738 | 45471822 | 0.400 | G:A |
| rs281262 | 45461501 | 0.267 | G:T | rs4356418 | 45471924 | 0.392 | C:G |
| rs175979 | 45462104 | 0.408 | G:A | rs4511474 | 45471972 | 0.392 | G:A |
| rs281265 | 45462894 | 0.392 | A:C | rs281296 | 45472302 | 0.400 | G:A |
| rs1025143 | 45472351 | 0.400 | T:C | rs962949 | 45472670 | 0.383 | A:G |
| rs1025144 | 45472372 | 0.392 | A:G | rs962950 | 45472708 | 0.383 | A:T |
| rs1025145 | 45472598 | 0.383 | A:G | rs281297 | 45472796 | 0.400 | T:C |

TABLE 89
Anchor Tag
Block 002
Chromosome 15
Rs1453862 (BP 45928633), Rs649496 (BP 45936550),
and Rs649122 (BP 45936614) LD block SNPs

| | | Minor | |
|---|---|---|---|
| | Base | Allele | |
| SNP ID (rs) | Position | Freq | Alleles |
| rs1453860 | 45925412 | 0.450 | G:T |
| rs1542043 | 45925710 | 0.458 | A:G |
| rs554501 | 45926097 | 0.450 | G:A |
| rs576606 | 45926247 | 0.458 | A:C |
| rs579314 | 45926594 | 0.442 | G:T |
| rs580014 | 45926628 | 0.275 | C:T |
| rs471659 | 45926871 | 0.442 | T:A |
| rs503062 | 45927967 | 0.442 | T:G |
| rs1453862 | 45928633 | 0.433 | T:C |
| rs1453861 | 45928750 | 0.442 | G:T |
| rs745409 | 45928766 | 0.117 | C:G |
| rs478369 | 45929236 | 0.433 | A:G |
| rs599398 | 45929608 | 0.025 | T:C |
| rs4338739 | 45929632 | 0.125 | T:C |
| rs597543 | 45930092 | 0.433 | A:G |
| rs10775136 | 45930295 | 0.408 | G:A |
| rs514389 | 45930865 | 0.150 | A:C |
| rs16960231 | 45932360 | 0.117 | A:G |
| rs11070617 | 45932546 | 0.408 | T:C |
| rs1224606 | 45933110 | 0.025 | T:C |
| rs499354 | 45933149 | 0.442 | C:T |
| rs660088 | 45933200 | 0.025 | A:G |
| rs1439332 | 45933650 | 0.008 | C:T |
| rs813061 | 45934268 | 0.442 | G:A |
| rs2028316 | 45934507 | 0.117 | T:C |
| rs9888695 | 45934677 | 0.008 | C:T |
| rs518949 | 45934754 | 0.433 | T:C |
| rs12324326 | 45935427 | 0.125 | C:T |
| rs664286 | 45935542 | 0.025 | A:G |
| rs785022 | 45935847 | 0.442 | C:A |
| rs649496 | 45936550 | 0.442 | C:T |
| rs649122 | 45936614 | 0.442 | T:C |
| rs570465 | 45936646 | 0.433 | G:C |
| rs633868 | 45937747 | 0.442 | A:G |

TABLE 90
Anchor Tag
Block 003
Chromosome 15
Rs2444043 (BP 53321634) LD block SNPs

| | | Minor | |
|---|---|---|---|
| | Base | Allele | |
| SNP ID (rs) | Position | Freq | Alleles |
| rs12438278 | 53305123 | 0.075 | T:C |
| rs17819036 | 53305586 | 0.133 | T:C |
| rs2899583 | 53305837 | 0.142 | G:C |
| rs16976191 | 53305852 | 0.050 | A:C |
| rs12439213 | 53306456 | 0.075 | T:C |
| rs16976194 | 53307051 | 0.308 | C:T |
| rs17819042 | 53307217 | 0.325 | A:G |
| rs10518805 | 53307474 | 0.075 | G:T |
| rs8039254 | 53309522 | 0.075 | C:T |
| rs12324775 | 53312081 | 0.075 | G:T |
| rs12439890 | 53312703 | 0.075 | T:C |
| rs12440550 | 53312844 | 0.075 | T:A |
| rs2444039 | 53319503 | 0.383 | G:T |
| rs2444043 | 53321634 | 0.458 | C:T |
| rs549774 | 53322312 | 0.458 | T:C |
| rs540476 | 53324725 | 0.458 | G:C |
| rs16976218 | 53325841 | 0.075 | G:A |
| rs17238220 | 53326005 | 0.075 | A:G |
| rs16976220 | 53326152 | 0.075 | G:A |
| rs7171029 | 53329096 | 0.075 | C:T |
| rs7171897 | 53329391 | 0.075 | G:T |
| rs16976227 | 53330175 | 0.075 | T:C |
| rs16976229 | 53330822 | 0.075 | C:G |
| rs16976231 | 53330876 | 0.075 | A:G |

TABLE 91
Anchor Tag
Block 004
Chromosome 15
Rs2414743 (BP 59808360), Rs8039022 (BP 59820338), and
Rs11853350 (BP 59834186) LD block SNPs

| | | Minor | |
|---|---|---|---|
| | Base | Allele | |
| SNP ID (rs) | Position | Freq | Alleles |
| rs6494294 | 59806856 | 0.417 | G:A |
| rs7164221 | 59807663 | 0.400 | C:T |
| rs2414743 | 59808360 | 0.417 | A:G |
| rs10431799 | 59809148 | 0.017 | C:T |
| rs1047470 | 59810432 | 0.283 | C:G |
| rs1047471 | 59810655 | 0.117 | T:C |
| rs2242025 | 59810691 | 0.017 | G:A |
| rs12909108 | 59810698 | 0.283 | A:G |
| rs12914538 | 59810810 | 0.283 | T:G |
| rs878835 | 59811276 | 0.017 | T:C |
| rs17303684 | 59811444 | 0.017 | A:T |
| rs3959694 | 59811467 | 0.017 | A:G |
| rs2899675 | 59812214 | 0.400 | T:G |
| rs12901234 | 59812272 | 0.267 | T:C |
| rs7168148 | 59813412 | 0.133 | A:G |
| rs12907123 | 59813519 | 0.275 | T:A |
| rs16944542 | 59814220 | 0.283 | A:T |
| rs17303698 | 59814416 | 0.017 | A:G |
| rs17303705 | 59814607 | 0.150 | A:G |
| rs12911757 | 59814696 | 0.417 | A:G |
| rs7179814 | 59814930 | 0.267 | A:G |
| rs11855705 | 59815470 | 0.133 | T:G |
| rs17237950 | 59816709 | 0.017 | G:C |
| rs17237957 | 59816754 | 0.017 | T:C |
| rs7171844 | 59817584 | 0.425 | G:A |
| rs7172964 | 59817663 | 0.417 | T:A |
| rs8027240 | 59818426 | 0.408 | A:G |
| rs8039022 | 59820338 | 0.392 | G:C |
| rs8043433 | 59820734 | 0.283 | C:T |
| rs8041905 | 59823892 | 0.425 | T:C |
| rs2243618 | 59824337 | 0.408 | G:C |
| rs8032851 | 59825712 | 0.258 | T:C |
| rs11854281 | 59826233 | 0.133 | T:C |
| rs8039554 | 59827295 | 0.425 | T:C |
| rs7173251 | 59828106 | 0.275 | C:T |
| rs17237964 | 59828451 | 0.017 | G:A |
| rs11854497 | 59828725 | 0.117 | G:A |
| rs6494295 | 59829347 | 0.425 | T:C |
| rs16944568 | 59831428 | 0.133 | A:G |
| rs4288942 | 59833080 | 0.433 | T:C |
| rs12323968 | 59833386 | 0.267 | C:T |
| rs6494296 | 59833902 | 0.433 | G:T |
| rs11856842 | 59834020 | 0.150 | T:C |
| rs11853350 | 59834186 | 0.425 | A:T |
| rs11854135 | 59834928 | 0.400 | A:G |
| rs12910688 | 59835481 | 0.383 | G:A |
| rs2414748 | 59836411 | 0.417 | C:T |
| rs11857925 | 59837128 | 0.392 | G:A |
| rs17271095 | 59837790 | 0.283 | T:C |
| rs4594200 | 59838771 | 0.433 | T:C |
| rs8026073 | 59839371 | 0.025 | C:G |
| rs12903441 | 59839498 | 0.375 | G:A |

TABLE 92
Anchor Tag
Block 005
Chromosome 15
Rs3883011 (BP 82889398), Rs3883013 (BP 82889661),
and Rs3883014 (BP 82889733) LD block SNPs

| | | Minor | |
|---|---|---|---|
| | Base | Allele | |
| SNP ID (rs) | Position | Freq | Alleles |
| rs3883011 | 82889398 | 0.250 | G:C |
| rs3883013 | 82889661 | 0.250 | T:C |
| rs3883014 | 82889733 | 0.250 | C:G |

TABLE 93
Anchor Tag
Block 006
Chromosome 15
Rs11074160 (BP 91631564) LD block SNPs

| | | Minor | |
|---|---|---|---|
| | Base | Allele | |
| SNP ID (rs) | Position | Freq | Alleles |
| rs4777642 | 91628980 | 0.083 | A:C |
| rs4777643 | 91629102 | 0.092 | T:C |
| rs4777812 | 91629275 | 0.183 | G:A |
| rs4777813 | 91629300 | 0.033 | A:G |
| rs7170522 | 91629636 | 0.092 | A:G |
| rs7179478 | 91630925 | 0.008 | A:G |
| rs11074160 | 91631564 | 0.092 | A:G |

TABLE 94
Anchor Tag
Block 001
Chromosome 16
Rs7202780 (BP 3500902) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

| | | | |
|---|---|---|---|
| rs7202780 | 3500902 | 0.408 | G:A |

TABLE 95
Anchor Tag
Block 002
Chromosome 16
Rs17312836 (BP 49298963), Rs748855 (BP 49308899), Rs3135503
(BP 49348751), and Rs11076540 (BP 49358148) LD block SNPs.
These tags are in separate LD blocks.

| | | Minor | |
|---|---|---|---|
| | Base | Allele | |
| SNP ID (rs) | Position | Freq | Alleles |
| rs1981760 | 49280575 | 0.225 | T:C |
| rs9933594 | 49281588 | 0.217 | C:G |
| rs4785448 | 49282290 | 0.225 | A:G |
| rs7194886 | 49282694 | 0.358 | C:T |
| rs4785224 | 49287947 | 0.358 | G:A |
| rs5743263 | 49288366 | 0.033 | T:C |
| rs5743266 | 49288597 | 0.383 | G:A |
| rs2076753 | 49290875 | 0.383 | G:T |
| rs2067085 | 49291360 | 0.392 | C:G |
| rs2111235 | 49291470 | 0.250 | G:A |
| rs2111234 | 49291534 | 0.250 | A:G |
| rs6500328 | 49294157 | 0.383 | A:G |
| rs8057341 | 49295481 | 0.258 | G:A |
| rs11649521 | 49296331 | 0.358 | G:A |
| rs13339578 | 49296606 | 0.258 | A:G |
| rs17221417 | 49297083 | 0.358 | C:G |
| rs11642646 | 49298687 | 0.375 | C:A |
| rs17312836 | 49298963 | 0.383 | A:C |
| rs13380741 | 49300439 | 0.358 | G:A |
| rs11647841 | 49300832 | 0.383 | G:A |
| rs2066842 | 49302125 | 0.367 | C:T |
| rs5743271 | 49302189 | 0.017 | A:G |
| rs5743273 | 49302517 | 0.008 | C:T |
| rs2066843 | 49302700 | 0.358 | C:T |
| rs1078327 | 49302734 | 0.008 | C:T |
| rs1861759 | 49303084 | 0.383 | T:G |
| rs2066844 | 49303427 | 0.108 | C:T |
| rs5743277 | 49303430 | 0.008 | C:T |
| rs4785225 | 49304047 | 0.258 | G:C |
| rs17313265 | 49305205 | 0.358 | C:T |
| rs751271 | 49308676 | 0.258 | G:T |
| rs748855 | 49308899 | 0.383 | A:G |
| rs1861758 | 49309288 | 0.383 | G:A |
| rs1861757 | 49310316 | 0.383 | T:C |
| rs10521209 | 49313210 | 0.408 | T:G |
| rs2066845 | 49314041 | 0.017 | G:C |
| rs5743289 | 49314275 | 0.267 | C:T |
| rs2076756 | 49314382 | 0.350 | A:G |
| rs3135499 | 49323628 | 0.417 | A:C |
| rs5743299 | 49324254 | 0.008 | C:T |
| rs8056611 | 49325148 | 0.492 | A:G |
| rs718226 | 49327064 | 0.433 | A:G |
| rs751919 | 49333246 | 0.417 | T:G |
| rs8060598 | 49339303 | 0.433 | T:C |
| rs3785142 | 49344648 | 0.492 | G:A |
| rs7342715 | 49344984 | 0.483 | G:A |
| rs12324931 | 49347659 | 0.108 | A:C |
| rs3135502 | 49348506 | 0.008 | A:C |
| rs3135503 | 49348751 | 0.433 | T:G |
| rs4785450 | 49349769 | 0.408 | C:T |
| rs11862720 | 49350205 | 0.083 | C:G |
| rs8062105 | 49353395 | 0.083 | T:C |
| rs11864698 | 49355708 | 0.083 | A:G |
| rs2066851 | 49356430 | 0.442 | A:G |
| rs11076540 | 49358148 | 0.425 | T:G |
| rs11859674 | 49360216 | 0.083 | G:A |
| rs1420873 | 49361257 | 0.083 | G:C |
| rs8053457 | 49361788 | 0.483 | G:A |
| rs1420872 | 49365280 | 0.425 | C:T |
| rs6500331 | 49366227 | 0.492 | A:G |
| rs16948811 | 49368619 | 0.083 | T:C |
| rs6500332 | 49370653 | 0.008 | C:T |
| rs17222902 | 49371498 | 0.067 | G:A |
| rs1420871 | 49372933 | 0.067 | C:T |
| rs17314341 | 49374161 | 0.433 | C:T |
| rs9938976 | 49374720 | 0.433 | A:G |
| rs8062540 | 49375433 | 0.433 | A:G |
| rs8047910 | 49376613 | 0.500 | A:A |

TABLE 96
Anchor Tag
Block 003
Chromosome 16
Rs254353 (BP 64469211) and Rs1094921 (BP 64499283)
LD block SNPs. These tags are in separate LD blocks.

| | | Minor | |
|---|---|---|---|
| | Base | Allele | |
| SNP ID (rs) | Position | Freq | Alleles |
| rs254353 | 64469211 | 0.283 | T:A |
| rs385488 | 64498101 | 0.408 | T:C |
| rs11864495 | 64498175 | 0.158 | T:C |
| rs397875 | 64498622 | 0.417 | G:A |
| rs1094921 | 64499283 | 0.442 | C:A |
| rs1968997 | 64499671 | 0.008 | A:C |
| rs1124555 | 64500333 | 0.150 | A:C |
| rs254371 | 64501151 | 0.408 | T:C |
| rs1974887 | 64502342 | 0.033 | G:A |
| rs9931226 | 64503969 | 0.033 | T:C |
| rs37162 | 64503998 | 0.433 | C:A |
| rs13331174 | 64505764 | 0.033 | C:T |
| rs11864429 | 64507109 | 0.033 | C:T |
| rs1094924 | 64507626 | 0.442 | G:C |
| rs11860691 | 64507744 | 0.033 | T:C |
| rs40189 | 64509923 | 0.408 | G:A |
| rs192264 | 64510490 | 0.433 | A:G |
| rs1094928 | 64511973 | 0.408 | G:T |

TABLE 97
Anchor Tag
Block 004
Chromosome 16
Rs4488477 (BP 73343602) and Rs4570874 (BP 73343663)
LD block SNPs

| | | Minor | |
|---|---|---|---|
| | Base | Allele | |
| SNP ID (rs) | Position | Freq | Alleles |
| rs4488477 | 73343602 | 0.217 | G:A |
| rs4570874 | 73343663 | 0.233 | A:T |

TABLE 98
Anchor Tag
Block 001
Chromosome 17
Rs16954363 (BP 522254) LD block SNPs

| | | Minor | | | | | |
|---|---|---|---|---|---|---|---|
| | Base | Allele | | | Base | Minor | |
| SNP ID (rs) | Position | Freq | Alleles | SNP ID (rs) | Position | Allele Freq | Alleles |
| rs10521104 | 451209 | 0.175 | A:G | rs2543777 | 507601 | 0.350 | T:C |
| rs10521105 | 451728 | 0.133 | C:G | rs1833459 | 508994 | 0.417 | A:G |
| rs4968055 | 451912 | 0.017 | G:A | rs16954271 | 509228 | 0.075 | C:A |
| rs7225041 | 451977 | 0.308 | G:C | rs394747 | 509245 | 0.492 | G:C |
| rs9915733 | 452521 | 0.208 | C:G | rs11247558 | 514705 | 0.167 | G:C |
| rs12051571 | 453558 | 0.125 | A:G | rs865702 | 514903 | 0.350 | A:T |
| rs4968093 | 453788 | 0.208 | G:A | rs415822 | 515320 | 0.358 | C:T |
| rs10521106 | 457457 | 0.492 | T:C | rs8074959 | 516366 | 0.017 | G:A |
| rs10521107 | 457906 | 0.492 | G:A | rs8069369 | 517138 | 0.075 | T:G |
| rs8081057 | 457940 | 0.242 | C:G | rs2279890 | 518381 | 0.075 | C:T |
| rs11247555 | 464169 | 0.333 | G:A | rs425961 | 520464 | 0.483 | A:G |
| rs4968057 | 464271 | 0.492 | T:A | rs8081506 | 520720 | 0.125 | G:A |
| rs16953962 | 464657 | 0.017 | C:T | rs368722 | 520795 | 0.108 | G:A |
| rs7218543 | 464870 | 0.050 | A:G | rs11655448 | 521732 | 0.008 | G:A |
| rs12601935 | 466075 | 0.492 | G:A | rs16954363 | 522254 | 0.075 | G:A |
| rs17677436 | 467064 | 0.492 | T:C | rs16954373 | 523267 | 0.017 | C:A |
| rs8066036 | 471047 | 0.075 | C:T | rs2279888 | 523909 | 0.075 | A:C |
| rs12939528 | 471141 | 0.492 | C:T | rs9898987 | 524640 | 0.008 | C:T |
| rs4968058 | 474421 | 0.208 | A:C | rs838369 | 524930 | 0.475 | C:T |
| rs9906432 | 475618 | 0.217 | T:C | rs17616098 | 525260 | 0.125 | T:C |
| rs8080970 | 476586 | 0.017 | T:C | rs9906369 | 525337 | 0.008 | G:A |
| rs331012 | 476622 | 0.025 | T:A | rs863331 | 525431 | 0.358 | C:T |
| rs16954064 | 477089 | 0.242 | T:C | rs16954395 | 526043 | 0.033 | C:T |
| rs12450330 | 478479 | 0.075 | T:C | rs11651244 | 526120 | 0.058 | T:C |
| rs8071277 | 480258 | 0.017 | G:A | rs12232517 | 526382 | 0.125 | G:A |
| rs17609440 | 480338 | 0.083 | G:A | rs11654989 | 526479 | 0.150 | G:C |
| rs2002015 | 481275 | 0.408 | G:T | rs8077845 | 531749 | 0.017 | A:G |
| rs8075960 | 482948 | 0.025 | T:C | rs7207469 | 532225 | 0.075 | T:G |
| rs11650715 | 483164 | 0.200 | G:A | rs385894 | 537828 | 0.292 | A:G |
| rs434307 | 500830 | 0.175 | T:C | rs423887 | 540350 | 0.492 | C:T |
| rs16954209 | 501263 | 0.017 | T:C | rs7218263 | 540569 | 0.075 | G:A |
| rs379998 | 503634 | 0.492 | A:G | rs7217716 | 543722 | 0.017 | G:A |
| rs448465 | 506638 | 0.492 | A:T | rs7207203 | 544060 | 0.050 | T:C |
| rs2160955 | 507354 | 0.492 | T:C | rs410157 | 544990 | 0.400 | C:T |
| rs366151 | 545061 | 0.492 | G:T | rs11652019 | 564619 | 0.150 | G:A |
| rs870183 | 546561 | 0.475 | G:A | rs2955626 | 564850 | 0.483 | C:G |
| rs11657201 | 546674 | 0.150 | A:G | rs684232 | 565715 | 0.400 | T:C |
| rs870181 | 547007 | 0.167 | C:T | rs461251 | 565912 | 0.417 | A:G |
| rs2657633 | 547252 | 0.500 | C:C | rs1237094 | 566592 | 0.417 | C:T |
| rs4968100 | 547485 | 0.200 | A:G | rs2740363 | 567161 | 0.358 | T:C |
| rs2543781 | 549219 | 0.492 | C:G | rs461521 | 569247 | 0.500 | A:A |
| rs17616389 | 554959 | 0.150 | G:A | rs16954846 | 571426 | 0.067 | A:G |
| rs838371 | 556025 | 0.483 | C:T | rs16954854 | 571615 | 0.067 | G:A |
| rs10521109 | 560251 | 0.183 | A:C | rs12453927 | 573112 | 0.050 | A:G |
| rs7220509 | 561244 | 0.075 | C:G | rs2543767 | 576425 | 0.050 | T:C |
| rs11871883 | 561549 | 0.125 | C:T | rs450289 | 576646 | 0.433 | T:G |
| rs17678731 | 562106 | 0.125 | T:G | rs1741275 | 577113 | 0.442 | G:C |
| rs12940579 | 563011 | 0.183 | A:G | | | | |

TABLE 99
Anchor Tag
Block 002
Chromosome 17
Rs8068990 (BP 9892826), Rs8072311 (BP 9903752), and Rs17810635
(BP 9905657) LD block SNPs. These tags are in separate LD blocks.

| | | Minor | |
|---|---|---|---|
| | Base | Allele | |
| SNP ID (rs) | Position | Freq | Alleles |
| rs7216101 | 9884592 | 0.100 | A:C |
| rs12603484 | 9886769 | 0.083 | C:T |
| rs11649799 | 9888344 | 0.300 | C:T |
| rs11650065 | 9889827 | 0.250 | A:G |
| rs11656239 | 9891392 | 0.192 | C:T |
| rs8068990 | 9892826 | 0.083 | A:T |
| rs8067422 | 9892967 | 0.083 | G:T |
| rs6503281 | 9894198 | 0.083 | T:A |
| rs8079564 | 9895345 | 0.083 | C:T |
| rs12150362 | 9896885 | 0.083 | C:T |
| rs8075721 | 9897046 | 0.092 | T:C |
| rs8070297 | 9897170 | 0.083 | G:A |
| rs8071931 | 9897179 | 0.075 | A:G |
| rs8072084 | 9903073 | 0.067 | T:C |
| rs12603691 | 9903134 | 0.067 | G:C |
| rs8072311 | 9903752 | 0.058 | C:A |
| rs7216650 | 9904138 | 0.067 | A:G |
| rs8081931 | 9904694 | 0.067 | T:G |
| rs17810635 | 9905657 | 0.058 | C:T |

TABLE 100
Anchor Tag
Block 001
Chromosome 18
Rs489570 (BP 8703965) LD block SNPs

| | | Minor | |
|---|---|---|---|
| | Base | Allele | |
| SNP ID (rs) | Position | Freq | Alleles |
| rs503087 | 8699655 | 0.292 | C:G |
| rs11873701 | 8701050 | 0.292 | T:A |
| rs1249489 | 8701942 | 0.292 | G:A |
| rs593582 | 8703088 | 0.242 | G:A |
| rs688046 | 8703774 | 0.242 | G:A |
| rs489570 | 8703965 | 0.233 | C:T |

TABLE 101
Anchor Tag
Block 002
Chromosome 18
Rs9950064 (BP 61691931) and Rs17075420 (BP 61700461) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs7232924 | 61646095 | 0.275 | T:A | rs17075336 | 61687176 | 0.050 | A:G |
| rs1484696 | 61646398 | 0.275 | G:A | rs17075338 | 61687357 | 0.033 | G:A |
| rs8098217 | 61647803 | 0.308 | A:G | rs2628203 | 61687743 | 0.258 | G:T |
| rs12455246 | 61649860 | 0.033 | C:T | rs2628204 | 61687798 | 0.258 | T:G |
| rs8086245 | 61650344 | 0.033 | T:C | rs7242044 | 61687859 | 0.342 | T:C |
| rs1385277 | 61650899 | 0.042 | C:A | rs1484699 | 61688050 | 0.258 | A:G |
| rs12605720 | 61652170 | 0.008 | A:G | rs12606432 | 61688511 | 0.292 | A:G |
| rs1484694 | 61652626 | 0.008 | A:C | rs12606480 | 61688917 | 0.342 | T:C |
| rs1484726 | 61652873 | 0.300 | G:C | rs9959875 | 61689321 | 0.033 | A:G |
| rs9789163 | 61652888 | 0.008 | A:C | rs9960373 | 61689748 | 0.033 | A:T |
| rs9963814 | 61655933 | 0.308 | T:C | rs4124017 | 61689835 | 0.292 | A:G |
| rs11665474 | 61656681 | 0.342 | G:A | rs9946001 | 61690326 | 0.258 | G:C |
| rs1955235 | 61657045 | 0.308 | C:T | rs2046407 | 61690373 | 0.292 | A:G |
| rs8089669 | 61668497 | 0.342 | C:A | rs9950064 | 61691931 | 0.033 | G:A |
| rs1484710 | 61669384 | 0.308 | T:C | rs1484700 | 61692220 | 0.258 | T:C |
| rs1032116 | 61672836 | 0.258 | C:T | rs7239998 | 61692692 | 0.033 | A:G |
| rs2333464 | 61673850 | 0.292 | A:C | rs7241631 | 61692867 | 0.033 | T:A |
| rs2009296 | 61674951 | 0.342 | G:A | rs9965345 | 61692892 | 0.033 | T:A |
| rs2244074 | 61675469 | 0.258 | C:T | rs9953248 | 61693003 | 0.033 | C:T |
| rs2244081 | 61675524 | 0.033 | C:A | rs1872330 | 61693625 | 0.342 | A:G |
| rs17061853 | 61676652 | 0.033 | G:A | rs17075388 | 61694288 | 0.050 | G:A |
| rs17075315 | 61678122 | 0.050 | T:C | rs7231247 | 61694325 | 0.308 | G:C |
| rs12966354 | 61678337 | 0.292 | G:A | rs4527121 | 61694534 | 0.308 | A:G |
| rs2587404 | 61679154 | 0.258 | A:C | rs8087953 | 61694859 | 0.050 | C:T |
| rs2291343 | 61680996 | 0.292 | G:A | rs2628205 | 61695345 | 0.050 | G:A |
| rs17711596 | 61681120 | 0.058 | C:T | rs9951873 | 61695690 | 0.033 | T:C |
| rs2587406 | 61683167 | 0.033 | C:A | rs12969226 | 61696322 | 0.292 | T:C |
| rs2587407 | 61683396 | 0.033 | G:C | rs2086874 | 61696374 | 0.258 | G:A |
| rs17075325 | 61684681 | 0.050 | C:T | rs17075416 | 61696560 | 0.050 | G:A |
| rs7245106 | 61684954 | 0.342 | T:C | rs9945997 | 61697352 | 0.033 | G:A |
| rs2628201 | 61686069 | 0.033 | G:C | rs2628207 | 61697366 | 0.292 | T:C |
| rs2017885 | 61686427 | 0.292 | G:C | rs2628209 | 61697531 | 0.292 | G:A |
| rs2587410 | 61686997 | 0.258 | T:C | rs2628210 | 61697973 | 0.258 | C:G |
| rs2628202 | 61687147 | 0.258 | G:A | rs17075420 | 61700461 | 0.033 | G:C |
| rs8091413 | 61700840 | 0.258 | G:C | rs2628213 | 61702060 | 0.258 | T:A |

TABLE 102
Anchor Tag
Block 001
Chromosome 19
Rs855614 (BP 43311582) LD block SNPs

| | | Minor | |
|---|---|---|---|
| | Base | Allele | |
| SNP ID (rs) | Position | Freq | Alleles |
| rs855603 | 43252531 | 0.183 | C:T |
| rs729625 | 43255222 | 0.183 | G:T |
| rs1622409 | 43255472 | 0.183 | G:A |
| rs855620 | 43256925 | 0.492 | C:T |
| rs855623 | 43259601 | 0.183 | A:G |
| rs855624 | 43259645 | 0.183 | A:C |
| rs1725461 | 43261478 | 0.183 | A:G |
| rs1725512 | 43263568 | 0.183 | A:G |
| rs4802233 | 43268585 | 0.183 | A:G |
| rs10411005 | 43269277 | 0.183 | T:G |
| rs1725489 | 43269866 | 0.492 | C:T |
| rs941040 | 43270154 | 0.183 | C:G |
| rs855640 | 43272184 | 0.183 | A:G |
| rs1725474 | 43276212 | 0.500 | A:A |
| rs1725472 | 43279885 | 0.500 | A:A |
| rs3810356 | 43283347 | 0.183 | G:T |
| rs16979801 | 43294054 | 0.100 | G:A |
| rs855632 | 43295099 | 0.417 | C:G |
| rs12985707 | 43296428 | 0.408 | C:T |
| rs941039 | 43296954 | 0.175 | C:T |
| rs3852911 | 43297276 | 0.408 | A:G |
| rs855633 | 43298355 | 0.175 | C:G |
| rs705507 | 43302561 | 0.417 | A:G |
| rs863020 | 43304833 | 0.175 | C:T |
| rs860623 | 43305194 | 0.175 | C:T |
| rs8104873 | 43308188 | 0.075 | G:C |
| rs855614 | 43311582 | 0.425 | G:A |
| rs861304 | 43313313 | 0.133 | A:G |
| rs7253312 | 43313963 | 0.408 | A:G |
| rs7253633 | 43314002 | 0.408 | G:A |
| rs2116862 | 43315732 | 0.408 | C:T |
| rs10403595 | 43316065 | 0.017 | A:G |
| rs4803801 | 43317948 | 0.175 | G:A |
| rs4803802 | 43318016 | 0.175 | G:A |
| rs2005054 | 43319040 | 0.175 | C:T |
| rs9941456 | 43319656 | 0.167 | C:A |
| rs9941467 | 43320867 | 0.175 | G:A |
| rs9941474 | 43320966 | 0.425 | C:T |
| rs11671853 | 43321083 | 0.142 | A:G |
| rs8111180 | 43322000 | 0.425 | A:G |
| rs8107385 | 43324592 | 0.400 | G:T |
| rs2304132 | 43324999 | 0.400 | C:T |
| rs8100128 | 43325830 | 0.425 | G:C |
| rs4802251 | 43326314 | 0.425 | C:T |
| rs7250034 | 43326734 | 0.167 | G:T |
| rs4803810 | 43328506 | 0.175 | C:A |
| rs7248428 | 43330263 | 0.175 | G:A |
| rs8109799 | 43331743 | 0.425 | T:C |
| rs332844 | 43333884 | 0.425 | T:C |
| rs332845 | 43334081 | 0.400 | G:A |
| rs332847 | 43334646 | 0.133 | G:A |
| rs332848 | 43334714 | 0.425 | C:T |
| rs332849 | 43335791 | 0.425 | G:A |
| rs10422665 | 43340910 | 0.175 | T:C |
| rs2010234 | 43343027 | 0.408 | A:G |
| rs10412762 | 43345453 | 0.175 | A:T |
| rs4802256 | 43346391 | 0.175 | A:G |

TABLE 103
Anchor Tag
Block 001
Chromosome 20
Rs6140121 (BP 6860028) and Rs6085753 (BP 6873667) LD
block SNPs

| | | Minor | |
|---|---|---|---|
| | Base | Allele | |
| SNP ID (rs) | Position | Freq | Alleles |
| rs6054610 | 6858864 | 0.167 | T:A |
| rs6054611 | 6859631 | 0.117 | T:C |
| rs6077093 | 6859696 | 0.167 | A:G |
| rs6077094 | 6859707 | 0.167 | A:G |
| rs6140121 | 6860028 | 0.167 | A:T |
| rs6117512 | 6860437 | 0.167 | G:A |
| rs6140123 | 6861603 | 0.167 | C:T |
| rs6085740 | 6861995 | 0.167 | T:G |
| rs6140124 | 6862617 | 0.050 | T:C |
| rs717087 | 6862914 | 0.167 | C:T |
| rs7261837 | 6864029 | 0.267 | C:T |
| rs6054612 | 6864285 | 0.167 | C:A |
| rs16992810 | 6864374 | 0.083 | A:G |
| rs4815950 | 6865578 | 0.483 | G:A |
| rs6054616 | 6868028 | 0.167 | G:C |
| rs2223667 | 6868193 | 0.267 | T:C |
| rs6038655 | 6868352 | 0.483 | A:C |
| rs2057039 | 6870810 | 0.167 | C:T |
| rs974099 | 6871536 | 0.350 | C:T |
| rs6085750 | 6873425 | 0.483 | A:G |
| rs6085753 | 6873667 | 0.167 | C:T |
| rs10485708 | 6874675 | 0.267 | T:C |
| rs6085756 | 6875335 | 0.483 | A:G |
| rs6038661 | 6875369 | 0.167 | G:A |
| rs6077103 | 6875825 | 0.483 | A:G |
| rs6085760 | 6876595 | 0.483 | C:T |
| rs6085761 | 6876620 | 0.483 | A:G |
| rs6085762 | 6876675 | 0.483 | T:G |
| rs6117521 | 6876906 | 0.483 | C:T |
| rs6085767 | 6878424 | 0.483 | A:G |
| rs7263691 | 6878578 | 0.083 | T:C |
| rs2143539 | 6879256 | 0.483 | G:A |
| rs1555134 | 6879300 | 0.267 | T:A |
| rs2326851 | 6879717 | 0.167 | A:G |
| rs2876053 | 6880154 | 0.083 | G:T |
| rs7267551 | 6880220 | 0.267 | T:C |
| rs2876054 | 6880252 | 0.083 | C:G |
| rs6107896 | 6880760 | 0.483 | A:G |
| rs2206690 | 6881369 | 0.483 | T:C |
| rs4815952 | 6882897 | 0.483 | T:C |

TABLE 104
Anchor Tag
Block 002
Chromosome 20
Rs819159 (BP 32343381) LD block SNPs

| | | Minor | |
|---|---|---|---|
| | Base | Allele | |
| SNP ID (rs) | Position | Freq | Alleles |
| rs6087561 | 32247488 | 0.108 | T:C |
| rs12480555 | 32248155 | 0.108 | A:G |
| rs6088423 | 32251802 | 0.108 | T:G |
| rs6088425 | 32255181 | 0.108 | T:A |
| rs6120568 | 32257161 | 0.108 | T:C |
| rs12625272 | 32257822 | 0.108 | C:G |
| rs6579152 | 32258687 | 0.108 | A:G |
| rs7354490 | 32260700 | 0.067 | C:G |
| rs6088429 | 32262391 | 0.108 | C:T |
| rs6059729 | 32270552 | 0.117 | A:G |
| rs6119473 | 32272910 | 0.117 | T:C |
| rs6142129 | 32283532 | 0.275 | A:G |
| rs6087565 | 32285120 | 0.108 | A:G |
| rs819178 | 32289453 | 0.108 | A:G |
| rs6088443 | 32295792 | 0.117 | C:T |
| rs819164 | 32303115 | 0.108 | A:G |
| rs819163 | 32306018 | 0.117 | T:G |
| rs6120580 | 32308191 | 0.117 | C:T |
| rs2378132 | 32309697 | 0.117 | A:G |
| rs819162 | 32315943 | 0.117 | A:T |
| rs6088454 | 32318238 | 0.117 | A:T |
| rs819144 | 32325217 | 0.117 | G:T |
| rs819145 | 32325488 | 0.117 | G:T |
| rs819177 | 32327964 | 0.117 | G:C |
| rs819176 | 32329505 | 0.117 | G:T |
| rs819175 | 32329842 | 0.117 | C:G |
| rs819172 | 32331167 | 0.117 | C:T |
| rs819133 | 32333975 | 0.117 | G:T |
| rs864702 | 32335211 | 0.117 | G:A |
| rs866027 | 32337971 | 0.117 | A:G |
| rs1205357 | 32341427 | 0.133 | T:C |
| rs819159 | 32343381 | 0.117 | T:A |
| rs819158 | 32344399 | 0.117 | A:G |
| rs819156 | 32344684 | 0.117 | C:A |
| rs819148 | 32352198 | 0.117 | A:G |
| rs819147 | 32353365 | 0.117 | T:C |
| rs819146 | 32354861 | 0.117 | T:G |
| rs1205350 | 32362000 | 0.117 | A:G |

TABLE 105
Anchor Tag
Block 001
Chromosome 21
Rs2827566 (BP 22848288), Rs2827573 (BP 22854664), Rs1392922
(BP 22864584), and Rs2827606 (BP 22865549) LD block SNPs

| | | Minor | |
|---|---|---|---|
| | Base | Allele | |
| SNP ID (rs) | Position | Freq | Alleles |
| rs2827566 | 22848288 | 0.258 | C:T |
| rs2827567 | 22848523 | 0.267 | G:T |
| rs9981283 | 22850922 | 0.092 | A:G |
| rs2088843 | 22853558 | 0.267 | T:G |
| rs2827573 | 22854664 | 0.258 | C:T |
| rs2827576 | 22856188 | 0.500 | A:A |
| rs2827587 | 22858858 | 0.500 | A:A |
| rs2827590 | 22859625 | 0.258 | G:A |
| rs2827602 | 22862870 | 0.242 | A:G |
| rs1392922 | 22864584 | 0.258 | C:T |
| rs2827604 | 22865197 | 0.258 | C:T |
| rs2827606 | 22865549 | 0.258 | T:G |
| rs1405763 | 22869446 | 0.483 | G:T |
| rs2827613 | 22871547 | 0.500 | A:A |

TABLE 106
Anchor Tag
Block 002
Chromosome 21
Rs2828495 (BP 24038081), Rs2828506 (BP 24048609), Rs2828512 (BP 24051399),
Rs2828516 (BP 24057342), and Rs1157277 (BP 24084426) LD block SNPs.
These tags are in separate LD blocks.

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs2247047 | 24034582 | 0.233 | T:A | rs207527 | 24058210 | 0.233 | A:G |
| rs2828491 | 24036609 | 0.308 | A:G | rs11910225 | 24058737 | 0.008 | T:G |
| rs2828492 | 24036798 | 0.142 | C:A | rs207528 | 24058994 | 0.242 | T:C |
| rs2828493 | 24037142 | 0.142 | C:T | rs1910980 | 24059560 | 0.233 | A:G |
| rs2828494 | 24038001 | 0.092 | G:A | rs1910981 | 24059646 | 0.250 | T:C |
| rs2828495 | 24038081 | 0.242 | G:A | rs2828518 | 24060429 | 0.500 | C:C |
| rs2828497 | 24042375 | 0.233 | A:G | rs17004430 | 24060640 | 0.017 | G:A |
| rs2828498 | 24043081 | 0.442 | C:T | rs207529 | 24060742 | 0.242 | T:C |
| rs2828500 | 24046446 | 0.467 | G:T | rs2097030 | 24076915 | 0.467 | T:A |
| rs11087889 | 24046508 | 0.458 | G:A | rs9305196 | 24077046 | 0.142 | C:A |
| rs2828501 | 24047046 | 0.458 | G:A | rs7510562 | 24077061 | 0.467 | T:C |
| rs2828502 | 24047144 | 0.233 | A:C | rs13051039 | 24077448 | 0.133 | C:G |
| rs2828503 | 24048282 | 0.467 | A:G | rs1701377 | 24078168 | 0.467 | G:A |
| rs2828504 | 24048326 | 0.092 | C:T | rs1701378 | 24078459 | 0.075 | A:G |
| rs2828506 | 24048609 | 0.467 | T:G | rs2828529 | 24079865 | 0.133 | G:A |
| rs2828507 | 24048749 | 0.467 | G:A | rs9981608 | 24080129 | 0.317 | A:G |
| rs2828508 | 24048953 | 0.467 | G:A | rs207478 | 24080140 | 0.467 | T:G |
| rs6516578 | 24049160 | 0.467 | T:C | rs11702120 | 24080351 | 0.042 | G:A |
| rs2828509 | 24049793 | 0.467 | G:A | rs2256749 | 24080847 | 0.092 | C:T |
| rs2828512 | 24051399 | 0.092 | A:T | rs207479 | 24081204 | 0.092 | C:A |
| rs9978561 | 24051715 | 0.142 | A:G | rs207480 | 24082565 | 0.092 | C:T |
| rs17004426 | 24051780 | 0.017 | C:T | rs207502 | 24083893 | 0.467 | T:C |
| rs11910832 | 24052024 | 0.367 | C:A | rs2828530 | 24083987 | 0.375 | C:T |
| rs12483137 | 24052635 | 0.467 | T:C | rs2828531 | 24084206 | 0.317 | G:T |
| rs2828513 | 24053854 | 0.092 | G:A | rs207503 | 24084246 | 0.467 | C:T |
| rs12482178 | 24054018 | 0.142 | G:A | rs1157277 | 24084426 | 0.317 | T:G |
| rs2568512 | 24054876 | 0.233 | G:T | rs1157278 | 24084464 | 0.325 | G:C |
| rs6516580 | 24055057 | 0.233 | T:C | rs2828532 | 24085113 | 0.058 | T:C |
| rs9979663 | 24055245 | 0.233 | C:T | rs207504 | 24085486 | 0.092 | G:T |
| rs9979807 | 24055472 | 0.233 | T:A | rs2828533 | 24085858 | 0.183 | C:A |
| rs2828515 | 24056599 | 0.142 | A:G | rs2828534 | 24086981 | 0.317 | G:A |
| rs9305191 | 24057156 | 0.142 | G:A | rs2828535 | 24087396 | 0.058 | T:C |
| rs2828516 | 24057342 | 0.092 | A:C | rs207506 | 24087928 | 0.467 | C:T |
| rs997724 | 24057640 | 0.158 | T:C | rs2828538 | 24092630 | 0.317 | A:T |
| rs1023371 | 24088593 | 0.008 | C:T | rs2828539 | 24092754 | 0.325 | A:C |
| rs207508 | 24089684 | 0.092 | C:T | rs2828540 | 24092872 | 0.325 | G:A |
| rs207509 | 24090493 | 0.467 | C:T | rs207472 | 24093103 | 0.092 | T:A |
| rs2828536 | 24090925 | 0.058 | G:T | rs207473 | 24093800 | 0.475 | A:T |
| rs2828537 | 24091341 | 0.058 | A:T | | | | |
| rs2828517 | 24057852 | 0.233 | T:C | | | | |

TABLE 107
Anchor Tag
Block 003
Chromosome 21
Rs2837979 (BP 41532183) LD block SNPs. Rs2837979 is
located between rs2837977 and rs9981747.

| | | Minor | |
|---|---|---|---|
| | Base | Allele | |
| SNP ID (rs) | Position | Freq | Alleles |
| rs2837977 | 41531739 | 0.367 | G:A |
| rs9981747 | 41534296 | 0.100 | G:A |

TABLE 108
Anchor Tag
Block 001
Chromosome 22
Rs6007450 (BP 43823566) and Rs8135904 (BP 43888956) LD block
SNPs. These tags are in separate LD blocks.

| | | Minor | |
|---|---|---|---|
| | Base | Allele | |
| SNP ID (rs) | Position | Freq | Alleles |
| rs6007450 | 43823566 | 0.092 | G:A |
| rs9615025 | 43824054 | 0.092 | G:A |
| rs132823 | 43857439 | 0.192 | G:A |
| rs9985182 | 43860378 | 0.375 | G:T |
| rs132824 | 43864883 | 0.150 | A:T |
| rs80428 | 43865937 | 0.375 | C:T |
| rs2013582 | 43866087 | 0.458 | T:C |
| rs9615046 | 43866811 | 0.467 | C:T |
| rs16993506 | 43868140 | 0.208 | G:C |
| rs132825 | 43868351 | 0.158 | C:T |
| rs132826 | 43868590 | 0.158 | G:A |
| rs132828 | 43869569 | 0.167 | C:T |
| rs4823448 | 43870386 | 0.458 | G:C |
| rs9615049 | 43871037 | 0.458 | G:A |
| rs6007476 | 43871519 | 0.008 | T:C |
| rs132829 | 43871608 | 0.158 | C:T |
| rs132830 | 43871660 | 0.375 | A:G |
| rs132831 | 43872096 | 0.058 | C:T |
| rs6006945 | 43872408 | 0.008 | C:G |
| rs13058199 | 43873747 | 0.250 | T:C |
| rs13054435 | 43873996 | 0.142 | G:A |
| rs7293135 | 43874018 | 0.467 | A:G |
| rs132832 | 43874100 | 0.367 | C:T |
| rs132834 | 43874734 | 0.367 | A:G |
| rs9626641 | 43874900 | 0.467 | T:C |
| rs12483927 | 43875368 | 0.208 | C:T |
| rs132839 | 43877726 | 0.367 | T:C |
| rs132846 | 43879730 | 0.367 | G:A |
| rs132847 | 43880168 | 0.383 | G:C |
| rs3788634 | 43880278 | 0.158 | G:T |
| rs132853 | 43881244 | 0.375 | C:T |
| rs132856 | 43881541 | 0.067 | C:G |
| rs132857 | 43881929 | 0.150 | A:G |
| rs132858 | 43883272 | 0.167 | C:T |
| rs132860 | 43884098 | 0.383 | A:G |
| rs132863 | 43884964 | 0.167 | A:C |
| rs132871 | 43886950 | 0.167 | T:C |
| rs132873 | 43887239 | 0.167 | C:T |
| rs132874 | 43887380 | 0.167 | A:G |
| rs132875 | 43887449 | 0.167 | C:A |
| rs132881 | 43888117 | 0.167 | C:A |
| rs8135904 | 43888956 | 0.458 | G:A |
| rs132883 | 43889048 | 0.167 | G:T |
| rs132884 | 43889523 | 0.167 | A:G |
| rs2138156 | 43890382 | 0.275 | C:T |
| rs132891 | 43891131 | 0.375 | T:C |
| rs9615052 | 43891506 | 0.450 | C:T |
| rs17548707 | 43891983 | 0.258 | C:T |
| rs9626648 | 43892044 | 0.008 | C:T |
| rs13058400 | 43893691 | 0.458 | C:T |
| rs17548742 | 43893987 | 0.217 | A:C |
| rs9615054 | 43895523 | 0.458 | T:G |
| rs226523 | 43897647 | 0.167 | T:C |
| rs226524 | 43897768 | 0.167 | T:C |
| rs170508 | 43898341 | 0.158 | T:C |
| rs3788638 | 43899026 | 0.458 | A:G |
| rs6006950 | 43899959 | 0.458 | A:G |
| rs2075952 | 43900039 | 0.008 | G:T |
| rs2673084 | 43901436 | 0.375 | C:G |
| rs1811817 | 43902058 | 0.375 | T:C |
| rs1058024 | 43903397 | 0.458 | G:C |
| rs172866 | 43903602 | 0.200 | T:A |
| rs226515 | 43904435 | 0.375 | G:A |
| rs9614616 | 43904449 | 0.200 | T:C |
| rs6007500 | 43904472 | 0.458 | A:G |
| rs226516 | 43904933 | 0.150 | G:A |
| rs226518 | 43907054 | 0.158 | T:G |

TABLE 109
Anchor Tag
Block 001
Chromosome 23
Rs6529723 (BP 4857004), Rs1900761 (BP 4863509), and
Rs5961620 (BP 4875328) LD block SNPs. These tags are in
separate LD blocks.

| | | Minor | |
|---|---|---|---|
| | Base | Allele | |
| SNP ID (rs) | Position | Freq | Alleles |
| rs6529723 | 4857004 | 0.011 | T:C |
| rs1900761 | 4863509 | 0.011 | G:A |
| rs5961612 | 4864493 | 0.456 | A:C |
| rs2044631 | 4865934 | 0.011 | G:T |
| rs6529728 | 4866740 | 0.1 | C:G |
| rs10521585 | 4871476 | 0.389 | A:G |
| rs5961617 | 4871915 | 0.011 | G:T |
| rs6529732 | 4872539 | 0.011 | A:C |
| rs5915943 | 4872589 | 0.456 | C:T |
| rs1868337 | 4873762 | 0.011 | G:A |
| rs1868335 | 4873958 | 0.011 | A:C |
| rs5961620 | 4875328 | 0.011 | T:C |
| rs10126275 | 4876746 | 0.389 | G:A |
| rs6413640 | 4877578 | 0.011 | G:T |
| rs1462645 | 4877784 | 0.011 | C:A |
| rs5915485 | 4878249 | 0.444 | A:T |
| rs5915487 | 4880410 | 0.011 | A:T |
| rs1037215 | 4881227 | 0.278 | G:C |
| rs2007032 | 4881457 | 0.111 | T:C |
| rs1037212 | 4881542 | 0.011 | G:A |
| rs5915957 | 4882080 | 0.011 | T:C |
| rs1900768 | 4883727 | 0.111 | G:A |
| rs5915488 | 4884196 | 0.389 | C:T |
| rs5915958 | 4884325 | 0.1 | G:A |
| rs5915489 | 4884958 | 0.1 | G:C |
| rs6638451 | 4887121 | 0.011 | G:A |
| rs5915961 | 4887893 | 0.011 | C:G |
| rs4826757 | 4888539 | 0.011 | T:A |
| rs996058 | 4890227 | 0.056 | C:T |
| rs1381757 | 4892911 | 0.011 | C:T |
| rs5915964 | 4893275 | 0.1 | A:G |
| rs1381756 | 4893301 | 0.311 | A:T |
| rs5961624 | 4893655 | 0.1 | G:A |
| rs5915965 | 4894325 | 0.111 | C:T |
| rs12688533 | 4894418 | 0.356 | G:A |
| rs5915966 | 4895036 | 0.1 | C:A |
| rs1157197 | 4895569 | 0.011 | C:T |
| rs5915491 | 4896470 | 0.1 | T:G |
| rs1975907 | 4898270 | 0.011 | T:C |
| rs1381754 | 4899869 | 0.111 | G:A |
| rs5915968 | 4901990 | 0.1 | A:G |
| rs5915972 | 4905491 | 0.1 | G:A |
| rs921225 | 4906089 | 0.356 | A:G |
| rs5915975 | 4908809 | 0.033 | T:C |
| rs10436759 | 4909133 | 0.1 | T:C |
| rs1599638 | 4909505 | 0.011 | T:A |
| rs1462633 | 4909529 | 0.011 | C:T |
| rs1462632 | 4909600 | 0.011 | G:A |
| rs936507 | 4910241 | 0.111 | C:T |
| rs936506 | 4910292 | 0.311 | A:C |
| rs5915493 | 4910477 | 0.011 | T:A |
| rs6529742 | 4910641 | 0.011 | A:C |
| rs11797188 | 4910706 | 0.011 | C:A |
| rs5915494 | 4910759 | 0.033 | C:T |
| rs6639310 | 4910817 | 0.1 | T:C |
| rs6639312 | 4910890 | 0.111 | G:A |
| rs6638460 | 4911510 | 0.011 | T:G |
| rs5916007 | 4937002 | 0.189 | C:T |
| rs6639315 | 4911949 | 0.1 | G:A |
| rs17219756 | 4912129 | 0.1 | C:G |
| rs5961627 | 4912183 | 0.1 | G:A |
| rs5961628 | 4912282 | 0.1 | A:G |
| rs5961629 | 4912382 | 0.1 | A:G |
| rs5961630 | 4912527 | 0.1 | A:G |
| rs1965270 | 4912965 | 0.011 | T:A |
| rs12216968 | 4913118 | 0.1 | T:C |
| rs17304854 | 4913662 | 0.1 | T:C |
| rs2126524 | 4913840 | 0.111 | G:T |
| rs2318434 | 4914116 | 0.111 | G:A |
| rs4826681 | 4917310 | 0.011 | G:A |
| rs6639325 | 4917402 | 0.1 | A:G |
| rs6639326 | 4917438 | 0.1 | C:T |
| rs5915982 | 4918042 | 0.1 | T:A |
| rs5915983 | 4918058 | 0.1 | A:G |
| rs5915984 | 4918097 | 0.1 | G:A |
| rs2169539 | 4918525 | 0.1 | T:G |
| rs3943335 | 4918536 | 0.1 | G:A |
| rs2169538 | 4918609 | 0.1 | G:A |
| rs5961631 | 4918716 | 0.111 | G:C |
| rs10856269 | 4919120 | 0.111 | C:T |
| rs10856270 | 4919288 | 0.111 | A:C |
| rs5915496 | 4919930 | 0.1 | G:A |
| rs6639338 | 4920972 | 0.1 | A:G |
| rs7472553 | 4921056 | 0.111 | T:C |
| rs1462631 | 4921457 | 0.011 | C:A |
| rs10482247 | 4921584 | 0.2 | C:T |
| rs5915498 | 4922001 | 0.044 | C:T |
| rs9306750 | 4922236 | 0.2 | T:C |
| rs5915499 | 4922269 | 0.011 | G:A |
| rs10218069 | 4922572 | 0.344 | G:A |
| rs5915987 | 4922765 | 0.1 | G:A |
| rs5915988 | 4922964 | 0.344 | T:C |
| rs5915992 | 4923138 | 0.3 | A:G |
| rs16997018 | 4923200 | 0.044 | A:C |
| rs5915993 | 4923332 | 0.3 | C:T |
| rs5915500 | 4923343 | 0.356 | T:C |
| rs5915994 | 4923379 | 0.3 | G:T |
| rs6638467 | 4925060 | 0.3 | T:C |
| rs6638468 | 4925234 | 0.3 | C:A |
| rs6638469 | 4925384 | 0.3 | A:G |
| rs5915999 | 4925513 | 0.3 | G:C |
| rs5915501 | 4925690 | 0.3 | T:C |
| rs5915502 | 4925793 | 0.3 | T:C |
| rs2996000 | 4925814 | 0.356 | G:A |
| rs7882176 | 4925871 | 0.044 | C:G |
| rs5916000 | 4925951 | 0.3 | G:A |
| rs5915503 | 4926017 | 0.3 | T:C |
| rs11094677 | 4926110 | 0.3 | C:T |
| rs5916003 | 4926692 | 0.3 | C:G |
| rs5915504 | 4927292 | 0.3 | A:G |
| rs12833835 | 4927608 | 0.111 | T:C |
| rs6639354 | 4929252 | 0.344 | A:G |
| rs6639356 | 4929922 | 0.3 | C:G |
| rs5961634 | 4931566 | 0.3 | G:A |
| rs6638475 | 4935086 | 0.1 | C:T |
| rs6639359 | 4935238 | 0.3 | C:T |

TABLE 110
Anchor Tag
Block 002
Chromosome 23
Rs2041633 (BP 116825004) LD block SNPs

| | | Minor | |
|---|---|---|---|
| | Base | Allele | |
| SNP ID (rs) | Position | Freq | Alleles |
| rs5956809 | 116810371 | 0.089 | A:G |
| rs4825548 | 116810925 | 0.167 | G:T |
| rs5956810 | 116810945 | 0.089 | C:G |
| rs6645983 | 116814855 | 0.089 | C:G |
| rs12009449 | 116817277 | 0.089 | A:G |
| rs12015049 | 116819137 | 0.089 | C:T |
| rs10465415 | 116824714 | 0.089 | T:A |
| rs2041633 | 116825004 | 0.211 | C:T |
| rs5956813 | 116826923 | 0.089 | A:C |
| rs6645987 | 116827145 | 0.089 | C:T |
| rs6645414 | 116828280 | 0.089 | C:T |
| rs6645415 | 116828686 | 0.089 | T:C |
| rs6603346 | 116829203 | 0.100 | T:A |
| rs5955987 | 116829795 | 0.089 | T:C |
| rs5955989 | 116830113 | 0.089 | C:G |
| rs6645993 | 116832144 | 0.089 | T:C |
| rs6645417 | 116833622 | 0.089 | T:C |
| rs12390839 | 116834677 | 0.089 | T:A |
| rs6645994 | 116835381 | 0.089 | C:T |
| rs6603347 | 116839006 | 0.089 | G:A |
| rs6603348 | 116839035 | 0.089 | C:T |
| rs7056344 | 116842536 | 0.089 | A:G |
| rs6603349 | 116845341 | 0.089 | A:C |
| rs6645420 | 116847110 | 0.089 | A:G |
| rs7357940 | 116849565 | 0.089 | T:C |
| rs6645424 | 116853574 | 0.089 | T:C |
| rs5956817 | 116854460 | 0.089 | C:T |
| rs6646008 | 116860244 | 0.089 | A:T |
| rs5956818 | 116860515 | 0.089 | A:G |
| rs6603352 | 116860629 | 0.089 | A:G |
| rs2430212 | 116869938 | 0.233 | T:C |
| rs6603355 | 116872547 | 0.089 | A:G |
| rs5956821 | 116873034 | 0.089 | C:T |
| rs2497858 | 116873323 | 0.089 | A:C |
| rs2430207 | 116876213 | 0.089 | C:T |
| rs16994947 | 116883254 | 0.011 | C:T |
| rs5955993 | 116885951 | 0.089 | G:C |
| rs6646017 | 116888512 | 0.089 | A:G |
| rs6646018 | 116889245 | 0.089 | T:C |
| rs6603357 | 116893319 | 0.089 | A:G |
| rs12396523 | 116898636 | 0.089 | T:C |
| rs2430209 | 116910730 | 0.089 | A:G |
| rs6645431 | 116914525 | 0.089 | T:G |
| rs12841154 | 116924860 | 0.089 | T:C |
| rs5956846 | 116928434 | 0.011 | C:T |
| rs5956852 | 116932722 | 0.089 | C:A |
| rs6646033 | 116940399 | 0.089 | T:C |
| rs5956862 | 116953622 | 0.089 | A:G |

TABLE 111
Singleton Tag
Block 001
Chromosome 1
Rs475929 (BP 74479600) LD block SNPs

| | | Minor | |
|---|---|---|---|
| | Base | Allele | |
| SNP ID (rs) | Position | Freq | Alleles |
| rs10493539 | 74427598 | 0.308 | G:A |
| rs574692 | 74435114 | 0.192 | A:G |
| rs576802 | 74436317 | 0.183 | T:A |
| rs12118620 | 74439976 | 0.083 | C:G |
| rs6700161 | 74442874 | 0.167 | T:C |
| rs518574 | 74443059 | 0.042 | A:G |
| rs524252 | 74444618 | 0.200 | C:T |
| rs2039407 | 74446391 | 0.025 | A:T |
| rs10493540 | 74446464 | 0.100 | C:T |
| rs17095047 | 74448734 | 0.025 | C:T |
| rs473834 | 74455055 | 0.200 | A:G |
| rs480267 | 74455745 | 0.200 | G:A |
| rs6663678 | 74456530 | 0.325 | G:C |
| rs6660831 | 74457009 | 0.325 | G:A |
| rs10890121 | 74459575 | 0.233 | C:T |
| rs10493541 | 74460905 | 0.025 | C:T |
| rs572180 | 74461220 | 0.200 | G:T |
| rs1333029 | 74461343 | 0.100 | G:A |
| rs485414 | 74461463 | 0.200 | A:G |
| rs1412823 | 74461787 | 0.325 | A:T |
| rs6684409 | 74474219 | 0.308 | G:A |
| rs477920 | 74479865 | 0.192 | A:G |
| rs6424577 | 74481288 | 0.208 | C:T |
| rs566621 | 74482899 | 0.183 | A:T |
| rs9728079 | 74486033 | 0.308 | A:G |
| rs3765660 | 74488829 | 0.242 | A:G |
| rs12030793 | 74489075 | 0.008 | T:A |
| rs569564 | 74492356 | 0.217 | A:C |
| rs570363 | 74492410 | 0.008 | A:G |
| rs1417892 | 74492672 | 0.100 | A:G |

TABLE 112
Singleton Tag
Block 002
Chromosome 1
Rs11164386 (BP 102290118) LD block SNPs

| | | Minor | |
|---|---|---|---|
| | Base | Allele | |
| SNP ID (rs) | Position | Freq | Alleles |
| rs4564159 | 102275442 | 0.233 | C:T |
| rs11164378 | 102276013 | 0.358 | C:G |
| rs4609450 | 102276096 | 0.233 | T:G |
| rs6670467 | 102278152 | 0.233 | A:G |
| rs4908216 | 102284535 | 0.158 | A:T |
| rs12088003 | 102284965 | 0.275 | G:A |
| rs11578152 | 102289042 | 0.425 | A:G |
| rs4445491 | 102290881 | 0.233 | C:G |
| rs10493979 | 102291255 | 0.342 | T:G |

TABLE 113
Singleton Tag
Block 001
Chromosome 2
Rs17012999 (BP 119032452) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs4849710 | 119023889 | 0.350 | A:G | rs487040 | 119061670 | 0.158 | T:C |
| rs6542470 | 119027351 | 0.350 | T:A | rs511923 | 119062080 | 0.092 | A:G |
| rs6719023 | 119028462 | 0.350 | G:A | rs571314 | 119063927 | 0.342 | A:G |
| rs1545135 | 119028762 | 0.350 | C:A | rs576023 | 119064461 | 0.342 | T:C |
| rs747724 | 119030500 | 0.350 | C:G | rs1026212 | 119065375 | 0.333 | G:A |
| rs747725 | 119030529 | 0.150 | T:C | rs10496550 | 119066217 | 0.058 | A:G |
| rs747761 | 119031085 | 0.075 | G:A | rs572585 | 119070708 | 0.150 | C:T |
| rs2018992 | 119031341 | 0.075 | G:A | rs538554 | 119075821 | 0.092 | G:A |
| rs10427226 | 119031447 | 0.075 | G:C | rs567885 | 119076739 | 0.083 | C:T |
| rs7603585 | 119031988 | 0.350 | T:C | rs9679477 | 119077839 | 0.050 | A:G |
| rs1996706 | 119034831 | 0.325 | G:A | rs681586 | 119077893 | 0.092 | G:A |
| rs12994932 | 119034981 | 0.442 | A:G | rs1451203 | 119079073 | 0.492 | A:T |
| rs6738326 | 119035045 | 0.342 | A:G | rs472326 | 119084040 | 0.150 | C:T |
| rs2118379 | 119036313 | 0.350 | C:T | rs476865 | 119084540 | 0.083 | C:T |
| rs7581452 | 119041585 | 0.075 | A:G | rs567729 | 119086166 | 0.367 | G:A |
| rs6732572 | 119042267 | 0.342 | G:A | rs1451206 | 119087426 | 0.483 | A:G |
| rs10496549 | 119043381 | 0.075 | A:G | rs643883 | 119090538 | 0.150 | G:C |
| rs6542472 | 119045668 | 0.417 | A:C | rs527581 | 119092033 | 0.083 | A:C |
| rs7604059 | 119047753 | 0.075 | A:G | rs563449 | 119093672 | 0.092 | G:A |
| rs1437687 | 119048897 | 0.342 | C:A | rs17776428 | 119095345 | 0.117 | G:A |
| rs7600523 | 119049174 | 0.075 | G:T | rs1156000 | 119101587 | 0.483 | A:T |
| rs17015272 | 119049439 | 0.075 | G:A | rs671909 | 119108094 | 0.367 | A:G |
| rs6542473 | 119049836 | 0.342 | G:C | rs561489 | 119109497 | 0.083 | A:C |
| rs17015427 | 119050793 | 0.008 | T:C | rs682878 | 119109503 | 0.092 | C:T |
| rs1437689 | 119051608 | 0.342 | C:T | rs651477 | 119111921 | 0.367 | T:C |
| rs7593247 | 119053819 | 0.475 | C:T | rs17776629 | 119114026 | 0.358 | T:C |
| rs17015669 | 119053915 | 0.075 | G:A | rs1349459 | 119115398 | 0.425 | C:G |
| rs1004624 | 119055186 | 0.342 | G:C | rs503585 | 119117156 | 0.158 | T:G |
| rs11886707 | 119056005 | 0.333 | C:T | rs6736836 | 119117323 | 0.425 | C:T |
| rs1465819 | 119056491 | 0.342 | G:C | rs162678 | 119121896 | 0.158 | T:C |
| rs11888583 | 119057891 | 0.342 | C:A | rs13412685 | 119122688 | 0.283 | C:T |
| rs10172336 | 119059193 | 0.500 | G:G | rs162679 | 119123380 | 0.158 | C:T |
| rs536979 | 119059685 | 0.092 | C:T | rs332084 | 119125234 | 0.092 | T:A |
| rs13017945 | 119060980 | 0.208 | C:T | rs332085 | 119127841 | 0.425 | C:T |
| rs332086 | 119129420 | 0.092 | G:T | rs563697 | 119130683 | 0.367 | A:G |

TABLE 114
Singleton Tag
Block 002
Chromosome 2
Rs16844553 (BP 160633928) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs7601374 | 160579557 | 0.475 | C:T | rs10176317 | 160606949 | 0.225 | C:A |
| rs12617388 | 160579957 | 0.183 | C:G | rs16844511 | 160607276 | 0.200 | C:T |
| rs2292391 | 160584675 | 0.225 | G:A | rs17828734 | 160607492 | 0.042 | C:A |
| rs12692574 | 160585212 | 0.042 | C:T | rs12692577 | 160607703 | 0.225 | C:A |
| rs3806603 | 160587315 | 0.458 | C:T | rs12472767 | 160607842 | 0.242 | T:C |
| rs16844479 | 160590846 | 0.200 | C:T | rs1877195 | 160608191 | 0.225 | A:T |
| rs925406 | 160592896 | 0.233 | C:T | rs1877194 | 160608239 | 0.233 | G:A |
| rs2136970 | 160594443 | 0.233 | C:T | rs16844522 | 160608342 | 0.200 | A:G |
| rs6735923 | 160595095 | 0.458 | C:T | rs7598738 | 160612729 | 0.233 | A:C |
| rs12692575 | 160595538 | 0.233 | A:G | rs11690178 | 160613549 | 0.033 | A:G |
| rs12692576 | 160595568 | 0.033 | G:A | rs7566824 | 160616281 | 0.225 | A:G |
| rs6724314 | 160595700 | 0.225 | C:A | rs6432567 | 160616389 | 0.033 | G:C |
| rs6739453 | 160595747 | 0.233 | C:T | rs7597243 | 160617416 | 0.225 | C:A |
| rs10497207 | 160598198 | 0.233 | T:C | rs12997701 | 160618501 | 0.225 | T:C |
| rs1995949 | 160598435 | 0.033 | G:T | rs4664302 | 160618922 | 0.233 | A:T |
| rs6715456 | 160599006 | 0.200 | C:T | rs6432568 | 160619334 | 0.033 | G:A |
| rs13032589 | 160600912 | 0.025 | T:C | rs4297833 | 160619367 | 0.033 | T:C |
| rs907245 | 160601879 | 0.033 | C:T | rs4664304 | 160619515 | 0.458 | G:A |
| rs907246 | 160602182 | 0.033 | C:T | rs769369 | 160620763 | 0.225 | G:A |
| rs13003929 | 160603929 | 0.033 | A:T | rs1995950 | 160626135 | 0.217 | A:G |
| rs12470907 | 160604232 | 0.225 | C:G | rs1511223 | 160628151 | 0.425 | A:C |
| rs13033829 | 160604666 | 0.233 | G:C | rs10929959 | 160631089 | 0.017 | T:C |
| rs6710996 | 160605030 | 0.200 | A:C | rs4665130 | 160632940 | 0.017 | G:T |
| rs1511221 | 160605039 | 0.225 | C:A | rs3828323 | 160633582 | 0.475 | T:C |
| rs2136976 | 160605437 | 0.233 | T:C | rs6432570 | 160633821 | 0.492 | C:T |
| rs2136975 | 160605483 | 0.233 | G:C | rs3792161 | 160634730 | 0.217 | C:T |
| rs2357689 | 160605790 | 0.233 | A:T | rs949753 | 160636691 | 0.217 | A:G |
| rs2136973 | 160606010 | 0.233 | C:A | rs12373732 | 160639960 | 0.217 | C:A |
| rs2175415 | 160606138 | 0.233 | C:G | rs1511217 | 160640600 | 0.217 | T:C |
| rs7576140 | 160606433 | 0.233 | A:G | rs6432571 | 160642750 | 0.017 | G:A |
| rs7589960 | 160606444 | 0.233 | A:T | rs10188272 | 160645311 | 0.017 | G:A |
| rs7576257 | 160606554 | 0.033 | A:G | rs12614986 | 160645917 | 0.233 | C:T |
| rs10176387 | 160606837 | 0.225 | G:A | rs10173701 | 160646105 | 0.217 | A:T |
| rs1511219 | 160606935 | 0.033 | T:A | rs2667020 | 160648854 | 0.017 | C:A |
| rs2667021 | 160650688 | 0.017 | G:A | rs6757188 | 160669130 | 0.225 | T:C |
| rs2175414 | 160651460 | 0.017 | G:A | rs10168568 | 160669824 | 0.008 | A:G |
| rs2136972 | 160652144 | 0.017 | G:A | rs3792167 | 160671089 | 0.225 | G:C |
| rs12692578 | 160655010 | 0.250 | T:C | rs2715923 | 160671228 | 0.158 | A:G |
| rs2715917 | 160657005 | 0.025 | G:C | rs16844623 | 160671401 | 0.008 | T:C |
| rs2221809 | 160657124 | 0.008 | T:C | rs3792168 | 160671979 | 0.500 | C:C |
| rs3749114 | 160658503 | 0.458 | T:A | rs1849050 | 160672945 | 0.050 | T:C |
| rs2715918 | 160658695 | 0.150 | G:A | rs2715931 | 160673145 | 0.158 | C:A |
| rs3828324 | 160660498 | 0.500 | C:C | rs2667000 | 160675861 | 0.150 | T:A |
| rs2667023 | 160660906 | 0.142 | T:C | rs1606116 | 160676022 | 0.017 | C:A |
| rs3792164 | 160662268 | 0.058 | G:A | rs1397709 | 160678219 | 0.150 | T:G |
| rs3792165 | 160662876 | 0.233 | A:G | rs12692580 | 160679406 | 0.225 | G:A |
| rs2715920 | 160663322 | 0.150 | C:T | rs7592418 | 160681291 | 0.225 | A:G |
| rs2667024 | 160664377 | 0.025 | A:G | rs2667002 | 160681999 | 0.142 | T:C |
| rs2667025 | 160664848 | 0.150 | T:C | rs12692581 | 160682989 | 0.233 | A:G |
| rs1319327 | 160665398 | 0.117 | T:A | rs1567537 | 160683075 | 0.142 | C:T |
| rs979129 | 160665658 | 0.492 | C:T | rs17829873 | 160683154 | 0.433 | G:T |
| rs12692579 | 160666646 | 0.225 | G:A | rs4664305 | 160684742 | 0.225 | T:C |
| rs1546512 | 160666814 | 0.025 | C:T | rs11687309 | 160685895 | 0.233 | T:C |
| rs1546514 | 160666973 | 0.025 | T:C | rs1397710 | 160685998 | 0.225 | A:C |
| rs2715921 | 160667335 | 0.150 | A:G | rs1397711 | 160686601 | 0.267 | G:A |
| rs1567538 | 160667868 | 0.025 | T:C | rs2715916 | 160686725 | 0.142 | G:A |
| rs7590560 | 160668550 | 0.150 | C:G | | | | |

TABLE 115
Singleton Tag
Block 003
Chromosome 2
Rs2198731 (BP 182195451) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs17225354 | 182179110 | 0.408 | T:C | rs3770110 | 182207873 | 0.383 | G:T |
| rs4667308 | 182181017 | 0.067 | G:A | rs6714061 | 182209834 | 0.458 | C:G |
| rs4667309 | 182181363 | 0.458 | T:C | rs16867437 | 182211149 | 0.150 | A:G |
| rs16867431 | 182182328 | 0.150 | A:G | rs6721763 | 182211494 | 0.458 | C:T |
| rs1038034 | 182183040 | 0.150 | A:G | rs3770109 | 182214134 | 0.150 | A:G |
| rs16867433 | 182183961 | 0.150 | A:T | rs2305581 | 182214809 | 0.150 | T:C |
| rs16867434 | 182185098 | 0.067 | T:C | rs3770108 | 182215070 | 0.467 | T:C |
| rs2305586 | 182185688 | 0.067 | T:C | rs767500 | 182215961 | 0.383 | C:T |
| rs13000886 | 182186262 | 0.008 | C:A | rs1375490 | 182215978 | 0.383 | A:C |
| rs4667310 | 182188349 | 0.150 | A:T | rs3770107 | 182216459 | 0.383 | T:G |
| rs3770116 | 182190149 | 0.150 | G:T | rs3770106 | 182216835 | 0.150 | A:G |
| rs12471434 | 182191214 | 0.050 | C:T | rs6757312 | 182218574 | 0.383 | G:A |
| rs1816990 | 182191613 | 0.167 | C:G | rs6728886 | 182218615 | 0.383 | T:C |
| rs7607758 | 182192417 | 0.417 | G:T | rs3770104 | 182219651 | 0.383 | T:C |
| rs11689738 | 182194452 | 0.400 | G:A | rs6707704 | 182220460 | 0.467 | A:G |
| rs1551033 | 182194682 | 0.400 | A:G | rs4667319 | 182220851 | 0.383 | A:G |
| rs1349197 | 182197657 | 0.392 | C:G | rs1839266 | 182222381 | 0.383 | G:C |
| rs11686132 | 182197789 | 0.008 | C:A | rs1449259 | 182223835 | 0.158 | A:G |
| rs11686167 | 182197810 | 0.392 | C:T | rs4479393 | 182223979 | 0.150 | A:G |
| rs13032116 | 182199487 | 0.392 | C:T | rs7562325 | 182224603 | 0.467 | T:C |
| rs12690516 | 182199548 | 0.458 | A:G | rs7601843 | 182224992 | 0.150 | G:T |
| rs3816521 | 182199834 | 0.392 | T:A | rs10490691 | 182226622 | 0.150 | C:T |
| rs2305583 | 182200073 | 0.392 | A:G | rs1047307 | 182227258 | 0.258 | G:A |
| rs17304344 | 182201301 | 0.392 | C:T | rs12614187 | 182227997 | 0.150 | C:T |
| rs1037624 | 182201340 | 0.383 | T:C | rs2290517 | 182228893 | 0.150 | T:G |
| rs10204136 | 182201417 | 0.392 | A:G | rs17226490 | 182229645 | 0.467 | C:G |
| rs1964512 | 182201569 | 0.017 | T:G | rs16867442 | 182229756 | 0.150 | A:C |
| rs3770112 | 182203047 | 0.392 | G:A | rs718449 | 182231069 | 0.150 | G:A |
| rs4667316 | 182203279 | 0.392 | C:T | rs16867443 | 182232200 | 0.150 | A:G |
| rs12469449 | 182205229 | 0.392 | T:C | rs13034078 | 182236952 | 0.467 | G:A |
| rs10490692 | 182206211 | 0.458 | C:T | rs12997453 | 182238765 | 0.467 | A:G |
| rs1375491 | 182206556 | 0.458 | A:G | rs12623737 | 182240028 | 0.150 | T:G |
| rs2887191 | 182206813 | 0.458 | C:T | rs971741 | 182240604 | 0.150 | C:A |
| rs3770111 | 182207822 | 0.150 | G:A | rs2368215 | 182242474 | 0.350 | C:T |
| rs1449256 | 182243184 | 0.342 | G:C | rs10514624 | 182243799 | 0.208 | T:C |
| rs10930971 | 182243636 | 0.192 | A:G | rs11680383 | 182244655 | 0.375 | T:C |

TABLE 116
Singleton Tag
Block 004
Chromosome 2
Rs7562389 (BP 231268001) LD block SNPs

| | | Minor | |
|---|---|---|---|
| | Base | Allele | |
| SNP ID (rs) | Position | Freq | Alleles |
| rs13418016 | 231267333 | 0.483 | T:C |
| rs10804366 | 231267639 | 0.475 | A:T |
| rs10933342 | 231267864 | 0.225 | A:T |
| rs7562389 | 231268001 | 0.467 | C:T |
| rs11674658 | 231268398 | 0.358 | T:C |

TABLE 117
Singleton Tag
Block 001
Chromosome 3
Rs17042155 (BP 16346233) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs712872 | 16264896 | 0.333 | C:T | rs842287 | 16292947 | 0.458 | C:T |
| rs2063777 | 16265171 | 0.175 | A:G | rs11925571 | 16293919 | 0.175 | G:A |
| rs2135227 | 16265735 | 0.175 | G:T | rs11926401 | 16294112 | 0.175 | G:A |
| rs6442591 | 16265771 | 0.175 | C:T | rs1566973 | 16295105 | 0.458 | T:C |
| rs842247 | 16266826 | 0.458 | C:T | rs1566972 | 16295364 | 0.458 | A:G |
| rs6763776 | 16268155 | 0.350 | G:T | rs9813083 | 16296645 | 0.175 | A:G |
| rs842246 | 16268429 | 0.467 | T:C | rs6763538 | 16298240 | 0.008 | C:T |
| rs842245 | 16268679 | 0.358 | T:C | rs842269 | 16299367 | 0.358 | G:A |
| rs842244 | 16269262 | 0.458 | G:A | rs842274 | 16302859 | 0.458 | T:G |
| rs6442592 | 16269429 | 0.175 | A:G | rs6777976 | 16302913 | 0.175 | C:T |
| rs2470508 | 16269898 | 0.367 | C:T | rs842273 | 16303360 | 0.467 | T:C |
| rs6770707 | 16270269 | 0.458 | C:G | rs12495804 | 16305517 | 0.050 | C:T |
| rs712874 | 16270639 | 0.367 | A:G | rs842272 | 16307050 | 0.458 | A:G |
| rs859702 | 16271672 | 0.367 | C:G | rs842270 | 16307992 | 0.458 | G:A |
| rs6442593 | 16271931 | 0.175 | G:A | rs11915850 | 16309318 | 0.358 | C:G |
| rs6790838 | 16272552 | 0.175 | T:C | rs842285 | 16312257 | 0.325 | C:G |
| rs842250 | 16273680 | 0.358 | T:C | rs13091727 | 16313702 | 0.458 | C:A |
| rs842251 | 16274095 | 0.458 | C:G | rs842284 | 16315672 | 0.458 | T:C |
| rs842252 | 16274972 | 0.358 | G:A | rs2292618 | 16318059 | 0.008 | C:G |
| rs842254 | 16275629 | 0.458 | C:T | rs7644052 | 16319839 | 0.008 | C:A |
| rs842255 | 16276568 | 0.367 | A:G | rs2672555 | 16324329 | 0.367 | C:T |
| rs2292615 | 16276847 | 0.008 | T:C | rs7615129 | 16324424 | 0.175 | C:T |
| rs552 | 16276963 | 0.175 | G:A | rs6442595 | 16327197 | 0.175 | A:G |
| rs859703 | 16278843 | 0.458 | G:A | rs860923 | 16331470 | 0.308 | G:A |
| rs842257 | 16279150 | 0.458 | A:G | rs689894 | 16332086 | 0.333 | A:G |
| rs2245721 | 16280678 | 0.458 | C:G | rs6900 | 16332498 | 0.175 | G:A |
| rs2245708 | 16281022 | 0.358 | T:C | rs1050604 | 16332712 | 0.008 | A:G |
| rs2276786 | 16281774 | 0.008 | A:C | rs842424 | 16332982 | 0.325 | T:A |
| rs1514892 | 16282047 | 0.458 | T:C | rs690241 | 16333588 | 0.492 | C:T |
| rs6804208 | 16285383 | 0.175 | G:A | rs574668 | 16334075 | 0.333 | G:A |
| rs842259 | 16287568 | 0.458 | C:T | rs9817227 | 16334240 | 0.183 | C:T |
| rs6796483 | 16288324 | 0.175 | G:A | rs9817725 | 16334532 | 0.175 | C:T |
| rs842289 | 16290651 | 0.358 | C:T | rs493626 | 16334965 | 0.492 | C:T |
| rs842288 | 16291319 | 0.358 | T:C | rs842423 | 16335130 | 0.325 | C:G |
| rs689716 | 16335349 | 0.492 | C:T | rs541299 | 16342126 | 0.450 | T:C |
| rs13320451 | 16335678 | 0.175 | G:A | rs563395 | 16342262 | 0.383 | G:A |
| rs555948 | 16335837 | 0.358 | A:C | rs568798 | 16342826 | 0.442 | C:A |
| rs11922434 | 16336067 | 0.175 | C:G | rs568833 | 16342840 | 0.400 | C:T |
| rs11922469 | 16336112 | 0.175 | C:A | rs690145 | 16343125 | 0.450 | C:T |
| rs478483 | 16337133 | 0.500 | G:G | rs519819 | 16343579 | 0.450 | T:C |
| rs567541 | 16339621 | 0.483 | G:A | rs690094 | 16343703 | 0.450 | T:C |
| rs690216 | 16339923 | 0.483 | C:A | rs690606 | 16345499 | 0.292 | G:A |
| rs483682 | 16340227 | 0.342 | G:A | rs581152 | 16345679 | 0.292 | T:C |
| rs2459641 | 16340490 | 0.325 | C:G | rs1607226 | 16348575 | 0.158 | C:T |
| rs6765960 | 16341640 | 0.483 | G:A | | | | |

TABLE 118
Singleton Tag
Block 002
Chromosome 3
Rs17005751 (BP 19217997) LD block SNPs

| | | | | | | | |
|---|---|---|---|---|---|---|---|
| | | Minor | | SNP | | Minor | |
| SNP | Base | Allele | | ID | Base | Allele | |
| ID (rs) | Position | Freq | Alleles | (rs) | Position | Freq | Alleles |

No LD
block

TABLE 119
Singleton Tag
Block 003
Chromosome 3
Rs17006139 (BP 19691079) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs1567948 | 19580149 | 0.408 | T:C | rs2222007 | 19655710 | 0.183 | G:T |
| rs9917819 | 19580521 | 0.075 | C:T | rs6804427 | 19658377 | 0.400 | G:A |
| rs9819921 | 19580839 | 0.325 | C:T | rs17006125 | 19659185 | 0.075 | T:C |
| rs17006069 | 19581028 | 0.075 | C:T | rs17006126 | 19660032 | 0.067 | G:A |
| rs4858383 | 19585353 | 0.342 | C:T | rs10049043 | 19660487 | 0.342 | T:C |
| rs949665 | 19588760 | 0.333 | G:T | rs17006128 | 19662549 | 0.075 | C:G |
| rs7652049 | 19588938 | 0.008 | T:C | rs17572637 | 19662888 | 0.117 | T:C |
| rs12493322 | 19589002 | 0.008 | A:T | rs9841179 | 19681808 | 0.325 | G:A |
| rs10510494 | 19589049 | 0.058 | A:G | rs2090177 | 19685007 | 0.408 | T:C |
| rs17543807 | 19589284 | 0.175 | A:G | rs2090178 | 19685225 | 0.300 | C:T |
| rs2203429 | 19589378 | 0.333 | C:T | rs7640905 | 19685337 | 0.008 | T:C |
| rs13098580 | 19590168 | 0.183 | T:C | rs12233409 | 19686818 | 0.008 | T:C |
| rs1033195 | 19591742 | 0.408 | T:C | rs6789046 | 19687913 | 0.408 | A:C |
| rs13075095 | 19593841 | 0.175 | T:C | rs10510493 | 19692203 | 0.067 | C:G |
| rs1983304 | 19597488 | 0.408 | A:T | rs954151 | 19692810 | 0.408 | T:C |
| rs12489594 | 19597865 | 0.183 | C:T | rs17006159 | 19699596 | 0.008 | T:C |
| rs17006078 | 19598041 | 0.058 | T:C | rs12054325 | 19700208 | 0.008 | T:C |
| rs17006083 | 19598228 | 0.033 | G:C | rs12106778 | 19701312 | 0.008 | A:C |
| rs17006085 | 19603004 | 0.067 | A:T | rs10222655 | 19702544 | 0.108 | T:A |
| rs6793281 | 19603701 | 0.333 | G:T | rs4858067 | 19703436 | 0.308 | A:T |
| rs9866549 | 19603993 | 0.342 | A:G | rs2886236 | 19714218 | 0.008 | C:G |
| rs1473604 | 19614854 | 0.408 | A:C | rs7627626 | 19715849 | 0.067 | C:T |
| rs9873476 | 19615749 | 0.183 | C:T | rs13087932 | 19715937 | 0.142 | G:T |
| rs7646927 | 19621331 | 0.233 | C:G | rs7644800 | 19717319 | 0.008 | G:A |
| rs17006096 | 19622633 | 0.067 | C:T | rs1395127 | 19720281 | 0.008 | A:G |
| rs2364998 | 19626539 | 0.400 | G:A | rs6784806 | 19726991 | 0.333 | T:C |
| rs17006097 | 19626744 | 0.075 | A:G | rs9831487 | 19727375 | 0.408 | T:C |
| rs6804525 | 19628391 | 0.308 | A:G | rs2062976 | 19730423 | 0.067 | A:G |
| rs17006105 | 19629176 | 0.008 | T:C | rs2062975 | 19730466 | 0.050 | C:T |
| rs6787710 | 19647577 | 0.408 | C:T | rs1506103 | 19732620 | 0.067 | C:T |
| rs9857864 | 19647868 | 0.408 | T:C | rs1506102 | 19732898 | 0.067 | T:C |
| rs9873199 | 19650495 | 0.408 | G:C | rs4858519 | 19735087 | 0.308 | C:A |
| rs7650101 | 19650756 | 0.408 | A:G | rs12491396 | 19736083 | 0.183 | T:C |
| rs2203428 | 19655412 | 0.067 | C:T | rs1506101 | 19736205 | 0.408 | T:A |
| rs1847913 | 19739740 | 0.075 | G:T | rs10510489 | 19749296 | 0.075 | T:G |
| rs6780502 | 19740371 | 0.183 | T:C | rs17006216 | 19757054 | 0.075 | G:T |
| rs17572742 | 19747551 | 0.067 | G:A | rs17006217 | 19758281 | 0.158 | T:C |
| rs9822740 | 19748284 | 0.333 | G:A | rs9310598 | 19758458 | 0.192 | G:A |
| rs4858088 | 19748986 | 0.017 | G:A | | | | |

TABLE 120
Singleton Tag
Block 004
Chromosome 3
Rs2033776 (BP 29439472) LD block SNPs

| | | Minor | |
|---|---|---|---|
| | Base | Allele | |
| SNP ID (rs) | Position | Freq | Alleles |
| rs7433749 | 29434998 | 0.108 | T:C |
| rs1438329 | 29435099 | 0.108 | G:A |
| rs2118600 | 29436242 | 0.325 | T:G |
| rs17666176 | 29436458 | 0.208 | A:G |
| rs17023466 | 29436628 | 0.058 | G:T |
| rs7640471 | 29437120 | 0.017 | T:G |
| rs7651092 | 29437312 | 0.308 | G:A |
| rs7634956 | 29438972 | 0.383 | T:C |
| rs12715155 | 29439827 | 0.317 | T:G |
| rs12629616 | 29440758 | 0.108 | C:T |
| rs9813729 | 29440902 | 0.008 | G:A |
| rs2053288 | 29440928 | 0.375 | T:C |
| rs9861686 | 29442935 | 0.317 | G:A |
| rs17023474 | 29443221 | 0.108 | T:A |
| rs17666332 | 29444679 | 0.267 | T:G |
| rs12629272 | 29445197 | 0.108 | C:G |
| rs12630203 | 29446789 | 0.108 | G:A |

TABLE 121
Singleton Tag
Block 005
Chromosome 3
Rs17054509 (BP 54893785) LD block SNPs

| | | Minor | |
|---|---|---|---|
| | Base | Allele | |
| SNP ID (rs) | Position | Freq | Alleles |
| rs2694111 | 54893548 | 0.392 | T:C |
| rs11716356 | 54894225 | 0.142 | A:G |
| rs1405330 | 54894312 | 0.283 | G:A |
| rs10510774 | 54894391 | 0.175 | A:G |
| rs2293662 | 54894520 | 0.142 | G:A |
| rs2694116 | 54895555 | 0.292 | T:G |
| rs2694115 | 54895597 | 0.392 | G:C |
| rs4263264 | 54895676 | 0.150 | T:G |
| rs3773594 | 54896943 | 0.142 | T:C |
| rs2360659 | 54897275 | 0.117 | G:A |
| rs2360660 | 54897353 | 0.117 | C:T |
| rs2970534 | 54897445 | 0.292 | A:G |
| rs13320480 | 54898523 | 0.008 | C:T |
| rs17252876 | 54901249 | 0.175 | C:A |
| rs17252966 | 54902941 | 0.317 | A:T |
| rs10510775 | 54903214 | 0.300 | C:A |
| rs1526594 | 54903802 | 0.142 | G:C |
| rs9868201 | 54904540 | 0.142 | T:C |
| rs9868221 | 54904586 | 0.142 | T:G |
| rs9848771 | 54904630 | 0.142 | G:A |
| rs6445708 | 54905739 | 0.142 | A:T |
| rs17253119 | 54905813 | 0.158 | A:G |
| rs17054524 | 54905834 | 0.142 | C:T |
| rs17054525 | 54907980 | 0.275 | G:A |
| rs17253231 | 54909094 | 0.158 | A:C |
| rs1969481 | 54909443 | 0.425 | A:G |
| rs17319169 | 54910580 | 0.158 | A:G |
| rs17054529 | 54911729 | 0.142 | T:G |
| rs6772996 | 54914162 | 0.125 | A:G |
| rs10514715 | 54914551 | 0.158 | G:A |
| rs9839518 | 54914743 | 0.008 | A:T |
| rs17054531 | 54916348 | 0.400 | C:T |
| rs9808962 | 54916481 | 0.292 | T:G |
| rs1918070 | 54916618 | 0.292 | T:C |
| rs2141034 | 54918069 | 0.117 | T:A |
| rs1358172 | 54918266 | 0.283 | G:A |
| rs12491410 | 54919483 | 0.400 | T:C |

TABLE 122
Singleton Tag
BLock 006
Chromosome 3
Rs1880214 (BP 61195849) LD block SNPs

| | | | | | | | |
|---|---|---|---|---|---|---|---|
| | | Minor | | SNP | | Minor | |
| SNP | Base | Allele | | ID | Base | Allele | |
| ID (rs) | Position | Freq | Alleles | (rs) | Position | Freq | Alleles |

No LD
Block

TABLE 123
Singleton Tag
BLock 007
Chromosome 3
Rs1444477 (BP 78679108) LD block SNPs

| | | Minor | |
|---|---|---|---|
| | Base | Allele | |
| SNP ID (rs) | Position | Freq | Alleles |
| rs9876636 | 78677509 | 0.075 | G:A |
| rs9876667 | 78677572 | 0.075 | G:A |
| rs7428104 | 78678025 | 0.075 | G:A |
| rs1470377 | 78678265 | 0.075 | C:T |
| rs1373524 | 78680972 | 0.075 | T:G |
| rs1444472 | 78683588 | 0.075 | C:T |
| rs9682630 | 78686977 | 0.075 | A:G |

TABLE 124
Singleton Tag
BLock 001
Chromosome 4
Rs6815823 (BP 13515096) LD block SNPs

| | | Minor | | SNP | | Minor | |
|---|---|---|---|---|---|---|---|
| SNP | Base | Allele | | ID | Base | Allele | |
| ID (rs) | Position | Freq | Alleles | (rs) | Position | Freq | Alleles |
| No LD | | | | | | | |
| Block | | | | | | | |

TABLE 125
Singleton Tag
BLock 002
Chromosome 4
SNP_A-2081938 (BP 48491108) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs1565217 | 48320646 | 0.208 | C:T | rs4235156 | 48403639 | 0.050 | A:G |
| rs1565218 | 48320675 | 0.025 | A:G | rs9884546 | 48407562 | 0.458 | T:C |
| rs992423 | 48322904 | 0.442 | C:T | rs7693461 | 48409187 | 0.458 | G:A |
| rs7697851 | 48324858 | 0.450 | T:C | rs7695813 | 48409755 | 0.050 | A:C |
| rs11945562 | 48327526 | 0.442 | C:T | rs17656125 | 48410376 | 0.075 | T:C |
| rs6824125 | 48329956 | 0.183 | C:A | rs4610307 | 48411351 | 0.050 | T:C |
| rs3811759 | 48331938 | 0.192 | A:G | rs17575720 | 48413133 | 0.450 | C:T |
| rs9993088 | 48335474 | 0.275 | C:A | rs17575727 | 48413966 | 0.083 | T:C |
| rs17609740 | 48335852 | 0.050 | G:A | rs11727472 | 48415657 | 0.050 | T:C |
| rs13114836 | 48336505 | 0.175 | C:G | rs11732827 | 48416449 | 0.117 | A:G |
| rs7694862 | 48336543 | 0.325 | C:T | rs7679210 | 48417592 | 0.450 | A:T |
| rs17472113 | 48336590 | 0.275 | T:A | rs6447646 | 48421395 | 0.050 | G:C |
| rs17472127 | 48337296 | 0.175 | A:G | rs17656189 | 48421611 | 0.050 | T:C |
| rs17609761 | 48337732 | 0.050 | C:T | rs4695391 | 48423837 | 0.492 | G:A |
| rs10031777 | 48339218 | 0.450 | T:C | rs7695730 | 48424805 | 0.050 | T:C |
| rs10018344 | 48339598 | 0.450 | C:G | rs3107023 | 48425333 | 0.050 | C:T |
| rs10029732 | 48339607 | 0.450 | A:G | rs17656212 | 48426044 | 0.050 | G:C |
| rs6811148 | 48357063 | 0.500 | A:A | rs776591 | 48430347 | 0.500 | C:C |
| rs7661515 | 48358280 | 0.450 | C:A | rs13127730 | 48431534 | 0.458 | C:T |
| rs16861344 | 48359872 | 0.050 | A:T | rs776590 | 48432078 | 0.492 | T:G |
| rs13134960 | 48366979 | 0.050 | A:G | rs17656255 | 48432578 | 0.050 | T:C |
| rs10938534 | 48367567 | 0.450 | G:A | rs6447648 | 48432988 | 0.050 | T:G |
| rs16861347 | 48371797 | 0.050 | A:G | rs2089503 | 48437415 | 0.458 | G:A |
| rs6855022 | 48374076 | 0.492 | C:A | rs776599 | 48439537 | 0.050 | C:G |
| rs937888 | 48384824 | 0.492 | C:A | rs776598 | 48440208 | 0.050 | C:T |
| rs10517225 | 48386742 | 0.050 | A:T | rs6856448 | 48440419 | 0.450 | T:C |
| rs7683398 | 48387081 | 0.450 | T:C | rs1507863 | 48443400 | 0.042 | G:C |
| rs952289 | 48387969 | 0.467 | A:C | rs1507864 | 48446556 | 0.117 | A:G |
| rs17656094 | 48402727 | 0.492 | G:A | rs2945330 | 48447454 | 0.500 | A:A |
| rs13110719 | 48402791 | 0.050 | G:A | rs776602 | 48452857 | 0.242 | A:C |
| rs1472859 | 48403265 | 0.042 | G:A | rs3107022 | 48458315 | 0.492 | C:T |
| rs4536901 | 48403268 | 0.425 | A:G | rs6843340 | 48460865 | 0.442 | C:T |
| rs7690681 | 48403431 | 0.050 | G:C | rs17575888 | 48467240 | 0.067 | C:T |
| rs4695388 | 48403558 | 0.050 | T:C | rs1876469 | 48468120 | 0.450 | C:T |
| rs776584 | 48474428 | 0.492 | G:C | rs7697522 | 48580301 | 0.500 | C:C |
| rs796555 | 48477523 | 0.500 | C:C | rs13106064 | 48581524 | 0.450 | C:T |
| rs776593 | 48478981 | 0.050 | A:G | rs4695400 | 48583608 | 0.492 | G:A |
| rs776594 | 48479134 | 0.050 | T:C | rs17610398 | 48589128 | 0.050 | C:T |
| rs776596 | 48482604 | 0.042 | C:G | rs1554258 | 48590045 | 0.450 | T:C |
| rs10938537 | 48484576 | 0.458 | C:G | rs10003273 | 48597465 | 0.450 | G:C |
| rs1712762 | 48485702 | 0.050 | A:G | rs1980193 | 48599891 | 0.500 | C:C |
| rs13119041 | 48486158 | 0.450 | A:C | rs17576077 | 48599919 | 0.267 | C:T |
| rs937889 | 48490190 | 0.500 | C:C | rs1554256 | 48604937 | 0.458 | T:C |
| rs776586 | 48490703 | 0.050 | G:A | rs7657698 | 48606103 | 0.492 | G:A |
| rs1712767 | 48504303 | 0.492 | G:A | rs7660206 | 48606715 | 0.450 | C:T |
| rs12505376 | 48507798 | 0.050 | T:A | rs2176460 | 48611234 | 0.500 | A:A |
| rs2354312 | 48511676 | 0.450 | G:A | rs13119414 | 48613033 | 0.500 | A:A |
| rs12509766 | 48526734 | 0.050 | C:A | rs7657332 | 48619080 | 0.500 | C:C |
| rs10049761 | 48527885 | 0.450 | G:T | rs1517675 | 48619864 | 0.050 | T:C |
| rs1996422 | 48528279 | 0.283 | A:G | rs1517674 | 48619903 | 0.458 | T:C |
| rs6830848 | 48531336 | 0.492 | G:T | rs6447662 | 48623575 | 0.250 | C:A |
| rs12650202 | 48533699 | 0.008 | G:C | rs13142540 | 48624515 | 0.475 | T:C |
| rs6829081 | 48534175 | 0.283 | A:T | rs13141523 | 48630197 | 0.442 | G:A |
| rs12650421 | 48540140 | 0.267 | A:C | rs2354937 | 48635331 | 0.342 | T:G |
| rs7670939 | 48544444 | 0.458 | A:G | rs2354938 | 48637984 | 0.317 | G:A |
| rs6447651 | 48550352 | 0.450 | C:T | rs4998802 | 48641838 | 0.258 | G:A |
| rs4695397 | 48553434 | 0.492 | C:A | rs11727343 | 48647125 | 0.467 | A:G |
| rs11732876 | 48561913 | 0.058 | T:C | rs13140483 | 48647690 | 0.025 | A:C |
| rs2354314 | 48562255 | 0.500 | C:C | rs13149083 | 48650873 | 0.458 | C:T |
| rs13106353 | 48569161 | 0.017 | G:A | rs7654032 | 48651107 | 0.458 | G:A |
| rs6447655 | 48570180 | 0.492 | G:A | rs7665209 | 48655615 | 0.475 | C:T |

TABLE 126
Singleton Tag
BLock 003
Chromosome 4
Rs17090112 (BP 61222559) LD block SNPs

| | | Minor | |
|---|---|---|---|
| | Base | Allele | |
| SNP ID (rs) | Position | Freq | Alleles |
| rs7436824 | 61202262 | 0.058 | G:A |
| rs2340729 | 61203755 | 0.050 | C:T |
| rs13141702 | 61203942 | 0.492 | A:T |
| rs12500687 | 61206629 | 0.042 | A:G |
| rs10031736 | 61207190 | 0.050 | C:T |
| rs7666247 | 61207328 | 0.125 | G:C |
| rs6835610 | 61208781 | 0.050 | G:A |
| rs1511120 | 61209158 | 0.050 | A:G |
| rs1039924 | 61212749 | 0.050 | A:G |
| rs1397664 | 61213661 | 0.050 | A:G |
| rs13109810 | 61218094 | 0.017 | A:G |
| rs6845040 | 61218111 | 0.050 | T:C |
| rs10014777 | 61221226 | 0.017 | C:A |
| rs17090111 | 61221805 | 0.025 | A:T |
| rs1355402 | 61222305 | 0.050 | C:T |
| rs2136955 | 61223700 | 0.025 | A:G |
| rs1553862 | 61224222 | 0.050 | C:A |
| rs17291517 | 61226622 | 0.125 | T:C |
| rs17291524 | 61226816 | 0.125 | A:G |
| rs1511133 | 61227704 | 0.175 | T:C |
| rs6551568 | 61227857 | 0.175 | C:G |
| rs1155572 | 61228411 | 0.050 | C:T |
| rs1155569 | 61228592 | 0.050 | A:G |
| rs6819235 | 61228699 | 0.025 | C:T |
| rs17219415 | 61229827 | 0.125 | A:C |
| rs10024492 | 61230052 | 0.175 | T:C |
| rs1553869 | 61230816 | 0.050 | C:T |
| rs1553866 | 61231274 | 0.025 | T:G |
| rs11929952 | 61231435 | 0.008 | G:T |
| rs9990993 | 61231446 | 0.033 | G:A |
| rs10517506 | 61232206 | 0.075 | A:C |
| rs10517503 | 61232486 | 0.175 | C:G |
| rs11723596 | 61232606 | 0.175 | G:A |
| rs1912682 | 61233554 | 0.125 | T:C |
| rs17090126 | 61233965 | 0.025 | G:A |
| rs17291580 | 61234098 | 0.125 | A:T |
| rs2340736 | 61234638 | 0.050 | C:T |
| rs2340737 | 61234726 | 0.050 | T:G |
| rs1553865 | 61234774 | 0.175 | T:C |
| rs6857178 | 61236328 | 0.175 | A:C |
| rs13151747 | 61236364 | 0.025 | G:A |
| rs7675101 | 61236399 | 0.025 | C:T |
| rs6551570 | 61236528 | 0.175 | G:A |
| rs2340738 | 61236774 | 0.050 | T:C |

TABLE 127
Singleton Tag
BLock 004
Chromosome 4
Rs10012057 (BP 112318188) LD block SNPs

| | | Minor | |
|---|---|---|---|
| | Base | Allele | |
| SNP ID (rs) | Position | Freq | Alleles |
| rs1487055 | 112302842 | 0.367 | A:C |
| rs6844132 | 112303004 | 0.367 | T:A |
| rs7668879 | 112304336 | 0.033 | C:T |
| rs7673921 | 112304527 | 0.033 | C:T |
| rs159480 | 112305038 | 0.092 | T:C |
| rs7675079 | 112305142 | 0.033 | C:G |
| rs9307361 | 112307047 | 0.025 | A:G |
| rs4833525 | 112309943 | 0.075 | C:T |
| rs159476 | 112309994 | 0.367 | T:C |
| rs12649290 | 112310428 | 0.025 | G:A |
| rs1386389 | 112310874 | 0.308 | A:C |
| rs10024441 | 112314562 | 0.283 | G:A |
| rs159471 | 112318290 | 0.083 | T:C |
| rs159470 | 112319343 | 0.358 | T:G |
| rs6850556 | 112320066 | 0.275 | G:A |
| rs6827504 | 112320122 | 0.358 | T:C |
| rs12645166 | 112321199 | 0.033 | T:C |
| rs17527016 | 112321323 | 0.283 | C:T |
| rs977013 | 112321686 | 0.283 | C:T |
| rs17588641 | 112321811 | 0.025 | C:T |
| rs159453 | 112323574 | 0.367 | T:C |
| rs6840447 | 112323694 | 0.367 | A:G |
| rs1601658 | 112327178 | 0.367 | T:C |
| rs1905510 | 112332343 | 0.367 | T:C |

TABLE 128
Singleton Tag
BLock 005
Chromosome 4
Rs17046129 (BP 114789761) LD block SNPs

| | | Minor | |
|---|---|---|---|
| | Base | Allele | |
| SNP ID (rs) | Position | Freq | Alleles |
| rs4834341 | 114767887 | 0.433 | A:G |
| rs17591997 | 114774077 | 0.083 | C:G |
| rs2158197 | 114774225 | 0.425 | T:G |
| rs17446418 | 114775610 | 0.283 | T:G |
| rs757174 | 114775746 | 0.442 | T:C |
| rs17592032 | 114776683 | 0.033 | T:C |
| rs17592039 | 114777998 | 0.033 | T:C |
| rs17446453 | 114779316 | 0.292 | C:T |
| rs874110 | 114780635 | 0.242 | G:A |
| rs10488891 | 114783078 | 0.008 | T:C |
| rs7684265 | 114783302 | 0.050 | T:C |
| rs4834347 | 114783538 | 0.283 | C:A |
| rs4834348 | 114783919 | 0.475 | T:G |
| rs6533690 | 114784348 | 0.442 | T:C |
| rs10488892 | 114784516 | 0.033 | A:G |
| rs7697831 | 114785409 | 0.242 | G:A |
| rs11098193 | 114787011 | 0.275 | C:T |
| rs1476542 | 114788668 | 0.033 | C:T |
| rs17046126 | 114789677 | 0.308 | C:T |

TABLE 129
Singleton Tag
Block 006
Chromosome 4
SNP_A-2177362 (BP 153149584) LD block SNPs

| | | Minor | |
|---|---|---|---|
| | Base | Allele | |
| SNP ID (rs) | Position | Freq | Alleles |
| rs361168 | 153147079 | 0.425 | G:C |
| rs361167 | 153148233 | 0.117 | G:A |
| rs13145151 | 153150151 | 0.292 | G:T |
| rs361166 | 153150396 | 0.400 | A:G |
| rs361165 | 153152305 | 0.117 | T:C |
| rs2062329 | 153153298 | 0.283 | G:A |
| rs361163 | 153157488 | 0.417 | G:A |
| rs361162 | 153157737 | 0.117 | A:T |
| rs715947 | 153159527 | 0.208 | C:A |
| rs361161 | 153160228 | 0.117 | T:C |
| rs901207 | 153161745 | 0.117 | T:C |
| rs1385780 | 153162304 | 0.117 | A:G |
| rs361160 | 153163048 | 0.117 | G:A |
| rs17028013 | 153163143 | 0.033 | T:A |
| rs361159 | 153163214 | 0.117 | G:A |

TABLE 130
Singleton Tag
Block 001
Chromosome 5
Rs16891296 (BP 33573443) LD block SNPs

| | | Minor | |
|---|---|---|---|
| | Base | Allele | |
| SNP ID (rs) | Position | Freq | Alleles |
| rs27739 | 33562198 | 0.308 | T:A |
| rs256605 | 33563775 | 0.450 | C:T |
| rs10521004 | 33565020 | 0.167 | T:C |
| rs7702210 | 33565155 | 0.167 | C:T |
| rs7722715 | 33565540 | 0.167 | T:G |
| rs256602 | 33566313 | 0.483 | C:T |
| rs190463 | 33566857 | 0.483 | G:A |
| rs256792 | 33567402 | 0.317 | C:T |
| rs25750 | 33568045 | 0.483 | C:T |
| rs27975 | 33569436 | 0.483 | T:C |
| rs28100 | 33569622 | 0.317 | C:G |
| rs25754 | 33570817 | 0.483 | G:A |
| rs25755 | 33571571 | 0.308 | G:T |
| rs25756 | 33571790 | 0.483 | T:C |
| rs27850 | 33571985 | 0.483 | G:A |
| rs27404 | 33572373 | 0.483 | G:C |
| rs17566960 | 33573459 | 0.075 | G:A |
| rs256645 | 33575123 | 0.417 | G:A |
| rs17488958 | 33575147 | 0.167 | T:C |
| rs17488979 | 33575185 | 0.008 | T:C |
| rs29967 | 33582189 | 0.417 | A:G |

TABLE 131
Singleton Tag
Block 002
Chromosome 5
Rs11953095 (BP 96425437) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| No LD Block | | | | | | | |

TABLE 132
Singleton Tag
Block 003
Chromosome 5
Rs1422010 (BP 143812852) LD block SNPs

| | | Minor | |
|---|---|---|---|
| | Base | Allele | |
| SNP ID (rs) | Position | Freq | Alleles |
| rs2017862 | 143799520 | 0.267 | G:A |
| rs1363220 | 143803021 | 0.208 | A:G |
| rs11956551 | 143812254 | 0.008 | G:A |
| rs4912974 | 143820908 | 0.408 | C:G |
| rs349688 | 143822542 | 0.400 | T:C |
| rs145716 | 143827411 | 0.408 | T:C |
| rs349686 | 143827636 | 0.400 | T:G |
| rs239471 | 143827963 | 0.400 | T:C |
| rs349685 | 143828085 | 0.400 | C:T |
| rs349682 | 143829551 | 0.308 | T:C |
| rs349681 | 143829583 | 0.400 | G:C |
| rs6580321 | 143831025 | 0.283 | G:A |
| rs712171 | 143831036 | 0.400 | G:A |
| rs712173 | 143831937 | 0.400 | G:A |
| rs712174 | 143831962 | 0.400 | G:A |
| rs349704 | 143835224 | 0.400 | A:G |
| rs349701 | 143835846 | 0.400 | G:C |
| rs349699 | 143836448 | 0.400 | C:G |
| rs349698 | 143836721 | 0.408 | T:C |
| rs349697 | 143837222 | 0.400 | A:G |
| rs349696 | 143837516 | 0.383 | C:A |
| rs349695 | 143838477 | 0.400 | C:T |
| rs349694 | 143839256 | 0.400 | G:A |
| rs358650 | 143840630 | 0.333 | A:G |
| rs2032862 | 143840846 | 0.400 | G:C |
| rs1422961 | 143841548 | 0.400 | T:A |
| rs10477241 | 143843889 | 0.383 | C:T |
| rs6868013 | 143844468 | 0.400 | C:G |
| rs6893431 | 143844615 | 0.400 | T:A |
| rs2305555 | 143845463 | 0.400 | A:G |
| rs6874554 | 143845980 | 0.383 | C:A |
| rs11749671 | 143846442 | 0.400 | A:G |
| rs6894880 | 143847920 | 0.383 | G:A |
| rs6882650 | 143849197 | 0.333 | A:G |
| rs7711675 | 143850732 | 0.375 | G:T |
| rs13356666 | 143854372 | 0.400 | G:A |
| rs467019 | 143860939 | 0.225 | G:T |
| rs358673 | 143861266 | 0.267 | G:T |
| rs3096072 | 143862645 | 0.400 | A:G |
| rs12332411 | 143870573 | 0.025 | A:T |
| rs17102012 | 143871043 | 0.025 | T:C |
| rs463245 | 143878381 | 0.400 | T:C |
| rs358656 | 143878647 | 0.350 | A:G |
| rs17773401 | 143878675 | 0.042 | A:G |
| rs358657 | 143878998 | 0.233 | C:T |
| rs13171242 | 143879681 | 0.292 | A:G |
| rs13189971 | 143879869 | 0.292 | C:T |
| rs13175530 | 143879928 | 0.292 | T:G |

TABLE 133
Singleton Tag
Block 004
Chromosome 5
Rs10041351 (BP 174633402) LD block SNPs

| | | Minor | |
|---|---|---|---|
| | Base | Allele | |
| SNP ID (rs) | Position | Freq | Alleles |
| rs10476151 | 174661009 | 0.392 | G:T |
| rs6888523 | 174662132 | 0.408 | A:G |
| rs1379278 | 174665184 | 0.192 | A:T |
| rs4868508 | 174667819 | 0.017 | C:T |
| rs7732899 | 174668830 | 0.117 | G:A |
| rs1017685 | 174669199 | 0.408 | T:A |
| rs11957065 | 174672306 | 0.042 | G:A |
| rs7446806 | 174675330 | 0.033 | T:C |
| rs1902602 | 174676120 | 0.375 | G:T |
| rs1902601 | 174676174 | 0.033 | T:G |
| rs11955914 | 174676704 | 0.042 | C:T |
| rs6873940 | 174677494 | 0.150 | G:A |
| rs12189407 | 174677963 | 0.133 | T:C |
| rs4867791 | 174680233 | 0.358 | T:C |
| rs11960475 | 174681339 | 0.400 | A:G |
| rs11960551 | 174681657 | 0.192 | G:A |
| rs12716312 | 174682737 | 0.150 | T:C |
| rs12659973 | 174683211 | 0.150 | T:C |
| rs13190572 | 174683531 | 0.150 | T:C |
| rs17063592 | 174686886 | 0.042 | C:A |
| rs2471016 | 174688914 | 0.450 | A:G |

TABLE 134
Singleton Tag
Block 001
Chromosome 6
Rs2875970 (BP 4807112) LD block SNPs

| | | Minor | |
|---|---|---|---|
| | Base | Allele | |
| SNP ID (rs) | Position | Freq | Alleles |
| rs1883327 | 4793933 | 0.367 | A:G |
| rs2235429 | 4797203 | 0.017 | T:C |
| rs4557570 | 4798390 | 0.025 | G:A |
| rs9504271 | 4798954 | 0.017 | G:C |
| rs808611 | 4803431 | 0.342 | A:G |
| rs808607 | 4806304 | 0.133 | C:T |
| rs808603 | 4808290 | 0.308 | C:T |
| rs6926191 | 4809002 | 0.083 | A:G |
| rs12198622 | 4809182 | 0.125 | C:T |
| rs808600 | 4809812 | 0.133 | A:G |
| rs808599 | 4809878 | 0.025 | C:G |
| rs808598 | 4810635 | 0.133 | C:A |
| rs9504276 | 4811820 | 0.017 | C:T |
| rs808589 | 4818364 | 0.300 | A:G |
| rs17282871 | 4819385 | 0.050 | G:A |
| rs808635 | 4821722 | 0.133 | A:G |
| rs808634 | 4823124 | 0.333 | T:C |
| rs808622 | 4828582 | 0.350 | T:C |
| rs13195561 | 4830599 | 0.017 | C:A |
| rs9504281 | 4832252 | 0.017 | T:C |
| rs808615 | 4834922 | 0.333 | T:C |
| rs9504284 | 4835273 | 0.017 | G:A |
| rs811251 | 4835470 | 0.017 | C:T |
| rs17355232 | 4835664 | 0.033 | G:A |
| rs17138931 | 4839000 | 0.017 | T:C |
| rs7768749 | 4840846 | 0.017 | G:A |
| rs6916304 | 4841183 | 0.017 | T:C |
| rs6916467 | 4841247 | 0.017 | T:C |
| rs9502249 | 4842133 | 0.017 | T:A |
| rs1891045 | 4842575 | 0.133 | A:G |
| rs4960046 | 4846920 | 0.133 | G:A |
| rs1557032 | 4848176 | 0.017 | A:G |
| rs1417657 | 4849379 | 0.350 | A:G |
| rs2326553 | 4850501 | 0.033 | A:G |
| rs12110776 | 4852140 | 0.025 | G:A |
| rs12110777 | 4852157 | 0.025 | G:A |
| rs13212871 | 4852283 | 0.033 | G:C |
| rs13199417 | 4852533 | 0.033 | A:G |
| rs4960047 | 4853286 | 0.142 | C:T |
| rs7742807 | 4854436 | 0.142 | C:T |
| rs1891046 | 4855369 | 0.142 | G:A |
| rs1891047 | 4855649 | 0.175 | T:C |
| rs10080391 | 4857440 | 0.033 | T:C |
| rs12195125 | 4858993 | 0.058 | A:G |
| rs7775887 | 4859743 | 0.150 | G:A |
| rs9405785 | 4860027 | 0.458 | A:G |
| rs9378918 | 4860158 | 0.150 | T:C |
| rs4144546 | 4860202 | 0.142 | T:C |
| rs4144547 | 4860422 | 0.175 | A:T |
| rs6924037 | 4860647 | 0.117 | A:G |
| rs9378919 | 4860840 | 0.117 | A:G |
| rs7770206 | 4862142 | 0.033 | C:G |
| rs7771387 | 4862156 | 0.142 | G:C |
| rs17138959 | 4862188 | 0.017 | G:C |
| rs11242983 | 4862352 | 0.200 | A:G |
| rs9392068 | 4862632 | 0.142 | T:C |
| rs17138966 | 4862779 | 0.017 | T:C |
| rs9378920 | 4863053 | 0.142 | T:C |
| rs7759221 | 4863700 | 0.142 | T:A |
| rs7738275 | 4863736 | 0.142 | C:G |
| rs7738459 | 4863882 | 0.142 | A:G |
| rs7739834 | 4863993 | 0.142 | T:C |
| rs3930304 | 4864216 | 0.142 | G:T |
| rs9392640 | 4864384 | 0.117 | T:A |
| rs3930303 | 4865084 | 0.250 | T:A |
| rs3863214 | 4865229 | 0.158 | A:G |
| rs9405786 | 4868183 | 0.117 | A:G |

TABLE 135
Singleton Tag
Block 002
Chromosome 6
rs259920 (BP 30133738) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs9261203 | 30113022 | 0.067 | A:G | rs9261258 | 30130787 | 0.067 | G:A |
| rs9261204 | 30113222 | 0.092 | A:G | rs9261260 | 30131331 | 0.042 | T:G |
| rs4711207 | 30113733 | 0.367 | C:A | rs3869070 | 30131847 | 0.408 | T:C |
| rs9261207 | 30114461 | 0.067 | T:C | rs9261261 | 30132344 | 0.067 | A:G |
| rs9405044 | 30114744 | 0.358 | G:A | rs9366752 | 30132656 | 0.275 | C:T |
| rs259934 | 30114855 | 0.317 | C:T | rs9261263 | 30132728 | 0.067 | C:G |
| rs259935 | 30115030 | 0.358 | G:A | rs6911724 | 30133158 | 0.067 | G:A |
| rs259937 | 30115472 | 0.058 | T:C | rs9261264 | 30133189 | 0.067 | T:C |
| rs10947047 | 30115690 | 0.025 | G:A | rs6911634 | 30133264 | 0.067 | C:T |
| rs11962771 | 30115963 | 0.025 | G:A | rs259919 | 30133482 | 0.400 | G:A |
| rs259938 | 30115982 | 0.358 | C:G | rs259920 | 30133738 | 0.058 | G:A |
| rs6915205 | 30116022 | 0.025 | C:T | rs6917477 | 30133963 | 0.067 | G:C |
| rs9261212 | 30116975 | 0.067 | T:C | rs16896944 | 30134125 | 0.025 | T:C |
| rs9393987 | 30117148 | 0.358 | C:T | rs12208867 | 30134215 | 0.042 | G:A |
| rs12661609 | 30117240 | 0.025 | G:A | rs10484549 | 30134269 | 0.042 | T:C |
| rs12664794 | 30117643 | 0.025 | A:G | rs9261265 | 30134329 | 0.067 | T:C |
| rs9261215 | 30117758 | 0.042 | A:C | rs3757327 | 30135392 | 0.067 | G:A |
| rs9261216 | 30118118 | 0.092 | A:G | rs3132130 | 30135431 | 0.067 | G:C |
| rs9380150 | 30118471 | 0.358 | T:C | rs3132129 | 30135839 | 0.067 | G:A |
| rs9261217 | 30118487 | 0.092 | T:C | rs3757329 | 30136403 | 0.067 | A:C |
| rs2855593 | 30118936 | 0.058 | C:T | rs9261266 | 30136531 | 0.042 | C:T |
| rs9261218 | 30119254 | 0.067 | G:A | rs7769930 | 30136786 | 0.067 | A:C |
| rs9261219 | 30119430 | 0.067 | G:A | rs1150742 | 30136810 | 0.317 | T:C |
| rs259939 | 30119560 | 0.358 | T:C | rs7770092 | 30136850 | 0.067 | C:T |
| rs9261220 | 30119842 | 0.067 | G:T | rs9261267 | 30136873 | 0.042 | G:A |
| rs259940 | 30119913 | 0.317 | A:G | rs7770505 | 30136892 | 0.067 | G:A |
| rs6940552 | 30120319 | 0.025 | G:A | rs7770557 | 30137088 | 0.067 | C:G |
| rs6905501 | 30123047 | 0.067 | T:C | rs1150741 | 30137630 | 0.317 | C:G |
| rs259942 | 30123146 | 0.150 | C:T | rs9261268 | 30138036 | 0.067 | G:A |
| rs6457140 | 30124789 | 0.067 | A:T | rs9261269 | 30138093 | 0.067 | G:A |
| rs2844800 | 30125735 | 0.058 | A:G | rs9261270 | 30138114 | 0.067 | G:A |
| rs259945 | 30127185 | 0.083 | T:A | rs9261271 | 30138168 | 0.067 | T:A |
| rs7761314 | 30130132 | 0.067 | C:T | rs1150740 | 30138668 | 0.083 | C:A |
| rs9261257 | 30130404 | 0.067 | G:A | rs9261275 | 30138907 | 0.017 | C:T |
| rs9261276 | 30139038 | 0.067 | C:T | rs3188482 | 30140652 | 0.025 | A:G |
| rs9261277 | 30139070 | 0.067 | T:C | rs16896970 | 30140896 | 0.042 | A:G |
| rs1150739 | 30139324 | 0.425 | T:C | rs3807030 | 30141669 | 0.067 | G:T |
| rs9261278 | 30139341 | 0.067 | C:T | rs3807031 | 30141863 | 0.308 | C:A |
| rs8321 | 30140501 | 0.142 | A:C | | | | |

TABLE 136
Singleton Tag
Block 003
Chromosome 6
Rs6926440 (BP 53511839) LD block SNPs

| | | Minor | |
|---|---|---|---|
| | Base | Allele | |
| SNP ID (rs) | Position | Freq | Alleles |
| rs1002269 | 53501158 | 0.050 | T:C |
| rs4715407 | 53501693 | 0.058 | G:A |
| rs2268329 | 53503322 | 0.050 | T:C |
| rs7758764 | 53504121 | 0.058 | G:A |
| rs12527352 | 53504732 | 0.008 | T:C |
| rs12664895 | 53505170 | 0.008 | A:G |
| rs16883966 | 53505685 | 0.050 | A:G |
| rs6933870 | 53505929 | 0.475 | G:C |
| rs6934367 | 53505974 | 0.058 | G:A |
| rs502862 | 53506518 | 0.392 | T:C |
| rs4712035 | 53509062 | 0.150 | C:G |
| rs4715408 | 53509118 | 0.050 | T:C |
| rs2397147 | 53509546 | 0.458 | T:C |
| rs3799698 | 53509862 | 0.050 | C:T |
| rs3799699 | 53509958 | 0.050 | C:T |
| rs547109 | 53510504 | 0.058 | A:C |
| rs547222 | 53510544 | 0.050 | T:C |
| rs606548 | 53510638 | 0.050 | C:T |
| rs2284650 | 53512241 | 0.042 | A:G |

TABLE 137
Singleton Tag
Block 004
Chromosome 6
Rs4119563 (BP 56229665) LD block SNPs

| | | Minor | |
|---|---|---|---|
| | Base | Allele | |
| SNP ID (rs) | Position | Freq | Alleles |
| rs9396184 | 56220911 | 0.275 | A:G |
| rs6919257 | 56221259 | 0.333 | G:T |
| rs9475643 | 56221540 | 0.317 | T:C |
| rs1925158 | 56222614 | 0.217 | A:G |
| rs994039 | 56222956 | 0.108 | A:C |
| rs994040 | 56223149 | 0.217 | C:G |
| rs6931381 | 56223185 | 0.092 | G:A |
| rs6911260 | 56223448 | 0.133 | G:T |
| rs12530020 | 56223853 | 0.208 | C:A |
| rs12665485 | 56224014 | 0.217 | C:T |
| rs4119562 | 56224546 | 0.217 | G:C |
| rs10948980 | 56225996 | 0.317 | C:G |
| rs10948981 | 56226958 | 0.217 | T:C |
| rs1925159 | 56227729 | 0.317 | A:T |
| rs724678 | 56228253 | 0.317 | T:C |
| rs724677 | 56228680 | 0.150 | G:A |
| rs9396185 | 56228789 | 0.108 | C:T |
| rs7760380 | 56228859 | 0.275 | G:T |
| rs1925160 | 56229398 | 0.217 | T:G |
| rs9296835 | 56229526 | 0.317 | G:A |
| rs4119563 | 56229665 | 0.217 | T:C |
| rs3846928 | 56230235 | 0.108 | A:G |
| rs9296836 | 56231285 | 0.158 | G:C |
| rs13195010 | 56231552 | 0.317 | G:A |
| rs3846930 | 56231657 | 0.158 | C:T |
| rs4119564 | 56231717 | 0.317 | T:C |
| rs3846931 | 56231793 | 0.217 | T:C |
| rs4119565 | 56231928 | 0.317 | T:G |
| rs9475645 | 56232107 | 0.317 | T:C |
| rs9464368 | 56232149 | 0.317 | T:C |
| rs10223442 | 56233685 | 0.200 | C:T |
| rs9370495 | 56234025 | 0.217 | C:A |
| rs6923130 | 56234255 | 0.317 | G:A |
| rs2147696 | 56234746 | 0.317 | A:T |
| rs10498810 | 56234862 | 0.108 | C:A |
| rs12525967 | 56234901 | 0.208 | C:T |
| rs1925164 | 56235226 | 0.467 | G:A |
| rs1925163 | 56235580 | 0.467 | G:A |
| rs4571565 | 56235790 | 0.317 | A:G |
| rs13202214 | 56236809 | 0.317 | T:G |
| rs12528835 | 56237664 | 0.467 | A:T |
| rs12663282 | 56238085 | 0.100 | G:A |
| rs4715601 | 56238615 | 0.150 | C:T |
| rs4715602 | 56238626 | 0.217 | T:C |
| rs9382598 | 56240581 | 0.217 | C:T |
| rs1925140 | 56241890 | 0.317 | T:A |
| rs13219390 | 56242691 | 0.317 | C:A |
| rs4336451 | 56242812 | 0.108 | T:G |
| rs9396188 | 56246042 | 0.317 | A:G |
| rs11757974 | 56246336 | 0.317 | G:T |
| rs10948986 | 56246715 | 0.317 | G:A |
| rs12110798 | 56248063 | 0.092 | A:G |
| rs2397212 | 56251190 | 0.208 | C:T |
| rs2397214 | 56254519 | 0.450 | C:T |

TABLE 138
Singleton Tag
Block 005
Chromosome 6
Rs6941591 (BP 113833795) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs6908767 | 113831706 | 0.183 | A:T | rs9320451 | 113857709 | 0.367 | T:C |
| rs9400623 | 113833204 | 0.083 | A:G | rs2049924 | 113858994 | 0.367 | G:A |
| rs6941591 | 113833795 | 0.008 | G:T | rs12175106 | 113859980 | 0.367 | C:T |
| rs10484461 | 113834023 | 0.117 | C:T | rs2294231 | 113861097 | 0.442 | T:C |
| rs9400624 | 113834202 | 0.192 | C:T | rs7776236 | 113861905 | 0.450 | C:T |
| rs9918379 | 113836873 | 0.100 | G:T | rs7738052 | 113862149 | 0.183 | G:A |
| rs10457266 | 113838618 | 0.450 | C:G | rs7758769 | 113862259 | 0.442 | T:C |
| rs9488172 | 113838672 | 0.008 | A:G | rs909543 | 113862599 | 0.083 | G:A |
| rs9320446 | 113839088 | 0.183 | T:C | rs909542 | 113863286 | 0.367 | C:G |
| rs9320447 | 113839404 | 0.083 | G:A | rs9481352 | 113864305 | 0.450 | G:T |
| rs6915099 | 113839695 | 0.175 | G:A | rs9320452 | 113864483 | 0.367 | T:C |
| rs6568788 | 113842736 | 0.192 | T:C | rs9285410 | 113864611 | 0.083 | G:A |
| rs9488175 | 113842784 | 0.008 | G:A | rs12214468 | 113865146 | 0.442 | G:A |
| rs17075128 | 113842860 | 0.050 | G:T | rs7759869 | 113866085 | 0.450 | C:A |
| rs9374395 | 113845964 | 0.083 | T:C | rs2881870 | 113866183 | 0.183 | C:A |
| rs9374397 | 113846286 | 0.192 | C:T | rs2348297 | 113866417 | 0.133 | C:A |
| rs9320449 | 113846476 | 0.192 | G:C | rs1033391 | 113866756 | 0.450 | C:T |
| rs9320450 | 113846499 | 0.183 | C:T | rs1033390 | 113867219 | 0.450 | C:T |
| rs6915133 | 113846745 | 0.192 | A:G | rs9374399 | 113868017 | 0.183 | G:A |
| rs12192120 | 113847844 | 0.267 | G:A | rs9320454 | 113868285 | 0.083 | T:C |
| rs7767942 | 113848107 | 0.083 | C:G | rs9320455 | 113868569 | 0.450 | C:G |
| rs7768104 | 113848230 | 0.192 | T:G | rs9384867 | 113870412 | 0.367 | G:A |
| rs4945495 | 113849335 | 0.183 | A:G | rs12195748 | 113870808 | 0.025 | T:C |
| rs9488180 | 113849492 | 0.008 | G:A | rs6928402 | 113872220 | 0.367 | G:T |
| rs7739755 | 113849909 | 0.192 | G:A | rs12203134 | 113874382 | 0.450 | T:G |
| rs2348294 | 113850776 | 0.192 | G:T | rs6903091 | 113874861 | 0.183 | A:G |
| rs11153416 | 113850821 | 0.192 | C:A | rs6903628 | 113875150 | 0.183 | A:G |
| rs718460 | 113851269 | 0.192 | A:G | rs10484459 | 113876160 | 0.450 | A:G |
| rs718459 | 113851635 | 0.183 | C:G | rs6914474 | 113876715 | 0.450 | A:C |
| rs9488182 | 113853427 | 0.008 | G:A | rs766330 | 113881089 | 0.458 | G:T |
| rs6568789 | 113853641 | 0.183 | G:C | rs4380793 | 113881706 | 0.442 | T:A |
| rs6568790 | 113855642 | 0.183 | A:C | rs12183323 | 113882154 | 0.008 | A:T |
| rs4945496 | 113855702 | 0.183 | G:A | rs11153420 | 113883104 | 0.442 | A:G |
| rs9481351 | 113855946 | 0.008 | A:G | rs9481358 | 113884012 | 0.442 | G:A |
| rs2179118 | 113884335 | 0.442 | C:T | rs7771550 | 113885343 | 0.442 | G:C |
| rs9488189 | 113884441 | 0.442 | C:A | rs7771810 | 113885735 | 0.442 | A:G |
| rs9488190 | 113884626 | 0.442 | G:T | rs7772108 | 113885884 | 0.442 | A:C |
| rs7766686 | 113884957 | 0.383 | A:T | rs7738898 | 113886489 | 0.442 | G:A |
| rs9398344 | 113884995 | 0.375 | G:A | | | | |

TABLE 139
Singleton Tag
Block 006
Chromosome 6
Rs9496804 (BP 144260788) LD block SNPs

| | | Minor | |
|---|---|---|---|
| | Base | Allele | |
| SNP ID (rs) | Position | Freq | Alleles |
| rs6570586 | 144251197 | 0.100 | T:C |
| rs4895623 | 144251895 | 0.025 | A:G |
| rs4896685 | 144257808 | 0.025 | A:G |
| rs12527718 | 144258438 | 0.025 | T:C |
| rs761541 | 144258666 | 0.083 | G:A |
| rs764667 | 144258772 | 0.025 | G:A |
| rs4896686 | 144259487 | 0.083 | T:A |
| rs7764938 | 144262097 | 0.233 | C:T |
| rs13202317 | 144263625 | 0.050 | G:A |
| rs9496805 | 144264623 | 0.058 | G:A |
| rs9484827 | 144266005 | 0.017 | T:G |
| rs2092716 | 144276593 | 0.075 | T:C |
| rs6570589 | 144281769 | 0.408 | G:T |
| rs9496815 | 144282654 | 0.017 | C:T |
| rs4896690 | 144285909 | 0.325 | A:G |
| rs9484833 | 144293054 | 0.492 | C:A |
| rs12665265 | 144296688 | 0.158 | C:T |
| rs7768375 | 144297918 | 0.333 | T:C |
| rs9321950 | 144298963 | 0.333 | T:C |
| rs12528289 | 144299137 | 0.400 | G:A |
| rs10484817 | 144299332 | 0.042 | A:C |
| rs6570592 | 144299937 | 0.492 | C:T |
| rs7761382 | 144300052 | 0.333 | C:T |
| rs1884087 | 144300312 | 0.333 | A:G |
| rs3736746 | 144301247 | 0.500 | A:A |

TABLE 140
Singleton Tag
Block 007
Chromosome 6
Rs17076363 (BP 147206578) LD block SNPs

| | | Minor | |
|---|---|---|---|
| | Base | Allele | |
| SNP ID (rs) | Position | Freq | Alleles |
| rs259385 | 147170024 | 0.467 | A:G |
| rs259375 | 147173789 | 0.217 | T:C |
| rs259374 | 147174147 | 0.217 | C:G |
| rs3861404 | 147175339 | 0.317 | C:T |
| rs9377028 | 147176900 | 0.317 | G:C |
| rs259370 | 147177937 | 0.467 | G:A |
| rs1052449 | 147178137 | 0.317 | A:C |
| rs7744139 | 147179922 | 0.433 | T:A |
| rs633409 | 147181811 | 0.150 | A:G |
| rs477801 | 147182462 | 0.433 | G:A |
| rs9386170 | 147184183 | 0.392 | C:A |
| rs9373511 | 147184359 | 0.392 | T:A |
| rs9485129 | 147184984 | 0.392 | T:A |
| rs9377030 | 147185680 | 0.392 | G:T |
| rs9377031 | 147185835 | 0.392 | C:T |
| rs259368 | 147185885 | 0.150 | A:G |
| rs9390431 | 147186040 | 0.392 | A:G |
| rs9403820 | 147187734 | 0.392 | A:T |
| rs508146 | 147188850 | 0.150 | A:G |
| rs1324677 | 147190509 | 0.100 | A:G |
| rs9399585 | 147191284 | 0.392 | T:C |
| rs9485132 | 147192206 | 0.433 | T:A |
| rs12211458 | 147198112 | 0.150 | A:G |
| rs1924695 | 147199920 | 0.442 | T:C |
| rs1324679 | 147202921 | 0.383 | G:A |
| rs1324681 | 147203942 | 0.383 | C:T |
| rs9373512 | 147204493 | 0.383 | C:T |
| rs10457058 | 147212027 | 0.150 | G:A |
| rs10457059 | 147212120 | 0.150 | G:A |
| rs12216213 | 147212438 | 0.150 | C:T |
| rs12196674 | 147213031 | 0.150 | T:G |
| rs9322078 | 147213193 | 0.383 | G:A |
| rs6909070 | 147213269 | 0.150 | T:C |
| rs6929822 | 147213727 | 0.150 | A:C |
| rs6915214 | 147214423 | 0.408 | T:A |
| rs6915391 | 147214513 | 0.442 | C:T |
| rs12208399 | 147215536 | 0.150 | A:G |
| rs6927572 | 147218297 | 0.392 | G:A |
| rs2328770 | 147218657 | 0.442 | A:G |
| rs12190780 | 147218703 | 0.150 | C:G |
| rs12191031 | 147218935 | 0.150 | G:T |
| rs6929209 | 147219159 | 0.442 | A:G |
| rs9497651 | 147220028 | 0.150 | T:C |
| rs9497653 | 147220130 | 0.150 | A:C |
| rs9377032 | 147220191 | 0.442 | C:T |
| rs9403822 | 147220222 | 0.383 | A:C |
| rs9497654 | 147220247 | 0.150 | T:G |
| rs9399586 | 147220715 | 0.375 | A:G |
| rs9399587 | 147221744 | 0.442 | A:T |
| rs6570778 | 147222278 | 0.442 | T:C |
| rs9322079 | 147224032 | 0.475 | G:A |
| rs3736886 | 147224801 | 0.158 | T:C |
| rs12202364 | 147225128 | 0.183 | C:T |
| rs12203958 | 147225359 | 0.150 | G:C |
| rs6927327 | 147225407 | 0.375 | G:C |
| rs9399588 | 147226034 | 0.425 | C:T |

TABLE 141
Singleton Tag
Block 001
Chromosome 7
Rs2079495 (BP 25276568) LD block SNPs

| | | Minor | |
|---|---|---|---|
| | Base | Allele | |
| SNP ID (rs) | Position | Freq | Alleles |
| rs17151656 | 25269383 | 0.208 | T:C |
| rs17317200 | 25269814 | 0.183 | C:T |
| rs10266599 | 25270714 | 0.017 | A:G |
| rs7802027 | 25271633 | 0.192 | T:C |
| rs10271456 | 25272149 | 0.017 | A:G |
| rs17317312 | 25273759 | 0.092 | C:A |
| rs986302 | 25274242 | 0.208 | T:C |
| rs4719814 | 25274496 | 0.258 | A:G |
| rs4722468 | 25275780 | 0.200 | T:C |
| rs10486456 | 25276343 | 0.025 | A:G |
| rs2079495 | 25276568 | 0.017 | A:T |
| rs3857726 | 25276631 | 0.017 | C:T |
| rs6958937 | 25276991 | 0.208 | T:G |
| rs3857727 | 25277255 | 0.192 | A:T |
| rs2391133 | 25277661 | 0.208 | A:G |
| rs10266720 | 25277813 | 0.017 | G:A |
| rs4722469 | 25278613 | 0.383 | A:C |

TABLE 142
Singleton Tag
Block 002
Chromosome 7
Rs2366858 (BP 79985273) LD block SNPs

| | | Minor | |
|---|---|---|---|
| | Base | Allele | |
| SNP ID (rs) | Position | Freq | Alleles |
| rs12055973 | 79984910 | 0.100 | G:T |
| rs11971792 | 79985814 | 0.100 | A:G |
| rs11982937 | 79987065 | 0.100 | C:G |
| rs11765496 | 79994802 | 0.100 | T:C |
| rs13239216 | 79995015 | 0.167 | A:G |
| rs954410 | 79996324 | 0.100 | G:A |
| rs954409 | 79996382 | 0.100 | C:G |
| rs1880957 | 79998880 | 0.100 | G:A |
| rs1527465 | 79999064 | 0.108 | G:A |
| rs1880958 | 79999164 | 0.108 | A:C |
| rs17154350 | 79999861 | 0.400 | C:G |
| rs16886849 | 80002614 | 0.100 | T:C |
| rs1527467 | 80004457 | 0.008 | G:A |
| rs1852538 | 80009652 | 0.008 | A:G |
| rs11768155 | 80010507 | 0.008 | A:G |
| rs12531778 | 80011267 | 0.458 | G:C |
| rs1527470 | 80015480 | 0.425 | A:G |
| rs1949971 | 80018993 | 0.008 | G:A |
| rs1358340 | 80019837 | 0.450 | C:A |
| rs2886792 | 80020912 | 0.008 | C:T |
| rs6966341 | 80023328 | 0.433 | T:C |
| rs3778673 | 80027226 | 0.108 | C:T |
| rs3778674 | 80027243 | 0.108 | C:A |
| rs17154380 | 80028256 | 0.108 | C:A |

TABLE 143
Singleton Tag
Block 001
Chromosome 8
Rs1238058 (BP 4265211) LD block SNPs

| | | Minor | |
|---|---|---|---|
| | Base | Allele | |
| SNP ID (rs) | Position | Freq | Alleles |
| rs7837221 | 4264705 | 0.008 | G:C |
| rs1217534 | 4265269 | 0.058 | T:G |
| rs10091211 | 4265530 | 0.225 | A:C |
| rs1238884 | 4265664 | 0.050 | G:A |

TABLE 144
Singleton Tag
Block 002
Chromosome 8
Rs1107050 (BP 34056049) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs10107220 | 33945205 | 0.192 | A:G | rs12681942 | 33980234 | 0.008 | G:T |
| rs11786029 | 33945583 | 0.083 | A:G | rs12679394 | 33980517 | 0.083 | C:A |
| rs17707910 | 33946209 | 0.042 | A:T | rs7823476 | 33981611 | 0.242 | A:G |
| rs11777303 | 33947338 | 0.042 | G:A | rs4458868 | 33982165 | 0.083 | A:G |
| rs12674668 | 33947371 | 0.083 | A:C | rs7462574 | 33982722 | 0.008 | T:C |
| rs17780750 | 33949281 | 0.042 | C:T | rs7463344 | 33983069 | 0.008 | G:C |
| rs10087815 | 33949448 | 0.108 | C:T | rs10503954 | 33983246 | 0.242 | C:T |
| rs17708017 | 33949588 | 0.042 | A:T | rs4493901 | 33984189 | 0.133 | C:T |
| rs13272274 | 33951284 | 0.083 | C:T | rs6468211 | 33984241 | 0.008 | A:G |
| rs12155673 | 33951569 | 0.192 | G:A | rs7464063 | 33984328 | 0.008 | A:G |
| rs17708047 | 33952925 | 0.042 | C:T | rs4493902 | 33984345 | 0.133 | C:G |
| rs12677417 | 33953933 | 0.083 | G:T | rs6415401 | 33984428 | 0.008 | G:A |
| rs7009719 | 33954366 | 0.358 | C:G | rs4625032 | 33984545 | 0.133 | T:C |
| rs7008747 | 33954428 | 0.408 | A:G | rs7462709 | 33985151 | 0.242 | G:T |
| rs7463110 | 33956575 | 0.450 | C:A | rs13252171 | 33986201 | 0.083 | A:C |
| rs13270569 | 33957977 | 0.083 | C:A | rs17781255 | 33986371 | 0.042 | G:A |
| rs11780381 | 33958129 | 0.042 | T:C | rs10086539 | 33986446 | 0.108 | G:A |
| rs7464251 | 33962061 | 0.008 | G:T | rs6468212 | 33987051 | 0.008 | T:C |
| rs10954945 | 33965126 | 0.083 | C:T | rs6415402 | 33988808 | 0.008 | A:C |
| rs10095900 | 33966794 | 0.008 | C:A | rs16881372 | 33988864 | 0.008 | G:T |
| rs4739564 | 33968192 | 0.242 | A:G | rs12678950 | 33991908 | 0.025 | G:A |
| rs7461792 | 33968985 | 0.242 | G:C | rs17708611 | 33992154 | 0.042 | G:A |
| rs10100986 | 33970290 | 0.008 | A:C | rs12677633 | 33992280 | 0.050 | C:T |
| rs17781020 | 33970400 | 0.358 | C:G | rs6468213 | 33994031 | 0.008 | C:A |
| rs13277692 | 33971950 | 0.083 | T:C | rs12546968 | 33995789 | 0.350 | A:G |
| rs7465297 | 33973410 | 0.242 | A:G | rs7387781 | 33999617 | 0.050 | T:G |
| rs7459878 | 33974241 | 0.008 | A:G | rs7386889 | 33999689 | 0.050 | G:T |
| rs10088032 | 33974459 | 0.008 | A:G | rs7386891 | 33999739 | 0.042 | A:T |
| rs7388319 | 33975544 | 0.008 | C:T | rs7011293 | 33999861 | 0.242 | C:T |
| rs13263773 | 33975644 | 0.008 | A:G | rs13255963 | 34001764 | 0.083 | T:C |
| rs7386717 | 33977934 | 0.008 | C:T | rs6468214 | 34001937 | 0.242 | A:C |
| rs6468210 | 33978375 | 0.008 | A:G | rs10088296 | 34002284 | 0.050 | G:A |
| rs10105179 | 33978741 | 0.008 | C:T | rs10088770 | 34002668 | 0.083 | A:G |
| rs7459636 | 33979345 | 0.008 | C:G | rs7014681 | 34004016 | 0.050 | A:T |
| rs4395895 | 34005681 | 0.108 | G:T | rs6999743 | 34055751 | 0.008 | T:C |
| rs6993412 | 34007445 | 0.242 | T:A | rs11780434 | 34055840 | 0.042 | G:A |
| rs7462405 | 34008153 | 0.083 | G:A | rs3892595 | 34055945 | 0.008 | G:A |
| rs10110830 | 34009748 | 0.108 | A:C | rs1107050 | 34056049 | 0.008 | C:T |
| rs13266685 | 34011971 | 0.025 | C:T | rs11778193 | 34056368 | 0.042 | T:C |
| rs11775654 | 34018525 | 0.133 | T:C | rs10954951 | 34056433 | 0.133 | A:G |
| rs10954947 | 34023001 | 0.050 | T:C | rs11984559 | 34056524 | 0.008 | C:A |
| rs10954948 | 34023204 | 0.050 | C:A | rs17782229 | 34056858 | 0.042 | T:C |
| rs11778168 | 34026227 | 0.042 | T:C | rs6468225 | 34057047 | 0.008 | G:A |
| rs6468220 | 34026894 | 0.008 | G:T | rs7006050 | 34057559 | 0.008 | A:G |
| rs10090438 | 34027518 | 0.050 | C:G | rs16875739 | 34058605 | 0.008 | C:T |
| rs11779346 | 34030297 | 0.242 | G:A | rs4495420 | 34058733 | 0.008 | C:T |
| rs12156335 | 34042207 | 0.083 | G:A | rs10107670 | 34059809 | 0.108 | G:A |
| rs7843517 | 34043002 | 0.242 | C:T | rs17709548 | 34059907 | 0.042 | A:G |
| rs16881395 | 34044175 | 0.008 | G:T | rs10503956 | 34060543 | 0.008 | G:A |
| rs9693056 | 34045718 | 0.050 | G:T | rs13277112 | 34060804 | 0.242 | G:A |
| rs10954950 | 34046772 | 0.242 | A:G | rs10103681 | 34062541 | 0.008 | A:C |
| rs4357281 | 34047190 | 0.108 | C:A | rs10091466 | 34062814 | 0.008 | G:A |
| rs10092240 | 34047829 | 0.008 | T:C | rs10110998 | 34064076 | 0.108 | A:G |
| rs13256886 | 34047992 | 0.083 | A:G | rs12155702 | 34064385 | 0.092 | C:A |
| rs10092725 | 34048176 | 0.050 | T:C | rs12155970 | 34064572 | 0.083 | T:C |
| rs6995729 | 34052095 | 0.008 | G:A | rs11778404 | 34066088 | 0.042 | C:T |
| rs11775180 | 34052891 | 0.242 | T:C | rs4739403 | 34066499 | 0.342 | G:A |
| rs10216480 | 34053154 | 0.008 | T:A | rs10954953 | 34067324 | 0.083 | T:G |
| rs10216776 | 34053171 | 0.008 | G:A | rs11784068 | 34068499 | 0.042 | A:C |
| rs10216404 | 34053291 | 0.008 | A:T | rs11992308 | 34069983 | 0.008 | T:C |
| rs10216786 | 34053535 | 0.008 | G:A | rs7465648 | 34070004 | 0.008 | A:G |
| rs10216502 | 34053684 | 0.008 | T:C | rs11991650 | 34070294 | 0.008 | A:G |
| rs16881423 | 34054540 | 0.008 | G:T | rs7460936 | 34070969 | 0.108 | T:C |
| rs4631464 | 34054635 | 0.083 | T:A | rs11781645 | 34071032 | 0.042 | A:C |
| rs4517117 | 34054691 | 0.083 | C:T | rs13258238 | 34071296 | 0.083 | A:G |
| rs4307341 | 34055240 | 0.083 | A:G | rs11989916 | 34072109 | 0.008 | C:A |
| rs10503955 | 34055306 | 0.042 | A:G | rs11993633 | 34072179 | 0.133 | A:T |
| rs7012473 | 34055450 | 0.008 | G:A | rs17709746 | 34072386 | 0.042 | T:C |
| rs17709764 | 34072638 | 0.042 | A:G | rs7830902 | 34083057 | 0.008 | G:T |
| rs17196272 | 34073123 | 0.042 | A:G | rs2729966 | 34084936 | 0.242 | C:G |
| rs10101028 | 34073329 | 0.267 | T:C | rs11779489 | 34085036 | 0.083 | G:T |
| rs11781918 | 34074819 | 0.083 | C:T | rs11777413 | 34086263 | 0.042 | A:C |
| rs7818248 | 34075391 | 0.242 | G:A | rs2643283 | 34087101 | 0.242 | T:C |
| rs7840675 | 34075414 | 0.108 | T:C | rs16881471 | 34087619 | 0.008 | C:G |
| rs13364903 | 34075927 | 0.108 | C:T | rs2643282 | 34089744 | 0.242 | A:C |
| rs13363649 | 34075971 | 0.242 | G:A | rs2729985 | 34095147 | 0.242 | G:T |
| rs10088298 | 34076960 | 0.108 | T:C | rs10093529 | 34095846 | 0.192 | T:A |
| rs10088740 | 34077274 | 0.108 | T:C | rs10112049 | 34097932 | 0.192 | C:T |
| rs12674520 | 34077713 | 0.083 | A:G | rs1874263 | 34099593 | 0.008 | G:A |
| rs12676808 | 34077870 | 0.083 | G:A | rs2729990 | 34100540 | 0.008 | T:C |
| rs7832148 | 34077954 | 0.092 | G:A | rs7843751 | 34101581 | 0.392 | A:G |
| rs4128481 | 34080271 | 0.242 | C:A | | | | |

TABLE 145
Singleton Tag
Block 003
Chromosome 8
Rs6472980 (BP 77732821) LD block SNPs

| | | Minor | |
|---|---|---|---|
| | Base | Allele | |
| SNP ID (rs) | Position | Freq | Alleles |
| rs10504628 | 77679977 | 0.050 | A:G |
| rs10504629 | 77680089 | 0.467 | G:A |
| rs16939322 | 77681683 | 0.058 | T:C |
| rs16939323 | 77682792 | 0.142 | G:T |
| rs10448038 | 77683860 | 0.142 | A:G |
| rs17348470 | 77684641 | 0.083 | G:A |
| rs16939324 | 77685358 | 0.025 | T:C |
| rs3213864 | 77685632 | 0.017 | A:G |
| rs1433963 | 77686075 | 0.042 | T:C |
| rs10957811 | 77686475 | 0.350 | A:T |
| rs10957812 | 77687776 | 0.117 | A:G |
| rs1433961 | 77689407 | 0.350 | A:T |
| rs9643428 | 77689735 | 0.017 | G:A |
| rs7845698 | 77689744 | 0.208 | G:C |
| rs1560846 | 77692021 | 0.208 | A:C |
| rs7842561 | 77697776 | 0.025 | T:A |
| rs16939325 | 77698209 | 0.058 | G:A |
| rs10504630 | 77699065 | 0.025 | C:T |
| rs6980742 | 77700214 | 0.058 | G:A |
| rs10504631 | 77701042 | 0.183 | A:G |
| rs16939326 | 77701234 | 0.025 | T:C |
| rs10504632 | 77701636 | 0.083 | C:T |
| rs721439 | 77701692 | 0.025 | C:T |
| rs17348969 | 77701877 | 0.083 | G:A |
| rs17349004 | 77702519 | 0.083 | T:G |
| rs6996695 | 77703136 | 0.042 | G:A |
| rs6472975 | 77703214 | 0.325 | G:A |
| rs1594820 | 77707775 | 0.333 | A:T |
| rs7817568 | 77708557 | 0.333 | G:C |
| rs12708084 | 77713999 | 0.383 | C:T |
| rs9773544 | 77714196 | 0.025 | A:G |
| rs11784020 | 77723029 | 0.333 | G:A |
| rs16939329 | 77726733 | 0.183 | G:C |
| rs17349214 | 77727853 | 0.333 | C:T |
| rs6472978 | 77728558 | 0.333 | C:T |
| rs6472979 | 77728720 | 0.333 | A:C |
| rs6992439 | 77728757 | 0.333 | T:C |
| rs1368848 | 77729020 | 0.183 | G:T |
| rs6996667 | 77729036 | 0.117 | T:C |
| rs726765 | 77729280 | 0.125 | T:A |
| rs7015611 | 77729703 | 0.033 | C:T |
| rs11989518 | 77730209 | 0.058 | A:G |
| rs1368847 | 77730316 | 0.492 | A:C |
| rs6472980 | 77732821 | 0.292 | C:G |
| rs16939332 | 77732854 | 0.125 | G:A |
| rs17434334 | 77734921 | 0.050 | A:C |
| rs17434383 | 77734971 | 0.050 | A:G |
| rs11777752 | 77736310 | 0.125 | C:T |
| rs919902 | 77736941 | 0.058 | T:C |

TABLE 146
Singleton Tag
Block 004
Chromosome 8
Rs7817645 (BP 85212373) LD block SNPs

| | | Minor | |
|---|---|---|---|
| | Base | Allele | |
| SNP ID (rs) | Position | Freq | Alleles |
| rs10958202 | 85167366 | 0.342 | A:G |
| rs6473522 | 85170809 | 0.292 | A:G |
| rs1866085 | 85174828 | 0.092 | G:A |
| rs10958203 | 85176327 | 0.342 | T:C |
| rs7825355 | 85178914 | 0.217 | A:G |
| rs10098485 | 85179726 | 0.025 | T:G |
| rs12675673 | 85180389 | 0.367 | C:G |
| rs969208 | 85182565 | 0.217 | G:A |
| rs7002217 | 85185040 | 0.217 | G:A |
| rs12679806 | 85187778 | 0.342 | G:A |
| rs7341568 | 85189484 | 0.217 | A:G |
| rs12674933 | 85189873 | 0.442 | T:C |
| rs16912463 | 85191055 | 0.008 | C:T |
| rs9298416 | 85192269 | 0.217 | A:G |
| rs10808857 | 85194559 | 0.442 | G:A |
| rs10738143 | 85194656 | 0.192 | G:A |
| rs7821100 | 85195457 | 0.217 | G:C |
| rs10504802 | 85195591 | 0.217 | C:T |
| rs12681393 | 85195633 | 0.217 | G:C |
| rs13269443 | 85196126 | 0.217 | C:G |
| rs7820224 | 85200889 | 0.217 | G:A |
| rs1866084 | 85202511 | 0.217 | T:A |
| rs12682316 | 85202674 | 0.108 | C:T |
| rs10958204 | 85203363 | 0.225 | G:A |
| rs7838770 | 85204262 | 0.208 | T:A |
| rs10481281 | 85211245 | 0.217 | A:C |
| rs12155772 | 85212368 | 0.008 | G:A |
| rs16912478 | 85213578 | 0.333 | C:T |
| rs10504800 | 85213945 | 0.108 | A:G |
| rs7017468 | 85214408 | 0.217 | C:T |
| rs7833023 | 85215796 | 0.283 | A:C |
| rs16912483 | 85216065 | 0.375 | T:C |

TABLE 147
Singleton Tag
Block 001
Chromosome 9
Rs10963149 (BP 1756892) LD block SNPs

| | | Minor | |
|---|---|---|---|
| | Base | Allele | |
| SNP ID (rs) | Position | Freq | Alleles |
| rs3858029 | 1746470 | 0.300 | A:C |
| rs3953809 | 1747039 | 0.300 | G:C |
| rs3953810 | 1747071 | 0.292 | G:A |
| rs4741599 | 1747693 | 0.300 | A:C |
| rs4741600 | 1747753 | 0.300 | C:T |
| rs7036840 | 1748555 | 0.067 | G:A |
| rs10810794 | 1748705 | 0.300 | C:G |
| rs486815 | 1749811 | 0.467 | C:T |
| rs10810799 | 1751505 | 0.300 | T:C |
| rs4741604 | 1752597 | 0.308 | T:C |
| rs10810809 | 1755756 | 0.292 | A:G |
| rs12682830 | 1756744 | 0.292 | A:G |
| rs10963156 | 1758656 | 0.283 | G:A |
| rs7847817 | 1759145 | 0.283 | T:C |
| rs4014158 | 1759325 | 0.283 | G:T |
| rs1817384 | 1759699 | 0.300 | A:T |
| rs10963164 | 1760930 | 0.292 | T:G |
| rs4740647 | 1760973 | 0.300 | A:G |
| rs10810815 | 1761793 | 0.292 | A:G |
| rs12554763 | 1761958 | 0.017 | C:G |
| rs4741605 | 1762869 | 0.292 | G:C |
| rs10963188 | 1763538 | 0.283 | G:T |
| rs10810825 | 1766776 | 0.333 | G:A |

TABLE 148
Singleton Tag
Block 002
Chromosome 9
Rs1413262 (BP 20127211) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 149
Singleton Tag
Block 003
Chromosome 9
Rs10124385 (BP 70986459) LD block SNPs

| | | Minor | |
|---|---|---|---|
| | Base | Allele | |
| SNP ID (rs) | Position | Freq | Alleles |
| rs1888076 | 70984988 | 0.325 | A:C |
| rs1888075 | 70985156 | 0.325 | A:G |
| rs1329778 | 70985467 | 0.208 | A:G |
| rs6560179 | 70985672 | 0.017 | T:A |
| rs1329777 | 70985828 | 0.017 | G:A |
| rs1329776 | 70986085 | 0.192 | T:C |
| rs10124385 | 70986459 | 0.017 | A:G |
| rs1472732 | 70986625 | 0.283 | G:T |

TABLE 150
Singleton Tag
Block 004
Chromosome 9
Rs17787401 (BP 76630755) LD block SNPs

| | | Minor | |
|---|---|---|---|
| | Base | Allele | |
| SNP ID (rs) | Position | Freq | Alleles |
| rs1417360 | 76627181 | 0.375 | C:A |
| rs11145060 | 76628007 | 0.017 | G:A |
| rs7862512 | 76629250 | 0.008 | C:T |
| rs12379855 | 76629356 | 0.017 | A:G |
| rs12237165 | 76629609 | 0.042 | C:T |
| rs954974 | 76629849 | 0.325 | G:T |
| rs17787401 | 76630755 | 0.008 | T:C |
| rs10113868 | 76631209 | 0.050 | T:C |
| rs11998737 | 76631299 | 0.025 | T:C |
| rs4585807 | 76631847 | 0.042 | T:A |
| rs12235152 | 76632028 | 0.050 | A:T |
| rs3924629 | 76632138 | 0.050 | T:C |
| rs10869818 | 76633587 | 0.250 | C:T |
| rs13295176 | 76633632 | 0.042 | T:C |
| rs10869819 | 76633916 | 0.050 | G:C |
| rs10869820 | 76634143 | 0.050 | A:G |
| rs7854027 | 76634303 | 0.258 | C:T |

TABLE 151
Singleton Tag
Block 005
Chromosome 9
Rs10978782 (BP 96588232) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 152
Singleton Tag
Block 006
Chromosome 9
Rs16934355 (BP 116997213) LD block SNPs

| | | Minor | |
|---|---|---|---|
| | Base | Allele | |
| SNP ID (rs) | Position | Freq | Alleles |
| rs997348 | 116969243 | 0.167 | C:G |
| rs2416591 | 116972203 | 0.400 | T:C |
| rs2416590 | 116975144 | 0.400 | C:T |
| rs10983541 | 116977767 | 0.175 | G:A |
| rs1413413 | 116978614 | 0.175 | T:C |
| rs4090221 | 116980932 | 0.225 | A:G |
| rs4614069 | 116981195 | 0.017 | G:A |
| rs12685948 | 116982685 | 0.150 | C:G |
| rs7027113 | 116983904 | 0.017 | G:A |
| rs7043225 | 116983917 | 0.017 | T:C |
| rs4837006 | 116984392 | 0.225 | C:G |
| rs16934343 | 116984991 | 0.017 | C:T |
| rs13283855 | 116985137 | 0.050 | T:C |
| rs10513287 | 116985304 | 0.400 | A:G |
| rs10818000 | 116985995 | 0.400 | A:G |
| rs10818001 | 116987023 | 0.150 | T:C |
| rs10818002 | 116987380 | 0.150 | C:T |
| rs17400793 | 116987862 | 0.150 | G:A |
| rs16934346 | 116988048 | 0.017 | G:A |
| rs7868099 | 116988182 | 0.208 | T:C |
| rs4837008 | 116988232 | 0.242 | G:A |
| rs4837009 | 116988579 | 0.225 | T:G |
| rs10818003 | 116989298 | 0.242 | A:C |
| rs12552667 | 116990621 | 0.225 | G:T |
| rs10983548 | 116990640 | 0.275 | G:A |
| rs1591256 | 116990685 | 0.158 | G:C |
| rs10983550 | 116991396 | 0.158 | G:A |
| rs10759904 | 116993046 | 0.158 | T:C |
| rs10983551 | 116993162 | 0.275 | C:T |
| rs7046318 | 116993221 | 0.117 | A:G |
| rs13298944 | 116993555 | 0.225 | G:T |
| rs17309781 | 116995623 | 0.075 | C:T |
| rs1413411 | 116996862 | 0.400 | A:G |
| rs1413410 | 116998708 | 0.400 | C:T |
| rs13291665 | 116999194 | 0.225 | A:T |
| rs7019490 | 116999585 | 0.100 | G:T |
| rs17309941 | 117002073 | 0.392 | A:G |
| rs13299059 | 117002110 | 0.225 | G:T |
| rs2416592 | 117002298 | 0.400 | A:T |
| rs4457407 | 117002585 | 0.008 | G:T |
| rs1334082 | 117002820 | 0.158 | C:T |

TABLE 153
Singleton Tag
Block 007
Chromosome 9
SNP_A-2281124 (BP 120455105) LD block SNPs

| | | Minor | |
|---|---|---|---|
| | Base | Allele | |
| SNP ID (rs) | Position | Freq | Alleles |
| rs959558 | 120448465 | 0.358 | G:C |
| rs991121 | 120449899 | 0.358 | G:C |
| rs2416760 | 120454193 | 0.358 | C:T |
| rs7861679 | 120455143 | 0.358 | T:C |
| rs10739570 | 120460776 | 0.342 | T:C |
| rs1324475 | 120463463 | 0.425 | C:T |
| rs1981021 | 120467947 | 0.350 | G:T |
| rs7357638 | 120468758 | 0.350 | A:G |
| rs10760109 | 120477130 | 0.017 | C:T |
| rs1158554 | 120480452 | 0.350 | A:G |
| rs1158553 | 120480528 | 0.350 | G:C |
| rs12235130 | 120483544 | 0.042 | G:A |
| rs10984971 | 120484062 | 0.042 | C:T |
| rs1324473 | 120484164 | 0.350 | C:T |
| rs1547267 | 120488290 | 0.342 | C:T |
| rs10984972 | 120489418 | 0.342 | C:T |
| rs4142158 | 120490373 | 0.342 | T:C |
| rs1886338 | 120491106 | 0.017 | T:C |
| rs10760110 | 120492117 | 0.342 | T:C |
| rs7024046 | 120498732 | 0.350 | A:G |
| rs10984974 | 120501110 | 0.350 | T:C |
| rs2274144 | 120501433 | 0.083 | A:G |
| rs1530370 | 120504106 | 0.350 | T:C |
| rs4837789 | 120505810 | 0.350 | A:G |
| rs1867254 | 120508632 | 0.350 | T:C |
| rs920745 | 120509497 | 0.350 | C:T |
| rs10491784 | 120511843 | 0.350 | C:A |
| rs1886337 | 120523330 | 0.350 | C:G |
| rs4837790 | 120525879 | 0.350 | T:C |
| rs10491783 | 120529709 | 0.350 | G:C |
| rs966396 | 120532835 | 0.350 | C:T |
| rs966397 | 120532866 | 0.350 | A:G |
| rs7849566 | 120540323 | 0.350 | C:A |
| rs10984984 | 120543030 | 0.058 | C:T |
| rs7044226 | 120545882 | 0.350 | C:G |
| rs10760112 | 120547124 | 0.350 | T:C |
| rs10760113 | 120553604 | 0.350 | C:T |
| rs10984994 | 120558323 | 0.342 | A:T |
| rs4617229 | 120560250 | 0.342 | G:A |
| rs2416799 | 120560420 | 0.342 | T:G |
| rs4837792 | 120563113 | 0.342 | A:G |
| rs4836830 | 120563222 | 0.342 | G:A |
| rs747819 | 120567995 | 0.342 | C:T |
| rs735110 | 120568494 | 0.342 | A:T |
| rs10818474 | 120569518 | 0.258 | T:C |
| rs10985007 | 120571229 | 0.133 | A:C |
| rs10985009 | 120572593 | 0.342 | C:T |
| rs7044106 | 120573616 | 0.300 | G:C |
| rs10985010 | 120577046 | 0.133 | G:A |
| rs10985011 | 120577149 | 0.142 | G:A |
| rs2900177 | 120577199 | 0.342 | T:C |

TABLE 154
Singleton Tag
Block 008
Chromosome 9
Rs2156323 (BP 133750375) LD block SNPs

| | | Minor | |
|---|---|---|---|
| | Base | Allele | |
| SNP ID (rs) | Position | Freq | Alleles |
| rs2510238 | 133728783 | 0.225 | A:G |
| rs2260095 | 133735510 | 0.192 | G:A |
| rs10993820 | 133737284 | 0.233 | A:G |
| rs12000549 | 133740068 | 0.175 | C:T |
| rs10821526 | 133741119 | 0.225 | T:C |
| rs739465 | 133741676 | 0.083 | G:A |
| rs2486350 | 133743236 | 0.167 | A:G |
| rs2428123 | 133743550 | 0.183 | T:C |
| rs735520 | 133744173 | 0.033 | C:T |
| rs1557678 | 133744675 | 0.183 | T:C |
| rs1557679 | 133744677 | 0.183 | T:C |
| rs1179486 | 133745953 | 0.192 | G:A |
| rs2486351 | 133746077 | 0.183 | C:T |
| rs10993823 | 133746103 | 0.175 | G:A |
| rs10993824 | 133746111 | 0.025 | T:A |
| rs2261150 | 133747810 | 0.208 | G:C |
| rs2510254 | 133748250 | 0.183 | A:G |
| rs10993826 | 133748599 | 0.183 | G:T |
| rs2073929 | 133750139 | 0.183 | G:C |
| rs3819500 | 133750353 | 0.183 | C:T |
| rs2156323 | 133750375 | 0.175 | G:A |
| rs3819502 | 133750632 | 0.183 | C:A |
| rs3819503 | 133750981 | 0.225 | C:A |
| rs10993829 | 133751295 | 0.225 | T:C |
| rs10993830 | 133751376 | 0.225 | G:C |
| rs1150061 | 133751583 | 0.033 | C:T |
| rs10993832 | 133752686 | 0.225 | C:A |
| rs12344583 | 133753074 | 0.233 | A:G |
| rs7023551 | 133754568 | 0.225 | A:G |
| rs17663678 | 133755593 | 0.225 | A:C |
| rs739466 | 133760981 | 0.192 | C:T |
| rs3780737 | 133762321 | 0.183 | C:T |
| rs10821527 | 133763529 | 0.183 | T:C |
| rs7038256 | 133764774 | 0.167 | C:T |
| rs7025939 | 133766672 | 0.167 | T:C |
| rs10993836 | 133768465 | 0.167 | A:G |
| rs3780741 | 133769640 | 0.167 | G:A |

TABLE 155
Singleton Tag
Block 001
Chromosome 10
Rs17369334 (BP 15563073) LD block SNPs

| | | Minor | |
|---|---|---|---|
| | Base | Allele | |
| SNP ID (rs) | Position | Freq | Alleles |
| rs4750648 | 15555068 | 0.433 | T:G |
| rs12263141 | 15555159 | 0.033 | G:C |
| rs4750649 | 15555183 | 0.400 | A:G |
| rs12256537 | 15555246 | 0.033 | A:T |
| rs7087978 | 15555488 | 0.400 | T:C |
| rs7086924 | 15555500 | 0.442 | A:G |
| rs12256803 | 15555675 | 0.042 | A:G |
| rs12256987 | 15555973 | 0.008 | A:T |
| rs12260246 | 15556408 | 0.033 | T:C |
| rs12265726 | 15556920 | 0.033 | G:C |
| rs12251291 | 15557229 | 0.033 | T:A |
| rs12251305 | 15557286 | 0.033 | T:C |
| rs12251360 | 15557324 | 0.033 | T:C |
| rs12256502 | 15557433 | 0.033 | G:A |
| rs2353401 | 15558038 | 0.433 | G:A |
| rs2167451 | 15559928 | 0.425 | T:C |
| rs17369299 | 15561885 | 0.075 | C:T |
| rs17369334 | 15563073 | 0.008 | T:G |
| rs10796293 | 15563343 | 0.492 | T:G |
| rs10752379 | 15563383 | 0.475 | T:A |
| rs10508486 | 15564168 | 0.483 | G:A |
| rs4748168 | 15565070 | 0.483 | A:C |
| rs4748169 | 15565098 | 0.483 | A:G |
| rs7893774 | 15566261 | 0.408 | C:T |

TABLE 156
Singleton Tag
Block 002
Chromosome 10
Rs1887218 (BP 18287669) LD block SNPs

| | | Minor | |
|---|---|---|---|
| | Base | Allele | |
| SNP ID (rs) | Position | Freq | Alleles |
| rs1277554 | 18276095 | 0.292 | G:A |
| rs1277553 | 18277548 | 0.375 | C:T |
| rs474778 | 18277852 | 0.375 | A:G |
| rs4747334 | 18279961 | 0.242 | G:C |
| rs2165899 | 18280472 | 0.333 | T:C |
| rs2120293 | 18280646 | 0.333 | A:G |
| rs7081872 | 18281903 | 0.008 | A:G |
| rs552511 | 18282011 | 0.375 | C:G |
| rs10508554 | 18282641 | 0.333 | A:G |
| rs10827902 | 18283150 | 0.317 | G:A |
| rs491995 | 18284014 | 0.383 | G:A |
| rs7085495 | 18284426 | 0.008 | C:T |
| rs7085502 | 18284438 | 0.042 | C:T |
| rs10827910 | 18285942 | 0.317 | C:G |
| rs10827911 | 18286736 | 0.317 | C:A |
| rs473919 | 18287409 | 0.167 | C:G |
| rs11011704 | 18287683 | 0.092 | G:T |
| rs7085542 | 18288101 | 0.317 | A:G |
| rs11593092 | 18288142 | 0.100 | G:A |
| rs11011709 | 18288361 | 0.225 | A:G |
| rs691159 | 18289670 | 0.300 | A:T |
| rs691148 | 18289738 | 0.308 | A:G |
| rs7074683 | 18289979 | 0.092 | G:A |
| rs11011721 | 18290010 | 0.050 | G:A |
| rs7095391 | 18290320 | 0.317 | A:T |
| rs10827920 | 18290344 | 0.050 | G:A |
| rs571979 | 18291613 | 0.358 | A:G |
| rs7896039 | 18292161 | 0.317 | A:G |
| rs12260894 | 18292217 | 0.092 | C:G |
| rs692077 | 18292386 | 0.317 | A:G |
| rs12761871 | 18292455 | 0.092 | G:A |
| rs12262907 | 18293098 | 0.308 | C:T |
| rs483822 | 18294323 | 0.350 | G:A |
| rs966720 | 18294360 | 0.317 | T:C |

TABLE 157
Singleton Tag
Block 003
Chromosome 10
Rs12268320 (BP 19402289) LD block SNPs

| | | Minor | |
|---|---|---|---|
| | Base | Allele | |
| SNP ID (rs) | Position | Freq | Alleles |
| rs2358280 | 19399634 | 0.483 | A:G |
| rs2358281 | 19399645 | 0.483 | G:A |
| rs2358284 | 19400104 | 0.483 | C:T |
| rs7089512 | 19400914 | 0.058 | G:T |
| rs6481736 | 19400930 | 0.483 | G:C |
| rs6481737 | 19400959 | 0.483 | C:A |
| rs11008347 | 19401029 | 0.108 | C:G |
| rs7090207 | 19401432 | 0.142 | G:A |
| rs7090216 | 19401457 | 0.483 | G:C |
| rs7074918 | 19401586 | 0.483 | A:G |
| rs3858196 | 19401829 | 0.483 | T:C |
| rs4748547 | 19402603 | 0.483 | C:T |
| rs4509661 | 19402938 | 0.483 | T:G |
| rs4465279 | 19403082 | 0.483 | T:G |
| rs2884422 | 19405052 | 0.483 | C:T |
| rs2884423 | 19405654 | 0.300 | G:A |
| rs4604782 | 19406353 | 0.050 | C:A |
| rs2358286 | 19406456 | 0.483 | C:G |
| rs2358287 | 19406558 | 0.408 | C:G |
| rs11592473 | 19407013 | 0.233 | G:A |
| rs2358288 | 19407406 | 0.483 | T:G |
| rs2358289 | 19408194 | 0.483 | A:G |
| rs2358290 | 19408230 | 0.292 | T:A |
| rs2358291 | 19408262 | 0.067 | A:G |
| rs2358292 | 19408328 | 0.067 | A:G |
| rs2358293 | 19408416 | 0.483 | G:C |
| rs2358294 | 19408531 | 0.058 | T:G |
| rs12242151 | 19408980 | 0.483 | A:G |
| rs12242254 | 19409106 | 0.300 | A:G |
| rs12220630 | 19409397 | 0.192 | C:A |
| rs10826925 | 19412239 | 0.242 | T:C |
| rs10740840 | 19413478 | 0.475 | T:G |
| rs4265498 | 19413725 | 0.483 | C:G |
| rs2358297 | 19413757 | 0.483 | G:A |
| rs11008418 | 19413834 | 0.125 | G:T |
| rs11008420 | 19414143 | 0.192 | T:C |
| rs10508574 | 19414346 | 0.483 | T:C |
| rs2358300 | 19415398 | 0.483 | G:A |
| rs11008423 | 19415497 | 0.142 | C:T |
| rs11008424 | 19415545 | 0.142 | A:G |
| rs2358301 | 19415718 | 0.475 | T:C |
| rs2358303 | 19416276 | 0.283 | G:A |
| rs2358304 | 19416322 | 0.142 | T:A |
| rs2358305 | 19416363 | 0.483 | A:C |
| rs10826935 | 19424238 | 0.433 | C:T |
| rs4748550 | 19425011 | 0.500 | C:C |
| rs4748551 | 19425033 | 0.500 | G:G |
| rs4748552 | 19425254 | 0.450 | A:C |
| rs4748553 | 19425426 | 0.433 | C:T |
| rs10826941 | 19426046 | 0.225 | C:T |
| rs7092121 | 19426862 | 0.500 | A:A |
| rs16918242 | 19427100 | 0.208 | T:C |
| rs2358310 | 19429152 | 0.500 | A:A |
| rs10826946 | 19429626 | 0.500 | A:A |
| rs7069170 | 19430052 | 0.500 | A:A |
| rs16915577 | 19432115 | 0.050 | A:G |
| rs10826949 | 19432655 | 0.450 | A:C |
| rs10159517 | 19432863 | 0.500 | C:C |
| rs10826950 | 19433381 | 0.450 | C:A |
| rs4748554 | 19433684 | 0.108 | A:T |
| rs10826951 | 19435163 | 0.500 | A:A |
| rs2358311 | 19435968 | 0.433 | G:A |
| rs8181341 | 19437127 | 0.500 | C:C |
| rs11008503 | 19437241 | 0.108 | G:C |
| rs11008507 | 19437986 | 0.167 | G:A |
| rs2358313 | 19439615 | 0.500 | C:C |
| rs12774563 | 19440631 | 0.025 | C:A |
| rs2358315 | 19440851 | 0.450 | T:C |

TABLE 158
Singleton Tag
Block 004
Chromosome 10
Rs1904756 (BP 60464000) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 159
Singleton Tag
Block 005
Chromosome 10
Rs16925318 (BP 69756962) LD block SNPs

| | | Minor | |
|---|---|---|---|
| | Base | Allele | |
| SNP ID (rs) | Position | Freq | Alleles |
| rs4746759 | 69735803 | 0.092 | T:C |
| rs12359690 | 69736559 | 0.017 | G:A |
| rs7100599 | 69739239 | 0.092 | G:A |
| rs7895740 | 69743675 | 0.092 | T:G |
| rs7904473 | 69745575 | 0.117 | T:C |
| rs11592777 | 69745922 | 0.008 | G:A |
| rs7084597 | 69746496 | 0.075 | T:C |
| rs10998066 | 69750259 | 0.100 | A:G |
| rs10998067 | 69751687 | 0.017 | C:G |
| rs1080639 | 69752036 | 0.058 | T:C |
| rs1867570 | 69753482 | 0.042 | T:C |
| rs7893969 | 69755535 | 0.467 | C:T |
| rs1867569 | 69756164 | 0.050 | C:T |
| rs10762224 | 69756319 | 0.133 | C:T |
| rs16925322 | 69758231 | 0.042 | T:C |
| rs1867568 | 69758631 | 0.075 | C:T |
| rs10998070 | 69759261 | 0.017 | G:T |
| rs16925325 | 69760269 | 0.075 | T:G |

TABLE 160
Singleton Tag
Block 006
Chromosome 10
Rs12254170 (BP 91287697) LD block SNPs

| | | Minor | |
|---|---|---|---|
| | Base | Allele | |
| SNP ID (rs) | Position | Freq | Alleles |
| rs12254170 | 91287697 | 0.342 | C:T |
| rs11185747 | 91289457 | 0.033 | T:A |
| rs12415606 | 91293960 | 0.067 | T:C |
| rs10881601 | 91295690 | 0.275 | G:A |
| rs12263957 | 91297762 | 0.275 | G:C |
| rs17123591 | 91297847 | 0.008 | G:C |
| rs12784228 | 91302317 | 0.192 | T:C |
| rs17123666 | 91302641 | 0.192 | T:C |
| rs12765829 | 91303425 | 0.192 | G:A |
| rs17123683 | 91304693 | 0.008 | C:A |
| rs12778320 | 91304787 | 0.192 | G:A |

TABLE 161
Singleton Tag
Block 007
Chromosome 10
Rs1965595 (BP 105758261) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs10883953 | 105714319 | 0.375 | C:T | rs2487998 | 105742202 | 0.025 | A:G |
| rs6584583 | 105716328 | 0.175 | C:T | rs2476952 | 105742219 | 0.175 | C:T |
| rs4917407 | 105717864 | 0.175 | G:A | rs2476953 | 105742493 | 0.175 | C:T |
| rs7097109 | 105718270 | 0.175 | A:G | rs11191894 | 105743048 | 0.175 | A:G |
| rs9988667 | 105719829 | 0.175 | A:G | rs1570646 | 105743547 | 0.175 | G:A |
| rs9325510 | 105719877 | 0.175 | G:A | rs6584585 | 105745029 | 0.175 | C:T |
| rs2066340 | 105720436 | 0.175 | G:A | rs1630099 | 105746657 | 0.175 | C:T |
| rs9988675 | 105721328 | 0.175 | A:G | rs17814732 | 105748322 | 0.375 | T:G |
| rs17116203 | 105721400 | 0.108 | A:G | rs2476955 | 105748455 | 0.175 | C:T |
| rs7922850 | 105723861 | 0.175 | A:G | rs10883960 | 105748660 | 0.175 | A:G |
| rs11191887 | 105724884 | 0.175 | A:C | rs805649 | 105749180 | 0.175 | A:G |
| rs11591965 | 105726004 | 0.175 | A:C | rs805650 | 105750065 | 0.175 | C:T |
| rs11596210 | 105726061 | 0.175 | G:A | rs805652 | 105750277 | 0.175 | C:T |
| rs10883958 | 105726274 | 0.200 | A:G | rs805653 | 105750374 | 0.175 | G:A |
| rs805667 | 105726469 | 0.175 | A:C | rs809302 | 105750988 | 0.175 | C:T |
| rs12355416 | 105726669 | 0.175 | G:A | rs805657 | 105752581 | 0.175 | G:A |
| rs805668 | 105727056 | 0.175 | T:C | rs3740469 | 105753016 | 0.175 | C:T |
| rs805669 | 105727681 | 0.175 | G:A | rs805658 | 105753614 | 0.192 | G:C |
| rs7894983 | 105728269 | 0.025 | T:C | rs805659 | 105754333 | 0.175 | T:C |
| rs811708 | 105729767 | 0.175 | G:T | rs805660 | 105755077 | 0.175 | T:G |
| rs2487987 | 105729904 | 0.175 | G:A | rs805662 | 105755735 | 0.025 | A:G |
| rs9420915 | 105730631 | 0.175 | A:G | rs17116263 | 105757799 | 0.025 | A:G |
| rs11191889 | 105731885 | 0.175 | T:C | rs1747674 | 105758243 | 0.175 | A:G |
| rs2487991 | 105733933 | 0.175 | C:A | rs805646 | 105759918 | 0.175 | A:G |
| rs2476961 | 105735637 | 0.175 | T:A | rs9971120 | 105760303 | 0.375 | C:T |
| rs2147270 | 105735691 | 0.375 | T:A | rs805647 | 105760999 | 0.175 | A:C |
| rs2476962 | 105735843 | 0.175 | T:A | rs1774594 | 105762403 | 0.175 | A:T |
| rs1747672 | 105737787 | 0.175 | C:T | rs1774599 | 105763257 | 0.175 | A:G |
| rs9420916 | 105737822 | 0.175 | C:T | rs10491048 | 105764417 | 0.025 | G:A |
| rs1612212 | 105738292 | 0.175 | G:A | rs1774600 | 105764724 | 0.175 | C:A |
| rs1774607 | 105738798 | 0.175 | T:G | rs10786782 | 105768119 | 0.033 | A:G |
| rs9419963 | 105739595 | 0.175 | A:G | rs2296217 | 105770125 | 0.017 | C:G |
| rs2476950 | 105741400 | 0.175 | C:T | rs805672 | 105770696 | 0.175 | C:A |
| rs2487997 | 105742015 | 0.175 | A:G | rs805673 | 105770716 | 0.175 | T:A |
| rs805674 | 105770826 | 0.183 | G:A | rs3124 | 105778895 | 0.175 | A:C |
| rs805675 | 105771156 | 0.175 | T:C | rs9425 | 105781345 | 0.175 | C:T |
| rs10883961 | 105771522 | 0.425 | T:C | rs805687 | 105781919 | 0.183 | C:G |
| rs4918077 | 105771701 | 0.375 | G:A | rs805688 | 105782067 | 0.208 | A:G |
| rs805679 | 105773227 | 0.175 | A:G | rs813943 | 105782195 | 0.175 | C:A |
| rs805680 | 105773636 | 0.175 | A:G | rs2147268 | 105783449 | 0.208 | T:C |
| rs805681 | 105773787 | 0.175 | G:A | rs11596890 | 105784845 | 0.108 | C:T |
| rs805682 | 105774009 | 0.175 | A:G | rs805714 | 105786728 | 0.017 | C:A |
| rs805683 | 105774113 | 0.175 | G:T | rs2282436 | 105787165 | 0.167 | A:C |
| rs809821 | 105777864 | 0.175 | A:G | rs2274100 | 105788862 | 0.167 | C:T |
| rs6787 | 105778736 | 0.183 | C:T | | | | |

TABLE 162
Singleton Tag
Block 001
Chromosome 11
Rs7117749 (BP 5453501) LD block SNPs

| | | Minor | |
|---|---|---|---|
| | Base | Allele | |
| SNP ID (rs) | Position | Freq | Alleles |
| rs10838178 | 5452540 | 0.308 | G:C |
| rs438996 | 5452894 | 0.383 | C:T |
| rs2647543 | 5453387 | 0.450 | A:C |
| rs365943 | 5454375 | 0.383 | A:G |

TABLE 163
Singleton Tag
Block 002
Chromosome 11
Rs11246628 (BP 51394606) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs2155154 | 51163430 | 0.492 | A:G | rs545033 | 51269029 | 0.033 | T:C |
| rs2186641 | 51163494 | 0.333 | T:C | rs488154 | 51270412 | 0.342 | T:C |
| rs10902321 | 51167354 | 0.433 | C:T | rs5017948 | 51270794 | 0.225 | T:A |
| rs1603756 | 51171349 | 0.483 | C:A | rs1391579 | 51272254 | 0.342 | A:G |
| rs4498978 | 51182640 | 0.425 | T:A | rs1694609 | 51272623 | 0.492 | C:T |
| rs10902328 | 51184421 | 0.158 | G:C | rs1783970 | 51273031 | 0.033 | G:A |
| rs1603764 | 51191194 | 0.425 | G:C | rs561171 | 51273577 | 0.492 | C:G |
| rs8188931 | 51194853 | 0.425 | T:C | rs7941855 | 51273607 | 0.092 | C:T |
| rs7294263 | 51196401 | 0.158 | A:G | rs10902344 | 51273731 | 0.075 | G:A |
| rs1939267 | 51207947 | 0.092 | C:T | rs11246549 | 51274538 | 0.075 | C:T |
| rs4450158 | 51221134 | 0.183 | G:A | rs12798438 | 51274891 | 0.158 | C:A |
| rs11246523 | 51226195 | 0.342 | C:T | rs11246551 | 51275965 | 0.058 | C:T |
| rs10794388 | 51228612 | 0.342 | A:G | rs489092 | 51276947 | 0.492 | A:G |
| rs8186362 | 51230448 | 0.425 | G:T | rs11518930 | 51277328 | 0.033 | T:G |
| rs11246529 | 51231886 | 0.342 | G:A | rs12225326 | 51278012 | 0.083 | C:A |
| rs1791422 | 51235092 | 0.342 | A:T | rs17491307 | 51282084 | 0.092 | T:G |
| rs568963 | 51240976 | 0.033 | A:T | rs484052 | 51282551 | 0.492 | T:C |
| rs554731 | 51244515 | 0.342 | C:T | rs510697 | 51283089 | 0.492 | C:T |
| rs542468 | 51245914 | 0.425 | T:C | rs11246555 | 51283148 | 0.075 | A:C |
| rs7481153 | 51247759 | 0.075 | C:T | rs17569863 | 51283221 | 0.092 | A:G |
| rs7478983 | 51247890 | 0.342 | T:G | rs12804084 | 51283666 | 0.183 | G:A |
| rs7121445 | 51248163 | 0.033 | G:A | rs7110107 | 51283679 | 0.092 | C:A |
| rs532210 | 51249087 | 0.425 | A:G | rs17570016 | 51284886 | 0.092 | A:T |
| rs530312 | 51249292 | 0.483 | A:G | rs574962 | 51285540 | 0.033 | G:T |
| rs11246539 | 51250294 | 0.067 | T:G | rs7128581 | 51285773 | 0.092 | T:G |
| rs499232 | 51250396 | 0.033 | C:A | rs10501331 | 51285867 | 0.075 | T:C |
| rs572049 | 51250799 | 0.342 | G:A | rs7106969 | 51285941 | 0.092 | G:A |
| rs1391576 | 51251508 | 0.175 | C:T | rs924304 | 51287253 | 0.067 | A:G |
| rs1791428 | 51256936 | 0.342 | T:A | rs554429 | 51288436 | 0.492 | C:T |
| rs11820332 | 51258111 | 0.092 | C:T | rs1846262 | 51293971 | 0.183 | C:T |
| rs11827854 | 51258363 | 0.092 | G:T | rs546192 | 51294128 | 0.492 | C:T |
| rs1939285 | 51259879 | 0.025 | C:T | rs490902 | 51294519 | 0.492 | T:C |
| rs573789 | 51260428 | 0.033 | C:A | rs17524068 | 51295739 | 0.092 | T:A |
| rs10902343 | 51268955 | 0.075 | T:C | rs527464 | 51297186 | 0.158 | T:C |
| rs1846265 | 51297968 | 0.017 | T:C | rs11246582 | 51336224 | 0.075 | A:G |
| rs508823 | 51302895 | 0.492 | T:A | rs7926161 | 51337266 | 0.333 | G:C |
| rs507015 | 51303111 | 0.433 | T:C | rs12804958 | 51339715 | 0.183 | A:C |
| rs483011 | 51303398 | 0.033 | G:T | rs11246586 | 51340858 | 0.075 | C:T |
| rs540337 | 51306657 | 0.342 | A:G | rs4085787 | 51343547 | 0.333 | C:T |
| rs561951 | 51308017 | 0.492 | A:G | rs7114240 | 51348231 | 0.167 | C:T |
| rs543099 | 51308193 | 0.033 | C:T | rs4881828 | 51348827 | 0.492 | C:A |
| rs11246573 | 51309234 | 0.075 | G:C | rs4881829 | 51348850 | 0.158 | C:T |
| rs491447 | 51309978 | 0.483 | G:A | rs11827030 | 51349164 | 0.092 | C:G |
| rs10431196 | 51311887 | 0.175 | C:G | rs7951522 | 51351595 | 0.158 | G:C |
| rs547549 | 51312424 | 0.500 | C:C | rs12798217 | 51354159 | 0.175 | C:A |
| rs11246574 | 51313853 | 0.092 | G:A | rs7394421 | 51356948 | 0.342 | C:T |
| rs492496 | 51313877 | 0.033 | C:T | rs4462324 | 51359141 | 0.008 | A:T |
| rs491762 | 51313916 | 0.033 | T:C | rs4391797 | 51359581 | 0.342 | T:C |
| rs5009058 | 51315633 | 0.167 | G:A | rs7395441 | 51365434 | 0.492 | A:C |
| rs538664 | 51317207 | 0.500 | A:A | rs9666605 | 51366060 | 0.033 | A:G |
| rs12577468 | 51317745 | 0.175 | G:A | rs7481776 | 51367205 | 0.167 | A:G |
| rs2201852 | 51318418 | 0.092 | G:C | rs11246602 | 51368666 | 0.125 | T:C |
| rs521874 | 51319510 | 0.492 | C:T | rs10902359 | 51369372 | 0.108 | T:C |
| rs11601538 | 51319942 | 0.075 | C:T | rs10794396 | 51371558 | 0.342 | T:C |
| rs11246578 | 51320589 | 0.075 | G:A | rs11246607 | 51372576 | 0.492 | C:T |
| rs10794390 | 51325396 | 0.325 | G:A | rs4434990 | 51373649 | 0.125 | G:A |
| rs1791436 | 51326365 | 0.342 | T:A | rs10902360 | 51373955 | 0.075 | C:T |
| rs530655 | 51326553 | 0.158 | C:T | rs11246610 | 51374837 | 0.125 | G:A |
| rs544585 | 51327007 | 0.500 | A:A | rs12805992 | 51375095 | 0.167 | A:G |
| rs504225 | 51327129 | 0.492 | C:T | rs7107202 | 51376006 | 0.150 | A:T |
| rs17554678 | 51329011 | 0.125 | A:T | rs7116094 | 51376095 | 0.092 | G:A |
| rs511379 | 51329624 | 0.033 | A:C | rs11820760 | 51377223 | 0.092 | A:C |
| rs547657 | 51331034 | 0.342 | G:C | rs10902363 | 51378505 | 0.492 | C:A |
| rs546690 | 51331155 | 0.492 | G:T | rs12785517 | 51380438 | 0.183 | A:T |
| rs11826952 | 51331428 | 0.092 | T:C | rs6598119 | 51381035 | 0.342 | A:G |
| rs7117992 | 51332127 | 0.092 | A:C | rs7944067 | 51382454 | 0.300 | G:T |
| rs7108904 | 51334382 | 0.092 | G:A | rs2078611 | 51383331 | 0.342 | G:T |
| rs7112290 | 51334648 | 0.158 | G:A | rs3886724 | 51383437 | 0.125 | A:G |
| rs10902366 | 51383586 | 0.167 | T:G | rs10794400 | 51402756 | 0.342 | C:T |
| rs11246618 | 51383794 | 0.167 | A:G | rs12802227 | 51413890 | 0.183 | G:C |
| rs12421819 | 51383815 | 0.058 | G:A | rs4881767 | 51418932 | 0.492 | T:C |
| rs4362132 | 51385578 | 0.500 | A:A | rs7482339 | 51419707 | 0.075 | T:G |
| rs11246624 | 51389186 | 0.125 | G:A | rs7484073 | 51420212 | 0.075 | G:A |
| rs11246638 | 51402122 | 0.033 | G:C | rs7479178 | 51423485 | 0.192 | A:G |

TABLE 164
Singleton Tag
Block 003
Chromosome 11
Rs17610514 (BP 55652374) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs1531765 | 55590708 | 0.342 | C:T | rs12276209 | 55610619 | 0.092 | C:A |
| rs11227133 | 55593196 | 0.242 | T:C | rs1384093 | 55610907 | 0.125 | G:T |
| rs17601473 | 55594009 | 0.125 | A:G | rs10895994 | 55611141 | 0.342 | A:C |
| rs17529401 | 55595074 | 0.125 | A:G | rs11227180 | 55612187 | 0.100 | G:C |
| rs17529415 | 55595444 | 0.125 | C:T | rs7935319 | 55612869 | 0.367 | A:C |
| rs17529512 | 55595785 | 0.125 | C:T | rs4939022 | 55613836 | 0.367 | T:C |
| rs11227145 | 55595854 | 0.100 | C:A | rs17530416 | 55613884 | 0.125 | C:G |
| rs17529574 | 55595904 | 0.125 | G:A | rs17530423 | 55614057 | 0.125 | C:T |
| rs10501347 | 55596209 | 0.125 | G:A | rs10895996 | 55614274 | 0.083 | C:T |
| rs17601787 | 55596607 | 0.125 | A:G | rs17602635 | 55614946 | 0.142 | A:G |
| rs17601815 | 55597032 | 0.125 | C:T | rs17602705 | 55615347 | 0.125 | G:T |
| rs17601864 | 55597313 | 0.125 | A:G | rs17530634 | 55615499 | 0.125 | A:G |
| rs10501348 | 55597728 | 0.125 | G:A | rs17530690 | 55615678 | 0.142 | C:A |
| rs17601948 | 55598856 | 0.125 | A:T | rs7935372 | 55615948 | 0.125 | T:C |
| rs11227155 | 55599088 | 0.192 | G:C | rs7943453 | 55616001 | 0.125 | G:A |
| rs7396759 | 55601430 | 0.292 | T:C | rs7947530 | 55616964 | 0.125 | G:C |
| rs17529886 | 55603875 | 0.125 | T:C | rs17602906 | 55617019 | 0.125 | A:G |
| rs17602031 | 55604305 | 0.125 | C:A | rs17602927 | 55617109 | 0.125 | T:C |
| rs17602087 | 55604342 | 0.125 | T:C | rs17603011 | 55617729 | 0.125 | A:G |
| rs12289998 | 55604474 | 0.292 | C:T | rs2945816 | 55618667 | 0.008 | T:C |
| rs17149981 | 55604521 | 0.067 | T:C | rs7116619 | 55619108 | 0.125 | C:T |
| rs17530095 | 55604918 | 0.125 | G:A | rs7122627 | 55619141 | 0.242 | A:G |
| rs17149998 | 55605015 | 0.142 | G:T | rs7116741 | 55619196 | 0.242 | C:T |
| rs1384089 | 55605592 | 0.125 | G:C | rs7120128 | 55619554 | 0.292 | C:G |
| rs17602278 | 55605691 | 0.125 | T:C | rs17603150 | 55619611 | 0.125 | T:C |
| rs17530228 | 55605732 | 0.125 | A:G | rs17150038 | 55619990 | 0.108 | A:T |
| rs17602368 | 55605888 | 0.125 | A:C | rs2460192 | 55620737 | 0.133 | T:G |
| rs4939020 | 55606111 | 0.367 | G:A | rs2449155 | 55621539 | 0.125 | T:C |
| rs7118155 | 55607107 | 0.342 | C:T | rs17603260 | 55621778 | 0.125 | T:G |
| rs17530318 | 55607598 | 0.125 | C:A | rs9888159 | 55622023 | 0.142 | G:T |
| rs17530360 | 55607700 | 0.125 | C:T | rs1905074 | 55623460 | 0.133 | C:T |
| rs12361511 | 55609746 | 0.367 | T:C | rs2512961 | 55629452 | 0.125 | T:C |
| rs1481999 | 55610284 | 0.342 | A:G | rs7933129 | 55630166 | 0.125 | C:G |
| rs1384091 | 55610501 | 0.125 | T:G | rs11227222 | 55632969 | 0.100 | T:G |
| rs7929106 | 55633429 | 0.125 | T:C | rs7122358 | 55649045 | 0.125 | A:G |
| rs7127957 | 55634124 | 0.225 | C:T | rs7108978 | 55650288 | 0.117 | G:T |
| rs12223014 | 55634866 | 0.100 | G:A | rs7103403 | 55650365 | 0.117 | T:A |
| rs7935728 | 55636031 | 0.125 | C:T | rs2512785 | 55650392 | 0.125 | T:C |
| rs7924763 | 55636140 | 0.125 | G:A | rs2512784 | 55650762 | 0.100 | A:G |
| rs7924778 | 55636172 | 0.125 | G:C | rs17150088 | 55651438 | 0.100 | T:G |
| rs7948146 | 55636366 | 0.125 | T:G | rs17531814 | 55651486 | 0.125 | T:G |
| rs7951765 | 55639957 | 0.125 | C:T | rs17531884 | 55652157 | 0.125 | T:G |
| rs7940696 | 55640073 | 0.125 | G:A | rs17531933 | 55652225 | 0.125 | T:C |
| rs7932753 | 55640127 | 0.125 | T:C | rs17531954 | 55652307 | 0.125 | T:C |
| rs2512958 | 55640257 | 0.225 | A:T | rs2220004 | 55652321 | 0.292 | G:T |
| rs17603331 | 55640870 | 0.125 | C:A | rs17610514 | 55652374 | 0.125 | A:G |
| rs7123005 | 55640947 | 0.125 | G:A | rs2512782 | 55652591 | 0.225 | T:C |
| rs7123036 | 55641010 | 0.125 | G:A | rs11227290 | 55653153 | 0.100 | A:G |
| rs1482009 | 55641266 | 0.100 | T:G | rs17610606 | 55653321 | 0.125 | A:G |
| rs2460212 | 55642731 | 0.100 | T:C | rs10896042 | 55655164 | 0.292 | G:C |
| rs17603373 | 55642795 | 0.125 | T:C | rs11606821 | 55656373 | 0.058 | A:G |
| rs17603457 | 55643783 | 0.125 | G:C | rs1482001 | 55657297 | 0.125 | T:C |
| rs7123543 | 55643984 | 0.125 | A:G | rs7949386 | 55657859 | 0.483 | G:A |
| rs12223248 | 55644209 | 0.100 | A:C | rs17532185 | 55657874 | 0.125 | G:T |
| rs4264166 | 55644347 | 0.125 | A:G | rs17610726 | 55658041 | 0.125 | A:G |
| rs7121269 | 55644520 | 0.125 | C:T | rs2512789 | 55658787 | 0.292 | C:T |
| rs2870138 | 55644830 | 0.125 | C:T | rs17150095 | 55659476 | 0.100 | A:G |
| rs17531417 | 55644883 | 0.125 | A:G | rs17150096 | 55659572 | 0.100 | C:T |
| rs2170446 | 55645142 | 0.125 | A:T | rs17610901 | 55660218 | 0.125 | T:G |
| rs17603555 | 55645664 | 0.125 | A:T | rs2512939 | 55660390 | 0.292 | C:T |
| rs7113418 | 55646155 | 0.125 | T:G | rs17150102 | 55660958 | 0.100 | A:C |
| rs17531522 | 55647025 | 0.125 | A:G | rs1947923 | 55661327 | 0.125 | T:C |
| rs1825158 | 55647759 | 0.483 | T:C | rs2512932 | 55664392 | 0.142 | A:G |
| rs17610073 | 55648013 | 0.125 | C:T | rs2512931 | 55664572 | 0.292 | C:T |
| rs17610113 | 55648342 | 0.125 | T:A | rs7113176 | 55666423 | 0.125 | G:A |
| rs7103673 | 55648555 | 0.125 | G:C | rs12223067 | 55671996 | 0.100 | C:A |
| rs7103694 | 55648602 | 0.125 | G:A | rs2512920 | 55672925 | 0.267 | T:G |
| rs7125861 | 55648805 | 0.125 | T:G | rs11227364 | 55673292 | 0.217 | C:A |
| rs11227372 | 55676258 | 0.100 | C:A | rs4616058 | 55706034 | 0.483 | C:T |
| rs1871848 | 55676838 | 0.142 | C:T | rs17145425 | 55706606 | 0.100 | A:G |
| rs1600822 | 55677783 | 0.483 | T:G | rs11227453 | 55706757 | 0.008 | T:A |
| rs12803369 | 55678553 | 0.142 | A:G | rs7120089 | 55706925 | 0.125 | A:G |
| rs2512919 | 55679364 | 0.125 | T:C | rs7104401 | 55706945 | 0.125 | C:G |
| rs2512938 | 55684167 | 0.008 | A:G | rs4519111 | 55707881 | 0.217 | G:T |
| rs11227400 | 55685757 | 0.217 | C:T | rs7120321 | 55710661 | 0.008 | G:A |
| rs7950386 | 55688089 | 0.100 | C:T | rs11227462 | 55710924 | 0.200 | T:C |
| rs10896084 | 55690189 | 0.292 | C:A | rs11227463 | 55712023 | 0.100 | C:T |
| rs4300408 | 55691016 | 0.483 | G:T | rs17150147 | 55713349 | 0.142 | T:G |
| rs11227417 | 55692086 | 0.483 | C:T | rs6591189 | 55714384 | 0.225 | T:C |
| rs12421584 | 55693986 | 0.100 | G:A | rs7948152 | 55714583 | 0.133 | T:C |
| rs4578407 | 55694530 | 0.292 | G:A | rs7940541 | 55715396 | 0.225 | G:A |
| rs17611329 | 55695469 | 0.125 | A:G | rs7930955 | 55715596 | 0.125 | T:G |
| rs7114729 | 55696970 | 0.300 | T:C | rs11227471 | 55715725 | 0.292 | A:C |
| rs10791857 | 55697460 | 0.292 | G:T | rs7112815 | 55716680 | 0.100 | T:C |
| rs17611401 | 55697750 | 0.125 | G:T | rs12290119 | 55717346 | 0.125 | G:A |
| rs17150117 | 55697762 | 0.100 | T:C | rs1384061 | 55718817 | 0.125 | A:G |
| rs7119253 | 55698150 | 0.125 | C:T | rs11227482 | 55719514 | 0.008 | T:G |
| rs11227428 | 55698548 | 0.100 | C:G | rs10750788 | 55721640 | 0.092 | C:T |
| rs7934132 | 55699039 | 0.100 | T:A | rs17612015 | 55722455 | 0.125 | T:C |
| rs4939026 | 55699187 | 0.292 | A:G | rs11227486 | 55722606 | 0.125 | A:C |
| rs12270268 | 55699287 | 0.125 | A:G | rs1026866 | 55722892 | 0.092 | G:C |
| rs11227431 | 55699420 | 0.483 | T:C | rs2035395 | 55723699 | 0.125 | G:A |
| rs17533114 | 55699898 | 0.125 | T:C | rs2035394 | 55723767 | 0.092 | G:C |
| rs17611627 | 55700224 | 0.125 | A:C | rs2035393 | 55723882 | 0.125 | G:A |
| rs10896103 | 55700624 | 0.292 | C:G | rs7949337 | 55724519 | 0.125 | C:T |
| rs4489763 | 55701050 | 0.100 | G:A | rs11227494 | 55726412 | 0.200 | T:C |
| rs11227434 | 55701827 | 0.100 | G:T | rs12287754 | 55727983 | 0.217 | C:T |
| rs10896106 | 55702342 | 0.225 | G:A | rs10791879 | 55728633 | 0.292 | T:C |
| rs10896107 | 55704724 | 0.483 | T:A | rs11227507 | 55728850 | 0.100 | G:A |
| rs7117504 | 55704760 | 0.125 | G:A | rs11227508 | 55728935 | 0.125 | C:G |
| rs7103672 | 55704814 | 0.125 | A:G | rs11227513 | 55729437 | 0.142 | C:T |
| rs11227449 | 55705985 | 0.100 | A:G | rs1481926 | 55729857 | 0.100 | C:T |
| rs11227518 | 55730849 | 0.125 | T:A | rs17612565 | 55776220 | 0.125 | A:G |
| rs10791880 | 55732478 | 0.292 | A:G | rs10501349 | 55777860 | 0.100 | G:A |
| rs969595 | 55732908 | 0.092 | C:T | rs2035396 | 55777990 | 0.125 | C:T |
| rs10431119 | 55733645 | 0.100 | C:T | rs2035397 | 55778277 | 0.125 | T:C |
| rs7935265 | 55733884 | 0.292 | A:G | rs2035398 | 55778363 | 0.125 | T:C |
| rs7936219 | 55733911 | 0.092 | C:T | rs1481928 | 55778510 | 0.292 | T:C |
| rs1481927 | 55735532 | 0.292 | A:G | rs12293515 | 55778961 | 0.125 | G:A |
| rs7130251 | 55735842 | 0.483 | C:T | rs11227667 | 55782010 | 0.200 | G:A |
| rs7122585 | 55736450 | 0.125 | T:C | rs7124120 | 55782197 | 0.117 | G:A |
| rs4644659 | 55738545 | 0.392 | C:G | rs7128214 | 55782335 | 0.292 | G:C |
| rs11227551 | 55739642 | 0.125 | C:T | rs12098877 | 55783235 | 0.125 | T:C |
| rs10896130 | 55739945 | 0.125 | G:A | rs10160392 | 55783691 | 0.125 | A:G |
| rs7947829 | 55750573 | 0.225 | A:G | rs10896152 | 55784303 | 0.483 | G:A |
| rs12270451 | 55756079 | 0.125 | G:A | rs10160540 | 55786053 | 0.125 | T:C |
| rs7121880 | 55756313 | 0.125 | G:C | rs7122505 | 55786930 | 0.225 | G:A |
| rs7122000 | 55756473 | 0.125 | G:A | rs12286575 | 55787905 | 0.125 | T:C |
| rs7122514 | 55756864 | 0.125 | G:A | rs11227693 | 55789468 | 0.483 | T:G |
| rs10791893 | 55756979 | 0.092 | G:C | rs9633989 | 55790139 | 0.483 | A:G |
| rs11227598 | 55757027 | 0.100 | G:A | rs10750794 | 55790408 | 0.292 | T:A |
| rs11227599 | 55757047 | 0.225 | G:A | rs11227698 | 55790539 | 0.200 | T:C |
| rs10896137 | 55759675 | 0.225 | T:C | rs10750795 | 55790554 | 0.292 | C:G |
| rs2128137 | 55760136 | 0.092 | G:A | rs11227702 | 55791350 | 0.108 | A:C |
| rs10896138 | 55760165 | 0.225 | G:A | rs11227719 | 55793363 | 0.342 | C:A |
| rs11822296 | 55761852 | 0.292 | C:T | rs11227721 | 55793597 | 0.225 | G:C |
| rs2904326 | 55764756 | 0.483 | G:A | rs10896157 | 55794197 | 0.100 | T:G |
| rs1905053 | 55768424 | 0.100 | C:T | rs12288235 | 55794450 | 0.125 | T:A |
| rs7937078 | 55770162 | 0.125 | T:C | rs10896159 | 55795532 | 0.342 | A:G |
| rs12280852 | 55770374 | 0.008 | A:G | rs11227729 | 55796477 | 0.100 | G:A |
| rs1531757 | 55772055 | 0.483 | A:G | rs12792349 | 55796904 | 0.142 | G:A |
| rs12292265 | 55773069 | 0.125 | G:A | rs2035392 | 55798299 | 0.125 | C:T |
| rs12287791 | 55773272 | 0.125 | T:G | rs7125690 | 55799556 | 0.125 | C:A |
| rs12279693 | 55774039 | 0.125 | T:C | rs12360890 | 55800180 | 0.342 | A:G |
| rs10896143 | 55774484 | 0.483 | T:C | rs7127287 | 55802715 | 0.292 | C:T |
| rs11227638 | 55776161 | 0.100 | A:T | rs7949188 | 55803257 | 0.008 | T:C |
| rs7933691 | 55803476 | 0.100 | G:A | rs12788665 | 55826389 | 0.142 | A:C |
| rs10896163 | 55805336 | 0.292 | A:G | rs11227808 | 55826923 | 0.100 | A:T |
| rs2170442 | 55805527 | 0.292 | A:C | rs2870434 | 55828133 | 0.417 | A:G |
| rs716103 | 55807836 | 0.125 | C:T | rs11227810 | 55828427 | 0.100 | A:G |
| rs716104 | 55807866 | 0.125 | T:C | rs12574347 | 55830221 | 0.142 | T:C |
| rs7951134 | 55808578 | 0.133 | T:A | rs1600812 | 55832831 | 0.092 | G:A |
| rs11227763 | 55809097 | 0.050 | T:C | rs7108801 | 55833176 | 0.125 | C:T |
| rs17150276 | 55810405 | 0.342 | T:C | rs1481925 | 55833386 | 0.125 | C:T |
| rs17540576 | 55810488 | 0.125 | C:G | rs1481924 | 55833500 | 0.125 | G:T |
| rs17540625 | 55810938 | 0.125 | G:A | rs1481923 | 55833562 | 0.125 | T:C |
| rs4939031 | 55813028 | 0.175 | C:A | rs11227836 | 55834399 | 0.200 | C:A |
| rs1156808 | 55813326 | 0.342 | G:C | rs7924726 | 55834507 | 0.125 | A:G |
| rs17150073 | 55813561 | 0.217 | A:C | rs7927921 | 55834545 | 0.125 | T:C |
| rs9943535 | 55813774 | 0.142 | A:G | rs7117192 | 55834978 | 0.100 | C:T |
| rs17150286 | 55813995 | 0.342 | A:G | rs11227842 | 55836102 | 0.100 | C:T |
| rs1842674 | 55814773 | 0.342 | G:A | rs1600811 | 55837376 | 0.292 | G:T |
| rs17540861 | 55815050 | 0.125 | T:A | rs1842675 | 55837400 | 0.100 | G:C |
| rs11600896 | 55815111 | 0.125 | C:T | rs7947384 | 55837836 | 0.125 | T:A |
| rs10501350 | 55815266 | 0.117 | G:A | rs11227849 | 55837989 | 0.100 | A:T |
| rs12223785 | 55815396 | 0.100 | T:A | rs11227850 | 55838083 | 0.100 | A:G |
| rs1481918 | 55817736 | 0.125 | A:C | rs11227854 | 55838320 | 0.100 | T:C |
| rs17613116 | 55817958 | 0.125 | T:G | rs11227857 | 55838688 | 0.100 | A:G |
| rs17613137 | 55818008 | 0.125 | C:T | rs17613435 | 55839477 | 0.125 | A:T |
| rs11227784 | 55818296 | 0.483 | G:T | rs17613463 | 55839511 | 0.133 | A:C |
| rs7106012 | 55820150 | 0.092 | T:C | rs11227868 | 55840481 | 0.100 | C:T |
| rs17150048 | 55820287 | 0.100 | A:G | rs1871846 | 55841187 | 0.292 | C:T |
| rs17613241 | 55820739 | 0.125 | A:G | rs17541516 | 55841609 | 0.142 | C:T |
| rs17541133 | 55820892 | 0.125 | T:G | rs7932412 | 55842192 | 0.125 | A:C |
| rs17613345 | 55822387 | 0.125 | C:G | rs960193 | 55842723 | 0.292 | G:T |
| rs10501351 | 55822528 | 0.125 | A:G | rs17541744 | 55843382 | 0.125 | C:T |
| rs17613394 | 55823238 | 0.142 | T:G | rs1384060 | 55843897 | 0.483 | A:G |
| rs12288127 | 55824631 | 0.292 | C:A | rs2054610 | 55844518 | 0.100 | T:G |
| rs11227802 | 55824677 | 0.483 | T:C | rs6591230 | 55845959 | 0.125 | T:C |
| rs12281604 | 55825038 | 0.008 | C:T | rs2128134 | 55847156 | 0.342 | A:G |
| rs11227917 | 55851898 | 0.100 | T:C | rs1481920 | 55873449 | 0.292 | C:T |
| rs17541821 | 55855722 | 0.125 | T:C | rs1586004 | 55874901 | 0.292 | C:T |
| rs7106248 | 55855880 | 0.125 | T:A | rs12794269 | 55874927 | 0.142 | G:A |
| rs7121384 | 55856315 | 0.008 | C:G | rs11228116 | 55876116 | 0.292 | A:C |
| rs11604721 | 55856577 | 0.008 | C:T | rs1351533 | 55876944 | 0.092 | T:C |
| rs11227964 | 55857067 | 0.300 | G:A | rs10896287 | 55878558 | 0.342 | C:G |
| rs17541876 | 55857463 | 0.125 | T:C | rs7931849 | 55878883 | 0.125 | G:T |
| rs2220001 | 55858634 | 0.292 | G:T | rs12364581 | 55878960 | 0.200 | T:A |
| rs6591233 | 55859229 | 0.292 | C:A | rs7950725 | 55879179 | 0.125 | T:C |
| rs7119614 | 55859403 | 0.150 | A:C | rs7950843 | 55879250 | 0.125 | T:C |
| rs12789200 | 55859579 | 0.142 | C:G | rs7940239 | 55880498 | 0.292 | T:C |
| rs11600863 | 55860066 | 0.342 | T:G | rs17614629 | 55882318 | 0.133 | C:G |
| rs7124023 | 55860427 | 0.125 | A:C | rs17614652 | 55882539 | 0.125 | C:T |
| rs12222334 | 55861033 | 0.142 | T:G | rs7942730 | 55884638 | 0.100 | A:T |
| rs1586006 | 55864015 | 0.417 | C:A | rs10896290 | 55884657 | 0.292 | A:G |
| rs12226794 | 55867535 | 0.100 | C:T | rs7928704 | 55885100 | 0.100 | T:C |
| rs11228064 | 55867648 | 0.200 | T:C | rs10896292 | 55885935 | 0.100 | T:C |
| rs17542168 | 55869744 | 0.125 | T:C | rs4939034 | 55888637 | 0.100 | C:T |
| rs1905055 | 55870092 | 0.292 | C:T | rs1945236 | 55892955 | 0.100 | G:A |
| rs10896271 | 55870151 | 0.342 | A:G | rs12577663 | 55894617 | 0.142 | G:A |
| rs17614327 | 55870340 | 0.125 | G:A | rs17542678 | 55895146 | 0.125 | G:A |
| rs17614415 | 55871788 | 0.142 | G:A | rs1945200 | 55895570 | 0.100 | T:A |
| rs17542365 | 55872133 | 0.125 | C:T | rs12804193 | 55897827 | 0.142 | T:C |
| rs1905054 | 55872520 | 0.342 | T:C | rs11607849 | 55897992 | 0.092 | T:C |
| rs17542414 | 55872887 | 0.125 | A:G | rs10896302 | 55898172 | 0.100 | A:C |
| rs17614481 | 55873169 | 0.125 | C:T | rs1945201 | 55898882 | 0.483 | C:T |

TABLE 165
Singleton Tag
Block 004
Chromosome 11
Rs558073 (BP 63959003) LD block SNPs

| | | Minor | |
|---|---|---|---|
| | Base | Allele | |
| SNP ID (rs) | Position | Freq | Alleles |
| rs553587 | 63958504 | 0.400 | G:A |
| rs4930353 | 63959223 | 0.292 | G:A |
| rs606063 | 63959305 | 0.417 | T:C |
| rs534874 | 63961597 | 0.417 | G:A |
| rs596308 | 63967228 | 0.317 | G:A |
| rs653565 | 63969419 | 0.283 | C:T |
| rs12800000 | 63970003 | 0.308 | G:T |
| rs12800420 | 63970207 | 0.042 | G:C |
| rs656785 | 63970210 | 0.142 | G:A |
| rs570024 | 63971608 | 0.292 | G:T |

TABLE 166
Singleton Tag
Block 005
Chromosome 11
Rs597227 (BP 120899984) LD block SNPs

| | | Minor | |
|---|---|---|---|
| | Base | Allele | |
| SNP ID (rs) | Position | Freq | Alleles |
| rs11218317 | 120893191 | 0.475 | A:T |
| rs4935775 | 120894712 | 0.408 | T:G |
| rs12285364 | 120898436 | 0.050 | C:T |
| rs12285564 | 120898660 | 0.050 | G:A |
| rs2298813 | 120898894 | 0.050 | G:A |
| rs11218319 | 120899497 | 0.400 | C:T |
| rs693855 | 120900823 | 0.383 | G:A |
| rs1784927 | 120901000 | 0.467 | C:T |

TABLE 167
Singleton Tag
Block 006
Chromosome 11
rs11220034 (BP 124,741,629) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 168
Singleton Tag
Block 001
Chromosome 12
Rs12227654 (BP 23580022) LD block SNPs

| | | Minor | |
|---|---|---|---|
| | Base | Allele | |
| SNP ID (rs) | Position | Freq | Alleles |
| rs11046968 | 23572538 | 0.292 | A:C |
| rs12227654 | 23580022 | 0.217 | G:A |
| rs7485662 | 23587383 | 0.458 | T:C |

TABLE 169
Singleton Tag
Block 002
Chromosome 12
Rs11114962 (BP 80548120) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs10778818 | 80512243 | 0.317 | G:A | rs1573889 | 80531034 | 0.400 | T:C |
| rs1922417 | 80513968 | 0.075 | G:A | rs7959273 | 80533261 | 0.075 | T:C |
| rs11114939 | 80515394 | 0.075 | T:G | rs7305940 | 80535956 | 0.383 | A:T |
| rs17008788 | 80515519 | 0.042 | T:C | rs12366337 | 80536249 | 0.042 | C:T |
| rs1882535 | 80515566 | 0.075 | G:A | rs11114954 | 80536919 | 0.042 | G:A |
| rs11114940 | 80517233 | 0.042 | T:C | rs11114957 | 80541069 | 0.042 | C:T |
| rs11114941 | 80518004 | 0.433 | A:G | rs9804711 | 80541750 | 0.267 | A:C |
| rs10746195 | 80518118 | 0.092 | G:A | rs10862335 | 80542084 | 0.042 | T:C |
| rs2141876 | 80518566 | 0.167 | T:C | rs9804913 | 80543465 | 0.383 | C:T |
| rs17041753 | 80518820 | 0.075 | C:T | rs2400959 | 80545680 | 0.075 | G:T |
| rs10161159 | 80519832 | 0.400 | A:G | rs1114962 | 80548120 | 0.117 | G:C |
| rs11114942 | 80519846 | 0.075 | A:G | rs10778820 | 80548733 | 0.075 | A:G |
| rs10082971 | 80520376 | 0.408 | A:C | rs11114963 | 80548827 | 0.075 | C:T |
| rs9308326 | 80521998 | 0.325 | T:C | rs10778821 | 80549599 | 0.375 | C:T |
| rs7309720 | 80522175 | 0.075 | G:A | rs4480617 | 80549676 | 0.183 | G:C |
| rs7295977 | 80522270 | 0.075 | A:C | rs4587799 | 80552522 | 0.183 | T:C |
| rs12826603 | 80522405 | 0.183 | G:A | rs11114972 | 80561443 | 0.408 | C:T |
| rs10862331 | 80522777 | 0.400 | C:T | rs17008872 | 80563153 | 0.008 | T:C |
| rs11114945 | 80523718 | 0.075 | C:T | rs7963022 | 80564395 | 0.183 | A:G |
| rs1922412 | 80524073 | 0.075 | C:T | rs12826173 | 80564780 | 0.183 | C:T |
| rs7138266 | 80525186 | 0.075 | C:G | rs10778824 | 80565494 | 0.408 | C:T |
| rs17008823 | 80525806 | 0.008 | G:A | rs11114974 | 80565778 | 0.075 | T:C |
| rs12367706 | 80526747 | 0.042 | G:A | rs10746199 | 80565817 | 0.075 | T:C |
| rs11114948 | 80527086 | 0.075 | C:A | rs11114975 | 80566854 | 0.117 | C:T |
| rs1922413 | 80527716 | 0.092 | G:A | rs2222886 | 80567201 | 0.075 | T:C |
| rs7960683 | 80528619 | 0.400 | G:A | rs7310828 | 80567739 | 0.075 | T:A |
| rs11114949 | 80528719 | 0.042 | T:A | rs1578964 | 80571079 | 0.075 | C:T |
| rs17041757 | 80528816 | 0.075 | A:G | rs1578963 | 80571107 | 0.075 | G:C |
| rs12228821 | 80529250 | 0.075 | G:A | rs1922407 | 80571245 | 0.408 | G:A |
| rs11114950 | 80529448 | 0.042 | T:C | rs1950143 | 80574051 | 0.075 | A:G |
| rs11114951 | 80529552 | 0.075 | G:A | rs12580614 | 80574172 | 0.008 | T:C |
| rs11611766 | 80529939 | 0.042 | G:A | rs1922545 | 80574252 | 0.383 | A:G |
| rs17008849 | 80529957 | 0.008 | T:C | | | | |

TABLE 170
Singleton Tag
Block 003
Chromosome 12
Rs7489074 (BP 130429948) LD block SNPs

| | | Minor | |
|---|---|---|---|
| | Base | Allele | |
| SNP ID (rs) | Position | Freq | Alleles |
| rs10744514 | 130427917 | 0.317 | T:G |
| rs7956380 | 130428151 | 0.317 | A:G |
| rs6486676 | 130428688 | 0.233 | G:A |
| rs7139192 | 130428797 | 0.317 | A:G |
| rs7489074 | 130429948 | 0.083 | A:G |
| rs6486678 | 130429999 | 0.233 | A:G |
| rs11061523 | 130431782 | 0.075 | G:A |
| rs10773892 | 130431807 | 0.342 | A:G |
| rs7397818 | 130431941 | 0.233 | A:T |
| rs7397555 | 130432018 | 0.317 | G:A |
| rs1246346 | 130434729 | 0.008 | A:G |
| rs12369807 | 130434931 | 0.025 | G:A |
| rs10848374 | 130435685 | 0.417 | T:C |

TABLE 171
Singleton Tag
Block 001
Chromosome 13
Rs17088665 (BP 63132563) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs9539832 | 63124896 | 0.183 | A:G | rs2197313 | 63160271 | 0.175 | G:A |
| rs9570968 | 63125765 | 0.008 | T:C | rs17088713 | 63160894 | 0.008 | C:T |
| rs9564088 | 63128349 | 0.008 | A:C | rs9570996 | 63160940 | 0.008 | A:T |
| rs1530485 | 63130342 | 0.008 | C:T | rs9570998 | 63161346 | 0.008 | G:T |
| rs1822963 | 63131518 | 0.008 | A:T | rs9570999 | 63161420 | 0.008 | G:A |
| rs9570971 | 63131686 | 0.008 | A:C | rs9571000 | 63161493 | 0.008 | A:G |
| rs9570972 | 63131946 | 0.008 | G:C | rs9571001 | 63161535 | 0.008 | G:A |
| rs9570973 | 63132877 | 0.008 | G:T | rs1443958 | 63161809 | 0.008 | G:A |
| rs2810864 | 63140115 | 0.008 | G:A | rs9571002 | 63162261 | 0.300 | T:G |
| rs981289 | 63140935 | 0.008 | A:C | rs2590913 | 63162447 | 0.058 | A:G |
| rs1443960 | 63141175 | 0.017 | G:A | rs7335478 | 63162805 | 0.175 | A:G |
| rs981674 | 63142497 | 0.175 | C:T | rs7336074 | 63162990 | 0.008 | C:T |
| rs9570978 | 63143019 | 0.008 | T:C | rs2590914 | 63163006 | 0.008 | G:A |
| rs1530407 | 63143709 | 0.175 | A:G | rs4609714 | 63163335 | 0.175 | A:G |
| rs1445554 | 63145373 | 0.167 | G:A | rs9564093 | 63164873 | 0.175 | C:A |
| rs2810865 | 63147225 | 0.175 | G:A | rs9571003 | 63164952 | 0.183 | C:T |
| rs1031184 | 63148381 | 0.167 | A:G | rs9564095 | 63165033 | 0.175 | A:G |
| rs12869422 | 63151379 | 0.008 | A:C | rs11148621 | 63166436 | 0.175 | C:T |
| rs1158920 | 63151414 | 0.175 | T:C | rs9539843 | 63166982 | 0.167 | G:T |
| rs2810867 | 63153341 | 0.175 | G:C | rs9539844 | 63167468 | 0.167 | A:G |
| rs4884503 | 63154669 | 0.008 | T:C | rs9539845 | 63168260 | 0.175 | T:C |
| rs4884504 | 63154782 | 0.008 | A:G | rs12430777 | 63168595 | 0.167 | G:A |
| rs9564091 | 63155174 | 0.008 | G:C | rs8002824 | 63168693 | 0.175 | A:G |
| rs9570985 | 63155518 | 0.008 | A:G | rs7988735 | 63169862 | 0.175 | T:C |
| rs9570986 | 63156139 | 0.008 | C:T | rs2121027 | 63170820 | 0.175 | C:T |
| rs9564092 | 63157472 | 0.008 | C:A | rs12862652 | 63171531 | 0.167 | C:T |
| rs9570989 | 63157768 | 0.008 | T:C | rs9539847 | 63171989 | 0.175 | C:G |
| rs9570990 | 63157896 | 0.008 | A:G | rs9539849 | 63172949 | 0.175 | C:T |
| rs2587586 | 63157973 | 0.175 | T:G | rs4310736 | 63174790 | 0.175 | C:T |
| rs9570991 | 63158551 | 0.008 | G:C | rs9539850 | 63176090 | 0.167 | A:G |
| rs9570992 | 63158697 | 0.008 | C:T | rs7998991 | 63177133 | 0.167 | G:C |
| rs6562324 | 63158774 | 0.175 | G:A | rs9539851 | 63177581 | 0.175 | A:T |
| rs1574697 | 63160094 | 0.175 | T:A | rs9539854 | 63181695 | 0.167 | T:A |
| rs9570995 | 63160213 | 0.008 | A:G | rs9539855 | 63182064 | 0.167 | G:A |
| rs7324807 | 63184124 | 0.167 | T:A | rs9564096 | 63186760 | 0.008 | A:T |
| rs11843593 | 63185384 | 0.167 | T:G | rs4589433 | 63188213 | 0.167 | C:G |
| rs11842160 | 63185984 | 0.167 | C:T | | | | |

TABLE 172
Singleton Tag
Block 002
Chromosome 13
Rs17062387 (BP 73697866) LD block SNPs

| | | Minor | |
|---|---|---|---|
| | Base | Allele | |
| SNP ID (rs) | Position | Freq | Alleles |
| rs7325565 | 73672256 | 0.092 | T:G |
| rs17062334 | 73672820 | 0.008 | C:G |
| rs7994198 | 73673276 | 0.008 | C:T |
| rs1337037 | 73674576 | 0.008 | A:G |
| rs17062348 | 73676802 | 0.008 | G:C |
| rs12875186 | 73684236 | 0.092 | A:T |
| rs12875916 | 73684644 | 0.100 | C:T |
| rs8002907 | 73685238 | 0.100 | A:C |
| rs11616399 | 73685863 | 0.100 | C:T |
| rs12868885 | 73688150 | 0.058 | G:A |
| rs7325884 | 73690101 | 0.008 | A:G |
| rs7326654 | 73690331 | 0.008 | C:T |
| rs7326493 | 73690352 | 0.100 | A:G |
| rs1857026 | 73691028 | 0.100 | C:A |
| rs12871788 | 73692474 | 0.100 | G:A |
| rs7985779 | 73692806 | 0.092 | C:T |
| rs12876993 | 73693808 | 0.100 | C:G |
| rs12863691 | 73694096 | 0.100 | T:C |
| rs12864047 | 73694109 | 0.092 | T:C |
| rs12862289 | 73694268 | 0.092 | G:A |
| rs12863374 | 73694783 | 0.092 | A:C |
| rs9592974 | 73695149 | 0.142 | A:G |
| rs17062383 | 73695246 | 0.008 | C:T |
| rs17062385 | 73696991 | 0.008 | C:G |
| rs13378302 | 73698575 | 0.142 | C:A |
| rs12857591 | 73699273 | 0.067 | C:T |
| rs9318250 | 73699707 | 0.067 | A:G |
| rs17062394 | 73701735 | 0.058 | A:G |
| rs12877408 | 73702574 | 0.100 | C:T |
| rs2039342 | 73702798 | 0.100 | C:T |
| rs2077879 | 73704117 | 0.067 | G:T |
| rs7989618 | 73704495 | 0.067 | C:T |
| rs9600246 | 73704922 | 0.108 | A:C |
| rs12874617 | 73705427 | 0.033 | C:A |
| rs12875669 | 73705446 | 0.100 | T:C |
| rs17062403 | 73706875 | 0.058 | C:G |
| rs9600247 | 73706947 | 0.058 | T:C |
| rs17062404 | 73707380 | 0.008 | G:A |
| rs2777656 | 73707901 | 0.067 | C:T |
| rs6562809 | 73714160 | 0.100 | G:A |
| rs6562810 | 73714424 | 0.067 | T:G |
| rs9600250 | 73715917 | 0.383 | T:G |
| rs1417991 | 73716421 | 0.100 | C:A |
| rs12856766 | 73718253 | 0.033 | C:A |
| rs7323293 | 73722855 | 0.058 | T:A |
| rs12873991 | 73724038 | 0.033 | G:C |
| rs12874912 | 73724153 | 0.033 | T:C |
| rs12856631 | 73725504 | 0.033 | A:C |
| rs12867440 | 73726308 | 0.033 | T:C |
| rs9530292 | 73729967 | 0.200 | A:G |
| rs7330880 | 73731144 | 0.067 | A:G |
| rs12865094 | 73731529 | 0.100 | C:T |
| rs7338571 | 73731874 | 0.200 | T:G |
| rs12865985 | 73731970 | 0.033 | G:A |
| rs12861293 | 73734399 | 0.033 | T:C |
| rs12862167 | 73734559 | 0.092 | T:C |
| rs7323169 | 73734738 | 0.008 | T:A |
| rs7323758 | 73735022 | 0.100 | T:A |

TABLE 173
Singleton Tag
Block 003
Chromosome 13
Rs16953855 (BP 96562206) LD block SNPs

| | | Minor | |
|---|---|---|---|
| | Base | Allele | |
| SNP ID (rs) | Position | Freq | Alleles |
| rs4771979 | 96533786 | 0.425 | G:T |
| rs8181806 | 96534515 | 0.100 | C:T |
| rs9556676 | 96534734 | 0.125 | C:T |
| rs7333925 | 96538339 | 0.125 | C:A |
| rs9556677 | 96539966 | 0.100 | A:C |
| rs9584494 | 96544189 | 0.008 | T:C |
| rs1501857 | 96545165 | 0.450 | G:A |
| rs1501856 | 96545657 | 0.425 | C:T |
| rs12857201 | 96552859 | 0.442 | G:T |
| rs2055424 | 96556772 | 0.442 | C:T |
| rs2055423 | 96556895 | 0.425 | T:C |
| rs11839914 | 96561724 | 0.442 | G:A |
| rs1995372 | 96562448 | 0.408 | C:T |
| rs9300389 | 96562982 | 0.392 | G:A |

TABLE 174
Singleton Tag
Block 001
Chromosome 14
Rs1961605 (BP 58535847) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 175
Singleton Tag
Block 002
Chromosome 14
Rs8010814 (BP 62261229) LD block SNPs

| | | Minor | |
|---|---|---|---|
| | Base | Allele | |
| SNP ID (rs) | Position | Freq | Alleles |
| rs12587728 | 62253679 | 0.492 | T:C |
| rs7158012 | 62254129 | 0.025 | C:G |
| rs7158179 | 62254225 | 0.025 | C:T |
| rs6573458 | 62255232 | 0.025 | C:T |
| rs7400889 | 62255256 | 0.442 | G:C |
| rs17100282 | 62256560 | 0.025 | T:C |
| rs6573459 | 62257760 | 0.017 | C:A |
| rs8022157 | 62257839 | 0.492 | G:A |
| rs8003224 | 62258092 | 0.017 | G:T |
| rs4902182 | 62258179 | 0.017 | G:C |
| rs4899094 | 62258568 | 0.017 | A:G |
| rs12100978 | 62258966 | 0.392 | G:A |
| rs11158449 | 62259523 | 0.492 | T:G |
| rs10483754 | 62260847 | 0.492 | A:G |
| rs1387756 | 62261074 | 0.017 | A:G |
| rs11158450 | 62261171 | 0.025 | A:G |
| rs2130445 | 62261865 | 0.017 | T:C |
| rs2171878 | 62261972 | 0.017 | C:T |
| rs2101552 | 62262701 | 0.017 | C:T |
| rs2101553 | 62263189 | 0.017 | G:T |
| rs17100302 | 62264085 | 0.008 | T:C |
| rs12892389 | 62265813 | 0.442 | G:T |
| rs12590351 | 62266138 | 0.017 | C:A |
| rs10141461 | 62267155 | 0.017 | T:C |
| rs8009892 | 62268637 | 0.017 | C:T |
| rs11627747 | 62270798 | 0.492 | T:C |
| rs7154700 | 62271524 | 0.017 | C:T |
| rs7157560 | 62275545 | 0.017 | C:T |
| rs7157416 | 62275595 | 0.017 | A:G |
| rs7141868 | 62275796 | 0.042 | T:C |
| rs7143550 | 62276704 | 0.467 | T:C |
| rs10147738 | 62277969 | 0.017 | G:A |
| rs10047855 | 62280098 | 0.417 | C:T |
| rs10047858 | 62280182 | 0.417 | C:T |

TABLE 176
Singleton Tag
Block 003
Chromosome 14
SNP_A-2099420 (BP 75059876) LD block SNPs

| | | Minor | |
|---|---|---|---|
| | Base | Allele | |
| SNP ID (rs) | Position | Freq | Alleles |
| rs175716 | 75055136 | 0.308 | A:G |
| rs175717 | 75055357 | 0.308 | T:A |
| rs175718 | 75056332 | 0.383 | A:G |
| rs175719 | 75056387 | 0.183 | T:C |
| rs175720 | 75057139 | 0.142 | G:A |
| rs2215909 | 75057463 | 0.042 | A:G |
| rs175721 | 75057884 | 0.150 | C:G |
| rs175722 | 75059039 | 0.183 | T:G |
| rs17783132 | 75059876 | 0.125 | G:A |

TABLE 177
Singleton Tag
Block 004
Chromosome 14
Rs2767531 (BP 87648625) LD block SNPs

| | | Minor | |
|---|---|---|---|
| | Base | Allele | |
| SNP ID (rs) | Position | Freq | Alleles |
| rs4904426 | 87647281 | 0.108 | G:A |
| rs4899942 | 87647844 | 0.108 | T:C |
| rs12185054 | 87648483 | 0.017 | A:G |
| rs2767532 | 87650462 | 0.467 | G:A |
| rs12897529 | 87652669 | 0.467 | A:G |
| rs7145673 | 87652974 | 0.242 | T:C |
| rs2755077 | 87654364 | 0.183 | G:T |
| rs11159841 | 87656873 | 0.467 | T:C |
| rs2767534 | 87656920 | 0.425 | T:C |
| rs7148140 | 87657494 | 0.042 | C:G |
| rs3850387 | 87664561 | 0.108 | G:A |
| rs2245688 | 87665626 | 0.467 | A:G |
| rs17124095 | 87669880 | 0.242 | G:A |
| rs17807943 | 87670907 | 0.108 | A:G |
| rs2295739 | 87672475 | 0.100 | C:T |
| rs6574991 | 87673255 | 0.467 | C:T |
| rs17124105 | 87673594 | 0.425 | C:T |
| rs1940551 | 87673612 | 0.467 | C:T |
| rs1940550 | 87673873 | 0.233 | A:G |
| rs10483989 | 87674141 | 0.017 | A:G |
| rs17124112 | 87675262 | 0.083 | C:A |
| rs17761052 | 87675363 | 0.108 | A:G |
| rs6574993 | 87676746 | 0.467 | A:G |
| rs1940532 | 87682170 | 0.467 | C:T |
| rs6574994 | 87684294 | 0.467 | G:A |
| rs2000612 | 87685145 | 0.233 | C:T |
| rs6574995 | 87688655 | 0.425 | T:C |
| rs4513034 | 87690859 | 0.233 | A:G |
| rs7141583 | 87691513 | 0.425 | T:C |
| rs8008120 | 87692120 | 0.467 | C:A |
| rs4904431 | 87694023 | 0.108 | A:G |
| rs4904432 | 87694066 | 0.108 | C:T |
| rs11628844 | 87694468 | 0.108 | G:A |
| rs8010969 | 87695996 | 0.192 | G:A |
| rs1570194 | 87696186 | 0.233 | G:A |
| rs10483990 | 87697310 | 0.108 | C:T |

TABLE 178
Singleton Tag
Block 001
Chromosome 15
Rs1877250 (BP 23673136) LD block SNPs

| | | Minor | |
|---|---|---|---|
| | Base | Allele | |
| SNP ID (rs) | Position | Freq | Alleles |
| rs1877250 | 23673136 | 0.475 | G:A |
| rs12900145 | 23676217 | 0.192 | T:C |
| rs882959 | 23677568 | 0.325 | A:G |

TABLE 179
Singleton Tag
Block 002
Chromosome 15
Rs8026608 (BP 38274294) LD block SNPs

| | | Minor | |
|---|---|---|---|
| | Base | Allele | |
| SNP ID (rs) | Position | Freq | Alleles |
| rs2277559 | 38240599 | 0.267 | G:A |
| rs2277560 | 38240789 | 0.483 | A:G |
| rs1565866 | 38243186 | 0.350 | G:T |
| rs3214012 | 38244421 | 0.267 | C:A |
| rs17668261 | 38244723 | 0.092 | A:C |
| rs1801389 | 38250072 | 0.092 | G:A |
| rs11636402 | 38253197 | 0.267 | A:T |
| rs6492932 | 38260892 | 0.267 | A:G |
| rs17668415 | 38264134 | 0.092 | T:A |
| rs1801376 | 38265123 | 0.267 | A:G |
| rs12148261 | 38265869 | 0.092 | G:A |
| rs7180335 | 38272746 | 0.317 | G:A |
| rs7165187 | 38273958 | 0.092 | G:A |
| rs12913994 | 38275753 | 0.350 | A:G |
| rs1047130 | 38276143 | 0.392 | G:A |
| rs12915249 | 38278003 | 0.258 | G:A |
| rs2172742 | 38279244 | 0.350 | G:A |
| rs2305653 | 38281790 | 0.350 | A:G |
| rs12899102 | 38282359 | 0.350 | A:C |
| rs7173181 | 38283683 | 0.092 | T:C |
| rs10518682 | 38285296 | 0.258 | A:G |
| rs11630664 | 38288278 | 0.350 | T:C |
| rs7182070 | 38294535 | 0.092 | T:G |
| rs2290551 | 38296892 | 0.350 | G:A |

TABLE 180
Singleton Tag
Block 003
Chromosome 15
Rs341369 (BP 58894143) LD block SNPs

| | | Minor | |
|---|---|---|---|
| | Base | Allele | |
| SNP ID (rs) | Position | Freq | Alleles |
| rs17270466 | 58884212 | 0.283 | C:T |
| rs10519072 | 58885275 | 0.033 | C:T |
| rs341420 | 58889259 | 0.008 | T:C |
| rs12441037 | 58889949 | 0.017 | T:C |
| rs341366 | 58890882 | 0.458 | A:G |
| rs341367 | 58891103 | 0.283 | A:C |
| rs16943131 | 58892187 | 0.017 | T:C |
| rs16943133 | 58892199 | 0.017 | T:A |
| rs8041822 | 58892849 | 0.025 | T:C |
| rs6494225 | 58892920 | 0.017 | T:G |
| rs8041070 | 58892933 | 0.017 | G:C |
| rs6494226 | 58893178 | 0.017 | C:T |
| rs6494227 | 58893410 | 0.017 | A:C |
| rs7165493 | 58894232 | 0.017 | A:G |
| rs7165688 | 58894276 | 0.017 | C:G |
| rs7165718 | 58894363 | 0.017 | A:C |
| rs17204496 | 58894905 | 0.042 | G:C |
| rs2414681 | 58896610 | 0.475 | C:A |
| rs2263569 | 58896671 | 0.283 | G:A |
| rs10519074 | 58897165 | 0.467 | C:A |
| rs10519076 | 58898181 | 0.125 | G:C |
| rs4775300 | 58898610 | 0.017 | A:G |
| rs7171681 | 58898620 | 0.300 | T:C |
| rs12913922 | 58899317 | 0.300 | T:C |
| rs2445927 | 58899658 | 0.292 | C:T |
| rs12899389 | 58899945 | 0.125 | C:A |
| rs10519077 | 58900014 | 0.325 | A:G |
| rs341373 | 58900506 | 0.333 | C:T |
| rs11630108 | 58900625 | 0.333 | C:G |
| rs6494228 | 58900942 | 0.333 | T:C |
| rs7169281 | 58901396 | 0.475 | T:C |
| rs9302216 | 58901695 | 0.333 | A:G |
| rs10152719 | 58901949 | 0.125 | C:T |
| rs341376 | 58903013 | 0.292 | G:A |
| rs341377 | 58903421 | 0.292 | C:G |
| rs341378 | 58904174 | 0.333 | C:T |
| rs341379 | 58904507 | 0.292 | A:G |
| rs341380 | 58904551 | 0.333 | G:T |
| rs341381 | 58904687 | 0.333 | A:G |
| rs341382 | 58905123 | 0.292 | T:C |
| rs16943172 | 58905157 | 0.125 | C:T |
| rs341383 | 58905272 | 0.333 | G:A |
| rs236220 | 58906418 | 0.333 | T:G |
| rs11632290 | 58907037 | 0.042 | C:A |
| rs17270501 | 58907943 | 0.058 | C:T |
| rs12439995 | 58908285 | 0.158 | C:G |
| rs12440095 | 58908509 | 0.100 | G:A |
| rs17270508 | 58910003 | 0.042 | A:C |
| rs341390 | 58910532 | 0.292 | G:A |

TABLE 181
Singleton Tag
Block 004
Chromosome 15
Rs16942965 (BP 87634875) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs12903987 | 87581976 | 0.358 | A:C | rs9806604 | 87627500 | 0.367 | C:T |
| rs13380353 | 87583192 | 0.017 | T:C | rs7163655 | 87628426 | 0.358 | T:G |
| rs12900602 | 87584401 | 0.033 | T:C | rs7163841 | 87628681 | 0.358 | A:G |
| rs12593363 | 87586120 | 0.350 | A:G | rs12904440 | 87630209 | 0.358 | G:T |
| rs718457 | 87586815 | 0.350 | G:A | rs3940291 | 87630785 | 0.358 | A:G |
| rs7164952 | 87587033 | 0.008 | G:A | rs2283432 | 87637232 | 0.350 | G:C |
| rs16942893 | 87587425 | 0.358 | C:G | rs11857960 | 87638143 | 0.008 | G:T |
| rs7178152 | 87588059 | 0.442 | T:G | rs2283431 | 87639049 | 0.350 | G:A |
| rs16942896 | 87590000 | 0.075 | C:G | rs11073855 | 87642642 | 0.075 | C:A |
| rs7184008 | 87590171 | 0.425 | G:A | rs7170109 | 87642717 | 0.433 | T:C |
| rs2108600 | 87592520 | 0.425 | A:G | rs11857748 | 87644748 | 0.008 | G:A |
| rs7183006 | 87593328 | 0.008 | A:C | rs8036314 | 87644809 | 0.075 | G:C |
| rs7167373 | 87595977 | 0.008 | C:T | rs2238304 | 87644954 | 0.433 | A:T |
| rs6496569 | 87596028 | 0.425 | G:A | rs2238303 | 87645239 | 0.350 | G:T |
| rs11857483 | 87597339 | 0.008 | C:T | rs2159081 | 87645692 | 0.350 | A:C |
| rs7168941 | 87599981 | 0.067 | G:A | rs12592258 | 87645853 | 0.350 | C:T |
| rs9302346 | 87600890 | 0.008 | T:C | rs7495763 | 87647883 | 0.033 | C:T |
| rs9806665 | 87601546 | 0.008 | G:A | rs2351003 | 87648739 | 0.358 | C:T |
| rs12898357 | 87602395 | 0.350 | A:G | rs2238300 | 87652584 | 0.375 | G:A |
| rs12900825 | 87602681 | 0.350 | G:C | rs8032440 | 87652595 | 0.083 | G:C |
| rs11854867 | 87604544 | 0.008 | A:C | rs1013444 | 87653632 | 0.350 | G:C |
| rs11858046 | 87604827 | 0.008 | G:A | rs1013445 | 87653648 | 0.350 | C:T |
| rs17803620 | 87605047 | 0.350 | C:T | rs7162771 | 87654472 | 0.367 | T:C |
| rs16942918 | 87605115 | 0.358 | G:A | rs8028380 | 87654550 | 0.075 | T:C |
| rs6496570 | 87606575 | 0.358 | G:C | rs16943011 | 87658702 | 0.367 | T:G |
| rs8040654 | 87606636 | 0.358 | A:G | rs1138465 | 87659606 | 0.358 | T:C |
| rs2159080 | 87614627 | 0.350 | G:A | rs976072 | 87660381 | 0.350 | A:G |
| rs11856246 | 87616464 | 0.008 | T:G | rs3087374 | 87660998 | 0.083 | C:A |
| rs11073853 | 87618693 | 0.050 | C:G | rs1801377 | 87661134 | 0.008 | G:T |
| rs2882557 | 87621518 | 0.350 | A:G | rs3176238 | 87661431 | 0.358 | C:T |
| rs12910397 | 87621960 | 0.008 | A:C | rs2307438 | 87661790 | 0.367 | A:C |
| rs1599857 | 87622736 | 0.350 | G:A | rs2307441 | 87662830 | 0.017 | T:C |
| rs17804009 | 87624958 | 0.358 | T:C | rs2302084 | 87663345 | 0.375 | A:G |
| rs1990229 | 87625262 | 0.350 | A:C | rs2246900 | 87663370 | 0.367 | T:C |
| rs3176222 | 87664196 | 0.008 | G:A | rs2351002 | 87670333 | 0.367 | G:C |
| rs11853304 | 87664795 | 0.008 | G:T | rs2074885 | 87670670 | 0.167 | G:A |
| rs3176214 | 87664885 | 0.008 | G:A | rs2351000 | 87670737 | 0.117 | A:G |
| rs2307449 | 87664932 | 0.383 | T:G | rs758130 | 87674801 | 0.383 | A:G |
| rs2307431 | 87665024 | 0.008 | G:A | rs1054875 | 87680130 | 0.375 | A:T |
| rs3176208 | 87665767 | 0.117 | T:G | rs3759920 | 87681628 | 0.008 | G:A |
| rs2307428 | 87666902 | 0.017 | T:G | rs7495044 | 87685173 | 0.458 | A:G |
| rs3176203 | 87667213 | 0.075 | A:G | rs758131 | 87685305 | 0.458 | T:A |
| rs3176201 | 87667403 | 0.008 | C:T | rs176641 | 87691186 | 0.417 | A:C |
| rs2072266 | 87667958 | 0.367 | A:G | rs875640 | 87698531 | 0.133 | C:A |
| rs2072267 | 87668158 | 0.450 | A:G | rs12595305 | 87700628 | 0.325 | T:C |
| rs12899409 | 87669260 | 0.367 | A:C | | | | |

TABLE 182
Singleton Tag
Block 001
Chromosome 16
Rs7190922 (BP 12725421) LD block SNPs

| | | Minor | |
|---|---|---|---|
| | Base | Allele | |
| SNP ID (rs) | Position | Freq | Alleles |
| rs12444818 | 12703651 | 0.083 | C:T |
| rs4781324 | 12705084 | 0.375 | T:C |
| rs1713492 | 12707657 | 0.017 | A:G |
| rs12443898 | 12708149 | 0.092 | C:A |
| rs4781325 | 12708775 | 0.025 | G:A |
| rs4781326 | 12708873 | 0.025 | A:G |
| rs1346491 | 12708927 | 0.292 | C:T |
| rs2865611 | 12709644 | 0.408 | G:T |
| rs9972703 | 12709965 | 0.300 | T:C |
| rs9930978 | 12711174 | 0.408 | T:C |
| rs11075143 | 12712854 | 0.408 | A:G |
| rs11643593 | 12715743 | 0.408 | T:G |
| rs12445596 | 12718185 | 0.083 | T:C |
| rs882908 | 12724741 | 0.300 | T:C |
| rs11075146 | 12725164 | 0.408 | T:G |
| rs7185373 | 12725403 | 0.275 | T:C |
| rs7190922 | 12725421 | 0.283 | C:T |
| rs9922287 | 12725532 | 0.108 | G:C |
| rs4780473 | 12726173 | 0.283 | C:G |
| rs1344167 | 12729540 | 0.267 | C:T |
| rs9938529 | 12733946 | 0.267 | C:T |
| rs4483832 | 12734286 | 0.392 | C:T |
| rs11075148 | 12736507 | 0.092 | C:T |
| rs11075149 | 12737048 | 0.267 | A:G |
| rs11075150 | 12737343 | 0.267 | A:G |
| rs12596748 | 12739017 | 0.392 | T:C |
| rs7202365 | 12740395 | 0.383 | G:C |
| rs2865616 | 12741084 | 0.275 | T:C |
| rs12596977 | 12742352 | 0.392 | G:A |
| rs11075151 | 12742381 | 0.092 | T:C |
| rs12447643 | 12742692 | 0.092 | G:A |
| rs8058253 | 12743764 | 0.392 | C:A |
| rs8057200 | 12744248 | 0.392 | A:G |
| rs8182206 | 12745788 | 0.425 | C:A |
| rs8182207 | 12745847 | 0.425 | G:A |
| rs8059402 | 12745931 | 0.425 | T:C |
| rs7190046 | 12746593 | 0.150 | T:C |

TABLE 183
Singleton Tag
Block 002
Chromosome 16
Rs1925499 (BP 19967712) LD block SNPs

| | | Minor | |
|---|---|---|---|
| | Base | Allele | |
| SNP ID (rs) | Position | Freq | Alleles |
| rs9923019 | 19964770 | 0.083 | C:T |
| rs1902813 | 19965162 | 0.225 | G:C |
| rs16969473 | 19965298 | 0.083 | A:G |
| rs1886715 | 19966317 | 0.225 | A:C |
| rs12926725 | 19967178 | 0.225 | C:T |
| rs1925501 | 19967344 | 0.225 | T:A |
| rs1925500 | 19967564 | 0.492 | T:G |
| rs1925499 | 19967712 | 0.008 | C:T |
| rs12445332 | 19968068 | 0.075 | T:C |
| rs2764772 | 19968154 | 0.300 | T:A |
| rs12448629 | 19968262 | 0.083 | C:T |
| rs12931939 | 19968331 | 0.208 | C:T |
| rs12448759 | 19968613 | 0.083 | G:A |
| rs16969480 | 19968822 | 0.083 | A:G |

TABLE 184
Singleton Tag
Block 003
Chromosome 16
Rs16970520 (BP 69353341) LD block SNPs

| | | Minor | |
|---|---|---|---|
| | Base | Allele | |
| SNP ID (rs) | Position | Freq | Alleles |
| rs6499336 | 69341002 | 0.133 | T:C |
| rs17248914 | 69342698 | 0.133 | A:G |
| rs16970484 | 69343470 | 0.133 | C:G |
| rs936305 | 69344133 | 0.358 | G:A |
| rs7194091 | 69344281 | 0.133 | T:C |
| rs7185508 | 69348115 | 0.008 | T:C |
| rs7185921 | 69348332 | 0.133 | T:C |
| rs16970513 | 69349418 | 0.133 | A:G |
| rs3785417 | 69350518 | 0.350 | A:G |
| rs12930674 | 69351532 | 0.342 | C:T |
| rs7188712 | 69352903 | 0.125 | C:T |
| rs2305689 | 69354016 | 0.350 | A:G |
| rs3760118 | 69356910 | 0.133 | T:A |

TABLE 185
Singleton Tag
Block 004
Chromosome 16
Rs6564907 (BP 80263472) LD block SNPs

| | | Minor | |
|---|---|---|---|
| | Base | Allele | |
| SNP ID (rs) | Position | Freq | Alleles |
| rs8063999 | 80261616 | 0.425 | A:G |
| rs4889361 | 80261646 | 0.425 | T:A |
| rs11644382 | 80264753 | 0.433 | G:A |
| rs4889362 | 80265290 | 0.417 | G:A |

TABLE 186
Singleton Tag
Block 001
Chromosome 17
Rs9908463 (BP 42507913) LD block SNPs

| | | Minor | |
|---|---|---|---|
| | Base | Allele | |
| SNP ID (rs) | Position | Freq | Alleles |
| rs16961883 | 42454424 | 0.083 | C:T |
| rs9284376 | 42457412 | 0.025 | T:G |
| rs2907245 | 42459045 | 0.092 | T:G |
| rs4968304 | 42495600 | 0.375 | C:A |
| rs12453505 | 42498464 | 0.025 | T:C |
| rs12603447 | 42501236 | 0.283 | A:G |
| rs2007723 | 42501788 | 0.283 | C:T |
| rs7221149 | 42503257 | 0.008 | A:T |
| rs9894300 | 42506518 | 0.100 | G:C |
| rs2316667 | 42506548 | 0.092 | T:C |
| rs9889451 | 42508874 | 0.283 | A:G |
| rs7220879 | 42510913 | 0.175 | A:C |
| rs4968305 | 42513282 | 0.092 | G:T |
| rs12603764 | 42513790 | 0.175 | G:A |
| rs16941569 | 42518077 | 0.100 | G:A |

TABLE 187
Singleton Tag
Block 001
Chromosome 18
Rs347546 (BP 48231042) LD block SNPs

| | | Minor | |
|---|---|---|---|
| | Base | Allele | |
| SNP ID (rs) | Position | Freq | Alleles |
| rs1222914 | 48219732 | 0.125 | G:C |
| rs1145247 | 48220316 | 0.383 | T:C |
| rs1373178 | 48221809 | 0.392 | G:T |
| rs1143713 | 48228687 | 0.142 | C:T |
| rs186984 | 48229059 | 0.125 | G:A |
| rs11082925 | 48230717 | 0.400 | G:T |
| rs347546 | 48231042 | 0.125 | C:T |

TABLE 188
Singleton Tag
Block 002
Chromosome 18
Rs714448 (BP 65799732) LD block SNPs

| | | Minor | |
|---|---|---|---|
| | Base | Allele | |
| SNP ID (rs) | Position | Freq | Alleles |
| rs7238367 | 65746618 | 0.283 | G:A |
| rs12953332 | 65748075 | 0.033 | C:T |
| rs10163940 | 65748313 | 0.008 | T:C |
| rs17081853 | 65748376 | 0.017 | A:G |
| rs4275943 | 65748829 | 0.033 | G:A |
| rs4599011 | 65748907 | 0.008 | C:T |
| rs7234496 | 65749159 | 0.008 | T:C |
| rs3937015 | 65750496 | 0.017 | C:T |
| rs10048373 | 65751363 | 0.017 | T:C |
| rs12962929 | 65751490 | 0.033 | C:G |
| rs11151547 | 65751971 | 0.392 | T:C |
| rs1823778 | 65752616 | 0.058 | C:G |
| rs10048309 | 65752945 | 0.025 | A:G |
| rs11665349 | 65752961 | 0.033 | C:T |
| rs3930581 | 65753240 | 0.008 | G:A |
| rs3934439 | 65754960 | 0.033 | A:G |
| rs17804058 | 65755221 | 0.083 | G:C |
| rs3934438 | 65755792 | 0.033 | G:A |
| rs12970649 | 65756787 | 0.033 | T:C |
| rs9319792 | 65761578 | 0.008 | C:T |
| rs4891794 | 65762523 | 0.033 | A:G |
| rs10782036 | 65764906 | 0.008 | G:A |
| rs8098352 | 65766941 | 0.008 | T:C |
| rs11664236 | 65770928 | 0.008 | C:G |
| rs1579582 | 65771253 | 0.008 | C:A |
| rs12967218 | 65771318 | 0.017 | T:C |
| rs2096987 | 65771673 | 0.017 | G:A |
| rs9965747 | 65771867 | 0.017 | G:A |
| rs12955015 | 65772734 | 0.017 | A:C |
| rs11151548 | 65773325 | 0.142 | G:T |
| rs4891383 | 65775153 | 0.008 | G:A |
| rs4891384 | 65775534 | 0.033 | C:T |
| rs1539964 | 65776265 | 0.383 | T:C |
| rs12955117 | 65779410 | 0.033 | C:T |
| rs11662781 | 65779636 | 0.017 | C:T |
| rs7243133 | 65780326 | 0.017 | C:T |
| rs7237856 | 65786630 | 0.033 | C:T |
| rs7237171 | 65786928 | 0.033 | T:C |
| rs7238633 | 65787044 | 0.017 | C:T |
| rs12955950 | 65788428 | 0.025 | T:C |
| rs6566449 | 65793230 | 0.017 | G:A |
| rs2117557 | 65794993 | 0.033 | T:C |
| rs13371099 | 65796200 | 0.008 | G:A |
| rs4891795 | 65797307 | 0.033 | G:A |
| rs8085823 | 65797919 | 0.033 | T:C |
| rs8085960 | 65798015 | 0.033 | A:C |
| rs7242373 | 65801516 | 0.008 | C:T |
| rs12968104 | 65801761 | 0.033 | C:G |
| rs12608079 | 65802030 | 0.033 | G:C |
| rs12604203 | 65802518 | 0.017 | G:A |
| rs7238142 | 65804232 | 0.017 | C:G |
| rs8099182 | 65806076 | 0.017 | T:C |
| rs12960085 | 65812406 | 0.008 | T:A |
| rs1004623 | 65815130 | 0.033 | T:A |
| rs2123444 | 65815430 | 0.017 | C:T |
| rs11151553 | 65817887 | 0.033 | A:G |
| rs17230566 | 65818011 | 0.258 | C:T |

TABLE 189
Anchor/Singleton Tag
Block 003
Chromosome 18
SNP_A-4205076 (BP 67249199) LD block SNPs

| | | Minor | |
|---|---|---|---|
| | Base | Allele | |
| SNP ID (rs) | Position | Freq | Alleles |
| rs2034783 | 67243613 | 0.408 | C:T |
| rs7238689 | 67244866 | 0.192 | A:G |
| rs11662202 | 67245775 | 0.117 | G:A |
| rs637451 | 67247130 | 0.300 | C:T |
| rs17084405 | 67247378 | 0.167 | G:A |
| rs7237387 | 67248089 | 0.167 | T:C |
| rs7227331 | 67249515 | 0.300 | G:A |
| rs12606509 | 67249771 | 0.117 | T:A |
| rs10514009 | 67250588 | 0.100 | T:C |
| rs7239929 | 67253878 | 0.317 | G:A |
| rs8082727 | 67254344 | 0.317 | T:A |

TABLE 190
Singleton Tag
Block 001
Chromosome 20
rs6096732 (BP 50,115,618) LD block SNPs

| | | Minor | |
|---|---|---|---|
| | Base | Allele | |
| SNP ID (rs) | Position | Freq | Alleles |
| rs6068048 | 50087983 | 0.175 | C:T |
| rs13039838 | 50089100 | 0.175 | T:C |
| rs6013359 | 50090456 | 0.175 | A:G |
| rs6021647 | 50091017 | 0.175 | G:C |
| rs6091430 | 50091644 | 0.033 | T:C |
| rs6021649 | 50092560 | 0.125 | A:G |
| rs17803688 | 50094168 | 0.042 | G:C |
| rs2426410 | 50094305 | 0.117 | G:T |
| rs11086365 | 50095820 | 0.117 | G:C |
| rs11086366 | 50096368 | 0.117 | A:C |
| rs2426412 | 50096662 | 0.108 | A:G |
| rs2426413 | 50096778 | 0.117 | G:A |
| rs2426414 | 50098113 | 0.108 | G:A |
| rs11699699 | 50099500 | 0.175 | G:A |
| rs1546985 | 50100713 | 0.167 | G:T |
| rs6021651 | 50101186 | 0.167 | T:C |
| rs6021652 | 50101461 | 0.167 | G:A |
| rs2426415 | 50101674 | 0.167 | A:G |
| rs6063743 | 50101829 | 0.167 | G:A |
| rs1546986 | 50102835 | 0.167 | A:G |
| rs6021656 | 50103163 | 0.167 | A:G |
| rs2426416 | 50103490 | 0.167 | T:C |
| rs6096730 | 50103536 | 0.167 | T:C |
| rs2426417 | 50103591 | 0.167 | A:G |
| rs6068058 | 50103800 | 0.108 | G:A |
| rs2208005 | 50104414 | 0.167 | T:G |
| rs2426418 | 50104571 | 0.167 | C:T |
| rs2426419 | 50104594 | 0.167 | T:A |
| rs6021658 | 50105671 | 0.167 | G:A |
| rs2426421 | 50106602 | 0.167 | C:T |
| rs2869622 | 50106623 | 0.167 | A:G |
| rs2426423 | 50107772 | 0.167 | A:G |
| rs6021661 | 50107933 | 0.417 | G:A |
| rs2904146 | 50108307 | 0.167 | C:A |
| rs2064728 | 50108559 | 0.167 | T:C |
| rs6021662 | 50109554 | 0.442 | C:A |
| rs2869623 | 50109776 | 0.192 | A:G |
| rs761852 | 50110092 | 0.158 | A:C |
| rs2869624 | 50110135 | 0.167 | T:C |
| rs6021667 | 50110775 | 0.167 | C:T |
| rs6123124 | 50110784 | 0.167 | A:G |
| rs6021668 | 50110836 | 0.167 | C:T |
| rs6126453 | 50111031 | 0.108 | T:A |
| rs11700140 | 50111452 | 0.108 | G:A |
| rs2144773 | 50112612 | 0.167 | T:G |
| rs6013373 | 50114140 | 0.167 | G:A |
| rs6021671 | 50115664 | 0.375 | T:G |
| rs11086368 | 50118060 | 0.108 | C:T |
| rs11086369 | 50118293 | 0.108 | A:C |
| rs6068063 | 50118387 | 0.042 | A:G |
| rs7274339 | 50118483 | 0.042 | G:A |
| rs7261259 | 50118844 | 0.042 | A:G |
| rs6021674 | 50118965 | 0.167 | C:T |
| rs8114444 | 50118977 | 0.042 | A:G |
| rs6068064 | 50119037 | 0.167 | C:T |

TABLE 191
Singleton Tag
Block 002
Chromosome 20
SNP_A-4235645 (BP 52465343) LD block SNPs

| | | Minor | |
|---|---|---|---|
| | Base | Allele | |
| SNP ID (rs) | Position | Freq | Alleles |
| rs6013997 | 52438045 | 0.108 | A:G |
| rs1407043 | 52438394 | 0.108 | A:C |
| rs6023185 | 52440136 | 0.108 | A:G |
| rs2426507 | 52452141 | 0.033 | T:C |
| rs6097963 | 52452442 | 0.017 | A:G |
| rs16999597 | 52452940 | 0.108 | A:G |
| rs17317237 | 52457406 | 0.092 | C:T |
| rs1321468 | 52462428 | 0.100 | C:T |
| rs1321465 | 52465568 | 0.100 | T:C |
| rs1321464 | 52465664 | 0.100 | T:C |
| rs6014011 | 52467322 | 0.100 | A:G |
| rs6023227 | 52470314 | 0.100 | G:A |

TABLE 192
Singleton Tag
Block 001
Chromosome 21
rs4578944 (BP 18684634) LD block SNPs

| | | | | | | | |
|---|---|---|---|---|---|---|---|
| | | Minor | | | | Minor | |
| | Base | Allele | | | Base | Allele | |
| SNP ID (rs) | Position | Freq | Alleles | SNP ID (rs) | Position | Freq | Alleles |

No LD Block

TABLE 193
Singleton Tag
Block 001
Chromosome 22
Rs16991923 (BP 31889795) LD block SNPs

| | | Minor | | | | Minor | |
|---|---|---|---|---|---|---|---|
| | Base | Allele | | | Base | Allele | |
| SNP ID (rs) | Position | Freq | Alleles | SNP ID (rs) | Position | Freq | Alleles |
| rs743793 | 31886300 | 0.458 | T:C | rs137358 | 31898878 | 0.442 | C:G |
| rs5994683 | 31886548 | 0.058 | G:C | rs7290315 | 31898921 | 0.158 | G:C |
| rs743794 | 31887237 | 0.125 | T:C | rs7284510 | 31899015 | 0.117 | A:G |
| rs9609729 | 31887441 | 0.458 | T:G | rs11913261 | 31899503 | 0.125 | A:G |
| rs1476005 | 31888171 | 0.125 | C:T | rs137361 | 31899670 | 0.442 | C:T |
| rs916244 | 31888432 | 0.375 | A:G | rs137362 | 31900225 | 0.400 | G:A |
| rs1015740 | 31888997 | 0.392 | G:A | rs13055004 | 31900374 | 0.367 | A:G |
| rs1476006 | 31889313 | 0.125 | C:T | rs137363 | 31900839 | 0.050 | A:G |
| rs16991923 | 31889795 | 0.008 | A:T | rs137366 | 31901623 | 0.400 | C:G |
| rs10483170 | 31890439 | 0.058 | C:G | rs2413169 | 31901813 | 0.125 | C:A |
| rs4821132 | 31891141 | 0.433 | C:T | rs137379 | 31902704 | 0.475 | T:C |
| rs9609731 | 31891915 | 0.058 | T:C | rs7410494 | 31903693 | 0.400 | A:G |
| rs2213457 | 31893114 | 0.058 | G:T | rs9609738 | 31905474 | 0.475 | C:T |
| rs16991928 | 31893129 | 0.117 | G:A | rs2097372 | 31907787 | 0.125 | C:T |
| rs12483930 | 31894008 | 0.042 | C:G | rs137404 | 31908822 | 0.400 | C:T |
| rs11089609 | 31894482 | 0.375 | A:G | rs137405 | 31909227 | 0.017 | A:G |
| rs137357 | 31898083 | 0.400 | T:C | rs137409 | 31909515 | 0.392 | A:G |
| rs5994688 | 31898628 | 0.433 | C:G | rs137410 | 31909724 | 0.400 | T:G |
| rs7290016 | 31898731 | 0.117 | G:A | rs137358 | 31898878 | 0.442 | C:G |

TABLE 194
Singleton Tag
Block 002
Chromosome 22
SNP_A-1904522 (BP 38602840) LD block SNPs

| | | Minor | |
|---|---|---|---|
| | Base | Allele | |
| SNP ID (rs) | Position | Freq | Alleles |
| rs8135811 | 38593317 | 0.058 | A:G |
| rs137947 | 38602893 | 0.233 | C:T |
| rs137948 | 38604030 | 0.242 | T:C |
| rs9611224 | 38606195 | 0.233 | C:T |

TABLE 195
Singleton Tag
Block 001
Chromosome 23
Rs6608322 (BP 148809819) LD block SNPs

| | | Minor | |
|---|---|---|---|
| | Base | Allele | |
| SNP ID (rs) | Position | Freq | Alleles |
| rs454971 | 148590921 | 0.289 | T:C |
| rs454992 | 148591086 | 0.289 | T:C |
| rs16992983 | 148592208 | 0.011 | C:G |
| rs411991 | 148592317 | 0.289 | A:G |
| rs381321 | 148593741 | 0.289 | A:G |
| rs446300 | 148595344 | 0.033 | C:T |
| rs5983897 | 148596616 | 0.044 | C:T |
| rs5983880 | 148597513 | 0.044 | G:C |
| rs5983899 | 148601478 | 0.044 | G:A |
| rs5983900 | 148602865 | 0.044 | T:C |
| rs5940683 | 148617772 | 0.067 | G:C |
| rs11117540 | 148620929 | 0.011 | G:A |
| rs7391980 | 148631865 | 0.044 | T:G |
| rs7878544 | 148633518 | 0.044 | T:G |
| rs4893116 | 148635991 | 0.022 | C:T |
| rs11798728 | 148636926 | 0.011 | A:T |
| rs5940671 | 148663753 | 0.011 | A:C |
| rs5945557 | 148806182 | 0.067 | T:C |
| rs7472068 | 148812100 | 0.156 | G:A |
| rs6608324 | 148817220 | 0.011 | G:A |
| rs12843532 | 148824960 | 0.100 | G:A |
| rs10856256 | 148831578 | 0.156 | G:C |
| rs6649494 | 148837759 | 0.011 | T:G |
| rs12854792 | 148849636 | 0.100 | T:C |
| rs12401236 | 148878901 | 0.067 | A:T |
| rs7391189 | 148890015 | 0.122 | A:C |
| rs6608308 | 148911534 | 0.467 | G:T |
| rs6649483 | 148947202 | 0.478 | T:C |
| rs12398223 | 148949407 | 0.067 | A:G |
| rs12400506 | 148983096 | 0.311 | T:C |

TABLE 196
Singleton Tag
Block 002
Chromosome 23
Rs4535928 (BP 152349432) LD block SNPs

| | | Minor | |
|---|---|---|---|
| | Base | Allele | |
| SNP ID (rs) | Position | Freq | Alleles |
| rs2239683 | 152346847 | 0.400 | T:A |
| rs17091314 | 152347439 | 0.067 | C:T |
| rs2980021 | 152348292 | 0.067 | G:A |
| rs4898359 | 152349008 | 0.467 | C:T |
| rs4243536 | 152351169 | 0.067 | G:A |
| rs5987097 | 152351531 | 0.067 | T:C |
| rs5986911 | 152351830 | 0.067 | A:T |
| rs5945145 | 152351895 | 0.389 | T:C |
| rs4898420 | 152352612 | 0.067 | G:A |
| rs5945146 | 152353999 | 0.067 | G:A |
| rs4898421 | 152355007 | 0.433 | G:A |
| rs5945307 | 152355314 | 0.433 | C:T |
| rs6643626 | 152355583 | 0.433 | T:C |

What is claimed is:

1. A method for determining whether a human subject has altered risk of developing endometriosis, comprising:
detecting in the genetic material of said subject the presence or absence of one or more protective or high-risk polymorphism selected from the group consisting of the polymorphisms of Table 1 or Table 2, or a polymorphism that is in linkage disequilibrium with a polymorphism of Table 1 or Table 2,
wherein the polymorphism is correlated with altered risk of endometriosis.

2. The method according to claim 1, wherein the polymorphism is correlated with presymptomatic risk of developing endometriosis in a human subject.

3. The method according to claim 1, further comprising the step of assessing altered risk of developing endometriosis by determining whether each of a set of independent variables has a unique predictive relationship to a dichotomous dependent variable.

4. The method according to claim 3, wherein the step of assessing altered risk of developing endometriosis utilizes an algorithm comprising a logistic regression analysis.

5. The method according to claim 1, wherein the polymorphism that is in linkage disequilibrium with a polymorphism of Table 1 or Table 2 is selected from the polymorphisms of Tables 3-196.

6. A method for determining whether a human subject has altered risk of developing endometriosis, comprising:
detecting in the genetic material of said subject the presence or absence of one or more protective or high-risk polymorphism selected from the group consisting of the polymorphisms of Table 1 or Table 2, or a polymorphism that is in linkage disequilibrium with a polymorphism of Table 1 or Table 2, wherein the polymorphism is correlated with altered risk of developing endometriosis, and
evaluating the risk associated with one or more non-genetic clinical factors selected from the group consisting of age at menarche, BMI and other factors associated with endometriosis.

7. The method according to claim 6, wherein the polymorphism is correlated with presymptomatic risk of developing endometriosis in a human subject.

8. The method according to claim 6, further comprising the step of assessing altered risk of developing endometriosis by determining whether each of a set of independent variables has a unique predictive relationship to a dichotomous dependent variable.

9. The method according to claim 8, wherein the step of assessing altered risk of developing endometriosis utilizes an algorithm comprising a logistic regression analysis.

10. The method according to claim 6, wherein the polymorphism that is in linkage disequilibrium with a polymorphism of Table 1 or Table 2 is selected from the polymorphisms of Tables 3-196.

11. An amplified polynucleotide containing the nucleotide sequence of a polymorphism selected from the polymorphisms of Table 1 or Table 2, or a polymorphism that is in linkage disequilibrium with a polymorphism of Table 1 or Table 2, or a complement thereof, wherein the amplified polynucleotide is greater than about 16 nucleotides in length.

12. The amplified polynucleotide of claim 11, wherein the polymorphism that is in linkage disequilibrium with a polymorphism of Table 1 or Table 2 is selected from the polymorphisms of Tables 3-196.

13. The amplified polynucleotide of claim 11, wherein the polymorphism is selected from the polymorphisms of Table 1 or Table 2.

14. An isolated polynucleotide that specifically hybridizes to a polynucleotide molecule containing the nucleotide sequence of a polymorphism selected from any one of the polymorphisms of Table 1 or Table 2, or a polymorphism that is in linkage disequilibrium with a polymorphism of Table 1 or Table 2, or a complement thereof.

15. The polynucleotide of claim 14, wherein the polynucleotide is from about 8-70 nucleotides in length.

16. The polynucleotide of claim 14, wherein the polynucleotide is an allele-specific probe.

17. The polynucleotide of claim 14, wherein the polynucleotide is an allele-specific primer.

18. The isolated polynucleotide of claim 14, wherein the polymorphism that is in linkage disequilibrium with a polymorphism of Table 1 or Table 2 is selected from the polymorphisms of Tables 3-196.

19. The polynucleotide of claim 18, wherein the polynucleotide is from about 8-70 nucleotides in length.

20. The polynucleotide of claim 18, wherein the polynucleotide is an allele-specific probe.

21. The polynucleotide of claim 18, wherein the polynucleotide is an allele-specific primer.

22. The isolated polynucleotide of claim 14, wherein the polymorphism is selected from the polymorphisms of Table 1 or Table 2.

23. The polynucleotide of claim 22, wherein the polynucleotide is from about 8-70 nucleotides in length.

24. The polynucleotide of claim 22, wherein the polynucleotide is an allele-specific probe.

25. The polynucleotide of claim 22, wherein the polynucleotide is an allele-specific primer.

26. A kit for detecting a nucleic acid polymorphism indicative of an altered risk in a symptomatic or presymptomatic endometriosis subject, comprising a polynucleotide according to claim 14, enzymes, buffers, and reagents used to detect genetic polymorphisms.

27. The kit according to claim 26, further comprising a questionnaire of non-genetic clinical factors.

28. A kit for detecting a nucleic acid polymorphism indicative of an altered risk in a symptomatic or presymptomatic endometriosis subject, comprising a polynucleotide according to claim 18, enzymes, buffers, and reagents used to detect genetic polymorphisms.

29. The kit according to claim 28, further comprising a questionnaire of non-genetic clinical factors.

30. A kit for detecting a nucleic acid polymorphism indicative of an altered risk in a symptomatic or presymptomatic endometriosis subject, comprising a polynucleotide according to claim 14, enzymes, buffers, and reagents used to detect genetic polymorphisms.

31. The kit according to claim 30, further comprising a questionnaire of non-genetic clinical factors.

32. A method of detecting in a nucleic acid molecule a polymorphism that is correlated altered risk of developing endometriosis, comprising:
contacting a test sample with a polynucleotide sequence that specifically hybridizes under stringent hybridization conditions to a polynucleotide sequence having one or more protective or high-risk polymorphism selected from the group consisting of the polymorphisms of Table 1 or Table 2, or a polymorphism that is in linkage disequilibrium with a polymorphism of Table 1 or Table 2, or a complement thereof, wherein the polymorphism is correlated with altered risk of developing endometriosis, and
detecting the formation of a hybridized duplex.

33. The method according to claim 32, wherein the polymorphism is correlated with presymptomatic risk of developing endometriosis in a human subject.

34. The method according to claim 32, further comprising the step of assessing altered risk of developing endometriosis by determining whether each of a set of independent variables has a unique predictive relationship to a dichotomous dependent variable.

35. The method according to claim 32, wherein the step of assessing altered risk of developing endometriosis utilizes an algorithm comprising a logistic regression analysis.

36. The method according to claim 32, wherein the polymorphism that is in linkage disequilibrium with a polymorphism of Table 1 or Table 2 is selected from the polymorphisms of Tables 3-196.

37. An apparatus for detecting endometriosis mutations comprising:
a DNA chip array comprising a plurality of polynucleotides attached to the array, wherein each polynucleotide contains a polymorphism selected from the group consisting of the polymorphisms set forth in Table 1 or Table 2, or a polymorphism that is in linkage disequilibrium with a polymorphism of Table 1 or Table 2, or a complement thereof,
and a device for detecting the SNPs.

38. The apparatus of claim 37, wherein the polymorphism that is in linkage disequilibrium with a polymorphism of Table 1 or Table 2 is selected from the polymorphisms of Tables 3-196.

39. The apparatus of claim 37, wherein the polymorphism is selected from the polymorphisms of Table 1 or Table 2.

40. A method for identifying a polymorphism that is correlated with altered risk of developing endometriosis, wherein the method comprises the step of identifying a polymorphism that is in linkage disequilibrium with a polymorphism set forth in Tables 1-196.

41. The method of claim 40, wherein the method comprises the step of identifying in the genetic material of a subject a polymorphism that is in linkage disequilibrium with a polymorphism set forth in Table 1 or Table 2.

42. The method of claim 40, wherein the method comprises the step of identifying in the genetic material of a subject a polymorphism that is in linkage disequilibrium with a polymorphism set forth in Tables 3-196.

43. A method of screening human subjects, said method comprising the following steps:
obtaining a genetic material sample of a human subject,
inspecting said genetic material,
detecting in said genetic material the presence or absence of at least one human gene associated genetic marker that is correlated with at least one endometriosis related condition of the following group of endometriosis related conditions comprising an endometriosis existence condition, an endometriosis nonexistence condition, a risk of developing endometriosis condition, and a risk of endometriosis progression condition, wherein said human gene of said human gene association of said genetic marker is selected from any human gene except for a human gene of the following group of human genes comprising ACE, ADAR, AEBP1, AhRR, AHSG, APOE, AR, BRAF, C3, CCL5, CDH1, CLDN4, COL1A2, COL3A1, COMT, CSRP1, CSTB, CTSD, CYP1A1, CYP1B1, CYP17A1, CYP19A1, CYP27B1, DUSP1, EF1alpha, EGFR, EGR1, ERalpha, ERbeta, ESR1, ESR2, FAS, FASLG, FLT-1, FSHR, FTL, GALT, GSN, GSTM1, GSTP1, GSTT1, GSTT1, HLA-A, HLA-B, HLA-C, HLA-DPB1, HLA-DQA1, HLA-DQB1, HLA-DRB1, HRF, HSD17B1, HSD17B1, IB-1, ICAM-1, IFNG, IGFBP3, IGL, IL-1R, IL-1RA, IL-2R, IL-6, IL-10, KRAS, LTA, MCAM, MMP1, MMP3, MMP7, NACA, NAT2, NOS3, NR3C1, NRIP1, P4HB, PAEP, PAI1, PCOLCE, PGR, PLAT (tPA), PLAU (uPA), PODXL, PPARgamma2, PSAP, PTEN, RACK1, RAD21, RANTES, RPS6, SERPINA1, SERPINE1, SFRP4, StAR, STAT6, STMY3, TAFI, TCN2, TGFbeta1, TNFalpha, TNFR2, TNFRSF1B, TSG, VEGF, VEGF, VEGFR, and
responding to said detection of said genetic marker.

44. The method of claim 43, wherein said responding step defines at least one responding step of the following group of responding steps comprising designating an assessed risk of predisposition of said human subject to an endometriosis related condition, assessing an altered risk of an endometriosis related condition by determining whether each of a set of independent variables has a unique predictive relationship to a dichotomous dependent variable, selecting an appropriate therapeutic that at least partially compensates for an endometriosis related condition, selecting said human subject as a recipient of a therapeutic that at least partially compensates for an endometriosis related condition, treating said human subject by administering to said human subject an appropriate therapeutic that at least partially compensates for an endometriosis related condition, developing an appropriate therapeutic that at least partially compensates for an endometriosis related condition, selecting said human subject for clinical trials involving the use of an appropriate therapeutic for treatment of an endometriosis related condition, and designating said human as having an increased risk of predisposition to an endometriosis related condition when said human subject exhibits at least one non-genetic clinical factor.

45. The method of claim 44, wherein said appropriate therapeutic further defines at least one therapeutic of the following group of therapeutics comprising at least one medical device, at least one pharmaceutical, and at least one medical device and at least one pharmaceutical.

46. The method according to claim 44, wherein assessing an altered risk of an endometriosis related condition comprises an algorithm comprising a logistic regression analysis.

47. The method according to claim 44, wherein said at least one non-genetic clinical factor comprises at least one non-genetic clinical factor of the following group of non-genetic clinical factor consisting of age at menarche, BMI and other endometriosis associated factors.

48. The method according to claim 43, wherein said correlation defines a Chi square contingency p value of no more than 0.01 between an altered risk of an endometriosis related condition population and a control population.

49. The method of claim 43, wherein said genetic marker defines at least one polymorphism of the group polymorphisms comprising the polymorphisms of Table 1 and Table 2, the polymorphisms in linkage disequilibrium with the polymorphisms of Table 1 and Table 2, a compliment of the polymorphisms of Table 1 and Table 2, and a compliment of the polymorphisms in linkage disequilibrium with the polymorphisms of Table 1 and Table 2.

50. The method according to claim 43, wherein said human subject is presymptomatic of an endometriosis related condition.
